(19) United States
(12) Patent Application Publication
(10) Pub. No.: US 2003/0219798 A1
(43) Pub. Date: Nov. 27, 2003

(54) Isoprenoid production

(76) Inventors: Ravi R. Gokarn, Minneapolis, MN (US); Holly Jessen, Chanhassen, MN (US); Mary Jo Zidwick, Wayzata, MN (US)

Correspondence Address: FISH & RICHARDSON P.C., 3300 DAIN RAUSCHER PLAZA, 60 SOUTH SIXTH STREET, MINNEAPOLIS, MN, 55402, US

(21) Appl. No.: 10/381,779

(22) PCT Filed: Sep. 28, 2001
(86) PCT No.: PCT/US01/30328

(51) Int. Cl. C12Q 1/68; C07H 21/04; C12P 7/02; C12N 9/10; C12N 1/21; C12P 21/02; C12N 15/74

(52) U.S. Cl. 435/6; 435/69.1; 435/193; 435/320.1; 435/325; 536/23.2; 435/252.3; 435/155

(57) ABSTRACT

The invention provides methods and materials related to the production of isoprenoids. Specifically, the invention provides isolated nucleic acids, substantially pure polypeptides, host cells, and methods and materials for producing various isoprenoid compounds.

### BACKGROUND

1. Technical Field

The invention relates to methods and materials involved in the production of isoprenoids.

2. Background Information

Isoprenoids are compounds that have at least one five-carbon isoprenoid unit. Examples of isoprenoid compounds include, without limitation, carotenoids, isoprenes, sterols, terpenes, and ubiquinones. Various enzymatic pathways in plants, animals, and microorganisms result in the synthesis of isoprenoid compounds. Typically, isopentenyl diphosphate (IPP), dimethylallyl diphosphate (DMAPP), or combinations thereof are polymerized to form isoprenoid compounds.

Two pathways can be used to produce IPP. The first pathway, known as the mevalonate-dependent pathway, produces IPP from 3-hydroxymethyl-3-methylglutaryl Coenzyme A (HMGCoA) in a series of reactions. The second pathway, known as the mevalonate-independent pathway, produces IPP from 1-deoxyxylulose-5-phosphate (DXP) in a series of reactions. One of those reactions involves the use of DXP synthase (DXS) to catalyze the condensation of pyruvate and glyceraldehyde-3-phosphate to form DXP.

Once made, IPP can be used to make various isoprenoid compounds. Specifically, enzymes known as polyprenyl diphosphate synthases catalyze polymerization reactions that combine IPP and DMAPP to form compounds known as polyprenyl diphosphates. For example, decaprenyl diphosphate synthase (DDS) catalyzes the consecutive condensation of IPP with allylic diphosphates to produce decaprenyl diphosphate. Decaprenyl diphosphate is a polyprenyl diphosphate that can be used to form the side chain of a ubiquinone known as CoQ(10). Other polyprenyl diphosphate syntheses include, without limitation, farnesyl-, geranyl-, and octapreneyl diphosphate synthases.

### SUMMARY

The invention relates to methods and materials involved in the production of isoprenoid compounds. Specifically, the invention provides nucleic acid molecules, polypeptides, host cells, and methods that can be used to produce isoprenoid compounds. Isoprenoid compounds are both biologically and commercially important. For example, the nutritional industry uses isoprenoid compounds as nutritional supplements, while the perfume industry uses isoprenoid compounds as fragrances. The nucleic acid molecules described herein can be used to engineer host cells having the ability to produce particular isoprenoid compounds. The polypeptides described herein can be used in cell-free systems to make particular isoprenoid compounds. The host cells described herein can be used in culture systems to produce large quantities of particular isoprenoid compounds.

In general, the invention features an isolated nucleic acid containing a nucleic acid sequence having a length and a percent identity to the sequence set forth in SEQ ID NO: 1 over the length, wherein the point defined by the length and the percent identity is within the area defined by points A, B, C, and D of FIG. 26, wherein point A has coordinates (3626, 100), point B has coordinates (3626, 65), point C has coordinates (50, 65), and point D has coordinates (12, 100). The point B can have coordinates (3626, 85). The point C can have coordinates (100, 65). The point C can have coordinates (50, 85). The point D can have coordinates (15, 100). The nucleic acid sequence can encode a polypeptide. The polypeptide can have DXS activity. The nucleic acid sequence can be as set forth in SEQ ID NO: 1.

In one embodiment, the invention features an isolated nucleic acid containing a nucleic acid sequence having a length and a percent identity to the sequence set forth in SEQ ID NO:2 over the length, wherein the point defined by the length and the percent identity is within the area defined by points A, B, C, and D of FIG. 26, wherein point A has coordinates (1926, 100), point B has coordinates (1926, 65), point C has coordinates (50, 65), and point D has coordinates (12, 100). The nucleic acid sequence can encode a polypeptide. The polypeptide can have DXS activity.

In another embodiment, the invention features an isolated nucleic acid containing a nucleic acid sequence, wherein the nucleic acid sequence encodes a polypeptide containing an amino acid sequence, wherein the amino acid sequence has a length and a percent identity to the sequence set forth in SEQ ID NO:3 over the length, wherein the point defined by the length and the percent identity is within the area defined by points A, B, C, and D of FIG. 26, wherein point A has coordinates (641, 100), point B has coordinates (641, 65), point C has coordinates (25, 65), and point D has coordinates (5, 100). The polypeptide can have DXS activity.

Another embodiment of the invention features an isolated nucleic acid containing a nucleic acid sequence having a length and a percent identity to the sequence set forth in SEQ ID NO:37 over the length, wherein the point defined by the length and the percent identity is within the area defined by points A, B, C, and D of FIG. 26, wherein point A has coordinates (1990, 100), point B has coordinates (1990, 65), point C has coordinates (50, 65), and point D has coordinates (16, 100). The point B can have coordinates (1990, 85). The point C can have coordinates (100, 55). The point C can have coordinates (50, 85). The point D can have coordinates (20, 100). The nucleic acid sequence can encode a polypeptide. The polypeptide can have DDS activity. The nucleic acid sequence can be as set forth in SEQ ID NO:37.

Another embodiment of the invention features an isolated nucleic acid containing a nucleic acid sequence having a length and a percent identity to the sequence set forth in SEQ ID NO:38 over the length, wherein the point defined by the length and the percent identity is within the area defined by points A, B, C, and D of FIG. 26, wherein point A has coordinates (1002, 100), point B has coordinates (1002, 65), point C has coordinates (50, 65), and point D has coordinates (16, 100). The nucleic acid sequence can encode a polypeptide. The polypeptide can have DDS activity.

Another embodiment of the invention features an isolated nucleic acid containing a nucleic acid sequence, wherein the nucleic acid sequence encodes a polypeptide containing an amino acid sequence, wherein the amino acid sequence has a length and a percent identity to the sequence set forth in SEQ ID NO:39 over the length, wherein the point defined by the length and the percent identity is within the area defined by points A, B, C, and D of FIG. 26, wherein point A has coordinates (333, 100), point B has coordinates (333, 65), point C has coordinates (25, 65), and point D has coordinates (5, 100). The polypeptide can have DDS activity.

Another embodiment of the invention features an isolated nucleic acid containing a nucleic acid sequence having a length and a percent identity to the sequence set forth in SEQ ID NO:40 over the length, wherein the point defined by the length and the percent identity is within the area defined by points A, B, C, and D of FIG. 26, wherein point A has coordinates (1833, 100), point B has coordinates (1833, 65), point C has coordinates (50, 65), and point D has coordinates (16, 100). The point B can have coordinates (1833, 85). The point C can have coordinates (100, 65). The point C can have coordinates (50, 85). The point D can have coordinates (20, 100). The nucleic acid sequence can encode a polypeptide. The polypeptide can have DDS activity. The nucleic acid sequence can be as set forth in SEQ ID NO:40.

Another embodiment of the invention features an isolated nucleic acid containing a nucleic acid sequence having a length and a percent identity to the sequence set forth in SEQ ID NO:41 over the length, wherein the point defined by the length and the percent identity is within the area defined by points A, B, C, and D of FIG. 26, wherein point A has coordinates (1014, 100), point B has coordinates (1014, 65), point C has coordinates (50, 65), and point D has coordinates (16, 100). The nucleic acid sequence can encode a polypeptide. The polypeptide can have DDS activity.

Another embodiment of the invention features an isolated nucleic acid containing a nucleic acid sequence, wherein the nucleic acid sequence encodes a polypeptide containing an amino acid sequence, wherein the amino acid sequence has a length and a percent identity to the sequence set forth in SEQ ID NO:42 over the length, wherein the point defined by the length and the percent identity is within the area defined by points A, B, C, and D of FIG. 26, wherein point A has coordinates (337, 100), point B has coordinates (337, 65), point C has coordinates (25, 65), and point D has coordinates (5, 100). The polypeptide can have DDS activity.

Another embodiment of the invention features an isolated nucleic acid containing a nucleic acid sequence having a length and a percent identity to the sequence set forth in SEQ ID NO:95 over the length, wherein the point defined by the length and the percent identity is within the area defined by points A, B, C, and D of FIG. 26, wherein point A has coordinates (2017, 100), point B has coordinates (2017, 65), point C has coordinates (50, 65), and point D has coordinates (16, 100). The point B can have coordinates (2017, 85). The point C can have coordinates (100, 65). The point C can have coordinates (50, 85). The point D can have coordinates (20, 100). The nucleic acid sequence can encode a polypeptide. The polypeptide can have DXR activity. The nucleic acid sequence can be as set forth in SEQ ID NO:95.

Another embodiment of the invention features an isolated nucleic acid containing a nucleic acid sequence having a length and a percent identity to the sequence set forth in SEQ ID NO:96 over the length, wherein the point defined by the length and the percent identity is within the area defined by points A, B, C, and D of FIG. 26, wherein point A has coordinates (1161, 100), point B has coordinates (1161, 65), point C has coordinates (50, 65), and point D has coordinates (16, 100). The nucleic acid sequence can encode a polypeptide. The polypeptide can have DXR activity.

Another embodiment of the invention features an isolated nucleic acid containing a nucleic acid sequence, wherein the nucleic acid sequence encodes a polypeptide containing an amino acid sequence, wherein the amino acid sequence has a length and a percent identity to the sequence set forth in SEQ ID NO:97 over the length, wherein the point defined by the length and the percent identity is within the area defined by points A, B, C, and D of FIG. 26, wherein point A has coordinates (386, 100), point B has coordinates (386, 65), point C has coordinates (25, 65), and point D has coordinates (5, 100). The polypeptide can have DXR activity.

Another embodiment of the invention features an isolated nucleic acid containing a nucleic acid sequence of at least 12 nucleotides, wherein the isolated nucleic acid hybridizes under hybridization conditions to the sense or antisense strand of a nucleic acid molecule, the sequence of the nucleic acid molecule being the sequence set forth in SEQ ID NO: 1, 2, 37, 38, 40, 41, 95, or 96. The nucleic acid sequence can be at least 50 nucleotides (e.g., at least 100, 200, 300, 400, 500, or more). The nucleic acid sequence can encode a polypeptide. The polypeptide can have DXS, DDS, or DXR activity.

In another aspect, the invention features a substantially pure polypeptide containing an amino acid sequence, wherein the amino acid sequence has a length and a percent identity to the sequence set forth in SEQ ID NO:3 over the length, wherein the point defined by the length and the percent identity is within the area defined by points A, B, C, and D of FIG. 26, wherein point A has coordinates (641, 100), point B has coordinates (641, 65), point C has coordinates (25, 65), and point D has coordinates (5, 100). The polypeptide can have DXS activity.

In another embodiment, the invention features a substantially pure polypeptide containing an amino acid sequence, wherein the amino acid sequence has a length and a percent identity to the sequence set forth in SEQ ID NO:39 over the length, wherein the point defined by the length and the percent identity is within the area defined by points A, B, C, and D of FIG. 26, wherein point A has coordinates (333, 100), point B has coordinates (333, 65), point C has coordinates (25, 65), and point D has coordinates (5, 100). The polypeptide can have DDS activity.

Another embodiment of the invention features a substantially pure polypeptide containing an amino acid sequence, wherein the amino acid sequence has a length and a percent identity to the sequence set forth in SEQ ID NO:42 over the length, wherein the point defined by the length and the percent identity is within the area defined by points A, B, C, and D of FIG. 26, wherein point A has coordinates (337, 100), point B has coordinates (337, 65), point C has coordinates (25, 65), and point D has coordinates (5, 100). The polypeptide can have DDS activity.

Another embodiment of the invention features a substantially pure polypeptide containing an amino acid sequence, wherein the amino acid sequence has a length and a percent identity to the sequence set forth in SEQ ID NO:97 over the length, wherein the point defined by the length and the percent identity is within the area defined by points A, B, C, and D of FIG. 26, wherein point A has coordinates (386, 100), point B has coordinates (386, 65), point C has coordinates (25, 65), and point D has coordinates (5, 100). The polypeptide can have DXR activity.

Another aspect of the invention features a host cell containing an isolated nucleic acid of claim 1, 9, 12, 14, 22, 25, 27, 35, 38, 40, 48, 51, or 53. The host cell can be prokaryotic. The host cell can be a Rhodobacter, Sphingomonas, or Escherichia cell. The host cell can contain an exogenous nucleic acid that encodes a polypeptide having DDS, DXS, ODS, SDS, DXR, 4-diphosphocytidyl-2C-methyl-D-erythritol synthase, 4-diphosphocytidyl-2C-methyl-D-erythritol kinase, or chorismate lyase activity. The host cell can contain an exogenous nucleic acid containing an UbiC sequence or LytB sequence. The host cell can contain an exogenous nucleic acid containing an UbiC sequence and LytB sequence. The host cell can contain a non-functional crtE sequence, ppsR sequence, or ccoN sequence. The host cell can contain a non-functional crtE sequence, ppsR sequence, and ccoN sequence.

Another embodiment of the invention features a host cell containing an exogenous nucleic acid and a non-functional crtE sequence, ppsR sequence, or ccoN sequence, wherein the exogenous nucleic acid is within a crtE, ppsR, or ccoN locus of the host cell.

Another embodiment of the invention features a host cell containing a genomic deletion, wherein the deletion comprises at least a portion of a crtE sequence, ppsR sequence, or ccoN sequence, and wherein the host cell comprises a non-functional crtE sequence, ppsR sequence, or ccoN sequence.

Another aspect of the invention features a method for increasing production of CoQ(10) in a cell having endogenous DDS activity. The method includes inserting a nucleic acid molecule containing a nucleic acid sequence that encodes a polypeptide having DDS activity into the cell such that production of CoQ(10) is increased. The nucleic acid molecule can contain an isolated nucleic acid of claim 14, 22, 25, 27, 35, 38, or 53. The production of CoQ(10) can be increased at least about 5 percent as compared to a control cell lacking the inserted nucleic acid molecule. The cell can be a Rhodobacter or Sphingomonas cell. The cell can be a membraneous bacterium or highly membraneous bacterium. The method can also include inserting a second nucleic acid molecule containing a nucleotide sequence that encodes a polypeptide having DXS activity into the cell. The second nucleic acid molecule can contain an isolated nucleic acid of claim 1, 9, or 12.

In another embodiment, the invention features a method for increasing production of CoQ(10) in a cell having endogenous DDS activity. The method includes inserting a nucleic acid molecule containing a nucleic acid sequence that encodes a polypeptide having DXS activity into the cell such that production of CoQ(10) is increased. The production of CoQ(10) can be increased at least about 5 percent as compared to a control cell lacking the inserted nucleic acid molecule. The cell can be a Rhodobacter or Sphingomonas cell. The nucleic acid molecule can contain an isolated nucleic acid of claim 1, 9, or 12. The cell can be a membraneous bacterium or highly membraneous bacterium. The method can also include inserting a second nucleic acid molecule containing a nucleotide sequence that encodes a polypeptide having DDS activity into the cell. The second nucleic acid molecule can contain an isolated nucleic acid of claim 14, 22, 25, 27, 35, 38, or 53.

Another embodiment of the invention features a method for increasing production of CoQ(10) in a membraneous bacterium. The method includes inserting a nucleic acid molecule containing a nucleic acid sequence that encodes a polypeptide having DDS activity into the bacterium such that production of CoQ(10) is increased.

Another embodiment of the invention features a method for increasing production of CoQ(10) in a highly membraneous bacterium. The method includes inserting a nucleic acid molecule containing a nucleic acid sequence that encodes a polypeptide having DDS activity into the highly membraneous bacterium such that production of CoQ(10) is increased.

Another embodiment of the invention features a method for making an isoprenoid. The method includes culturing a cell under conditions wherein the cell produces the isoprenoid, wherein the cell contains at least one exogenous nucleic acid that encodes at least one polypeptide, wherein the cell produces more of the isoprenoid than a comparable cell lacking the at least one exogenous nucleic acid. The cell can be a Rhodobacter or Sphingomonas cell. The isoprenoid can be CoQ(10). The at least one polypeptide can have DDS, DXS, ODS, SDS, DXR, 4-diphosphocytidyl-2C-methyl-D-erythritol synthase, 4-diphosphocytidyl-2C-methyl-D-erythritol kinase, or chorismate lyase activity. The at least one polypeptide can be a UbiC polypeptide or a LytB polypeptide. The cell can contain a non-functional crtE sequence, ppsR sequence, or ccoN sequence. The cell can contain a non-functional crtE sequence, ppsR sequence, and ccoN sequence. The cell can contain a genomic deletion, wherein the deletion contains at least a portion of a crtE sequence, ppsR sequence, or ccoN sequence, and wherein the cell contains a non-functional crtE sequence, ppsR sequence, or ccoN sequence.

Another embodiment of the invention features a method for making an isoprenoid. The method includes culturing a genetically modified cell under conditions wherein the cell produces the isoprenoid. The isoprenoid can be CoQ(10). The cell can contain an exogenous nucleic acid. The cell can contain a genomic deletion.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1 is a diagram of a pathway for producing CoQ(10).

FIG. 2 is a listing of a nucleic acid sequence that encodes a Sphingomonas trueperi (ATCC 12417) polypeptide having DXS activity (SEQ ID NO: 1). The start codon is the ATG at nucleotide number 182, and the stop codon is the TAA at nucleotide number 2107. The probable ribosome binding site is at nucleotide numbers 175-178. This sequence contains an open reading frame as well as 5′ and 3′ untranslated sequences.

FIG. 3 is a listing of a nucleic acid sequence that encodes a Sphingomonas trueperi (ATCC 12417) polypeptide having DXS activity (SEQ ID NO:2). This sequence corresponds to the open reading frame.

FIG. 4 is a listing of an amino acid sequence of a Sphingomonas trueperi (ATCC 12417) polypeptide having DXS activity (SEQ ID NO:3).

FIG. 5 is a sequence pile-up of 14 nucleic acid sequences that encode polypeptides having DXS activity. STdxsdna represents the nucleic acid sequence set forth in SEQ ID NO:2; CRdxsdna represents a nucleic acid sequence from Chlamydomonas reinhardtii (GenBank accession number AJ007559; SEQ ID NO:4); CJdxsdna represents a nucleic acid sequence from Campylobacter jejuni (GenBank accession number AL139074; SEQ ID NO:5); PAdxsdna represents a nucleic acid sequence from Pseudomonas aeruginosa (GenBank accession number AE004821; SEQ ID NO:6); LEdxsdna represents a nucleic acid sequence from Lycopersicon esculentum (GenBank accession number AF143812; SEQ ID NO:7); MTdxsdna represents a nucleic acid sequence from Mycobacterium tuberculosis (GenBank accession number Z96072; SEQ ID NO:8); RSdxs1dna represents a nucleic acid sequence from a Rhodobacter sphaeroides dxs1 gene (SEQ ID NO:9); RSdxs2dna represents a nucleic acid sequence from a Rhodobacter sphaeroides dxs2 gene (SEQ ID NO: 10); SPCCdxsdna represents a nucleic acid sequence from Synechococcus PCC6301 (GenBank accession number Y18874; SEQ ID NO:11); ECdxsdna represents a nucleic acid sequence from Escherichia coli (GenBank accession number AF035440; SEQ ID NO: 12); NMdxsdna represents a nucleic acid sequence from Neisseria meningitidis (GenBank accession number AL162753; SEQ ID NO: 13); HIdxsdna represents a nucleic acid sequence from Haemophilus influenza (GenBank accession number U32822; SEQ ID NO:14); SSdxsdna represents a nucleic acid sequence from Streptomyces sp. CL190 (GenBank(accession number AB026631; SEQ ID NO:16); and HPdxsdna represents a nucleic acid sequence from Helicobacter pylori 26695 (GenBank accession number AE000552; SEQ ID NO: 17).

FIG. 6 is a sequence pile-up of 21 amino acid sequences of polypeptides having DXS activity. STdxsp represents an amino acid sequence set forth in SEQ ID NO:3; AAdxsp represents an amino acid sequence from Aquifex aeolicus (GenBank accession number O67036; SEQ ID NO:18); BSdxsp represents an amino acid sequence from Bacillus subtilis (GenBank accession number P54523; SEQ ID NO: 19); CRdxsp represents an amino acid sequence from Chlamydomonas reinhardtii (GenBank accession number CAA07554; SEQ ID NO:20); CJdxsp represents an amino acid sequence from Campylobacter jejuni (GenBank accession number CAB72788; SEQ ID NO:21); PAdxsp represents an amino acid sequence from Pseudomonas aeruginosa (GenBank accession number AAG07431; SEQ ID NO: 15); LEdxsp represents an amino acid sequence from Lycopersicon esculentum (GenBank accession number AAD38941; SEQ ID NO:22); MLdxsp represents an amino acid sequence from Mycobacterium leprae (GenBank accession number Q50000; SEQ ID NO:23); MTdxsp represents an amino acid sequence from Mycobacterium tuberculosis (GenBank accession number CAB09493; SEQ ID NO:24); RCdxsp represents an amino acid sequence from Rhodobacter capsulatus (GenBank accession number P26242; SEQ ID NO:25); RSdxs1p represents an amino acid sequence encoded by a Rhodobacter sphaeoides dxs1 gene (SEQ ID NO:26); RSdxs2p represents an amino acid sequence encoded by a Rhodobacter sphaeroides dxs2 gene (SEQ ID NO:27); SPCCdxsp represents an amino acid sequence from Synechococcus PCC6301 (GenBank accession number CAB60078; SEQ ID NO:28); SPdxsp represents an amino acid sequence from Synechocystis PCC6803 (GenBank accession number P73067; SEQ ID NO:29); TMdxsp represents an amino acid sequence from Thermotoga maritima (GenBank accession number Q9X291; SEQ ID NO:30); ECdxsp represents an amino acid sequence from Escherichia coli (GenBank accession number D64771; SEQ ID NO:31); NMdxsp represents an amino acid sequence from Neisseria meningitidis (GenBank accession number CAB83880; SEQ ID NO:32); Hldxsp represents an amino acid sequence from Haemophilus influenza (GenBank accession number B64172; SEQ ID NO:33); PFdxsp represents an amino acid sequence from Plasmodium falciparum (GenBank accession number AAD03740; SEQ ID NO:34); SSdxsp represents an amino acid sequence from Streptomyces sp. CL 190 (GenBank accession number BAA85847; SEQ ID NO:35); and HPdxsp represents an amino acid sequence from Helicobacter pylori 26695 (GenBank accession number AAD07422; SEQ ID NO:36).

FIG. 7 is a listing of a nucleic acid sequence that encodes a Rhodobacter sphaeroides (ATCC 17023) polypeptide having DDS activity (SEQ ID NO:37). The start codon is the ATG at nucleotide number 372, and the stop codon is the TGA at nucleotide number 1373. The probable ribosome binding site is at nucleotide numbers 363-366. This sequence contains an open reading frame as well as 5′ and 3′ untranslated sequences.

FIG. 8 is a listing of a nucleic acid sequence that encodes a Rhodobacter sphaeroides (ATCC 17023) polypeptide having DDS activity (SEQ ID NO:38). This sequence corresponds to the open reading frame.

FIG. 9 is a listing of an amino acid sequence of a Rhodobacter sphaeroides (ATCC 17023) polypeptide having DDS activity (SEQ ID NO:39).

FIG. 10 is a listing of a nucleic acid sequence that encodes a Sphingomonas trueperi (ATCC 12417) polypeptide having DDS activity (SEQ ID NO:40). The start codon is the ATG at nucleotide number 605, and the stop codon is the TGA at nucleotide number 1618. The probable ribosome binding site is at nucleotide numbers 590-594. This sequence contains an open reading frame as well as 5′ and 3′ untranslated sequences.

FIG. 11 is a listing of a nucleic acid sequence that encodes a Sphingomonas trueperi (ATCC 12417) polypeptide having DDS activity (SEQ ID NO:41). This sequence corresponds to the open reading frame.

FIG. 12 is a listing of an amino acid sequence of a Sphingomonas trueperi (ATCC 12417) polypeptide having DDS activity (SEQ ID NO:42). This sequence corresponds to the open reading frame.

FIG. 13 is a sequence pile-up of five nucleic acid sequences that encode polypeptides having DDS activity. RSddsdna represents the nucleic acid sequence set forth in SEQ ID NO:38; STddsdna represents the nucleic acid sequence set forth in SEQ ID NO:41; SPddsdna represents a nucleic acid sequence from Schizosaccharomyces pombe (GenBank accession number D84311; SEQ ID NO:43); GSddsdna represents a nucleic acid sequence from Gluconobacter suboxydans (GenBank accession number AB006850; SEQ ID NO:44); and RCddsdna represents a nucleic acid sequence from Rhodobacter capsulatus (U.S. Pat. No. 6,103,488; SEQ ID NO:45).

FIG. 14 is a sequence pile-up of five amino acid sequences of polypeptides having DDS activity. RSddsp represents the amino acid sequence set forth in SEQ ID NO:39; STddsp represents the amino acid sequence set forth in SEQ ID NO:42; GSddsp represents an amino acid sequence from Gluconobacter suboxydans (GenBank accession number BAA32241; SEQ ID NO:46); SPddsp represents an amino acid sequence from Schizosaccharomyces pombe (GenBank accession number CAB66154; SEQ ID NO:47); and RCddsp represents an amino acid sequence from Rhodobacter capsulatus (U.S. Pat. No. 6,103,488; SEQ ID NO:48).

FIG. 15 is a sequence pile-up of three amino acid sequences of polypeptides having DXS activity. Hpdxsp represents the amino acid sequence set forth in SEQ ID NO:36; Ecdxsp represents the amino acid sequence set forth in SEQ ID NO:31; and Hidxsp represents the amino acid sequence set forth in SEQ ID NO:33.

FIG. 16 is a sequence pile-up of four amino acid sequences of polypeptides having DDS, ODS (octaprenyl diphosphate synthase), or SDS (solanesyl diphosphate synthase) activity. Rcsdsp represents an amino acid sequence from Rhodobacter capsulatus having SDS activity (SEQ ID NO:49); Rpodsp represents an amino acid sequence from Rickettsia prowazeki having ODS activity (SEQ ID NO:50); Gsddsp represents the amino acid sequence set forth in SEQ ID NO:46; and Ecodsp represents an amino acid sequence from Escherichia coli ispB having ODS activity (SEQ ID NO:51).

FIG. 17 is a sequence pile-up of five amino acid sequences of polypeptides having DDS, ODS, or SDS activity. Rpodsp represents the amino acid sequence set forth in SEQ ID NO:50; Gsddsp represents the amino acid sequence set forth in SEQ ID NO:46; Ecodsp represents the amino acid sequence set forth in SEQ ID NO:51; Hiodsp represents an amino acid sequence from Haemophilus influenze having ODS activity (SEQ ID NO:52); and Rcsdsp represents the amino acid sequence set forth in SEQ ID NO:49.

FIG. 18 is a diagram of a construct designated appUC18-SHDXS.

FIG. 19 is a diagram of a construct designated appUC18-RSdds.

FIG. 20 is a diagram of a construct designated appUC18-SHDDS.

FIG. 21 is a mass chromatogram obtained from a MG1655 PUC18 specimen.

FIG. 22 is a mass chromatogram obtained from a MG1655 PUC18-DDS specimen.

FIG. 23 is a mass spectra obtained from a MG1655 PUC18 specimen.

FIG. 24 is a mass spectra obtained from a MG1655 PUC18-DDS specimen.

FIG. 25 is a mass spectra obtained from a MG1655 PUC18-DDS specimen.

FIG. 26 is a graph plotting length and percent identity with points A, B, C, and D defining an area indicated by shading.

FIG. 27 is a sequence pile-up of seven amino acid sequences of polypeptides having DXR activity. Bsdxrp represents an amino acid sequence from Bacillus subtilis (SEQ ID NO:98); Hmdxrp represents an amino acid sequence from Haemophilus influenzae (SEQ ID NO:99); Ecdxrp represents an amino acid sequence from Escherishia coli (SEQ ID NO: 100); Zmdxrp represents an amino acid sequence from Zymonas mobilis (SEQ ID NO:101); Sldxrp represents an amino acid sequence from Synechococcus leopoliensis (SEQ ID NO: 102); Ssdxrp represents an amino acid sequence from Synechocystis sp. PCC6803 (SEQ ID NO: 103); and Mtdxrp represents an amino acid sequence from Mycobacterium tuberculosis (SEQ ID NO: 104).

FIG. 28 is a listing of a nucleic acid sequence that encodes a Sphingomonas trueperi polypeptide having DXR activity (SEQ ID NO:95). The start codon is the GTG at either nucleotide number 575 or 578, and the stop codon is the TGA at nucleotide number 1733. This sequence contains an open reading frame as well as 5′ and 3′ untranslated sequences.

FIG. 29 is a listing of a nucleic acid sequence that encodes a Sphingomonas trueperi polypeptide having DXR activity (SEQ ID NO:96). This sequence corresponds to the open reading frame.

FIG. 30 is a listing of an amino acid sequence of a Sphingomonas trueperi polypeptide having DXR activity (SEQ ID NO:97).

FIG. 31 is a sequence pile-up of twelve nucleic acid sequences that encode polypeptides having DXR activity. Stdxrcds represents the nucleic acid sequence set forth in SEQ ID NO:96; Padxrd represents a nucleic acid sequence from Pseudomonas aeruginosa (SEQ ID NO: 105); Zmdxrd represents a nucleic acid sequence from Zygomonas mobilis (SEQ ID NO: 106); Sgdxrd represents a nucleic acid sequence from Streptomyces griseolosporeus (SEQ ID NO: 107); Nmdxrd represents a nucleic acid sequence from Neisseria meningitidis (SEQ ID NO: 108); Ecdxrd represents a nucleic acid sequence from Escherishia coli (SEQ ID NO: 109); Sldxrd represents a nucleic acid sequence from Synechococcus leopoliensis (SEQ ID NO: 110); Mldxrd represents a nucleic acid sequence from Mycobacterium leprae (SEQ ID NO: 111); Pmdxrd represents a nucleic acid sequence from Pasteurella multocida (SEQ ID NO:112); Atdxrd represents a nucleic acid sequence from Arabidopsis thaliana (SEQ ID NO: 113); Cjdxrd represents a nucleic acid sequence from Campylobacter jejuni (SEQ ID NO:114); and Pfdxrd represents a nucleic acid sequence from Plasmodium falciparum (SEQ ID NO: 115).

FIG. 32 is a sequence pile-up of sixteen amino acid sequences of polypeptides having DXR activity. Stdxlp represents the amino acid sequence set forth in SEQ ID NO:97; Zmdxrp represents an amino acid sequence from Zymononas mobilis (SEQ ID NO: 116); Padxrp represents an amino acid sequence from Pseudomonas aeruginosa (SEQ ID NO:117); Ecdxrp represents an amino acid sequence from Escherishia coli (SEQ ID NO:118); Nmdxrp represents an amino acid sequence from Neisseria meningitidis (SEQ ID NO: 119); Hidxrp represents an amino acid sequence from Haemophilus influenzae (SEQ ID NO: 120); Ssdxrp represents an amino acid sequence from Synechocystis sp. PCC6803 (SEQ ID NO:121); Pmdxrp represents an amino acid sequence from Pasteurella multocida (SEQ ID NO:122); Sldxrp represents an amino acid sequence from Synechococcus leopoliensis (SEQ ID NO: 123); Sgdxrp represents an amino acid sequence from Streptomyces griseolosporeus (SEQ ID NO: 124); Bsdxrp represents an amino acid sequence from Bacillus subtilis (SEQ ID NO: 125); Mldxrp represents an amino acid sequence from Mycobacterium leprae (SEQ ID NO: 126); Mtdxrp represents an amino acid sequence from Mycobacterium tuberculosis (SEQ ID NO: 127); Atdxrp represents an amino acid sequence from Arabidopsis thaliana (SEQ ID NO:128); Cjdxrp represents an amino acid sequence from Campylobacter jejuni (SEQ ID NO: 130); and Pfdxrp represents an amino acid sequence from Plasmodium falciparum (SEQ ID NO:131).

### DETAILED DESCRIPTION

The invention provides methods and materials related to the production of isoprenoids. Specifically, the invention provides isolated nucleic acids, substantially pure polypeptides, host cells, and methods and materials for producing various isoprenoid compounds. For the purpose of this invention, an isoprenoid compound is any compound containing a five-carbon isoprenoid unit. Examples of isoprenoid compounds include, without limitation, carotenoids, isoprenes, sterols, terpenes, and ubiquinones. Such isoprenoid compounds can be used in a wide range of applications. For example, isoprenoid compounds produced as described herein can be used in industrial, pharmaceutical, or cosmetic products.

In general terms, carotenoids are lipophilic pigments typically found in photosynthetic plants and bacteria. Examples of carotenoids include, without limitation, carotenes, xanthophylls, hydrocarbon carotenoids, hydroxy carotenoid derivatives, epoxy carotenoid derivatives, furanoxy carotenoid derivatives, and oxy carotenoid derivatives. Isoprenes are oily hydrocarbons that can be obtained by distilling caoutchouc or guttaipercha. Examples of isoprenes include, without limitation, rubber, vitamin A, and vitamin K. Sterols are steroid-based alcohols typically having a hydrocarbon side-chain of eight to ten carbon atoms at the 17-beta position and a hydroxyl group at the 3-beta position. Examples of sterols include, without limitation, ergosterol, cholesterol, and stigmasterol. Terpenes are lipid species typically found in plants in great abundance. Examples of terpenes include, without limitation, dolichol, squalene, and limonene. Ubiquinones are 2,3-dimethoxy-5-methylbenzoquinone derivatives having a side chain containing at least one isoprenoid unit. Typically, ubiquinone is referred to as Coenzyme Q (CoQ). In addition, the number of isoprenoid units of a side chain of a particular ubiquinone is used to identify that particular ubiquinone. For example, a ubiquinone with six isoprenoid units is referred to as CoQ(6), while a ubiquinone with ten isoprenoid units is referred to as CoQ(10). It is noted that CoQ(10) also is referred to as ubidecarenone. Examples of ubiquinones include, without limitation, CoQ(6), CoQ(8), CoQ(10), and CoQ(12).

Isoprenoid compounds can be pyruvate-derived products. The term “pyruvate-derived product” as used herein refers to any compound that is synthesized from pyruvate within no more than 25 enzymatic steps. Thus, an isoprenoid compound is not a pyruvate-derived product if that isoprenoid compound is synthesized from pyruvate in more than 25 enzymatic steps. An enzymatic step is a single chemical reaction catalyzed by a polypeptide having enzymatic activity. The term “polypeptide having enzymatic activity” as used herein refers to any polypeptide that catalyzes a chemical reaction of other substances without itself being destroyed or altered upon completion of the reaction. Typically, a polypeptide having enzymatic activity catalyzes the formation of one or more products from one or more substrates. Such polypeptides can have any type of enzymatic activity including, without limitation, the enzymatic activity associated with an enzyme such as DXS, DDS, ODS, SDS, DXR (1-deoxy-D-xylulose 5-phosphate reductoisomerase), ispD (4-diphosphocytidyl-2C-methyl-D-erythritol synthase), and ispE (4-diphosphocytidyl-2C-methyl-D-erythritol kinase).

A polypeptide having a particular enzymatic activity can be a polypeptide that is either naturally-occurring or non-naturally-occurring. A naturally-occurring polypeptide is any polypeptide having an amino acid sequence as found in nature, including wild-type and polymorphic polypeptides. Such naturally-occurring polypeptides can be obtained from any species including, without limitation, animal (e.g., mammalian), plant, fungal, and bacterial species. A non-naturally-occurring polypeptide is any polypeptide having an amino acid sequence that is not found in nature. Thus, a non-naturally-occurring polypeptide can be a mutated version of a naturally-occurring polypeptide, or an engineered polypeptide. For example, a non-naturally-occurring polypeptide having DDS activity can be a mutated version of a naturally-occurring polypeptide having DDS activity that retains at least some DDS activity. A polypeptide can be mutated by, for example, sequence additions, deletions, substitutions, or combinations thereof.

Examples of isoprenoid compounds that are pyruvate-derived products include, without limitation, CoQ(6), CoQ(7), CoQ(S), CoQ(9), CoQ(10), astaxanthin, canthaxanthin, lutein, zeaxanthin, beta-carotene, lycopene, capsanthin, bixin, norbixin, crocetin, zeta-carotene, vitamin E, giberellins, abscisic acid, ergosterol, geraniol, and latex.

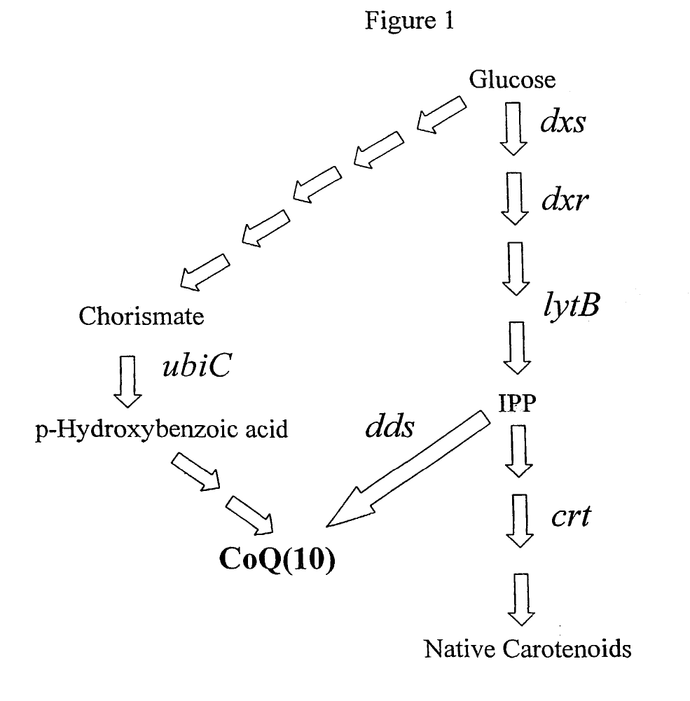

As depicted in FIG. 1, multiple polypeptide can be used to convert glucose CoQ(10). For example, polypeptides having DXS, DXR, LytB, and DDS activity can be used to convert glucose CoQ(10). Such polypeptides can be obtained and used to make CoQ(10) as described herein.

1. Nucleic Acids

The term “nucleic acid” as used herein encompasses both RNA and DNA, including cDNA, genomic DNA, and synthetic (e.g., chemically synthesized) DNA. The nucleic acid can be double-stranded or single-stranded. Where single-stranded, the nucleic acid can be the sense strand or the antisense strand. In addition, nucleic acid can be circular or linear.

The term “isolated” as used herein with reference to nucleic acid refers to a naturally-occurring nucleic acid that is not immediately contiguous with both of the sequences with which it is immediately contiguous (one on the 5′ end and one on the 3′ end) in the naturally-occurring genome of the organism from which it is derived. For example, an isolated nucleic acid can be, without limitation, a recombinant DNA molecule of any length, provided one of the nucleic acid sequences normally found immediately flanking that recombinant DNA molecule in a naturally-occurring genome is removed or absent. Thus, an isolated nucleic acid includes, without limitation, a recombinant DNA that exists as a separate molecule (e.g., a cDNA or a genomic DNA fragment produced by PCR or restriction endonuclease treatment) independent of other sequences as well as recombinant DNA that is incorporated into a vector, an autonomously replicating plasmid, a virus (e.g., a retrovirus, adenovirus, or herpes virus), or into the genomic DNA of a prokaryote or eukaryote. In addition, an isolated nucleic acid can include a recombinant DNA molecule that is part of a hybrid or fusion nucleic acid sequence.

The term “isolated” as used herein with reference to nucleic acid also includes any non-naturally-occurring nucleic acid since non-naturally-occurring nucleic acid sequences are not found in nature and do not have immediately contiguous sequences in a naturally-occurring genome. For example, non-naturally-occurring nucleic acid such as an engineered nucleic acid is considered to be isolated nucleic acid. Engineered nucleic acid can be made using common molecular cloning or chemical nucleic acid synthesis techniques. Isolated non-naturally-occurring nucleic acid can be independent of other sequences, or incorporated into a vector, an autonomously replicating plasmid, a virus (e.g., a retrovirus, adenovirus, or herpes virus), or the genomic DNA of a prokaryote or eukaryote. In addition, a non-naturally-occurring nucleic acid can include a nucleic acid molecule that is part of a hybrid or fusion nucleic acid sequence.

It will be apparent to those of skill in the art that a nucleic acid existing among hundreds to millions of other nucleic acid molecules within, for example, cDNA or genomic libraries, or gel slices containing a genomic DNA restriction digest is not to be considered an isolated nucleic acid.

The term “exogenous” as used herein with reference to nucleic acid and a particular cell refers to any nucleic acid that does not originate from that particular cell as found in nature. Thus, all non-naturally-occurring nucleic acid is considered to be exogenous to a cell once introduced into the cell. It is important to note that non-naturally-occurring nucleic acid can contain nucleic acid sequences or fragments of nucleic acid sequences that are found in nature provided the nucleic acid as a whole does not exist in nature. For example, a nucleic acid molecule containing a genomic DNA sequence within an expression vector is non-naturally-occurring nucleic acid, and thus is exogenous to a cell once introduced into the cell, since that nucleic acid molecule as a whole (genomic DNA plus vector DNA) does not exist in nature. Thus, any vector, autonomously replicating plasmid, or virus (e.g., retrovirus, adenovirus, or herpes virus) that as a whole does not exist in nature is considered to be non-naturally-occurring nucleic acid. It follows that genomic DNA fragments produced by PCR or restriction endonuclease treatment as well as cDNAs are considered to be non-naturally-occulting nucleic acid since they exist as separate molecules not found in nature. It also follows that any nucleic acid containing a promoter sequence and polypeptide-encoding sequence (e.g., cDNA or genomic DNA) in an arrangement not found in nature is non-naturally-occurring nucleic acid.

Nucleic acid that is naturally-occurring can be exogenous to a particular cell. For example, an entire chromosome isolated from a cell of person X is an exogenous nucleic acid with respect to a cell of person Y once that chromosome is introduced into Y's cell.

The invention provides isolated nucleic acid that contains a nucleic acid sequence having (1) a length, and (2) a percent identity to an identified nucleic acid sequence over that length. The invention also provides isolated nucleic acid that contains a nucleic acid sequence encoding a polypeptide that contains an amino acid sequence having (1) a length, and (2) a percent identity to an identified amino acid sequence over that length. Typically, the identified nucleic acid or amino acid sequence is a sequence referenced by a particular sequence identification number, and the nucleic acid or amino acid sequence being compared to the identified sequence is referred to as the target sequence. For example, an identified sequence can be the sequence set forth in SEQ ID NO: 1.

A length and percent identity over that length for any nucleic acid or amino acid sequence is determined as follows. First, a nucleic acid or amino acid sequence is compared to the identified nucleic acid or amino acid sequence using the BLAST 2 Sequences (Bl2seq) program from the stand-alone version of BLASTZ containing BLASTN version 2.0.14 and BLASTP version 2.0.14. This stand-alone version of BLASTZ can be obtained from the University of Wisconsin library as well as at www.fr.com or www.ncbi.nlm.nih.gov. Instructions explaining how to use the B12seq program can be found in the readme file accompanying BLASTZ. B12seq performs a comparison between two sequences using either the BLASTN or BLASTP algorithm. BLASTN is used to compare nucleic acid sequences, while BLASTP is used to compare amino acid sequences. To compare two nucleic acid sequences, the options are set as follows: -i is set to a file containing the first nucleic acid sequence to be compared (e.g., C:\seq1.txt); -j is set to a file containing the second nucleic acid sequence to be compared (e.g., C:\seq2.txt); -p is set to blastn; -o is set to any desired file name (e.g., C:\output.txt); -q is set to −1; -r is set to 2; and all other options are left at their default setting. For example, the following command can be used to generate an output file containing a comparison between two sequences: C:\Bl2seq -i c:\seq1 .txt -j c:\seq2.txt -p blastn -o c:\output.txt -q -1-r 2. To compare two amino acid sequences, the options of Bl2seq are set as follows: -i is set to a file containing the first amino acid sequence to be compared (e.g., C:\seq1.txt); -j is set to a file containing the second amino acid sequence to be compared (e.g., C:\seq2.txt); -p is set to blastp; -o is set to any desired file name (e.g., C:\output.txt); and all other options are left at their default setting. For example, the following command can be used to generate an output file containing a comparison between two amino acid sequences: C:\Bl2seq -i c:\seq1.txt j c:\seq2.txt -p blastp -o c:\output.txt. If the target sequence shares homology with any portion of the identified sequence, then the designated output file will present those regions of homology as aligned sequences. If the target sequence does not share homology with any portion of the identified sequence, then the designated output file will not present aligned sequences. Once aligned, a length is determined by counting the number of consecutive nucleotides or amino acid residues from the target sequence presented in alignment with sequence fi-om the identified sequence starting with any matched position and ending with any other matched position. A matched position is any position where an identical nucleotide or amino acid residue is presented in both the target and identified sequence. Gaps presented in the target sequence are not counted since gaps are not nucleotides or amino acid residues. Likewise, gaps presented in the identified sequence are not counted since target sequence nucleotides or amino acid residues are counted, not nucleotides or amino acid residues from the identified sequence.

The percent identity over a determined length is determined by counting the number of matched positions over that length and dividing that number by the length followed by multiplying the resulting value by 100. For example, if (1) a 1000 nucleotide target sequence is compared to the sequence set forth in SEQ ID NO: 1, (2) the Bl2seq program presents 200 nucleotides from the target sequence aligned with a region of the sequence set forth in SEQ ID NO: 1 where the first and last nucleotides of that 200 nucleotide region are matches, and (3) the number of matches over those 200 aligned nucleotides is 180, then the 1000 nucleotide target sequence contains a length of 200 and a percent identity over that length of 90 (i.e. 180÷200*100=90).

It will be appreciated that a single nucleic acid or amino acid target sequence that aligns with an identified sequence can have many different lengths with each length having its own percent identity. For example, a target sequence containing a 20 nucleotide region that aligns with an identified sequence as follows has many different lengths including those listed in Table 1.

| | |
|---|---|
| | 1 20 |
| Target Sequence: | AGGTCGTGTACTGTCAGTCA |
| | \| \|\| \|\|\| \|\|\|\| \|\|\|\| \| |
| Identified Sequence: | ACGTGGTGAACTGCCAGTGA |

TABLE I

| Starting | Ending | | Matched | Percent |
|---|---|---|---|---|
| Position | Position | Length | Positions | Identity |
| 1 | 20 | 20 | 15 | 75.0 |
| 1 | 18 | 18 | 14 | 77.8 |
| 1 | 15 | 15 | 11 | 73.3 |
| 6 | 20 | 15 | 12 | 80.0 |
| 6 | 17 | 12 | 10 | 83.3 |
| 6 | 15 | 10 | 8 | 80.0 |
| 8 | 20 | 13 | 10 | 76.9 |
| 8 | 16 | 9 | 7 | 77.8 |

It is noted that the percent identity value is rounded to the nearest tenth. For example, 78.11, 78.12, 78.13, and 78.14 is rounded down to 78.1, while 78.15, 78.16, 78.17, 78.18, and 78.19 is rounded up to 78.2. It is also noted that the length value will always be an integer.

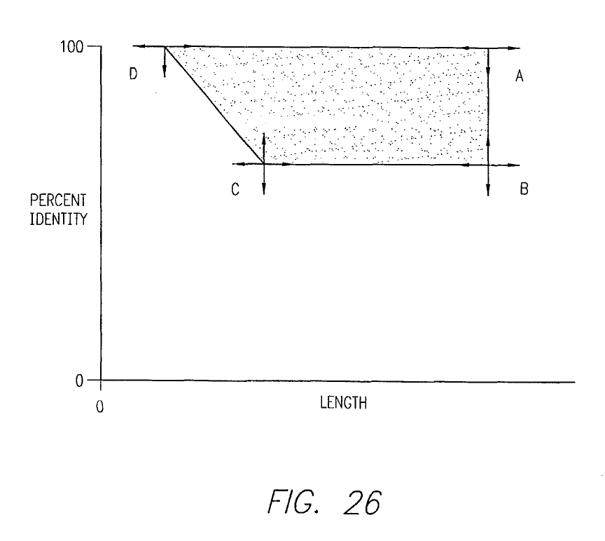

The invention provides an isolated nucleic acid containing a nucleic acid sequence that has at least one length and percent identity over that length as determined above such that the point defined by that length and percent identity is within the area defined by points A, B, C, and D of FIG. 26. In addition, the invention provides an isolated nucleic acid containing a nucleic acid sequence that encodes a polypeptide containing an amino acid sequence that has at least one length and percent identity over that length as determined above such that the point defined by that length and percent identity is within the area defined by points A, B, C, and D of FIG. 26. The point defined by a length and percent identity over that length is that point on the X/Y coordinate of FIG. 26 where the X axis is the length and the Y axis is the percent identity. Thus, the point defined by a nucleic acid sequence with a length of 200 and a percent identity of 90 has coordinates (200, 90). For the purpose of this invention, any point that falls on point A, B, C, or D is considered within the area defined by points A, B, C, and D of FIG. 26. Likewise, any point that falls on a line that defines the area defined by points A, B, C, and D is considered within the area defined by points A, B, C, and D of FIG. 26.

It will be appreciated that the term “the area defined by points A, B, C, and D of FIG. 26” as used herein refers to that area defined by the lines that connect point A with point B, point B with point C, point C with point D, and point D with point A. Points A, B, C, and D can define an area having any shape defined by four points (e.g., square, rectangle, or rhombus). In addition, two or more points can have the same coordinates. For example, points B and C can have identical coordinates. In this case, the area defined by points A, B, C, and D of FIG. 26 is triangular. If three points have identical coordinates, then the area defined by points A, B, C, and D of FIG. 26 is a line. In this case, any point that falls on that line would be considered within the area defined by points A, B, C, and D of FIG. 26. If all four points have identical coordinates, then the area defined by points A, B, C, and D of FIG. 26 is a point. In all cases, simple algebraic equations can be used to determine whether a point is within the area defined by points A, B, C, and D of FIG. 26.

It is noted that FIG. 26 is a graphical representation presenting possible positions of points A, B, C, and D. The shaded area illustrated in FIG. 26 represents one possible example, while the arrows indicate that other positions for points A, B, C, and D are possible. In fact, points A, B, C, and D can have any X coordinate and any Y coordinate. For example, point A can have an X coordinate equal to the number of nucleotides or amino acid residues in an identified sequence, and a Y coordinate of 100. Point B can have an X coordinate equal to the number of nucleotides or amino acid residues in an identified sequence, and a Y coordinate less than or equal to 100 (e.g., 50, 55, 65, 70, 75, 80, 85, 90, 95, and 99). Point C call have an X coordinate equal to a percent (e.g., 1, 2, 5, 10, 15, or more percent) of the number of nucleotides or amino acid residues in an identified sequence, and a Y coordinate less than or equal to 100 (e.g., 50, 55, 65, 70, 75, 80, 85, 90, 95, and 99). Point D can have an X coordinate equal to the length of a typical PCR primer (e.g., 12, 13, 14, 15, 16, 17, or more) or antigenic polypeptide (e.g., 5, 6, 7, 8, 9, 10, 11, 12, or more), and a Y coordinate less than or equal to 100 (e.g., 50, 55, 65, 70, 75, 80, 85, 90, 95, and 99).

An isolated nucleic acid containing a nucleic acid sequence having a length and a percent identity to the sequence set forth in SEQ ID NO: 1 over that length is within the scope of the invention provided the point defined by that length and percent identity is within the area defined by points A, B, C, and D of FIG. 26; where point A has an X coordinate less than or equal to 3626, and a Y coordinate less than or equal to 100; where point B has an X coordinate less than or equal to 3626, and a Y coordinate greater than or equal to 65; where point C has an X coordinate greater than or equal to 50, and a Y coordinate greater than or equal to 65; and where point D has an X coordinate greater than or equal to 12, and a Y coordinate less than or equal to 100. For example, the X coordinate for point A can be 3626, 3600, 3500, 3000, 2500, or less; and the Y coordinate for point A can be 100, 99, 95, 90, 85, 80, 75, or less. The X coordinate for point B can be 3626, 3600, 3500, 3000, 2500, or less; and the Y coordinate for point B can be 65, 70, 75, 80, 85, 90, 95, 99 or more. The X coordinate for point C can be 50, 60, 70, 80, 90, 100, 150, 200, or more; and the Y coordinate for point C can be 65, 70, 75, 80, 85, 90, 95, 99 or more. The X coordinate for point D can be 12, 13, 14, 15, 16, 17, 18, 19, 20, 25, 30, 40, 50, 75, 100, or more; and the Y coordinate for point D can be 100, 99, 95, 90, 85, 80, 75, or less. In one embodiment, point A can be (3626, 100), point B can be (3626, 95), point C can be (1900, 95), and point D can be (1900, 100).

An isolated nucleic acid containing a nucleic acid sequence having a length and a percent identity to the sequence set forth in SEQ ID NO:2 over that length is within the scope of the invention provided the point defined by that length and percent identity is within the area defined by points A, B, C, and D of FIG. 26; where point A has an X coordinate less than or equal to 1926, and a Y coordinate less than or equal to 100; where point B has an X coordinate less than or equal to 1926, and a Y coordinate greater than or equal to 65; where point C has an X coordinate greater than or equal to 50, and a Y coordinate greater than or equal to 65; and where point D has an X coordinate greater than or equal to 12, and a Y coordinate less than or equal to 100. For example, the X coordinate for point A can be 1926, 1900, 1850, 1800, 1750, or less; and the Y coordinate for point A can be 100, 99, 95, 90, 85, 80, 75, or less. The X coordinate for point B can be 1926, 1900, 1850, 1800, 1750, or less; and the Y coordinate for point B can be 65, 70, 75, 80, 85, 90, 95, 99 or more. The X coordinate for point C can be 50, 60, 70, 80, 90, 100, 150, 200, or more; and the Y coordinate for point C can be 65, 70, 75, 80, 85, 90, 95, 99 or more. The X coordinate for point D can be 12, 13, 14, 15, 16, 17, 18, 19, 20, 25, 30, 40, 50, 75, 100, or more; and the Y coordinate for point D can be 100, 99, 95, 90, 85, 80, 75, or less. In one embodiment, point A can be (1926, 100), point B can be (1926, 95), point C can be (1000, 95), and point D can be (1000, 100).

An isolated nucleic acid containing a nucleic acid sequence that encodes a polypeptide containing an amino acid sequence having a length and a percent identity to the sequence set forth in SEQ ID NO:3 over that length is within the scope of the invention provided the point defined by that length and percent identity is within the area defined by points A, B, C, and D of FIG. 26; where point A has an X coordinate less than or equal to 641, and a Y coordinate less than or equal to 100; where point B has an X coordinate less than or equal to 641, and a Y coordinate greater than or equal to 50; where point C has an X coordinate greater than or equal to 25, and a Y coordinate greater than or equal to 50; and where point D has an X coordinate greater than or equal to 5, and a Y coordinate less than or equal to 100. For example, the X coordinate for point A can be 641, 635, 630, 625, 620, or less; and the Y coordinate for point A can be 100, 99, 95, 90, 85, 80, 75, or less. The X coordinate for point B can be 641, 635, 630, 625, 620, or less; and the Y coordinate for point B can be 50, 55, 60, 65, 70, 75, 80, 85, 90, 95, 99 or more. The X coordinate for point C can be 25, 30, 35, 40, 50, 60, 70, 80, 90, 100, 150, 200, or more; and the Y coordinate for point C can be 50, 55, 60, 65, 70, 75, 80, 85, 90, 95, 99 or more. The X coordinate for point D can be 5, 6, 7, 8, 9, 10, 15, 20, 25, 30, 40, 50, 75, 100, or more; and the Y coordinate for point D can be 100, 99, 95, 90, 85, 80, 75, or less. In one embodiment, point A can be (641, 100), point B can be (641, 95), point C can be (400, 95), and point D can be (400, 100).

An isolated nucleic acid containing a nucleic acid sequence having a length and a percent identity to the sequence set forth in SEQ ID NO:37 over that length is within the scope of the invention provided the point defined by that length and percent identity is within the area defined by points A, B, C, and D of FIG. 26; where point A has an X coordinate less than or equal to 1990, and a Y coordinate less than or equal to 100; where point B has an X coordinate less than or equal to 1990, and a Y coordinate greater than or equal to 65; where point C has an X coordinate greater than or equal to 50, and a Y coordinate greater than or equal to 65; and where point D has an X coordinate greater than or equal to 12, and a Y coordinate less than or equal to 100. For example, the X coordinate for point A can be 1990, 1950, 1900, 1850, 1800, 1750, or less; and the Y coordinate for point A can be 100, 99, 95, 90, 85, 80, 75, or less. The X coordinate for point B can be 1990, 1950, 1900, 1850, 1800, 1750, or less; and the Y coordinate for point B can be 65, 70, 75, 80, 85, 90, 95, 99 or more. The X coordinate for point C can be 50, 60, 70, 80, 90, 100, 150, 200, or more; and the Y coordinate for point C can be 65, 70, 75, 80, 85, 90, 95, 99 or more. The X coordinate for point D can be 12, 13, 14, 15, 16, 17, 18, 19, 20, 25, 30, 40, 50, 75, 100, or more; and the Y coordinate for point D can be 100, 99, 95, 90, 85, 80, 75, or less. In one embodiment, point A can be (1990, 100), point B can be (1990, 95), point C can be (1000, 95), and point D can be (1000, 100).

An isolated nucleic acid containing a nucleic acid sequence having a length and a percent identity to the sequence set forth in SEQ ID NO:38 over that length is within the scope of the invention provided the point defined by that length and percent identity is within the area defined by points A, B, C, and D of FIG. 26; where point A has an X coordinate less than or equal to 1002, and a Y coordinate less than or equal to 100; where point B has an X coordinate less than or equal to 1002, and a Y coordinate greater than or equal to 65; where point C has an X coordinate greater than or equal to 50, and a Y coordinate greater than or equal to 65; and where point D has an X coordinate greater than or equal to 12, and a Y coordinate less than or equal to 100. For example, the X coordinate for point A can be 1002, 950, 900, 850, 800, 750, or less; and the Y coordinate for point A can be 100, 99, 95, 90, 85, 80, 75, or less. The X coordinate for point B can be 1002, 950, 900, 850, 800, 750, or less; and the Y coordinate for point B can be 65, 70, 75, 80, 85, 90, 95, 99 or more. The X coordinate for point C can be 50, 60, 70, 80, 90, 100, 150, 200, or more; and the Y coordinate for point C can be 65, 70, 75, 80, 85, 90, 95, 99 or more. The X coordinate for point D can be 12, 13, 14, 15, 16, 17, 18, 19, 20, 25, 30, 40, 50, 75, 100, or more; and the Y coordinate for point D can be 100, 99, 95, 90, 85, 80, 75, or less. In one embodiment, point A can be (1002, 100), point B can be (1002, 95), point C can be (500, 95), and point D can be (500, 100).

An isolated nucleic acid containing a nucleic acid sequence that encodes a polypeptide containing an amino acid sequence having a length and a percent identity to the sequence set forth in SEQ ID NO:39 over that length is within the scope of the invention provided the point defined by that length and percent identity is within the area defined by points A, B, C, and D of FIG. 26; where point A has an X coordinate less than or equal to 333, and a Y coordinate less than or equal to 100; where point B has an X coordinate less than or equal to 333, and a Y coordinate greater than or equal to 50; where point C has an X coordinate greater than or equal to 25, and a Y coordinate greater than or equal to 50; and where point D has an X coordinate greater than or equal to 5, and a Y coordinate less than or equal to 100. For example, the X coordinate for point A can be 333, 330, 325, 320, 315, or less; and the Y coordinate for point A can be 100, 99, 95, 90, 85, 80, 75, or less. The X coordinate for point B can be 333, 330, 325, 320, 315, or less; and the Y coordinate for point B can be 50, 55, 60, 65, 70, 75, 80, 85, 90, 95, 99 or more. The X coordinate for point C can be 25, 30, 35, 40, 50, 60, 70, 80, 90, 100, 150, 200, or more; and the Y coordinate for point C can be 50, 55, 60, 65, 70, 75, 80, 85, 90, 95, 99 or more. The X coordinate for point D can be 5, 6, 7, 8, 9, 10, 15, 20, 25, 30, 40, 50, 75, 100, or more; and the Y coordinate for point D can be 100, 99, 95, 90, 85, 80, 75, or less. In one embodiment, point A can be (333, 100), point B can be (333, 95), point C can be (150, 95), and point D can be (150, 100).

An isolated nucleic acid containing a nucleic acid sequence having a length and a percent identity to the sequence set forth in SEQ ID NO:40 over that length is within the scope of the invention provided the point defined by that length and percent identity is within the area defined by points A, B, C, and D of FIG. 26; where point A has an X coordinate less than or equal to 1833, and a Y coordinate less than or equal to 100; where point B has an X coordinate less than or equal to 1833, and a Y coordinate greater than or equal to 65; where point C has an X coordinate greater than or equal to 50, and a Y coordinate greater than or equal to 65; and where point D has an X coordinate greater than or equal to 12, and a Y coordinate less than or equal to 100. For example, the X coordinate for point A can be 1833, 1800, 1750, 1700, 1650, or less; and the Y coordinate for point A can be 100, 99, 95, 90, 85, 80, 75, or less. The X coordinate for point B can be 1833, 1800, 1750, 1700, 1650, or less; and the Y coordinate for point B can be 65, 70, 75, 80, 85, 90, 95, 99 or more. The X coordinate for point C can be 50, 60, 70, 80, 90, 100, 150, 200, or more; and the Y coordinate for point C cal be 65, 70, 75, 80, 85, 90, 95, 99 or more. The X coordinate for point D can be 12, 13, 14, 15, 16, 17, 18, 19, 20, 25, 30, 40, 50, 75, 100, or more; and the Y coordinate for point D can be 100, 99, 95, 90, 85, 80, 75, or less. In one embodiment, point A can be (1833, 100), point B can be (1833, 95), point C can be (900, 95), and point D can be (900, 100).

An isolated nucleic acid containing a nucleic acid sequence having a length and a percent identity to the sequence set forth in SEQ ID NO:41 over that length is within the scope of the invention provided the point defined by that length and percent identity is within the area defined by points A, B, C, and D of FIG. 26; where point A has an X coordinate less than or equal to 1014, and a Y coordinate less than or equal to 100; where point B has an X coordinate less than or equal to 1014, and a Y coordinate greater than or equal to 65; where point C has an X coordinate greater than or equal to 50, and a Y coordinate greater than or equal to 65; and where point D has an X coordinate greater than or equal to 12, and a Y coordinate less than or equal to 100. For example, the X coordinate for point A can be 1014, 950, 900, 800, 700, 600, or less; and the Y coordinate for point A can be 100, 99, 95, 90, 85, 80, 75, or less. The X coordinate for point B can be 1014, 950, 900, 800, 700, 600, or less; and the Y coordinate for point B can be 65, 70, 75, 80, 85, 90, 95, 99 or more. The X coordinate for point C can be 50, 60, 70, 80, 90, 100, 150, 200, or more; and the Y coordinate for point C can be 65, 70, 75, 80, 85, 90, 95, 99 or more. The X coordinate for point D can be 12, 13, 14, 15, 16, 17, 18, 19, 20, 25, 30, 40, 50, 75, 100, or more; and the Y coordinate for point D can be 100, 99, 95, 90, 85, 80, 75, or less. In one embodiment, point A can be (1014, 100), point B can be (1014, 95), point C can be (500, 95), and point D can be (500, 100).

An isolated nucleic acid containing a nucleic acid sequence that encodes a polypeptide containing an amino acid sequence having a length and a percent identity to the sequence set forth in SEQ ID NO:42 over that length is within the scope of the invention provided the point defined by that length and percent identity is within the area defined by points A, B, C, and D of FIG. 26; where point A has an X coordinate less than or equal to 337, and a Y coordinate less than or equal to 100; where point B has an X coordinate less than or equal to 337, and a Y coordinate greater than or equal to 50; where point C has an X coordinate greater than or equal to 25, and a Y coordinate greater than or equal to 50; and where point D has an X coordinate greater than or equal to 5, and a Y coordinate less than or equal to 100. For example, the X coordinate for point A can be 337, 335, 330, 325, 320, 315, or less; and the Y coordinate for point A can be 100, 99, 95, 90, 85, 80, 75, or less. The X coordinate for point B can be 337, 335, 330, 325, 320, 315, or less; and the Y coordinate for point B can be 50, 55, 60, 65, 70, 75, 80, 85, 90, 95, 99 or more. The X coordinate for point C can be 25, 30, 35, 40, 50, 60, 70, 80, 90, 100, 150, 200, or more; and the Y coordinate for point C can be 50, 55, 60, 65, 70, 75, 80, 85, 90, 95, 99 or more. The X coordinate for point D can be 5, 6, 7, 8, 9, 10, 15, 20, 25, 30, 40, 50, 75, 100, or more; and the Y coordinate for point D can be 100, 99, 95, 90, 85, 80, 75, or less. In one embodiment, point A can be (337, 100), point B can be (337, 95), point C can be (150, 95), and point D can be (150, 100).

An isolated nucleic acid containing a nucleic acid sequence having a length and a percent identity to the sequence set forth in SEQ ID NO:95 over that length is within the scope of the invention provided the point defined by that length and percent identity is within the area defined by points A, B, C, and D of FIG. 26; where point A has an X coordinate less than or equal to 2017, and a Y coordinate less than or equal to 100; where point B has an X coordinate less than or equal to 2017, and a Y coordinate greater than or equal to 65; where point C has an X coordinate greater than or equal to 50, and a Y coordinate greater than or equal to 65; and where point D has an X coordinate greater than or equal to 12, and a Y coordinate less than or equal to 100. For example, the X coordinate for point A can be 2017, 2000, 1900, 1950, 1800, 1700, 1600, or less; and the Y coordinate for point A can be 100, 99, 95, 90, 85, 80, 75, or less. The X coordinate for point B can be 2017, 2000, 1900, 1950, 1800, 1700, 1600, or less; and the Y coordinate for point B can be 65, 70, 75, 80, 85, 90, 95, 99 or more. The X coordinate for point C can be 50, 60, 70, 80, 90, 100, 150, 200, 500, 1000, 1500, or more; and the Y coordinate for point C can be 65, 70, 75, 80, 85, 90, 95, 99 or more. The X coordinate for point D can be 12, 13, 14, 15, 16, 17, 18, 19, 20, 25, 30, 40, 50, 75, 100, 250, 500, 1000, 1500, or more; and the Y coordinate for point D can be 100, 99, 95, 90, 85, 80, 75, or less. In one embodiment, point A can be (2017, 100), point B can be (2017, 95), point C can be (1800, 95), and point D can be (1800, 100).

An isolated nucleic acid containing a nucleic acid sequence having a length and a percent identity to the sequence set forth in SEQ ID NO:96 over that length is within the scope of the invention provided the point defined by that length and percent identity is within the area defined by points A, B, C, and D of FIG. 26; where point A has an X coordinate less than or equal to 1161, and a Y coordinate less than or equal to 100; where point B has an X coordinate less than or equal to 1161, and a Y coordinate greater than or equal to 65; where point C has an X coordinate greater than or equal to 50, and a Y coordinate greater than or equal to 65; and where point D has an X coordinate greater than or equal to 12, and a Y coordinate less than or equal to 100. For example, the X coordinate for point A can be 1161, 1050, 1000, 950, 900, 800, 700, 600, or less; and the Y coordinate for point A can be 100, 99, 95, 90, 85, 80, 75, or less. The X coordinate for point B can be 1161, 1050, 1000, 950, 900, 800, 700, 600, or less; and the Y coordinate for point B can be 65, 70, 75, 80, 85, 90, 95, 99 or more. The X coordinate for point C can be 50, 60, 70, 80, 90, 100, 150, 200, 250, 500, 1000, or more; and the Y coordinate for point C can be 65, 70, 75, 80, 85, 90, 95, 99 or more. The X coordinate for point D can be 12, 13, 14, 15, 16, 17, 18, 19, 20, 25, 30, 40, 50, 75, 100, 250, 500, 1000, or more; and the Y coordinate for point D can be 100, 99, 95, 90, 85, 80, 75, or less. In one embodiment, point A can be (1161, 100), point B can be (1161, 95), point C can be (1000, 95), and point D can be (1000, 100).

An isolated nucleic acid containing a nucleic acid sequence that encodes a polypeptide containing an amino acid sequence having a length and a percent identity to the sequence set forth in SEQ ID NO:97 over that length is within the scope of the invention provided the point defined by that length and percent identity is within the area defined by points A, B, C, and D of FIG. 26; where point A has an X coordinate less than or equal to 386, and a Y coordinate less than or equal to 100; where point B has an X coordinate less than or equal to 0.386, and a Y coordinate greater than or equal to 50; where point C has an X coordinate greater than or equal to 25, and a Y coordinate greater than or equal to 50; and where point D has an X coordinate greater than or equal to 5, and a Y coordinate less than or equal to 100. For example, the X coordinate for point A can be 386, 380, 375, 370, 375, 360, 365, 350, 325, 300, or less; and the Y coordinate for point A can be 100, 99, 95, 90, 85, 80, 75, or less. The X coordinate for point B can be 386, 380, 375, 370, 375, 360, 365, 350, 325, 300, or less; and the Y coordinate for point B can be 50, 55, 60, 65, 70, 75, 80, 85, 90, 95, 99 or more. The X coordinate for point C can be 25, 30, 35, 40, 50, 60, 70, 80, 90, 100, 150, 200, 300, 350, or more; and the Y coordinate for point C can be 50, 55, 60, 65, 70, 75, 80, 85, 90, 95, 99 or more. The X coordinate for point D can be 5, 6, 7, 8, 9, 10, 15, 20, 25, 30, 40, 50, 75, 100, 200, 300, 350, or more; and the Y coordinate for point D can be 100, 99, 95, 90, 85, 80, 75, or less. In one embodiment, point A can be (386, 100), point B can be (386, 95), point C can be (350, 95), and point D can be (350, 100).

The invention also provides isolated nucleic acid that is at least about 12 bases in length (e.g., at least about 13, 14, 15, 16, 17, 18, 19, 20, 25, 30, 40, 50, 60, 100, 250, 500, 750, 1000, 1500, 2000, 3000, 4000, or 5000 bases in length) and hybridizes, under hybridization conditions, to the sense or antisense strand of a nucleic acid having the sequence set forth in SEQ ID NO:1, 2, 37, 38, 40, 41, 95, or 96. The hybridization conditions can be moderately or highly stringent hybridization conditions.

For the purpose of this invention, moderately stringent hybridization conditions mean the hybridization is performed at about 42° C. in a hybridization solution containing 25 mM KPO₄ (pH 7.4), 5×SSC, 5×Denhart's solution, 50 μg/mL denatured, sonicated salmon sperm DNA, 50% formamide, 10% Dextran sulfate, and 1-15 ng/mL probe (about 5×10⁷ cpm/μg), while the washes are performed at about 50° C. with a wash solution containing 2×SSC and 0.1% sodium dodecyl sulfate.

Highly stringent hybridization conditions mean the hybridization is performed at about 42° C. in a hybridization solution containing 25 mM KPO₄ (pH 7.4), 5×SSC, 5×Denhart's solution, 50 μg/mL denatured, sonicated salmon sperm DNA, 50% formamide, 10% Dextran sulfate, and 1-15 ng/mL probe (about 5×10⁷ cpm/μg), while the washes are performed at about 65° C. with a wash solution containing 0.2×SSC and 0.1% sodium dodecyl sulfate.

Isolated nucleic acid within the scope of the invention can be obtained using any method including, without limitation, common molecular cloning and chemical nucleic acid synthesis techniques. For example, PCR can be used to obtain an isolated nucleic acid containing a nucleic acid sequence sharing similarity to the sequence set forth in SEQ ID NO:1, 2, 37, 38, 40, 41, 95, or 96. PCR refers to a procedure or technique in which target nucleic acid is amplified in a manner similar to that described in U.S. Pat. No. 4,683,195, and subsequent modifications of the procedure described therein. Generally, sequence information from the ends of the region of interest or beyond are used to design oligonucleotide primers that are identical or similar in sequence to opposite strands of a potential template to be amplified. Using PCR, a nucleic acid sequence can be amplified from RNA or DNA. For example, a nucleic acid sequence can be isolated by PCR amplification from total cellular RNA, total genomic DNA, and cDNA as well as from bacteriophage sequences, plasmid sequences, viral sequences, and the like. When using RNA as a source of template, reverse transcriptase can be used to synthesize complimentary DNA strands.

An isolated nucleic acid within the scope of the invention also can be obtained by mutagenesis. For example, an isolated nucleic acid containing a sequence set forth in SEQ ID NO:1, 2, 37, 38, 40, 41, 95, or 96 can be mutated using common molecular cloning techniques (e.g., site-directed mutagenesis). Possible mutations include, without limitation, deletions, insertions, and substitutions, as well as combinations of deletions, insertions, and substitutions.

In addition, nucleic acid and amino acid databases (e.g., GenBank®) can be used to obtain an isolated nucleic acid within the scope of the invention. For example, any nucleic acid sequence having some homology to a sequence set forth in SEQ ID NO: 1, 2, 37, 38, 40, 41, 95, or 96, or any amino acid sequence having some homology to a sequence set forth in SEQ ID NO:3, 39, 42, or 97 can be used as a query to search GenBank®.

Further, nucleic acid hybridization techniques can be used to obtain an isolated nucleic acid within the scope of the invention. Briefly, any nucleic acid having some homology to a sequence set forth in SEQ ID NO: 1, 2, 37, 38, 40, 41, 95, or 96 can be used as a probe to identify a similar nucleic acid by hybridization under conditions of moderate to high stringency. Once identified, the nucleic acid then can be purified, sequenced, and analyzed to determine whether it is within the scope of the invention as described herein.

Hybridization can be done by Southern or Northern analysis to identify a DNA or RNA sequence, respectively, that hybridizes to a probe. The probe can be labeled with a biotin, digoxygenin, an enzyme, or a radioisotope such as ³²P. The DNA or RNA to be analyzed can be electrophoretically separated on an agarose or polyacrylamide gel, transferred to nitrocellulose, nylon, or other suitable membrane, and hybridized with the probe using standard techniques well known in the art such as those described in sections 7.39-7.52 of Sambrook et al., (1989) Molecular Cloning, second edition, Cold Spring harbor Laboratory, Plainview, N. Y. Typically, a probe is at least about 20 nucleotides in length. For example, a probe corresponding to a 20 nucleotide sequence set forth in SEQ ID NO:1, 2, 37, 38, 40, 41, 95, or 96 can be used to identify an identical or similar nucleic acid. In addition, probes longer or shorter than 20 nucleotides can be used.

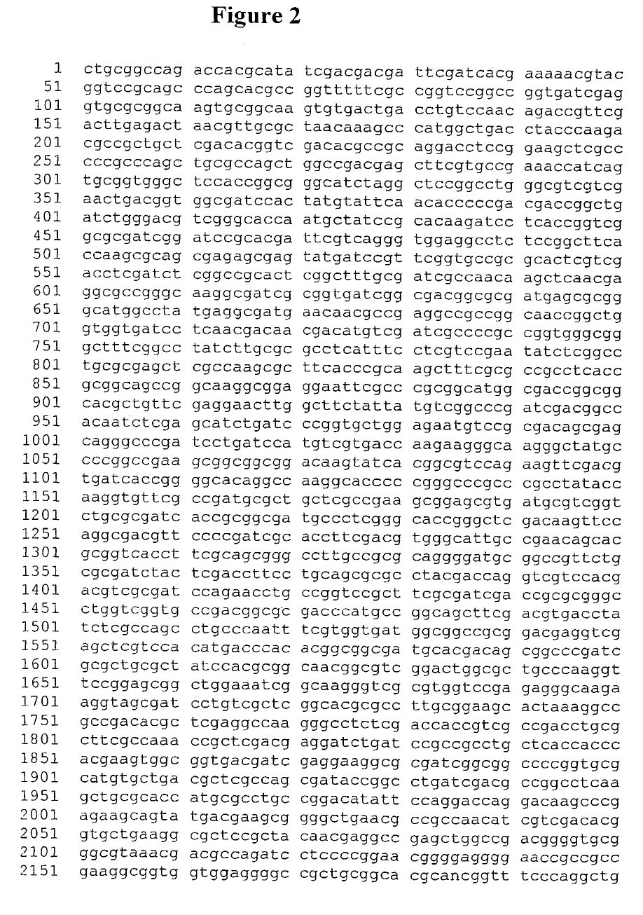

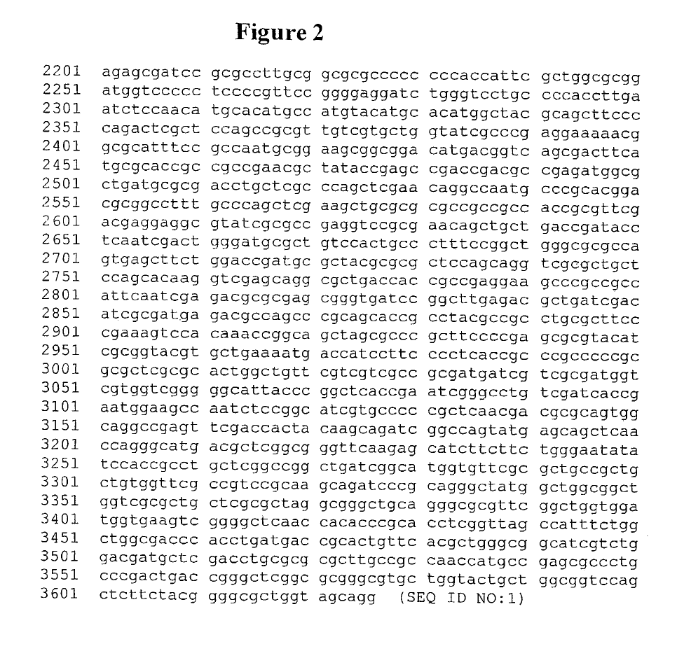

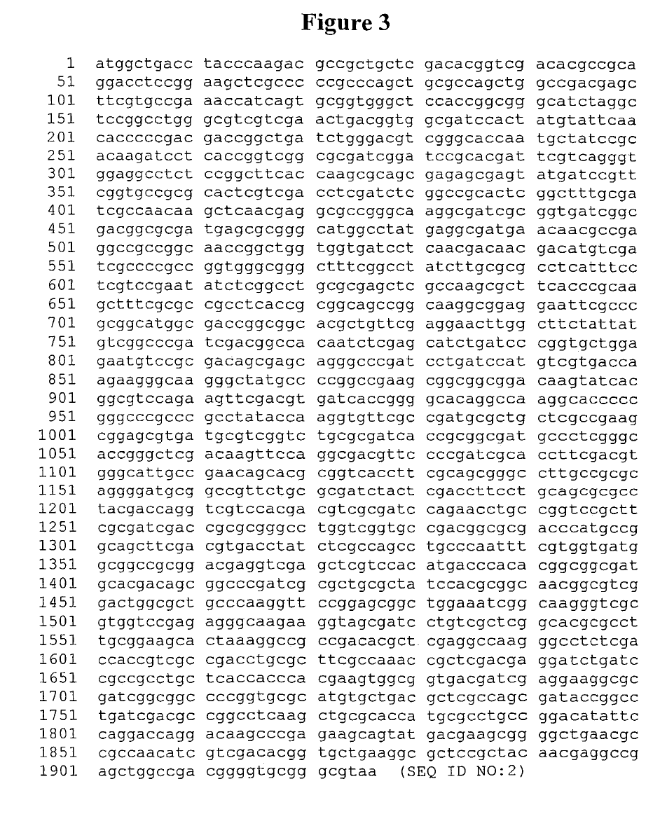

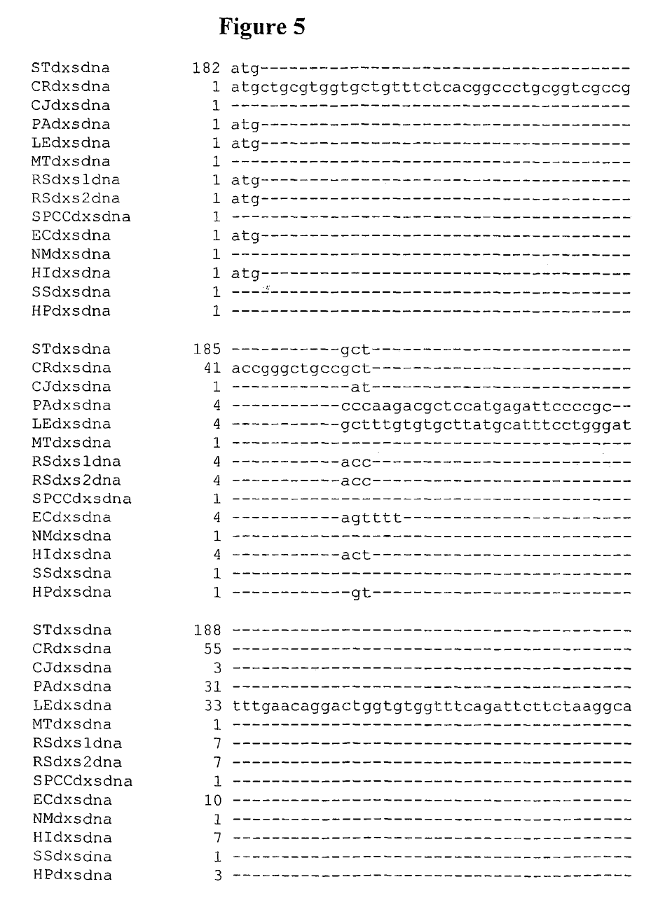

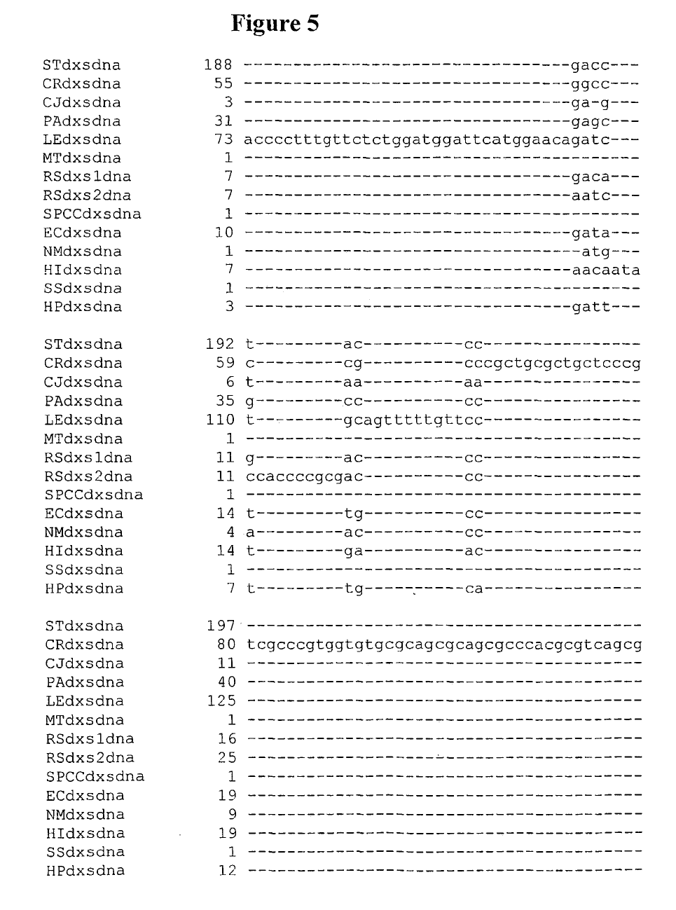

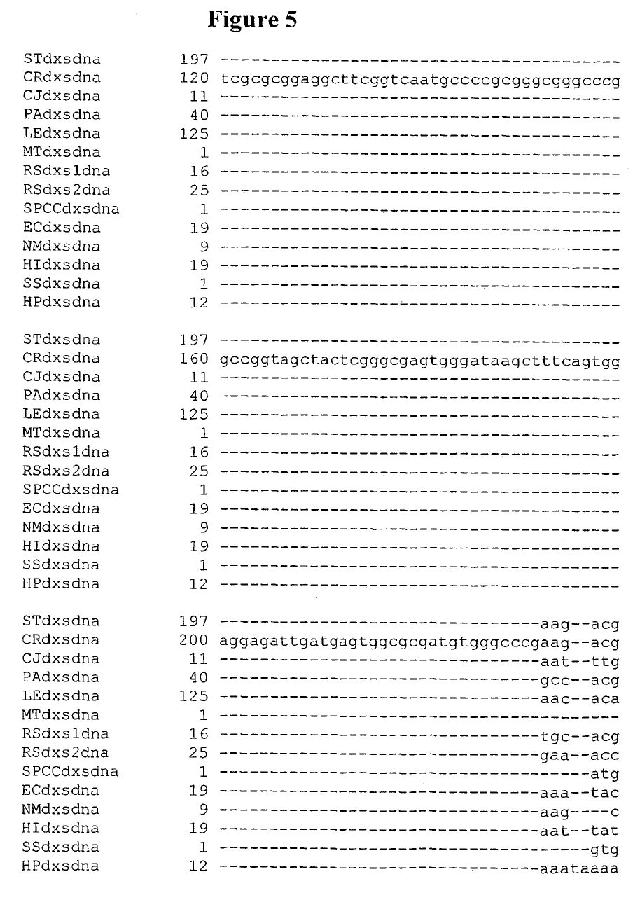

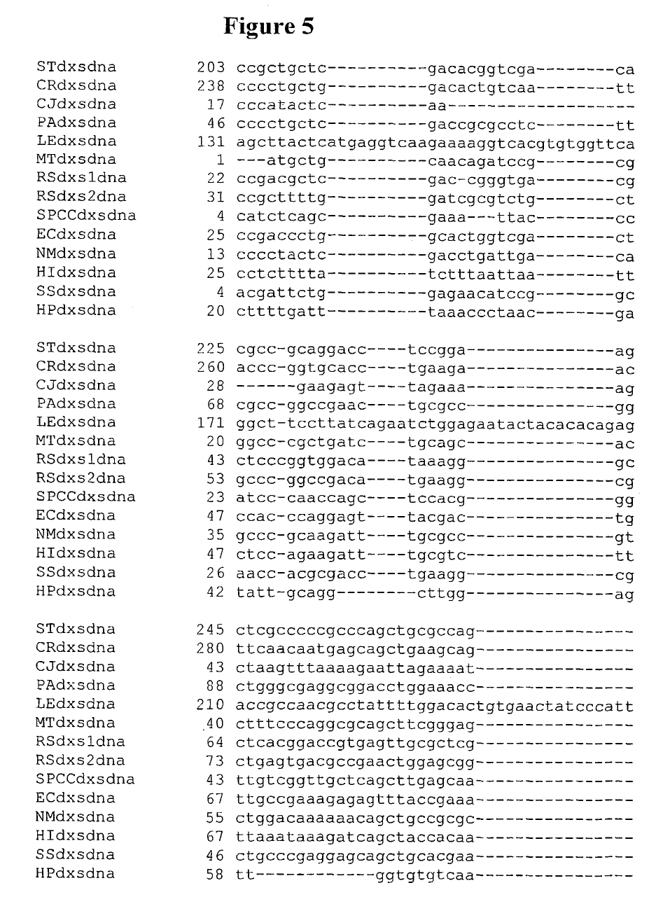

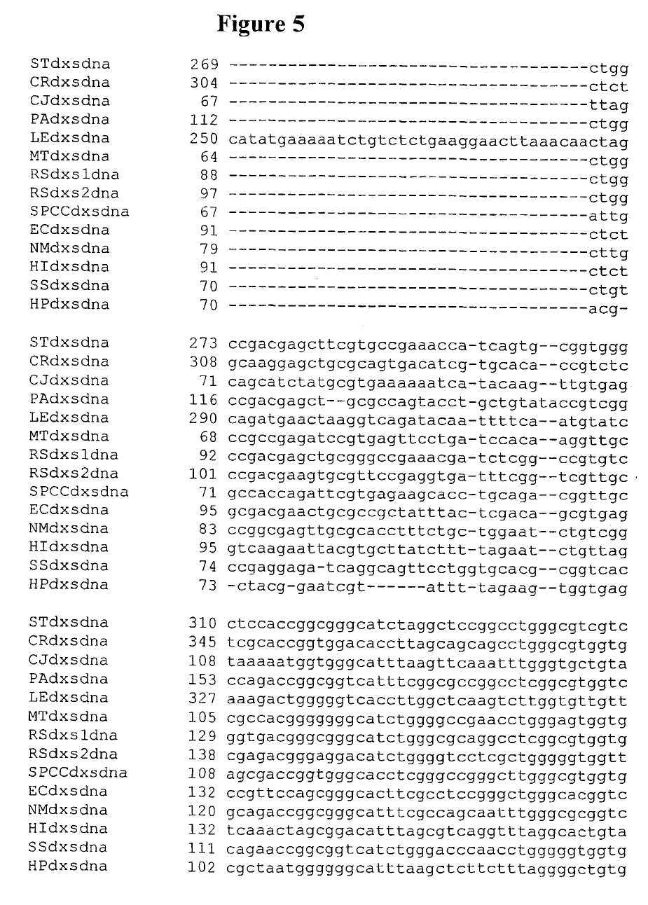

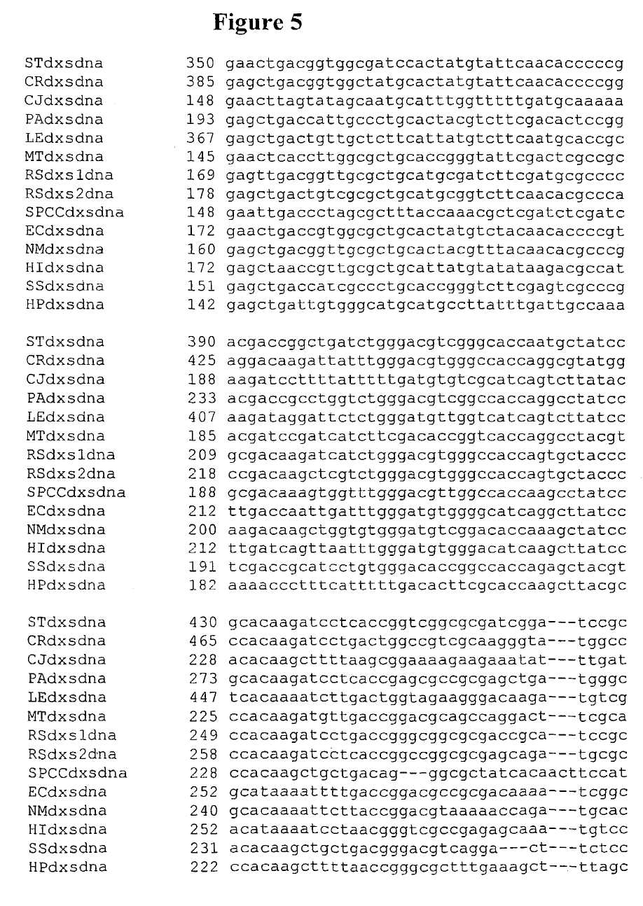

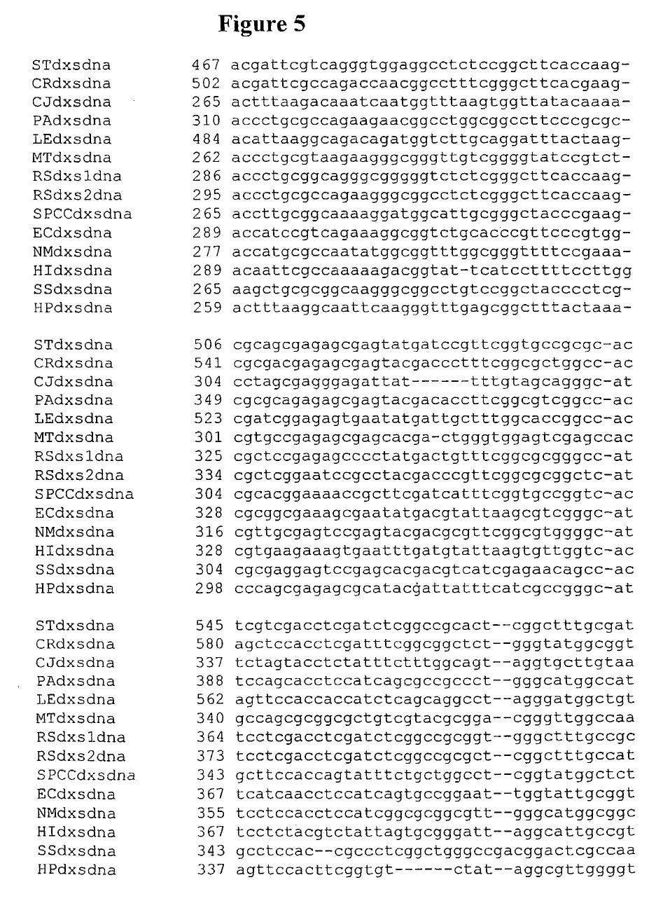

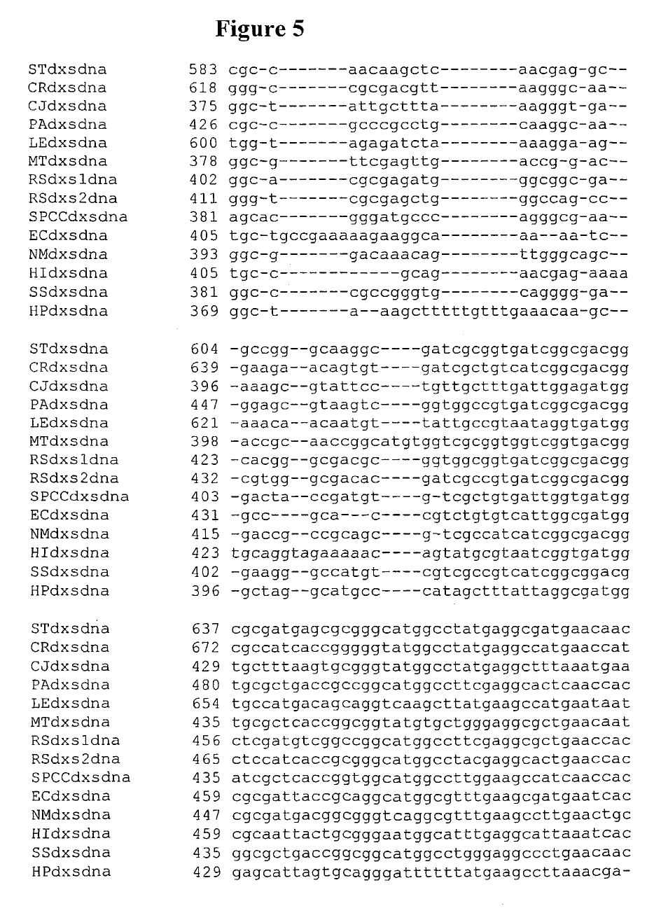

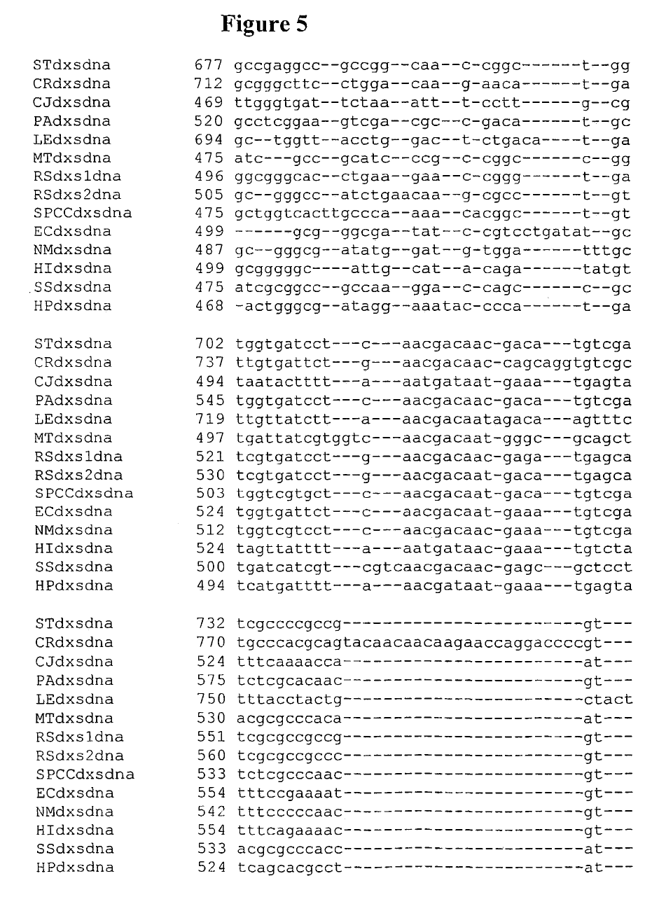

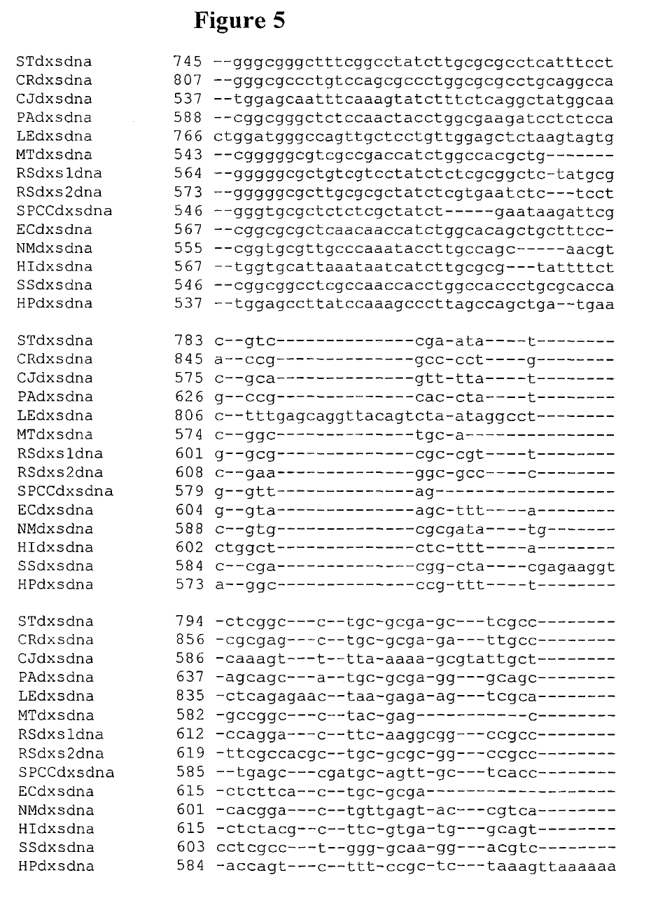

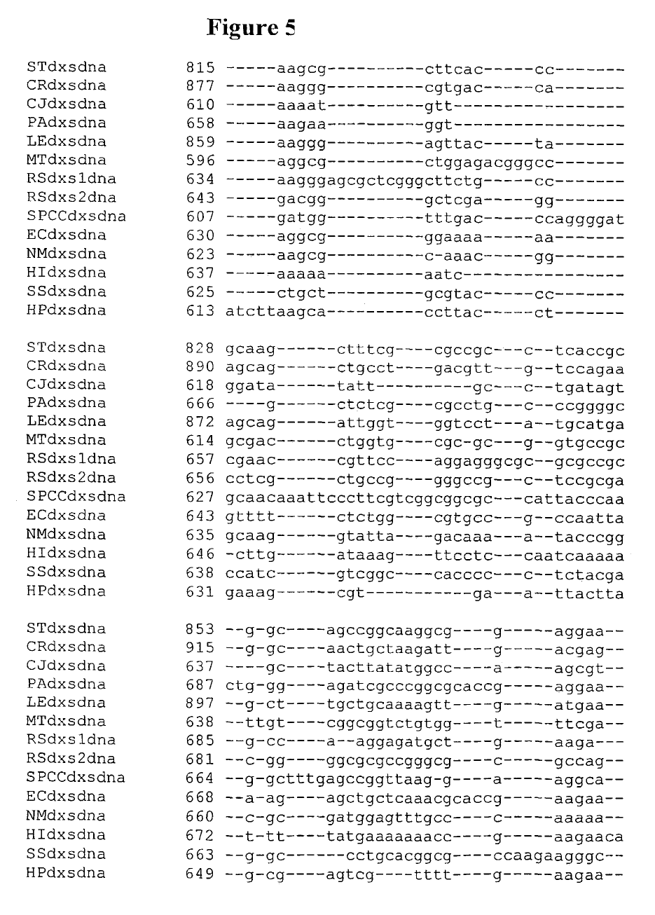

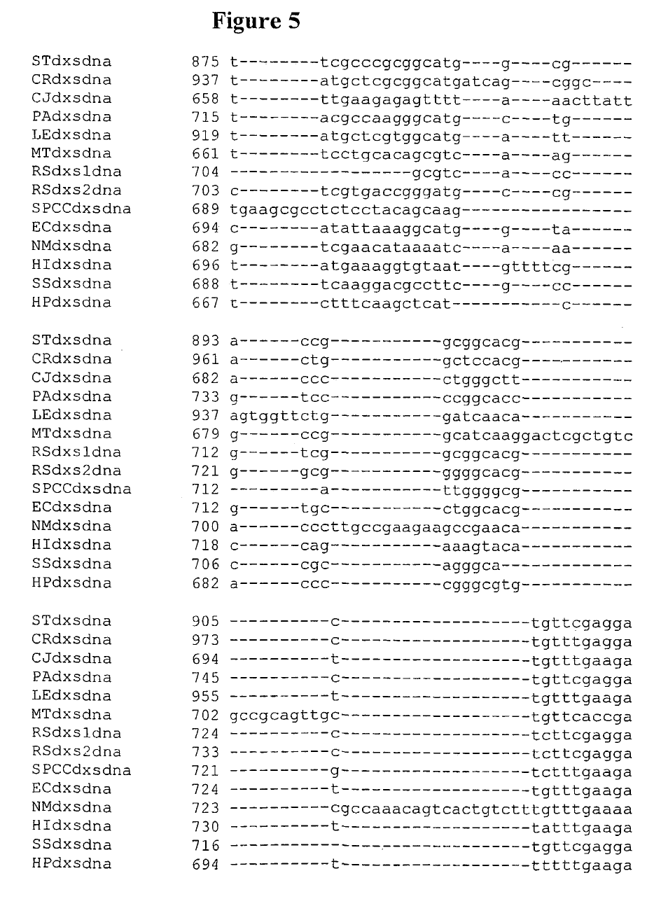

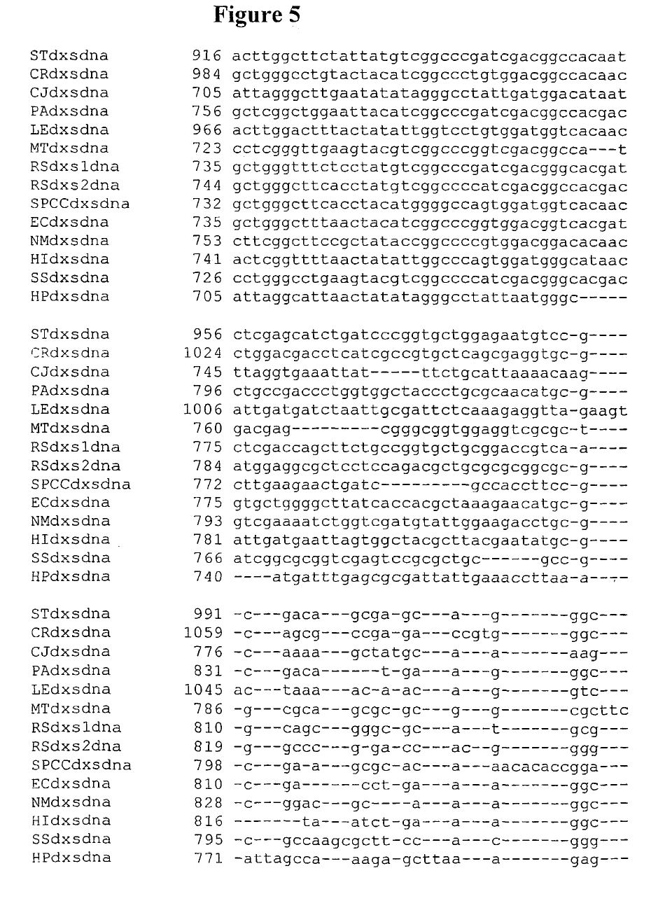

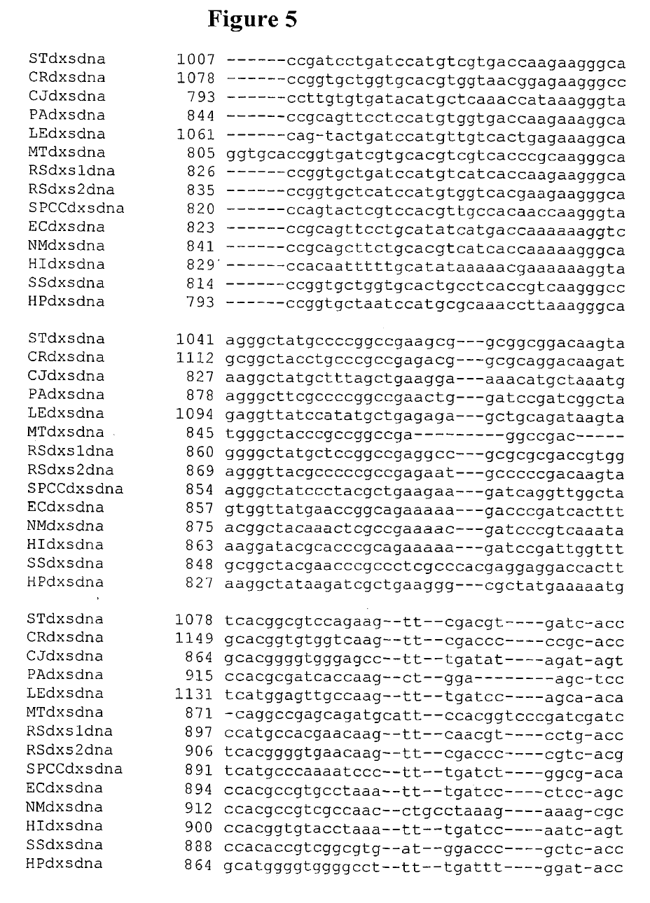

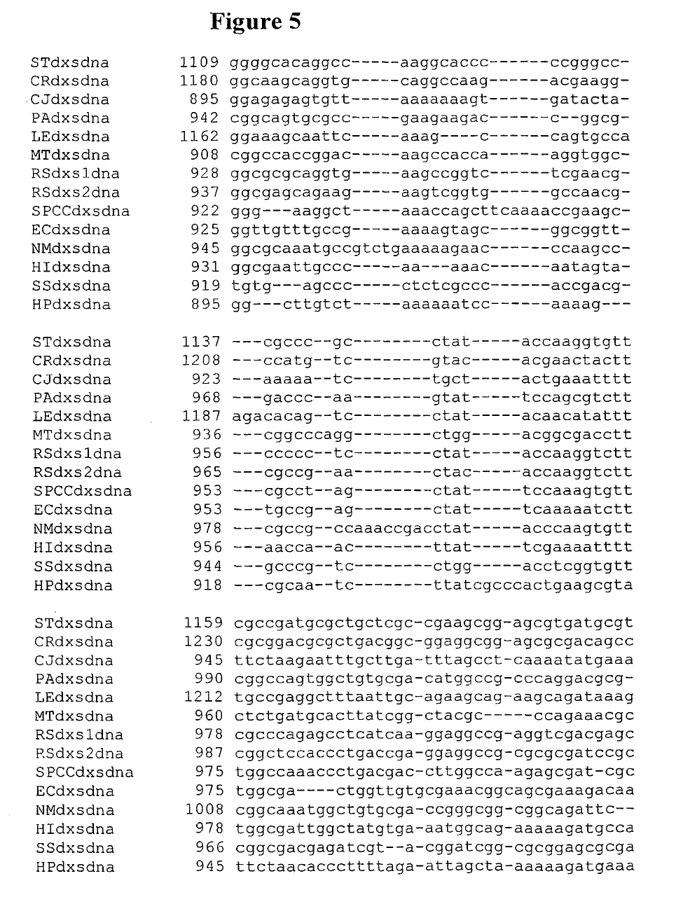

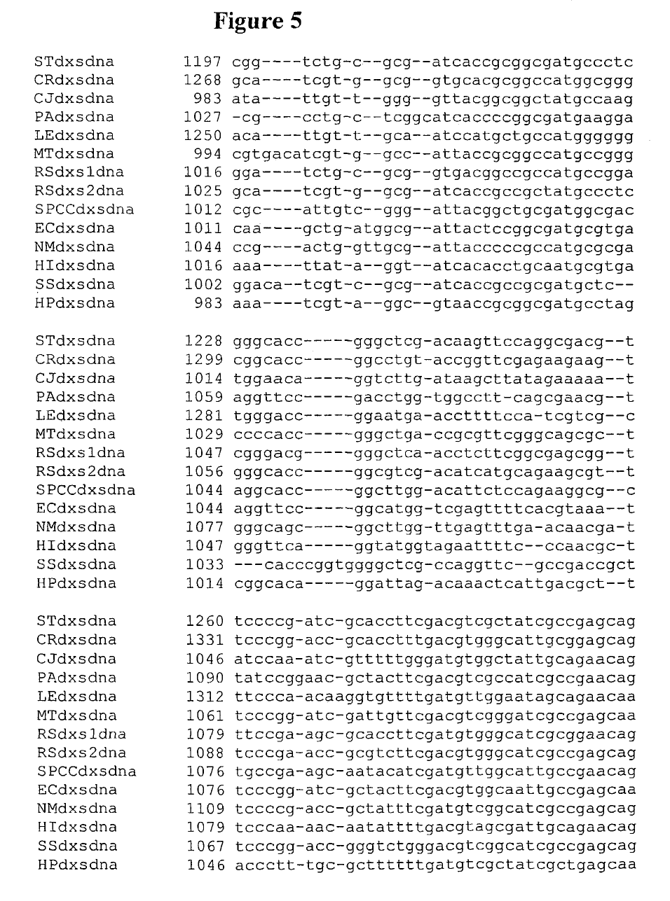

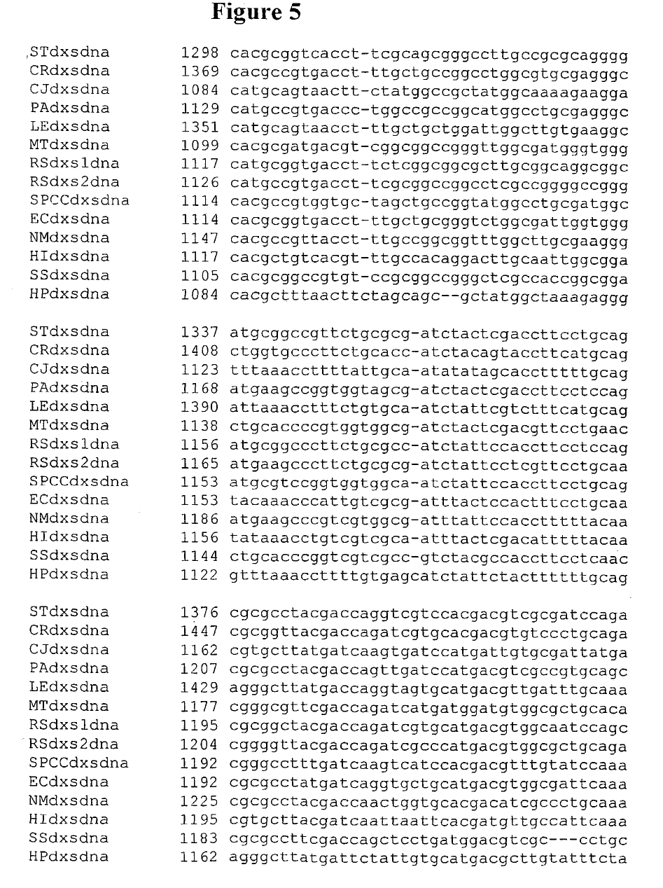

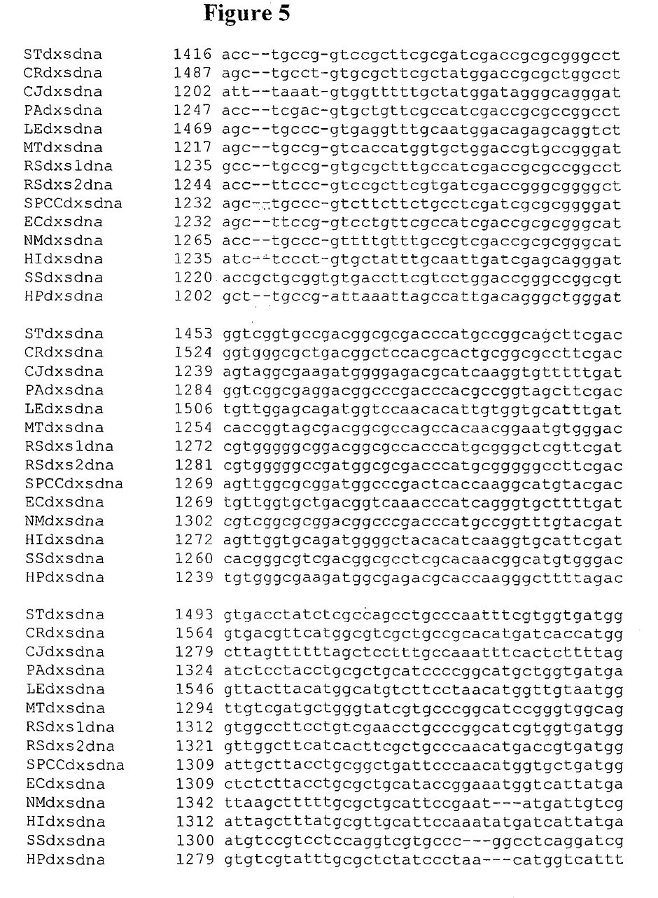

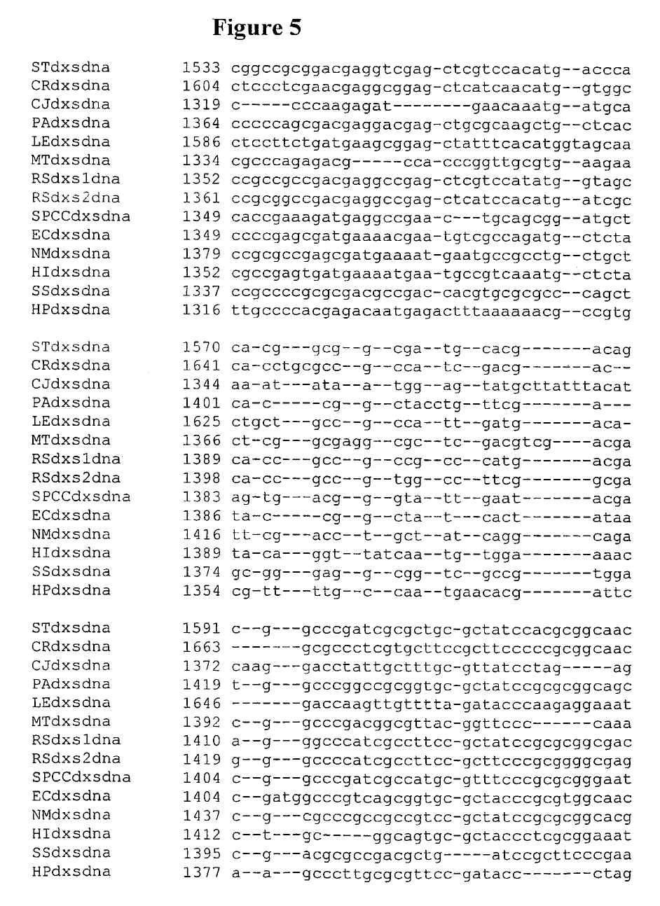

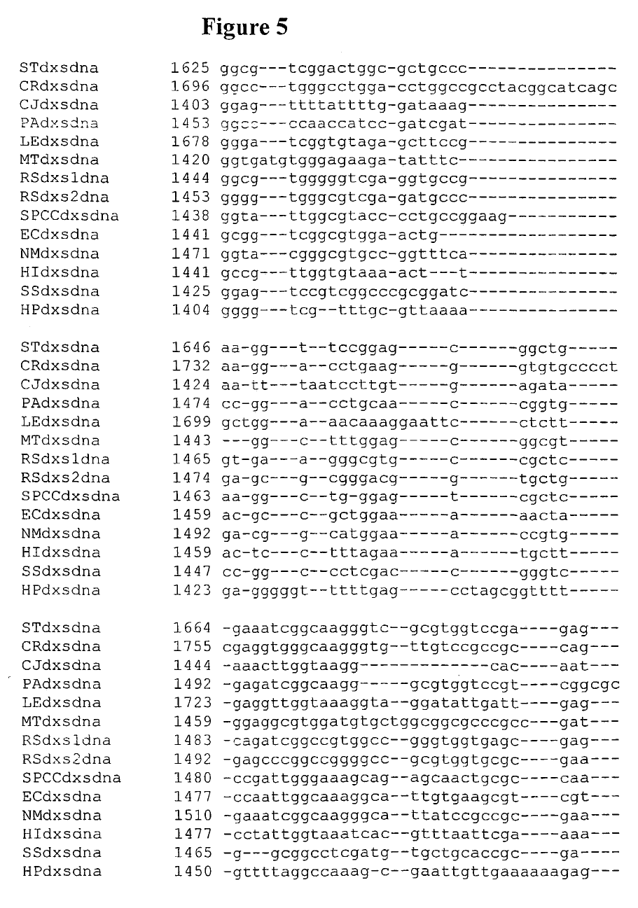

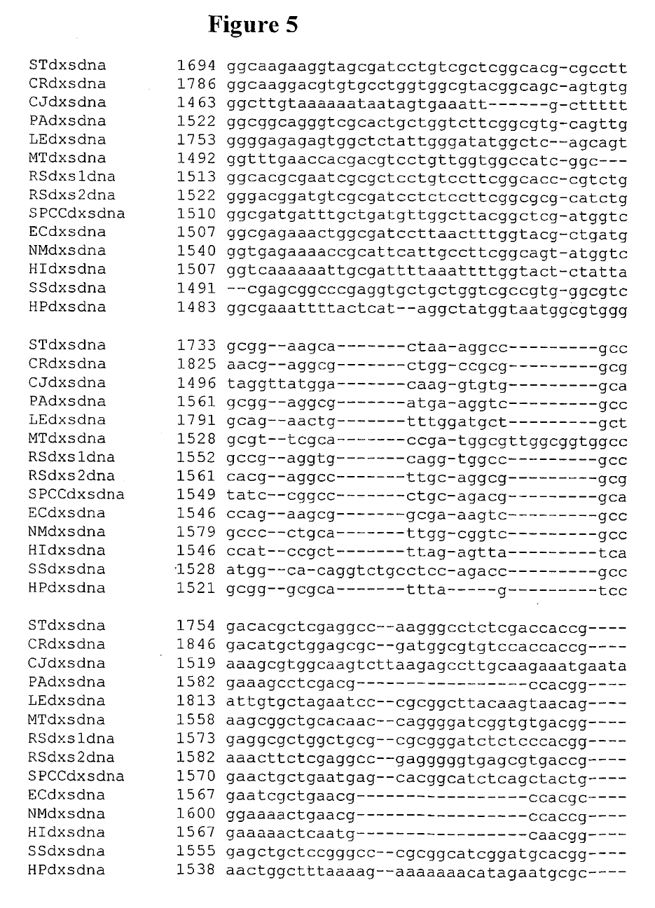

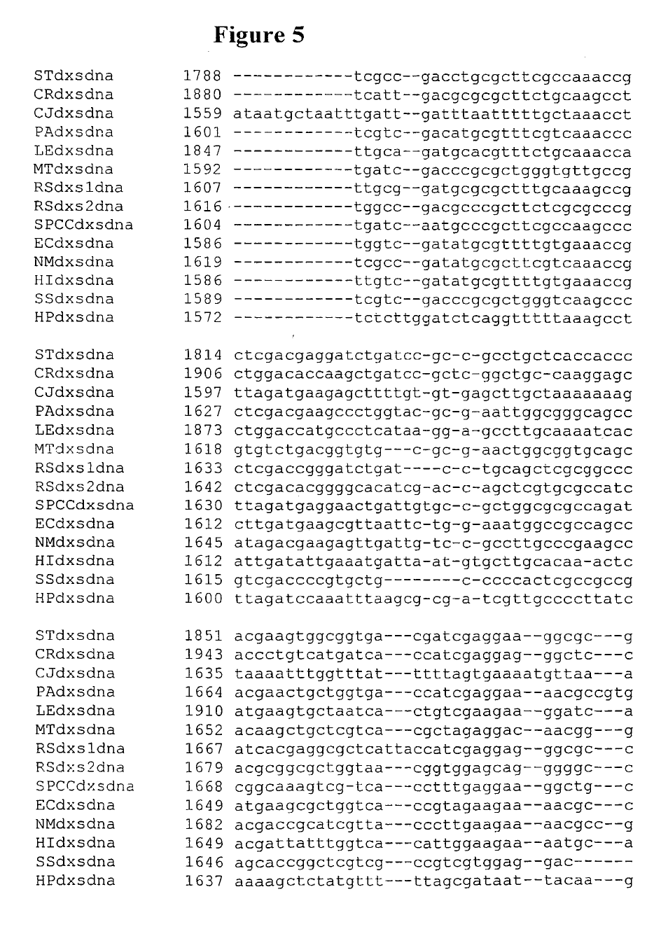

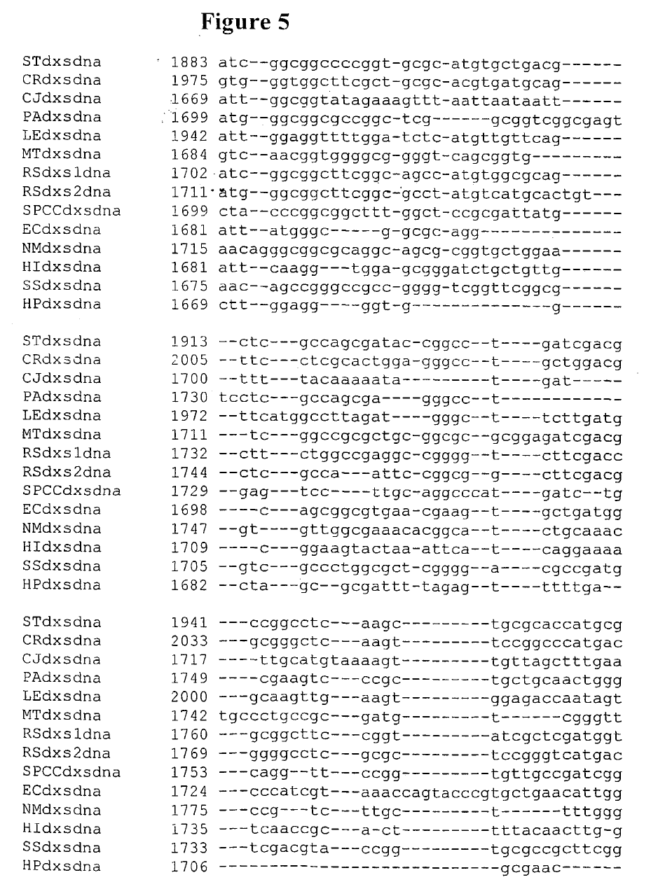

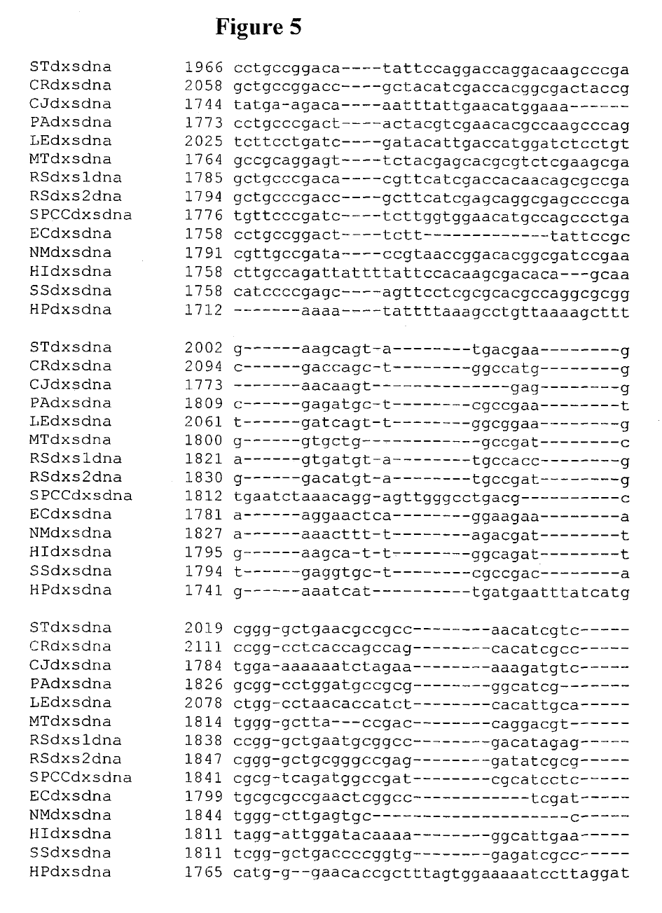

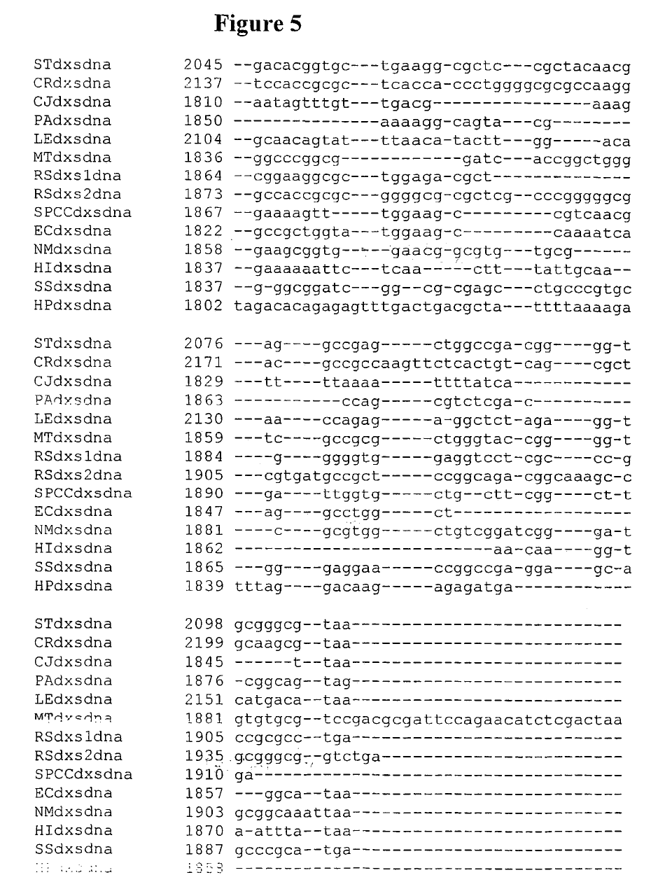

The invention provides isolated nucleic acid that contains the entire nucleic acid sequence depicted in FIG. 2, 3, 7, 8, 10, 11, 28, or 29. In addition, the invention provides isolated nucleic acid that contains a portion of the nucleic acid sequence depicted in FIG. 2, 3, 7, 8, 10, 11, 28, or 29. For example, the invention provides isolated nucleic acid that contains a 15 nucleotide sequence identical to any 15 nucleotide sequence depicted in FIG. 2, 3, 7, 8, 10, 11, 28, or 29 including, without limitation, the sequence starting at nucleotide number 1 and ending at nucleotide number 15, the sequence starting at nucleotide number 2 and ending at nucleotide number 16, the sequence starting at nucleotide number 3 and ending at nucleotide number 17, and so forth. It will be appreciated that the invention also provides isolated nucleic acid that contains a nucleotide sequence that is greater than 15 nucleotides (e.g., 16, 17, 18, 19, 20, 21, 22, 23, 24, 25, 26, 27, 28, 29, 30, or more nucleotides) in length and identical to any portion of the sequence depicted in FIG. 2, 3, 7, 8, 10, 11, 28, or 29. For example, the invention provides isolated nucleic acid that contains a 25 nucleotide sequence identical to any 25 nucleotide sequence depicted in FIG. 2, 3, 7, 8, 10, 11, 28, or 29 including, without limitation, the sequence starting at nucleotide number 1 and ending at nucleotide variations. For example, the STdxsdna sequence can contain one variation provided in FIG. 5 or more than one (e.g., 2, 3, 4, 5, 6, 7, 8, 9, 10, 15, 20, 25, 50, 100, or more) of the variations provided in FIG. 5. It is noted that the full-length nucleic acid sequences depicted in FIG. 5 can encode polypeptides having DXS activity. It also is noted that the nucleic acid sequence depicted in FIG. 2 contains the nucleic acid sequence depicted in FIG. 3.

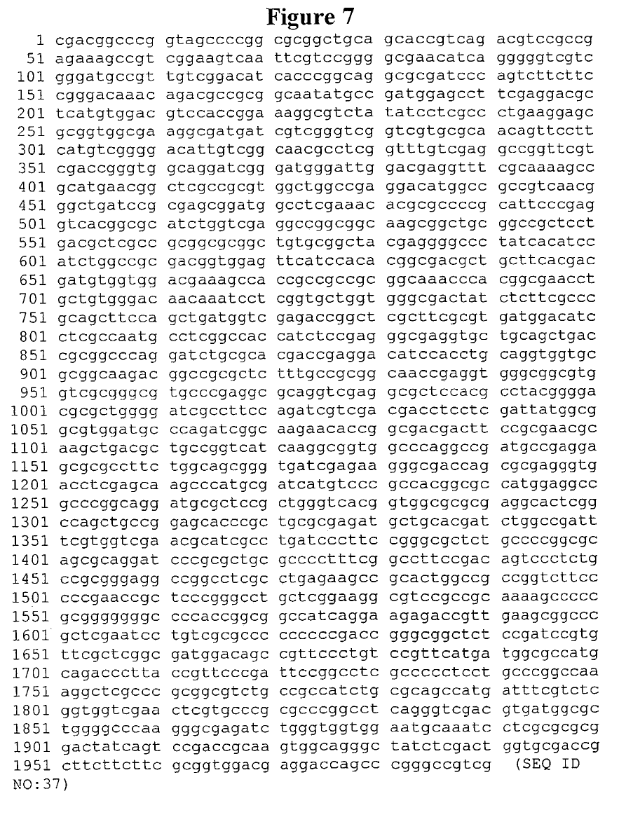

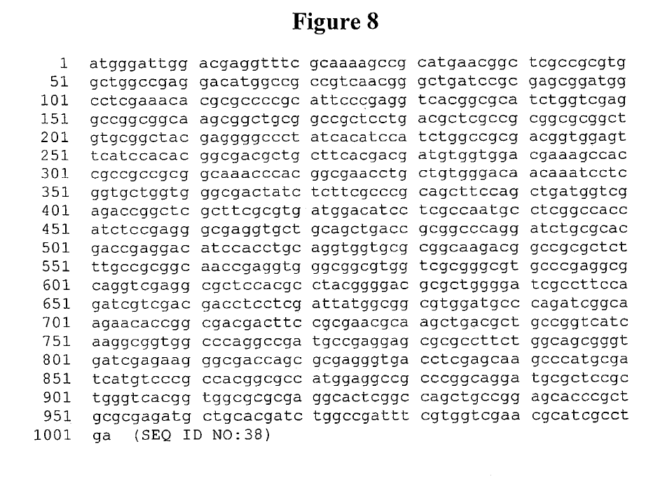

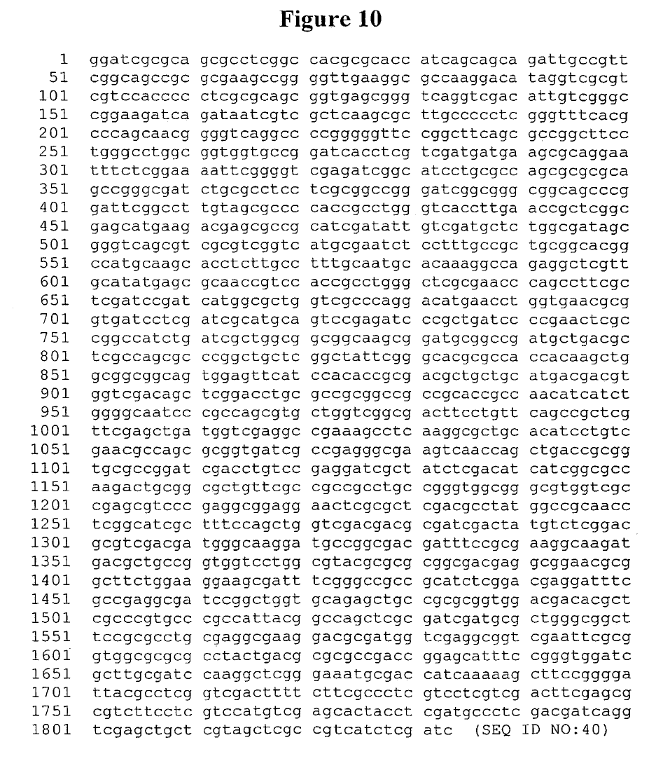

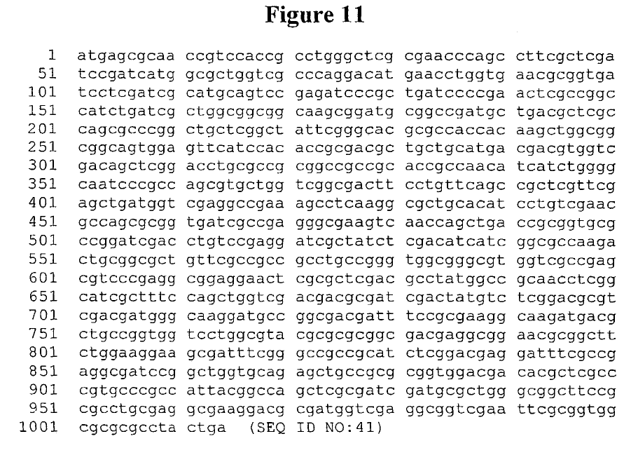

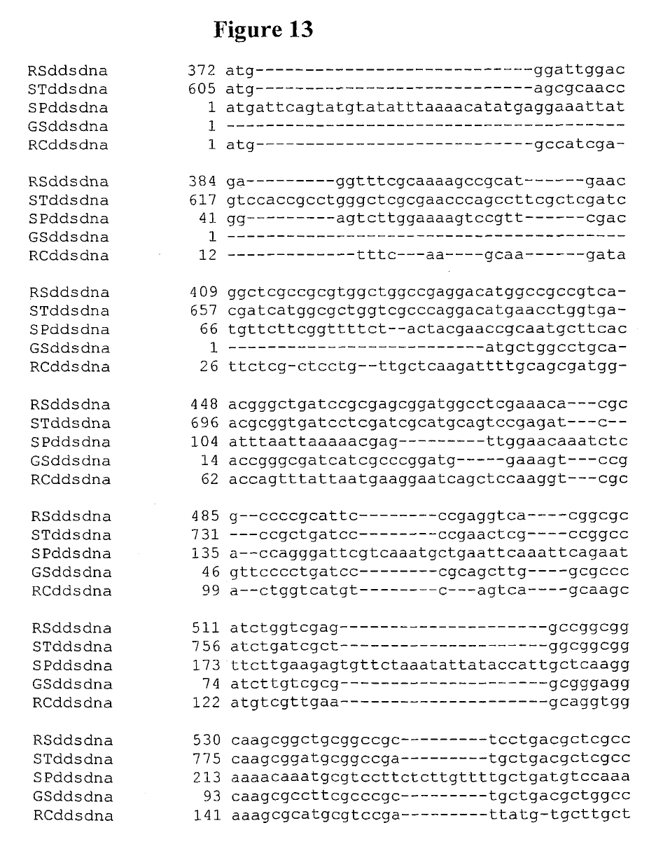

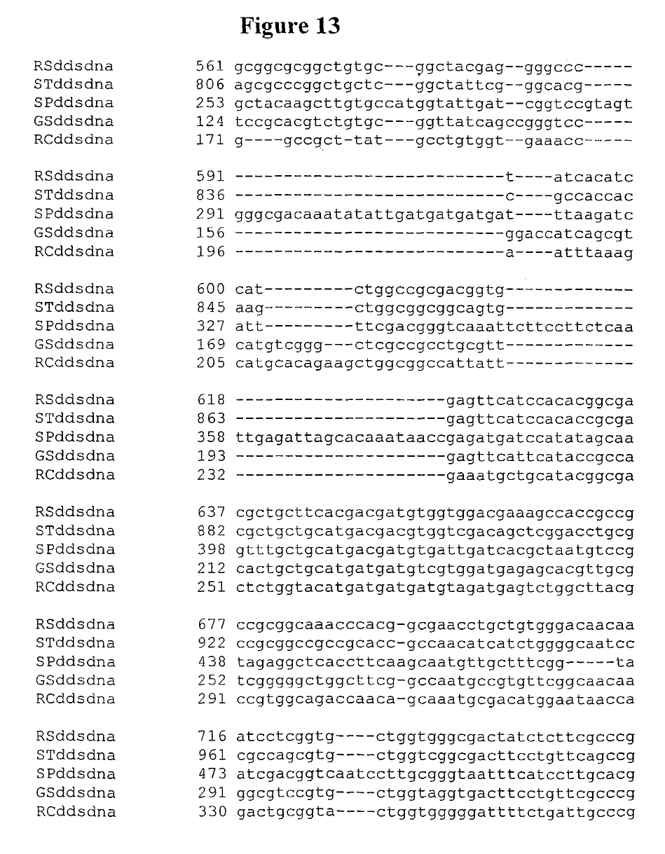

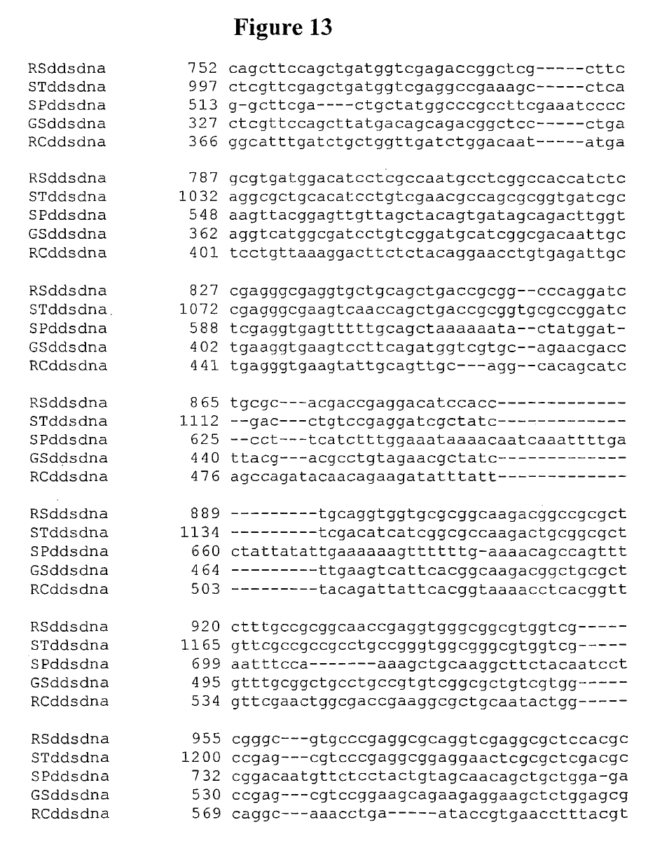

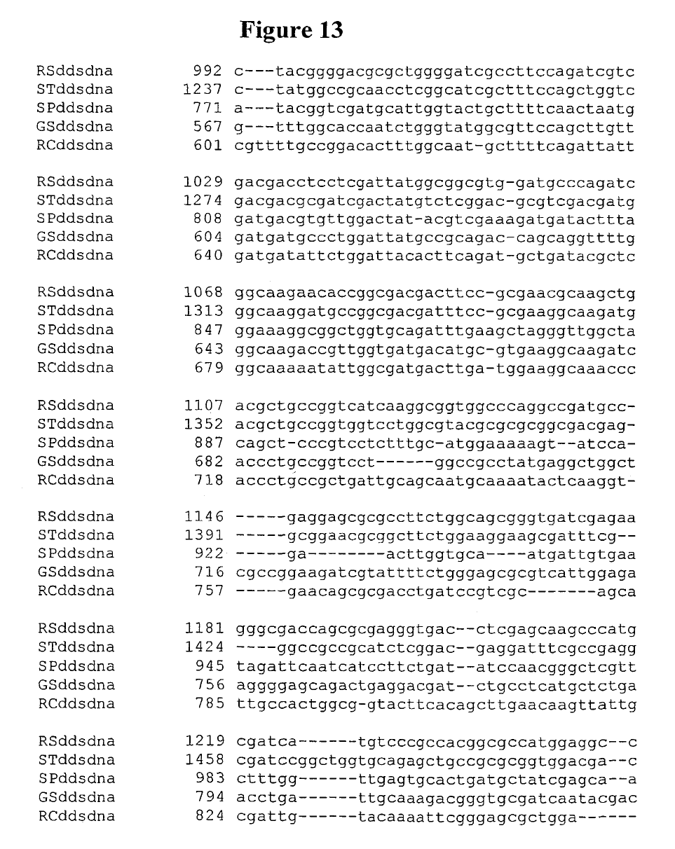

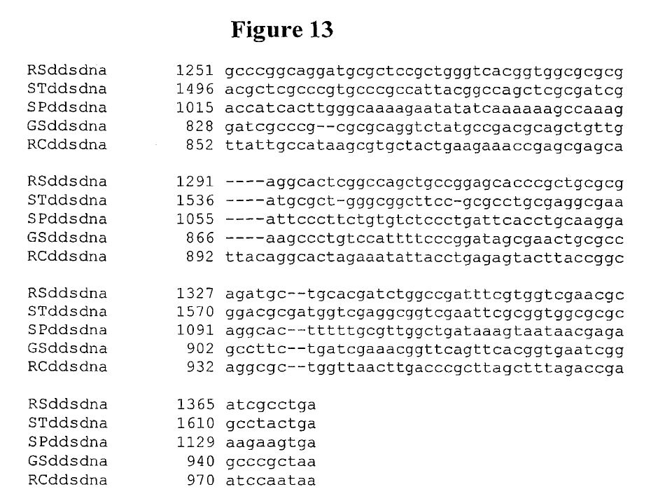

FIG. 13 depicts the nucleic acid sequence depicted in FIG. 8 (designated RSddsdna) and the nucleic acid sequence depicted in FIG. 11 (designated STddsdna) aligned with each other as well as aligned with three other nucleic acid sequences. Examples of variations of the RSddsdna sequence include, without limitation, any variation of the RSddsdna sequence provided in FIG. 13. Examples of variations of the STddsdna sequence include, without limitation, any variation of the STddsdna sequence provided in FIG. 13. Such variations are provided in FIG. 13 in that a comparison of the nucleotide (or lack thereof) at a particular position of the RSddsdna sequence or the STddsdna sequence with the nucleotide (or lack thereof) at the same position of any of the other nucleic acid sequences depicted in FIG. 13 provides a list of specific changes for the RSddsdna sequence and the STddsdna sequence. For example, the “a” at position 511 of the RSddsdna sequence or the “a” at position 756 of the STddsdna sequence can be substituted with an “t” as indicated in FIG. 13. Again, it will be appreciated that the RSddsdna sequence as well as the STddsdna sequence can contain any number of variations as well as any combination of types of variations. For example, the RSddsdna sequence can contain one variation provided in FIG. 13 or more than one (e.g., 2, 3, 4, 5, 6, 7, 8, 9, 10, 15, 20, 25, 50, 100, or more) of the variations provided in FIG. 13. Likewise, the STddsdna sequence can contain one variation provided in FIG. 13 or more than one (e.g., 2, 3, 4, 5, 6, 7, 8, 9, 10, 15, 20, 25, 50, 100, or more) of the variations provided in FIG. 13. It is noted that the full-length nucleic acid sequences depicted in FIG. 13 can encode polypeptides having DDS activity. It also is noted that the nucleic acid sequence depicted in FIG. 7 contains the nucleic acid sequence depicted in FIG. 8 and that the nucleic acid sequence depicted in FIG. 10 contains the nucleic acid sequence depicted in FIG. 11.

The nucleic acid sequence depicted in FIG. 7 contains a nucleic acid sequence that encodes a R. sphaeroides (ATCC 17023) polypeptide having DDS activity. Another variant of this nucleic acid sequence is the nucleic acid sequence of a clone isolated from R. sphaeroides (ATCC 35053). Briefly, a R. sphaeroides (ATCC 35053) clone was identified and found to contain a sequence identical to the nucleic acid sequence depicted in FIG. 7 with the following three exceptions. The R. sphaeroides (ATCC 35053) clone has a “t” at position 885 rather than a “c”, a “c” inserted after the “c” at position 1620, and a “c” inserted after the “c” at position 1733.

The nucleic acid depicted in FIG. 8 also contains a nucleic acid sequence that encodes a R. sphaeroides (ATCC 17023) polypeptide having DDS activity. Another variant of this nucleic acid sequence is the nucleic acid sequence of a clone isolated from R. sphaeroides (ATCC 35053). Briefly, a R. sphaeroides (ATCC 35053) clone was identified and found to contain a sequence identical to the nucleic acid sequence depicted in FIG. 8 with the following exception. The R. sphaeroides (ATCC 35053) clone has a “t” at position 514 rather than a “c”.

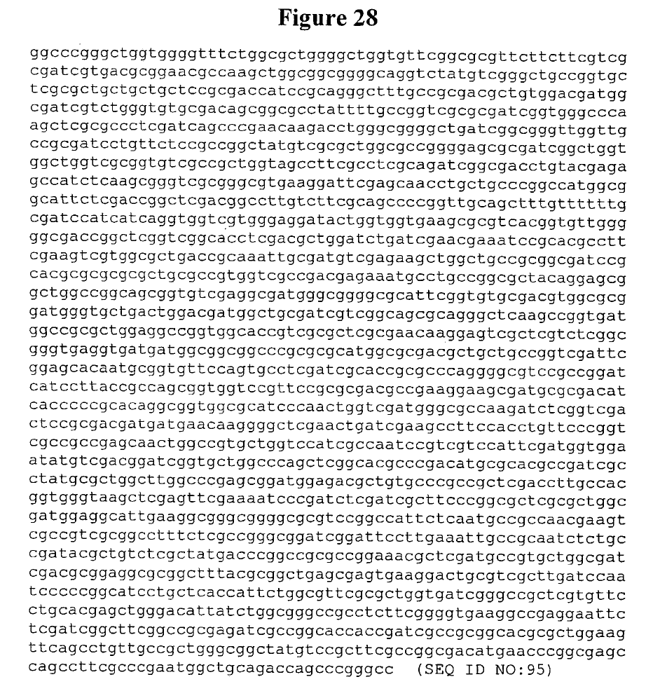

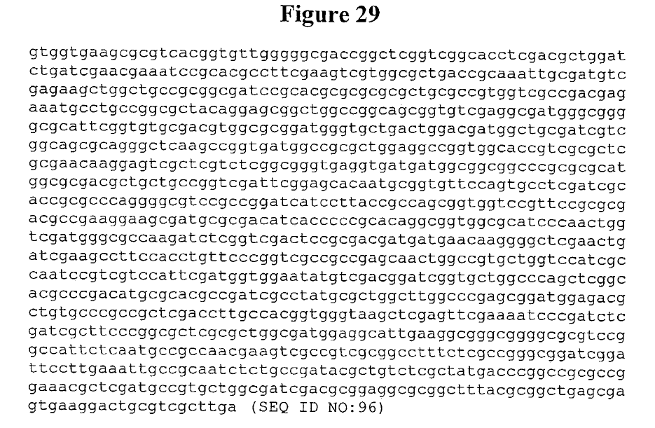

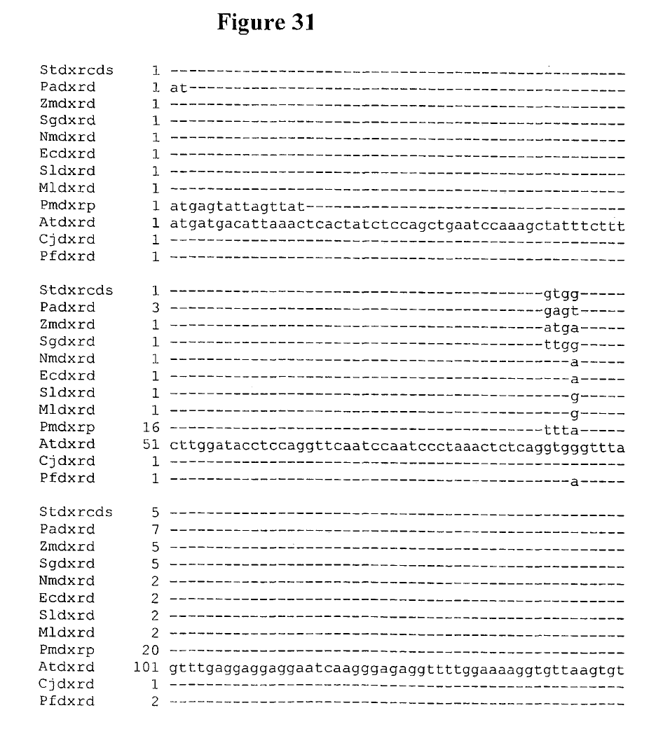

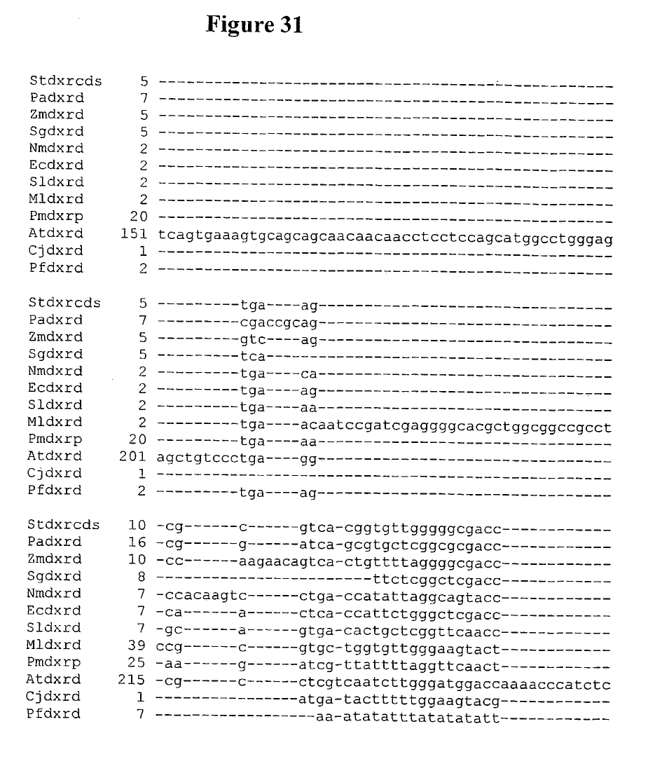

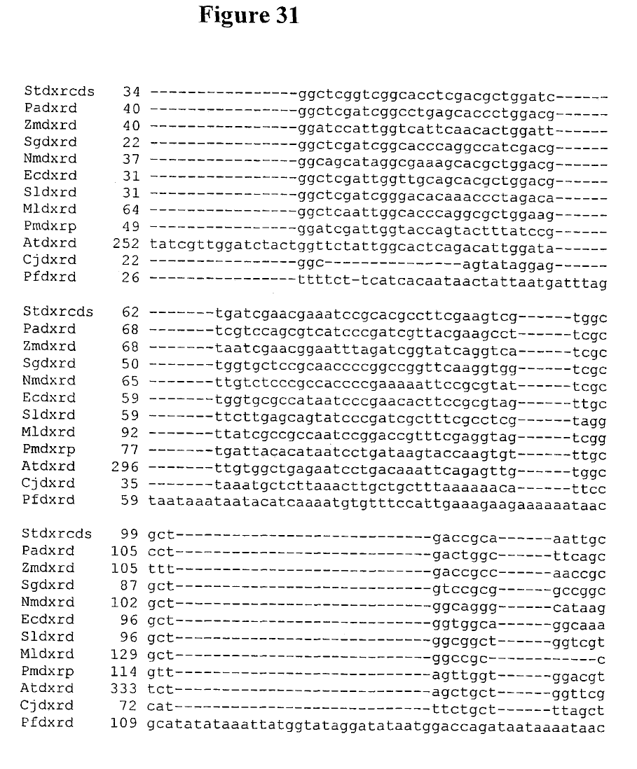

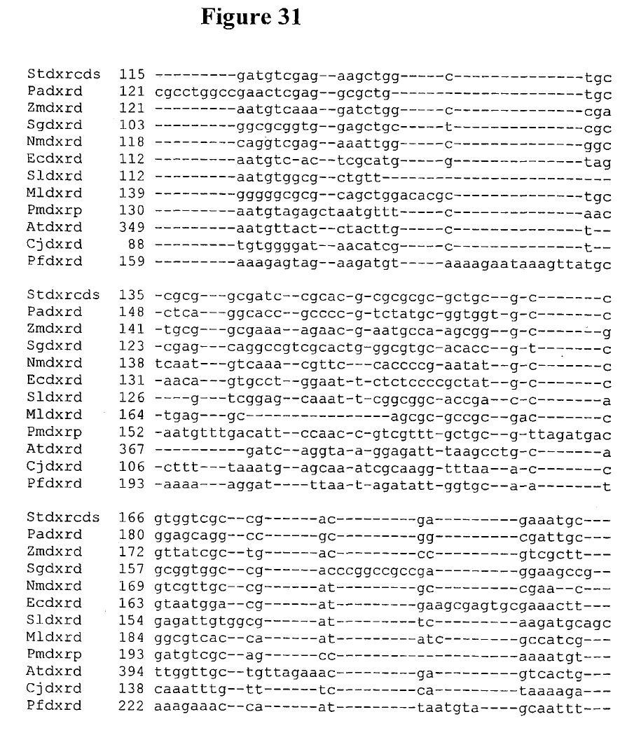

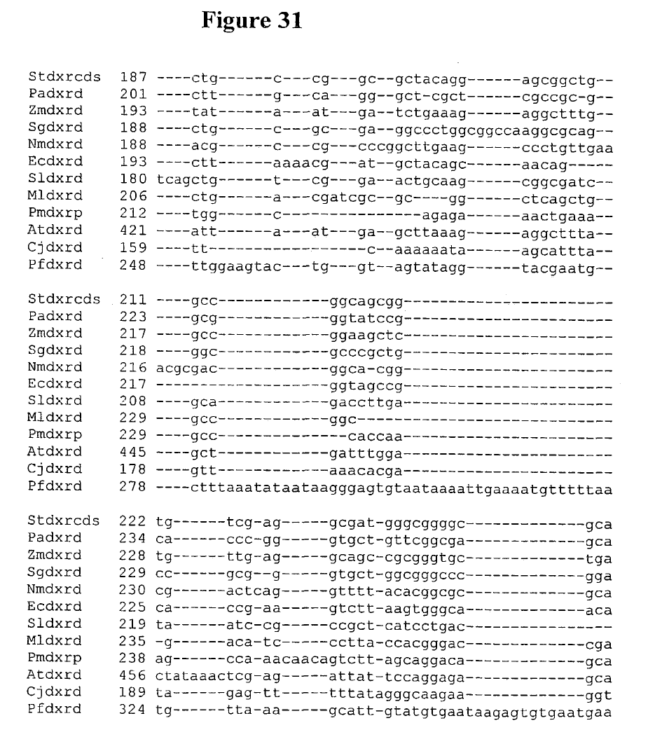

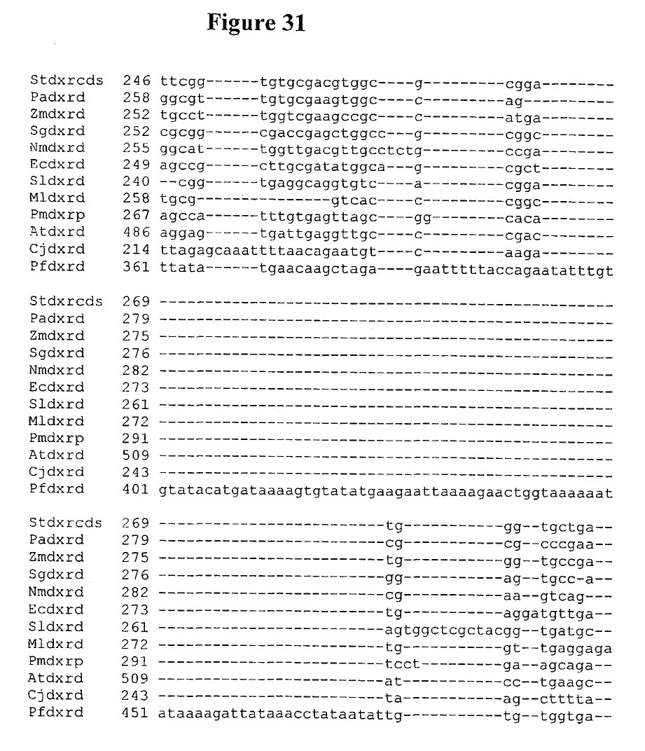

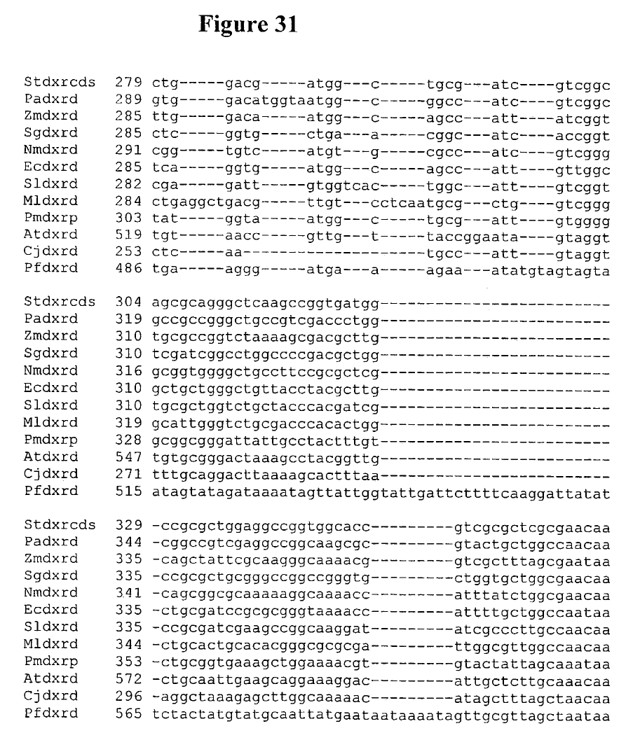

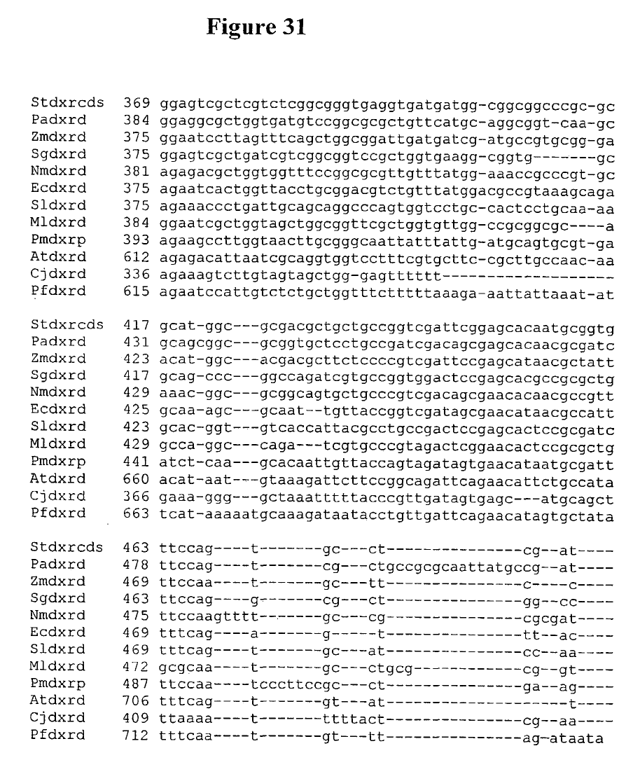

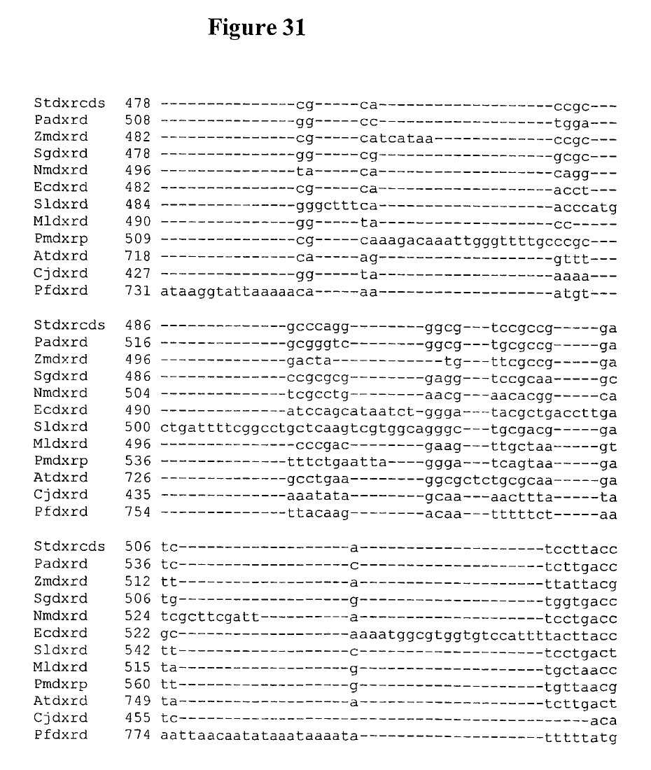

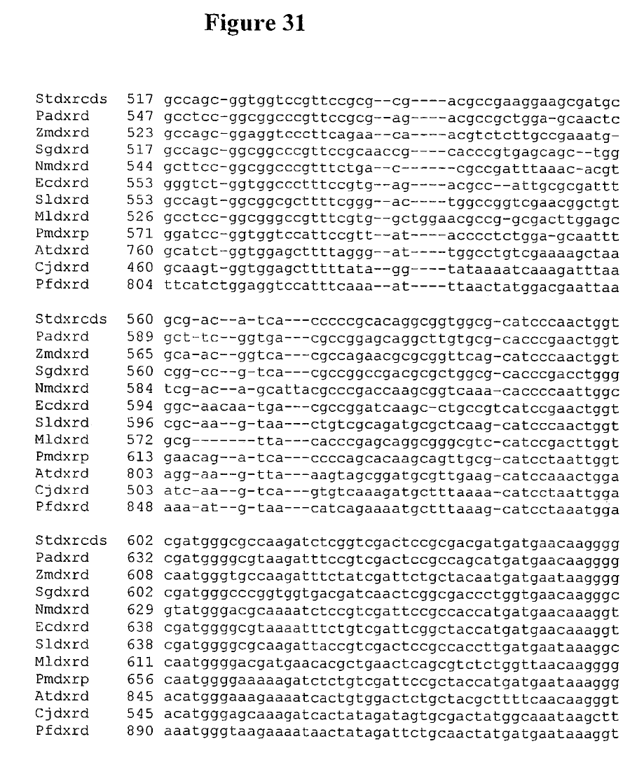

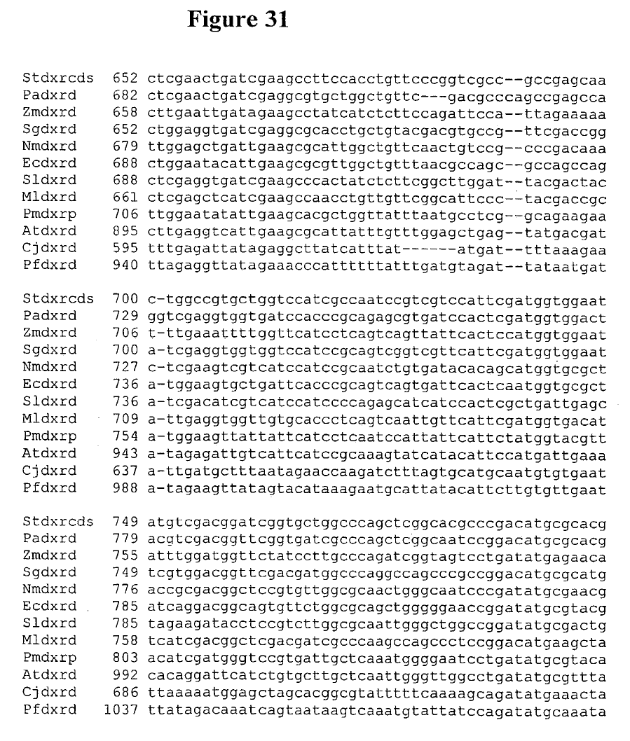

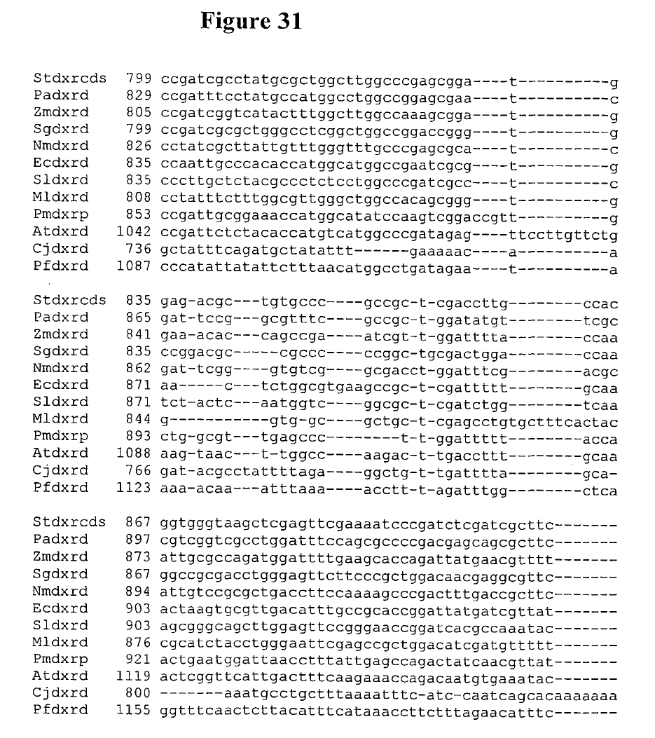

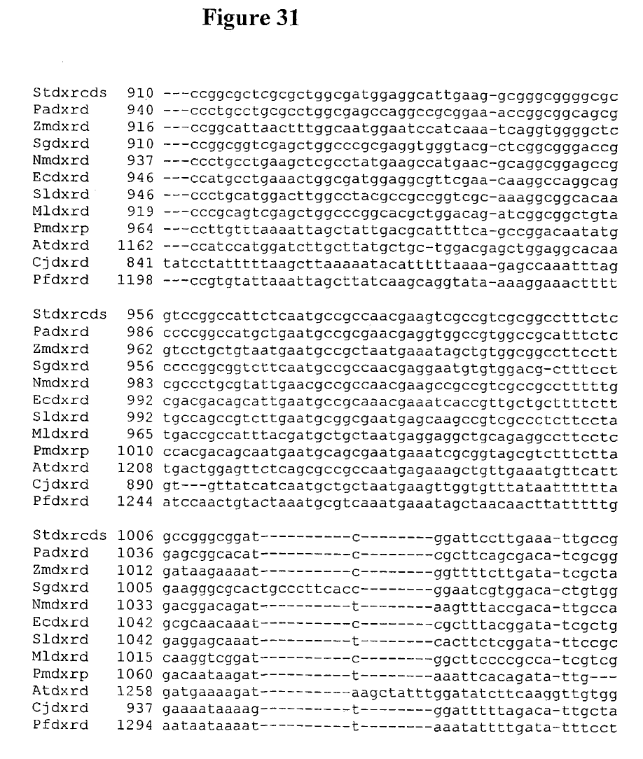

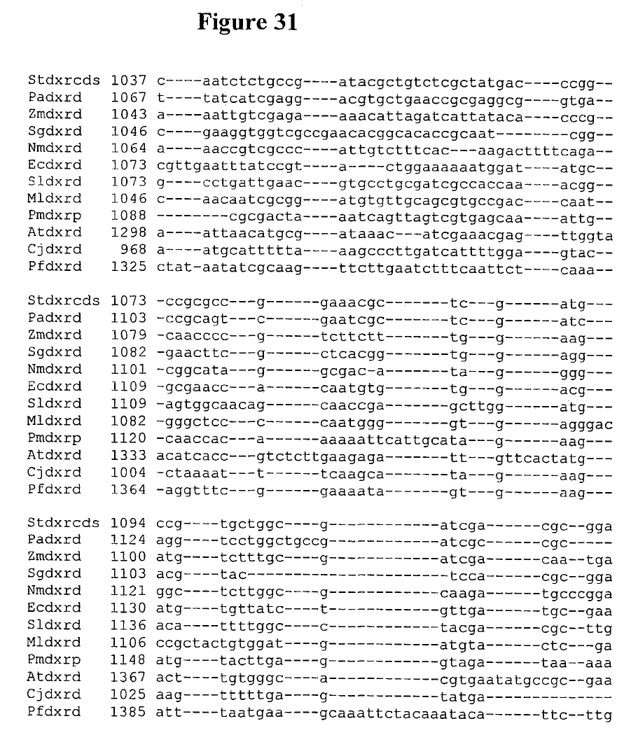

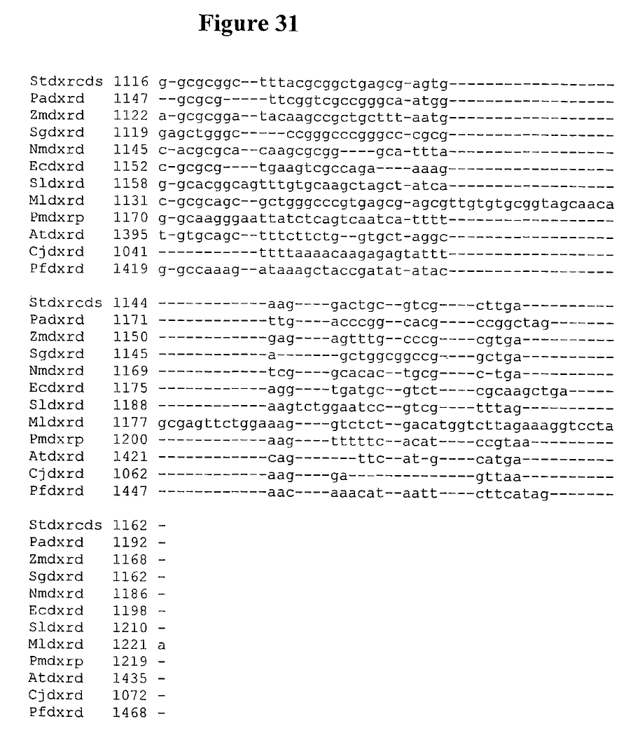

FIG. 31 depicts the nucleic acid sequence depicted in FIG. 29 (designated Stdxrcds) aligned with eleven other nucleic acid sequences. Examples of variations of the Stdxrcds sequence include, without limitation, any variation of the Stdxrcds sequence provided in FIG. 31. Such variations are provided in FIG. 31 in that a comparison of the nucleotide (or lack thereof) at a particular position of the Stdxrcds sequence with the nucleotide (or lack thereof) at the same position of any of the other nucleic acid sequences depicted in FIG. 31 provides a list of specific changes for the Stdxrcds sequence. Again, it will be appreciated that the Stdxrcds sequence can contain any number of variations as well as any combination of types of variations. For example, the Stdxrcds sequence can contain one variation provided in FIG. 31 or more than one (e.g., 2, 3, 4, 5, 6, 7, 8, 9, 10, 15, 20, 25, 50, 100, or more) of the variations provided in FIG. 31. It is noted that the full-length nucleic acid sequences depicted in FIG. 31 can encode polypeptides having DXR activity. It also is noted that the nucleic acid sequence depicted in FIG. 29 contains the nucleic acid sequence depicted in FIG. 28.

The invention also provides isolated nucleic acid that contains a variant of a portion of the nucleic acid sequence depicted in FIG. 2, 3, 7, 8, 10, 11, 28, or 29 as described herein.

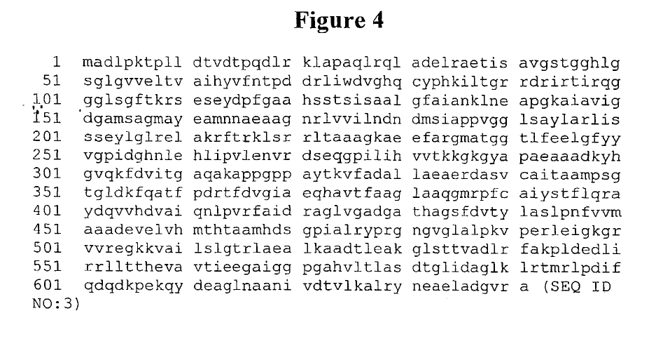

The invention provides isolated nucleic acid that contains a nucleic acid sequence that encodes the entire amino acid sequence depicted in FIG. 4, 9, 12, or 30. In addition, the invention provides isolated nucleic acid that contains a nucleic acid sequence that encodes a portion of the amino acid sequence depicted in FIG. 4, 9, 12, or 30. For example, the invention provides isolated nucleic acid that contains a nucleic acid sequence that encodes a 15 amino acid sequence identical to any 15 amino acid sequence depicted in FIG. 4, 9, 12, or 30 including, without limitation, the sequence starting at amino acid residue number 1 and ending at amino acid residue number 15, the sequence starting at amino acid residue number 2 and ending at amino acid residue number 16, the sequence starting at amino acid residue number 3 and ending at amino acid residue number 17, and so forth. It will be appreciated that the invention also provides isolated nucleic acid that contains a nucleic acid sequence that encodes an amino acid sequence that is greater than 15 amino acid residues (e.g., 16, 17, 1S, 19, 20, 21, 22, 23, 24, 25, 26, 27, 28, 29, 30, or more amino acid residues) in length and identical to any portion of the sequence depicted in FIG. 4, 9, 12, or 30. For example, the invention provides isolated nucleic acid that contains a nucleic acid sequence that encodes a 25 amino acid sequence identical to any 25 amino acid sequence depicted in FIG. 4, 9, 12, or 30 including, without limitation, the sequence starting at amino acid residue number 1 and ending at amino acid residue number 25, the sequence starting at amino acid residue number 2 and ending at amino acid residue number 26, the sequence starting at amino acid residue number 3 and ending at amino acid residue number 27, and so forth. Additional examples include, without limitation, isolated nucleic acids that contain a nucleic acid sequence that encodes an amino acid sequence that is 50 or more amino acid residues (e.g., 100, 150, 200, 250, 300, 350, or more amino acid residues) in length and identical to any portion of the sequence depicted in FIG. 4, 9, 12, or 30. Such isolated nucleic acids can include, without limitation, those isolated nucleic acids containing a nucleic acid sequence that encodes an amino acid sequence represented in a single line of sequence depicted in FIG. 4, 9, 12, or 30 since each line of sequence depicted in these figures, with the exception of the last line, provides a 50 amino acid sequence.

In addition, the invention provides isolated nucleic acid that contains a nucleic acid sequence that encodes an amino acid sequence having a variation of the amino acid sequence depicted in FIG. 4, 9, 12, or 30. For example, the invention provides isolated nucleic acid containing a nucleic acid sequence encoding an amino acid sequence depicted in FIG. 4, 9, 12, or 30 that contains a single insertion, a single deletion, a single substitution, multiple insertions, multiple deletions, multiple substitutions, or any combination thereof (e.g., single deletion together with multiple insertions). The invention provides multiple examples of isolated nucleic acid containing a nucleic acid sequence encoding an amino acid sequence having a variation of an amino acid sequence depicted in FIG. 4, 9, 12, or 30.

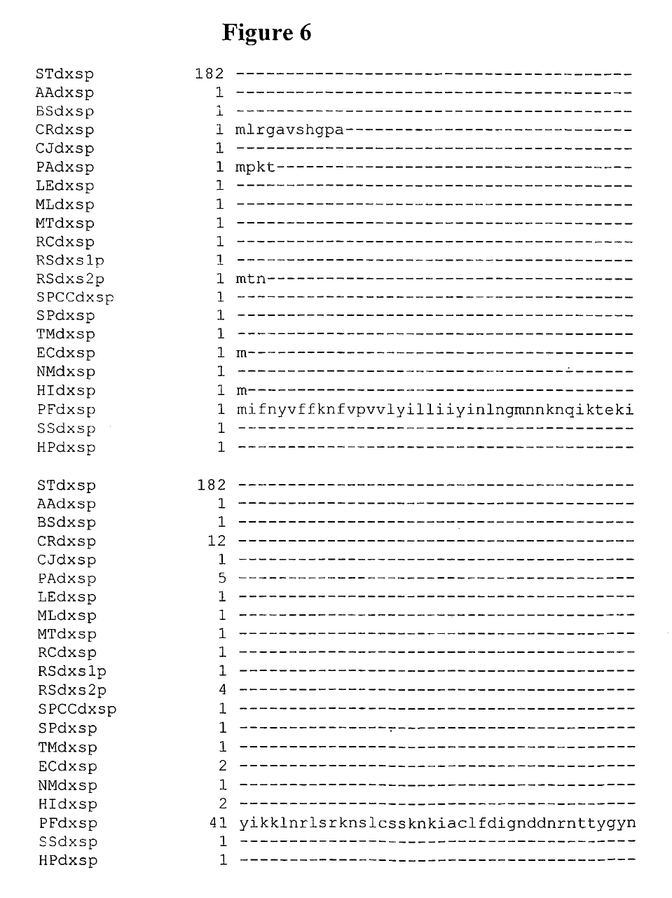

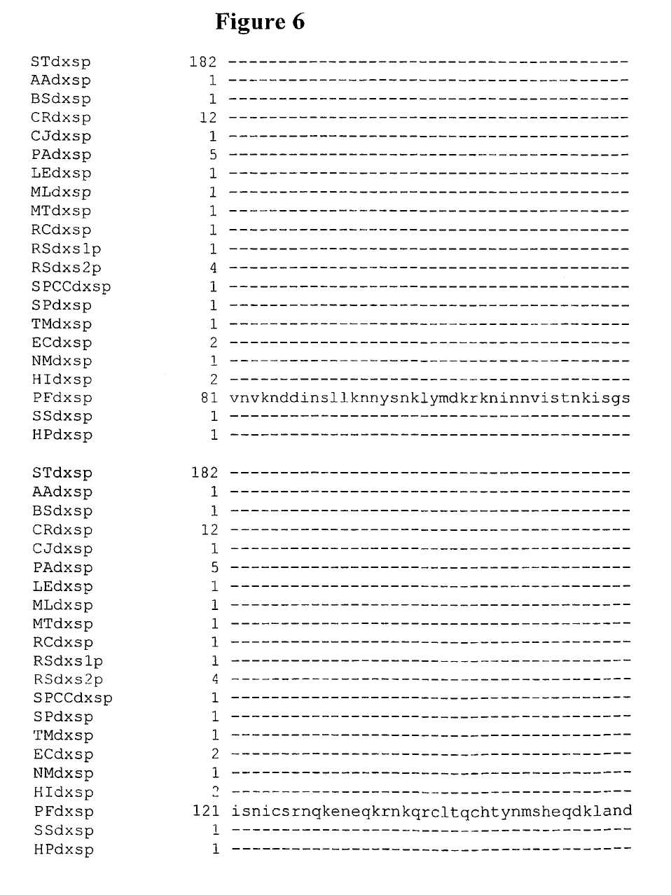

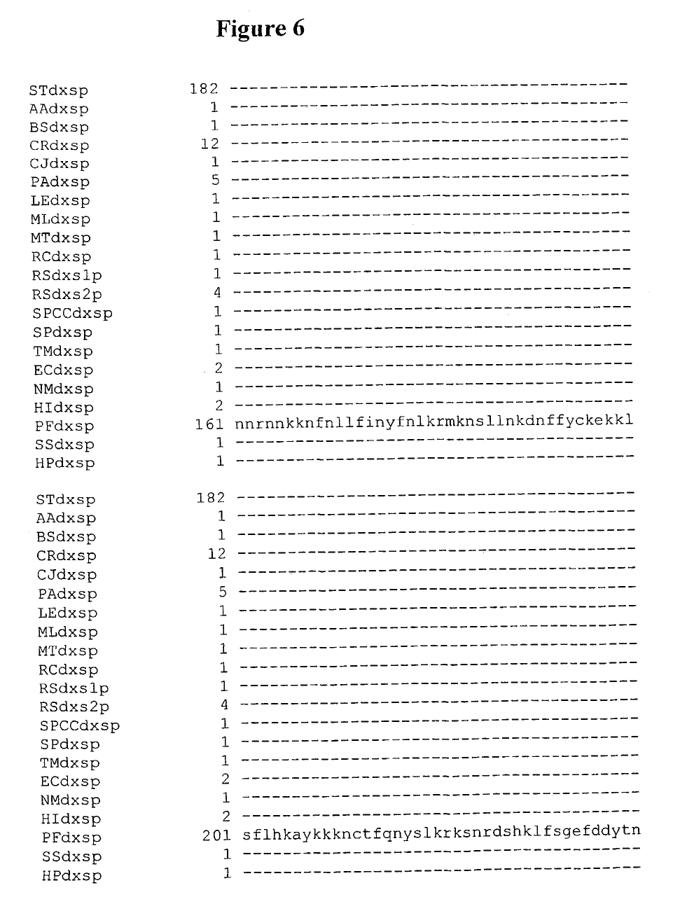

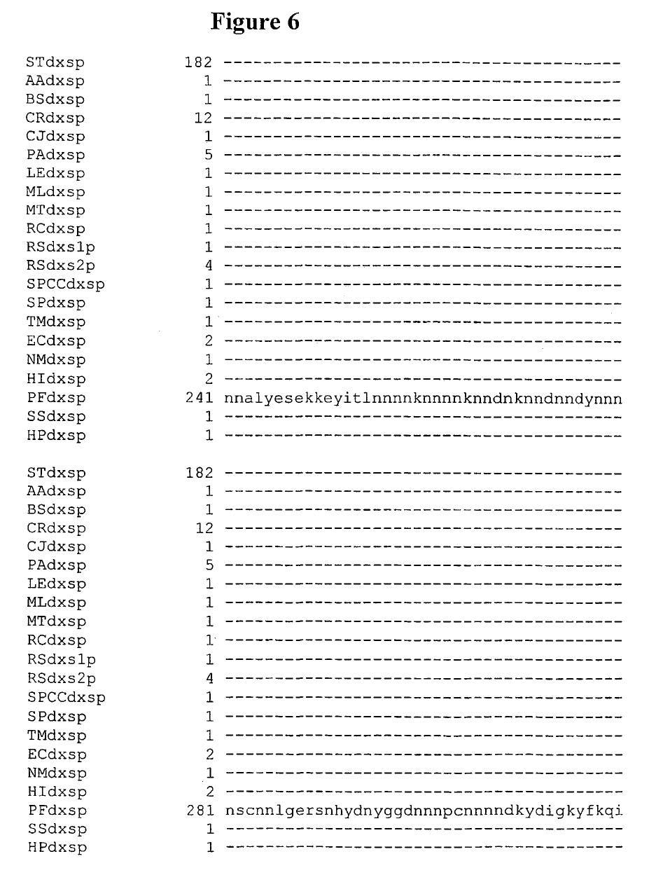

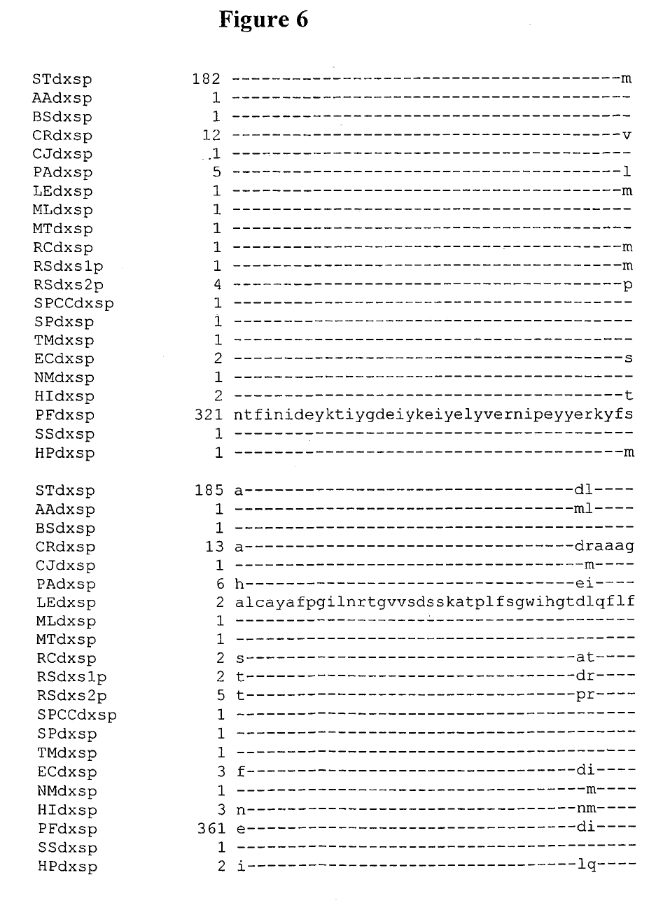

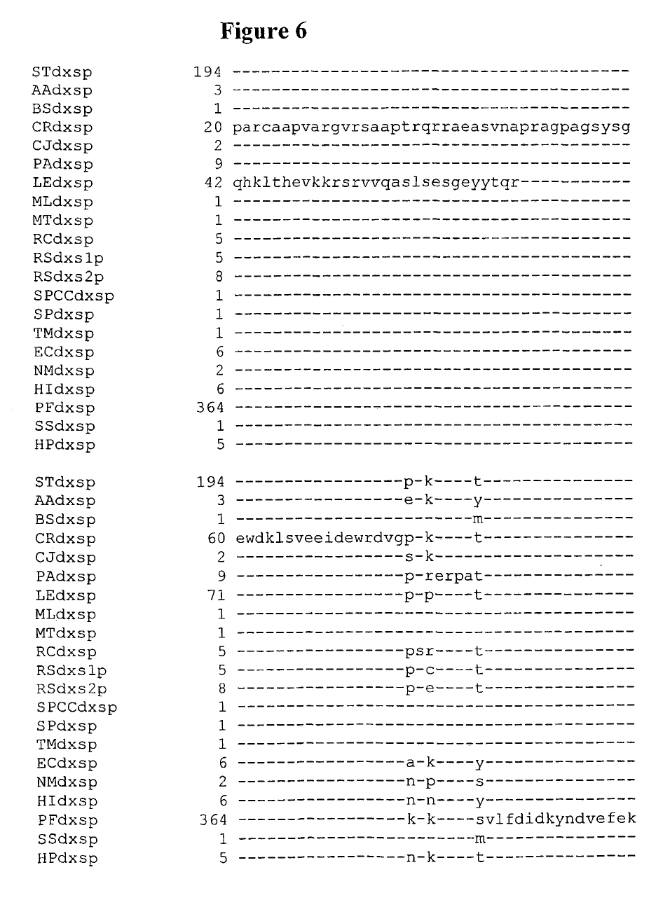

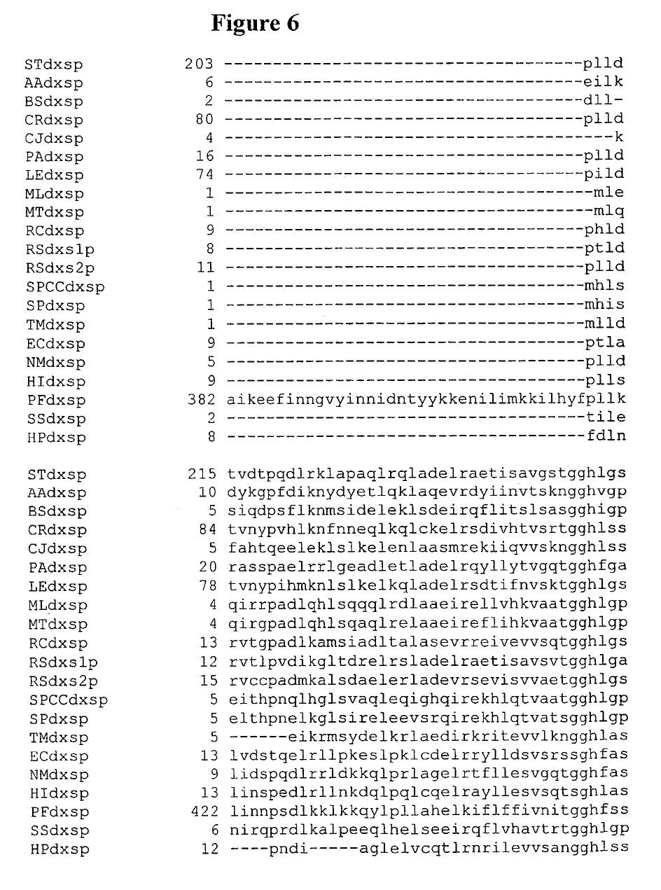

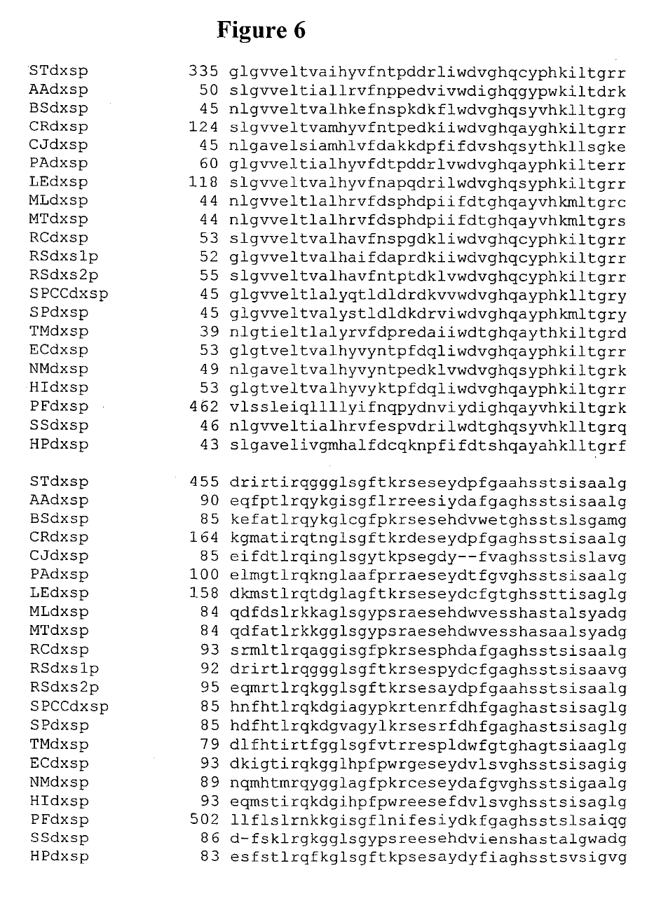

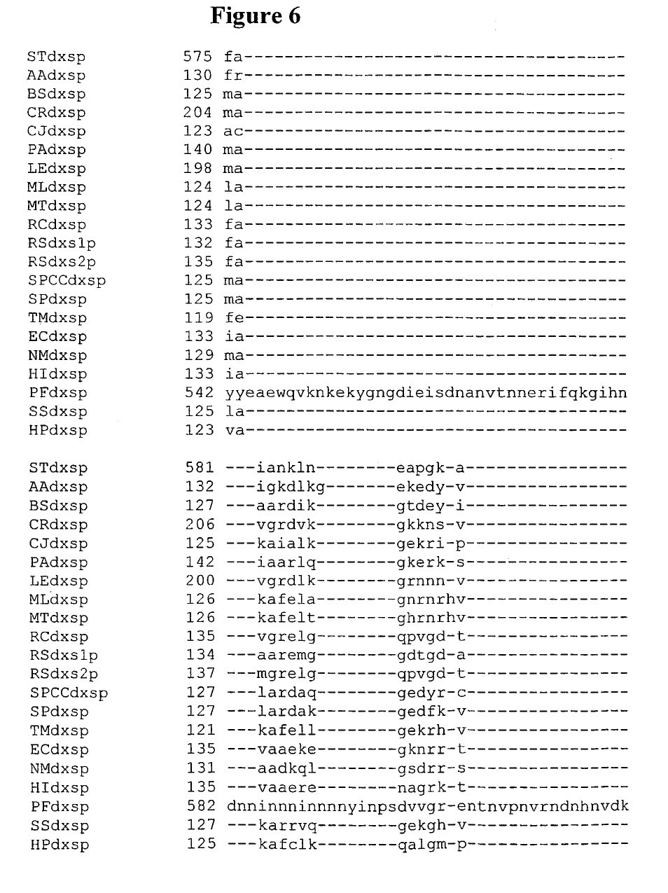

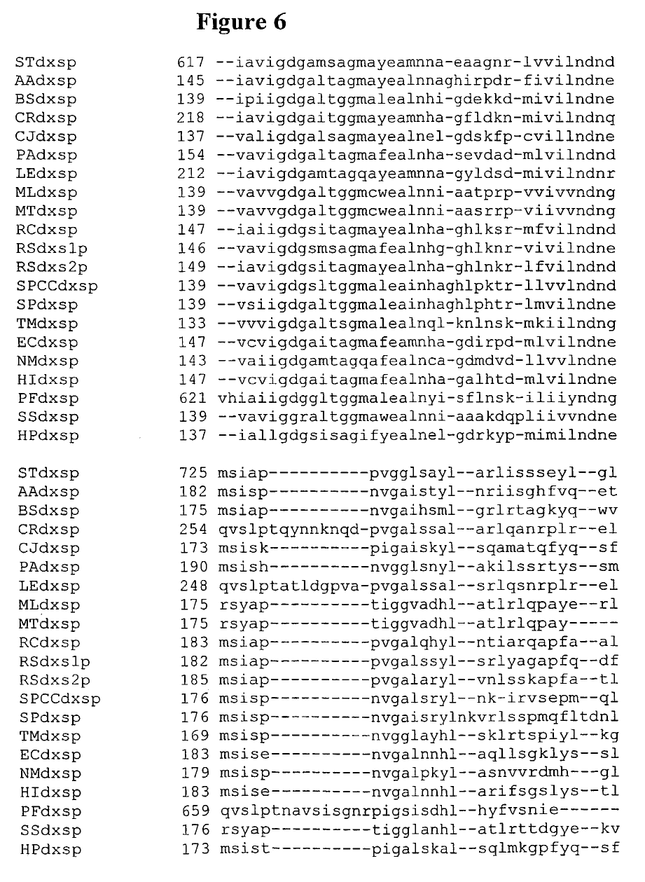

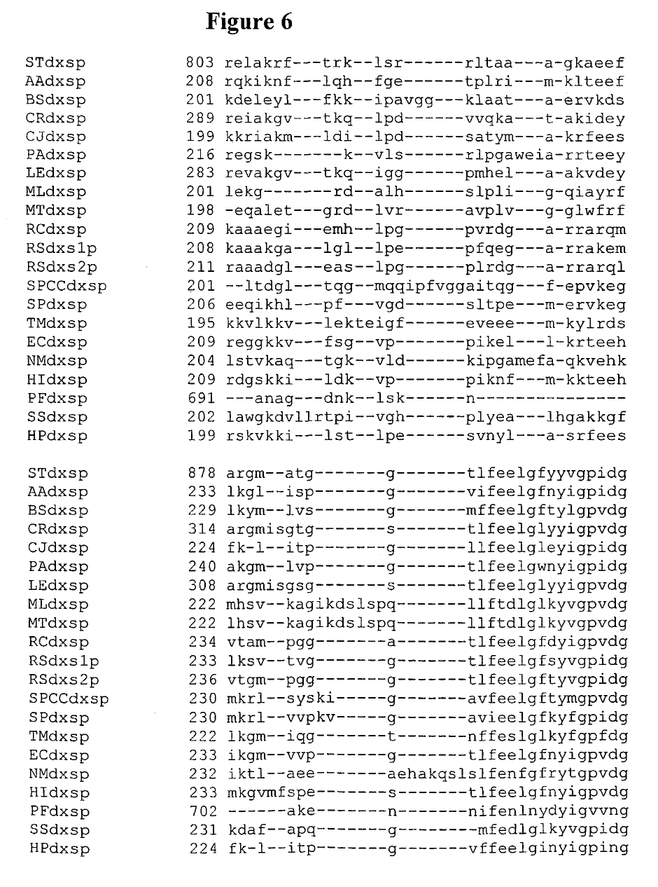

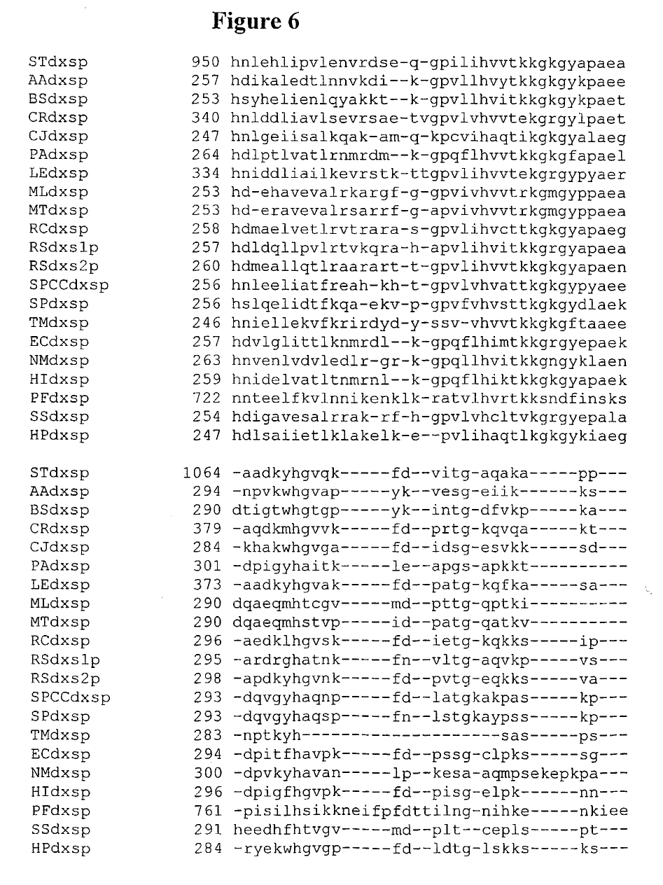

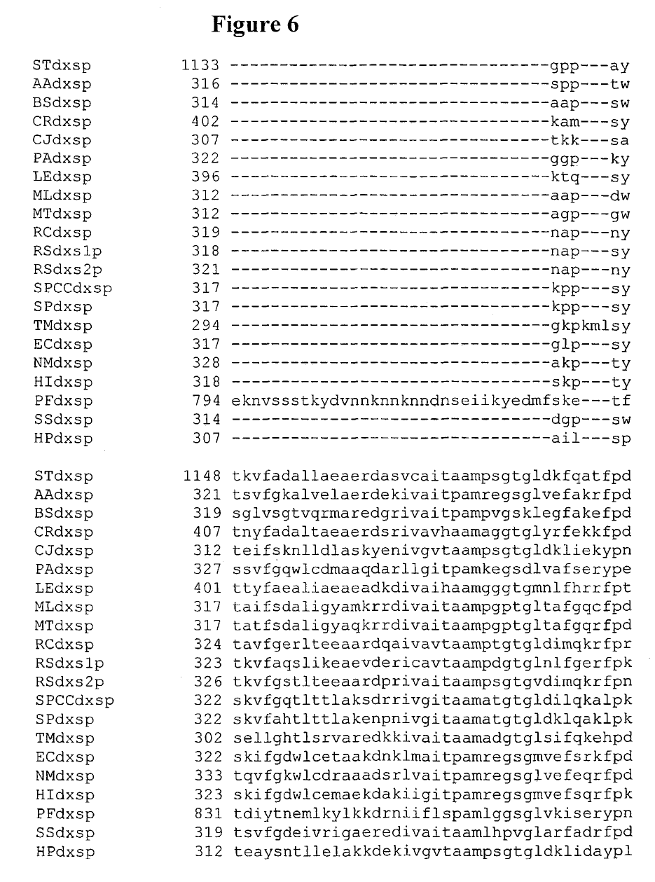

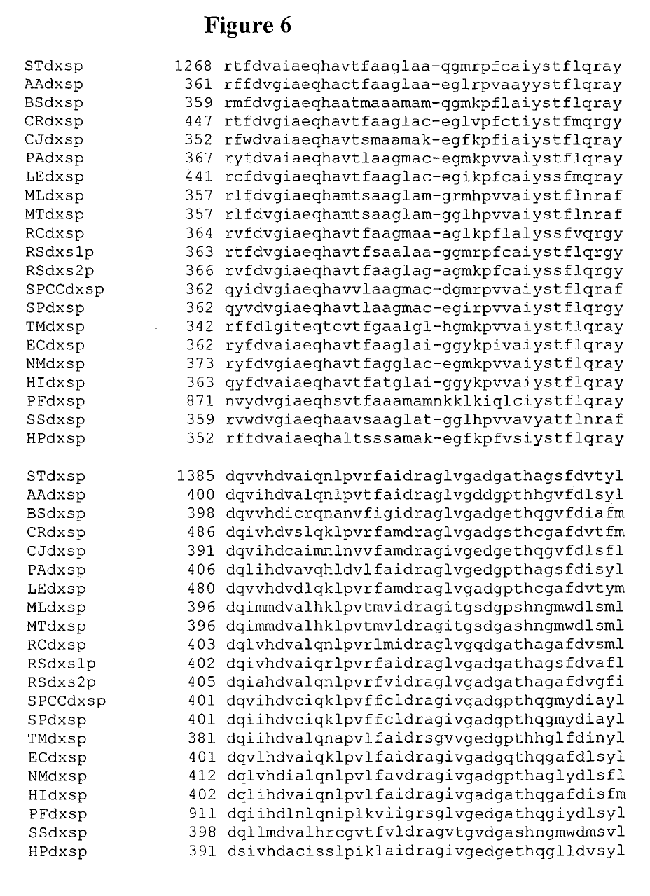

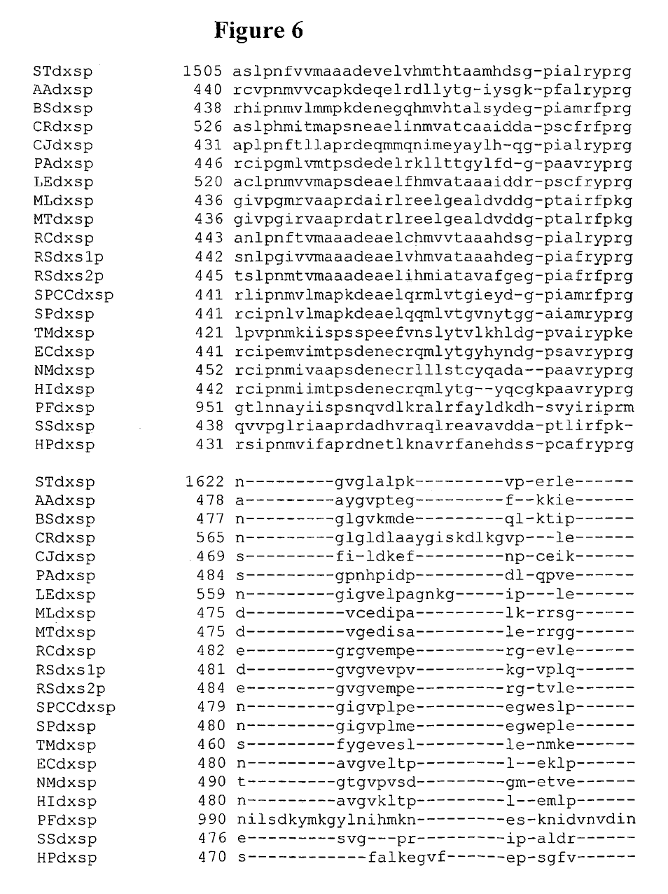

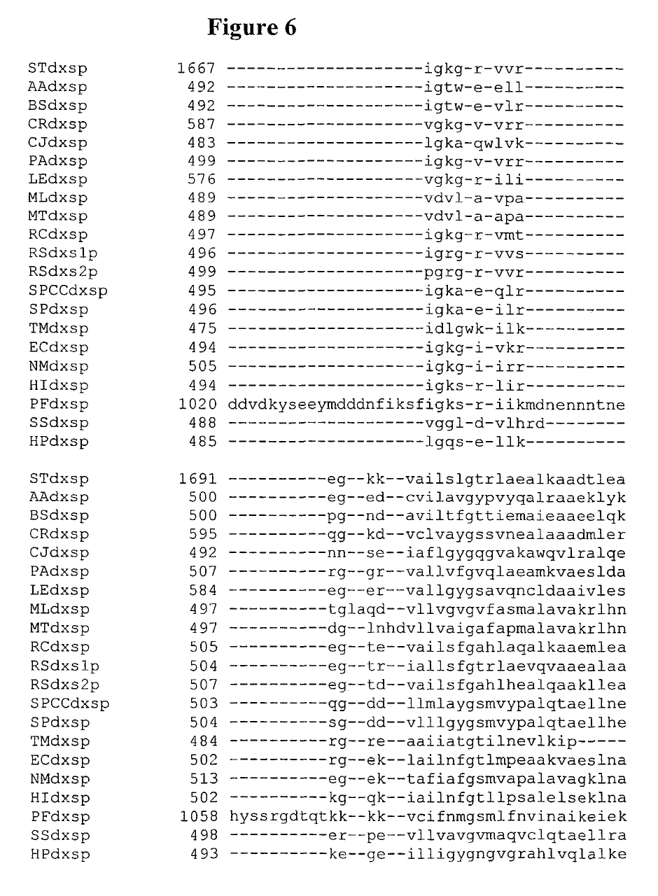

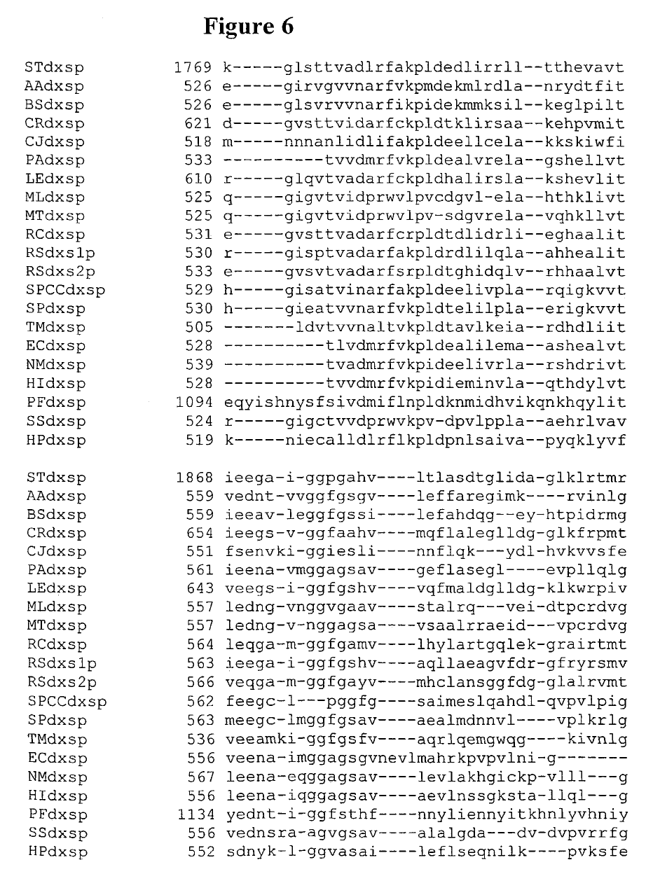

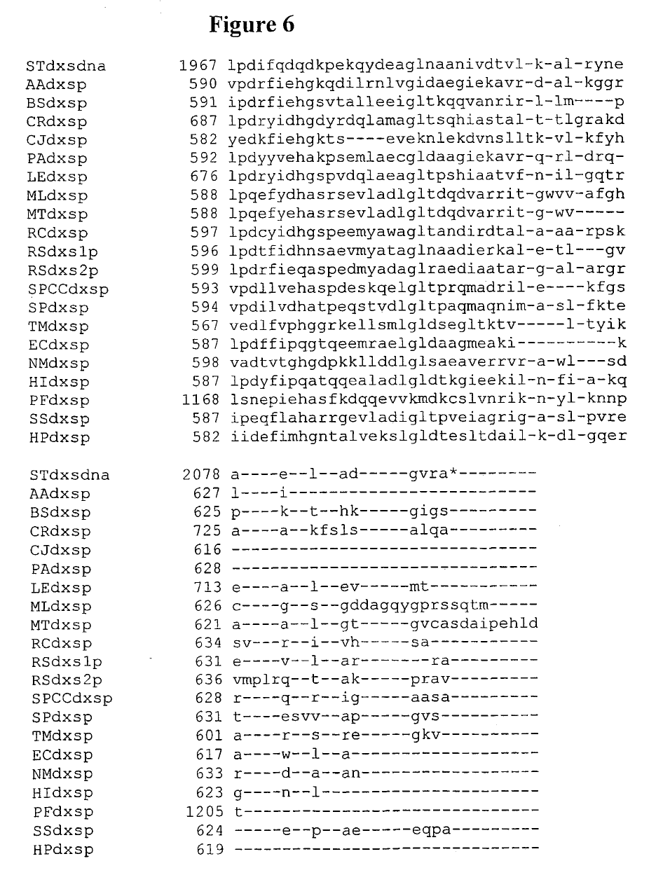

FIG. 6 depicts the amino acid sequence depicted in FIG. 4 (designated STdxsp) aligned with 20 other amino acid sequences. Examples of variations of the STdxsp sequence include, without limitation, any variation of the STdxsp sequence provided in FIG. 6. Such variations are provided in FIG. 6 in that a comparison of the amino acid residue (or lack thereof) at a particular position of the STdxsp sequence with the amino acid residue (or lack thereof) at the same position of any of the other 20 amino acid sequences depicted in FIG. 6 provides a list of specific changes for the STdxsp sequence. For example, the “t” at position 1148 of the STdxsp sequence can be substituted with an “s” as indicated in FIG. 6. As also indicated in FIG. 6, the “f” at position 575 of the STdxsp sequence can be substituted with an “m”, “a”, “l”, “i”, “y”, or “v”. For FIG. 6, the nucleic acid numbering of FIG. 2 is used to number the amino acid residue positions of the STdxsp sequence. Thus, the first amino acid residue of the STdxsp sequence starts with number 182 and proceeds in increments of three. It will be appreciated that the STdxsp sequence can contain any number of variations as well as any combination of types of variations. For example, the STdxsp sequence can contain one variation provided in FIG. 6 or more than one (e.g., 2, 3, 4, 5, 6, 7, 8, 9, 10, 15, 20, 25, 50, 100, or more) of the variations provided in FIG. 6. It is noted that the 21 full-length amino acid sequences depicted in FIG. 6 can be polypeptides having DXS activity.

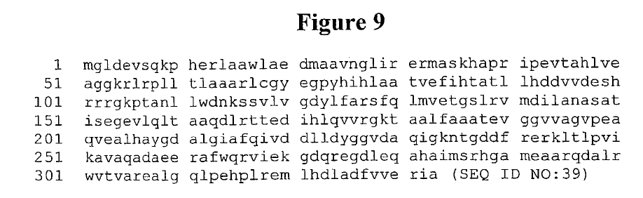

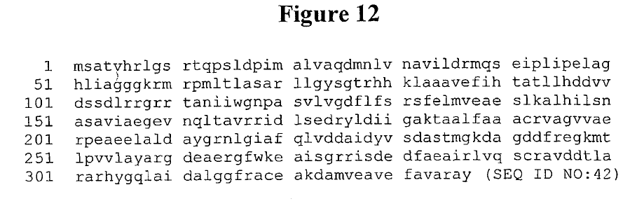

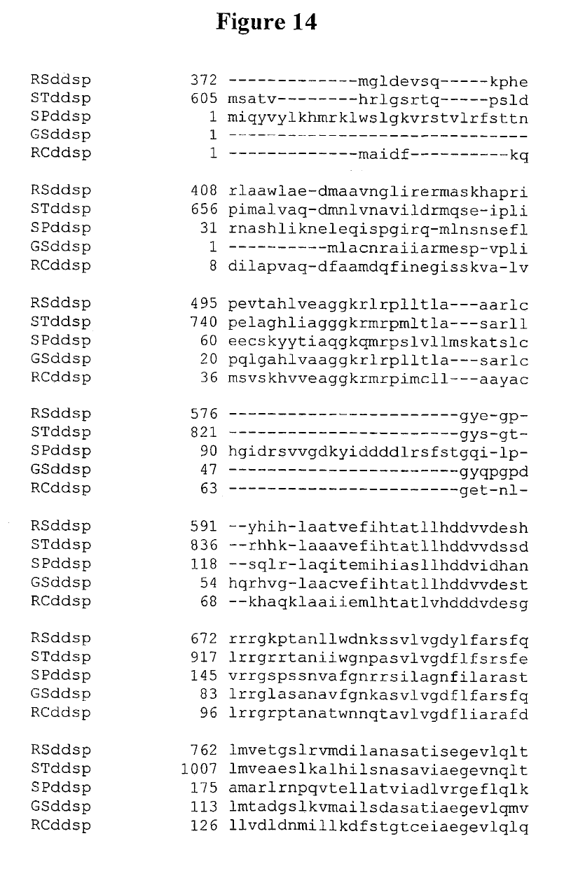

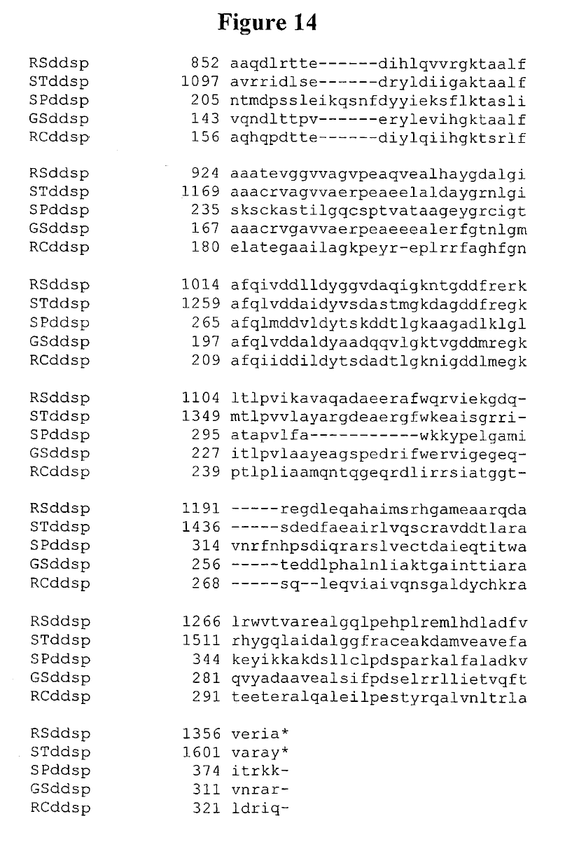

FIG. 14 depicts the amino acid sequence depicted in FIG. 9 (designated RSddsp) and the amino acid sequence depicted in FIG. 12 (designated STddsp) aligned with each other as well as aligned with three other amino acid sequences. For FIG. 14, the nucleic acid numbering of FIG. 7 is used to number the amino acid residue positions of the RSddsp sequence, and the nucleic acid numbering of FIG. 10 is used to number the amino acid residue positions of the STddsp sequence. Thus, the first amino acid residue of the RSddsp and STddsp sequences each start with a number other than 1 and proceed in increments of three. Examples of variations of the RSddsp sequence include, without limitation, any variation of the RSddsp sequence provided in FIG. 14. Examples of variations of the STddsp sequence include, without limitation, any variation of the STddsp sequence provided in FIG. 14. Such variations are provided in FIG. 14 in that a comparison of the amino acid residue (or lack thereof) at a particular position of the RSddsp sequence or the STddsp sequence with the amino acid residue (or lack thereof) at the same position of any of the other amino acid sequences depicted in FIG. 14 provides a list of specific changes for the RSddsp sequence and the STddsp sequence. For example, the “1” at position 762 of the RSddsp sequence or the “1” at position 1007 of the STddsp sequence can be substituted with an “a” as indicated in FIG. 14. Again, it will be appreciated that the RSddsp sequence as well as the STddsp sequence can contain any number of variations as well as any combination of types of variations. For example, the RSddsp sequence can contain one variation provided in FIG. 14 or more than one (e.g., 2, 3, 4, 5, 6, 7, 8, 9, 10, 15, 20, 25, 50, 100, or more) of the variations provided in FIG. 14. Likewise, the STddsp sequence can contain one variation provided in FIG. 14 or more than one (e.g., 2, 3, 4, 5, 6, 7, 8, 9, 10, 15, 20, 25, 50, 100, or more) of the variations provided in FIG. 14. It is noted that the five full-length amino acid sequences depicted in FIG. 14 can be polypeptides having DDS activity.

The amino acid sequence depicted in FIG. 9 represents a R. sphaeroides (ATCC 17023) polypeptide having DDS activity. Another variant of this amino acid sequence is the amino acid sequence encoded by a clone isolated from R. sphaeroides (ATCC 35053). Briefly, a R. sphaeroides (ATCC 35053) clone was identified and found to encode an amino acid sequence identical to the amino acid sequence depicted in FIG. 9 with the following exception. The R. sphaeroides (ATCC 35053) clone has a “y” at position 172 rather than an “h”.

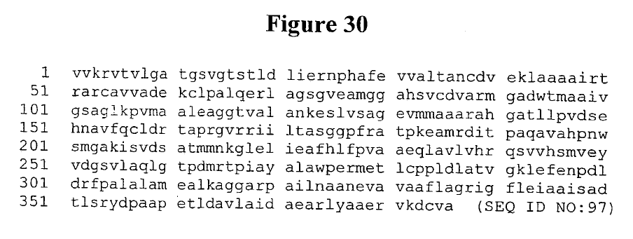

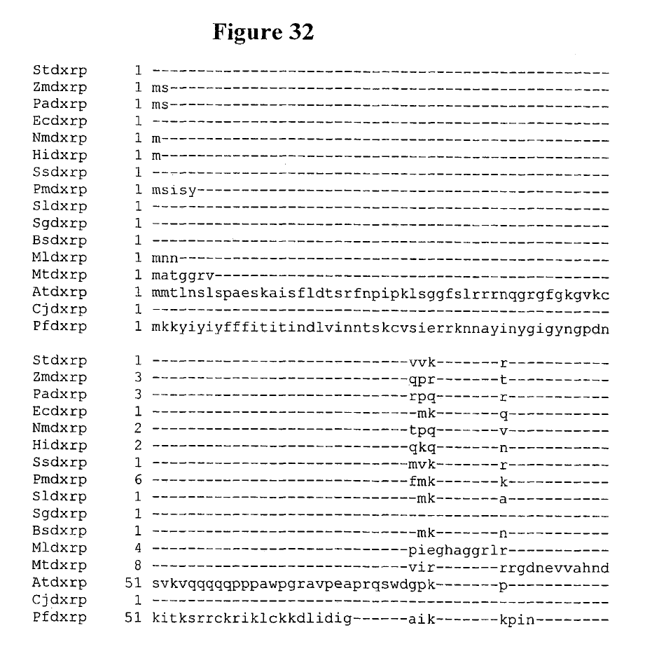

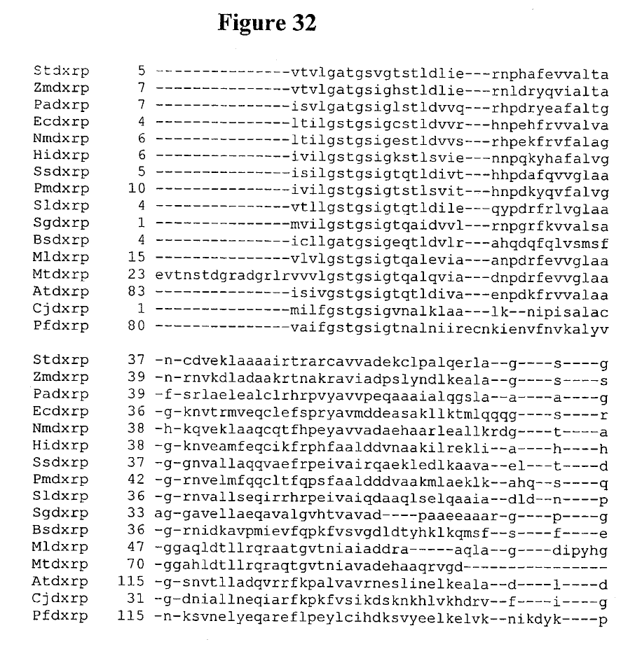

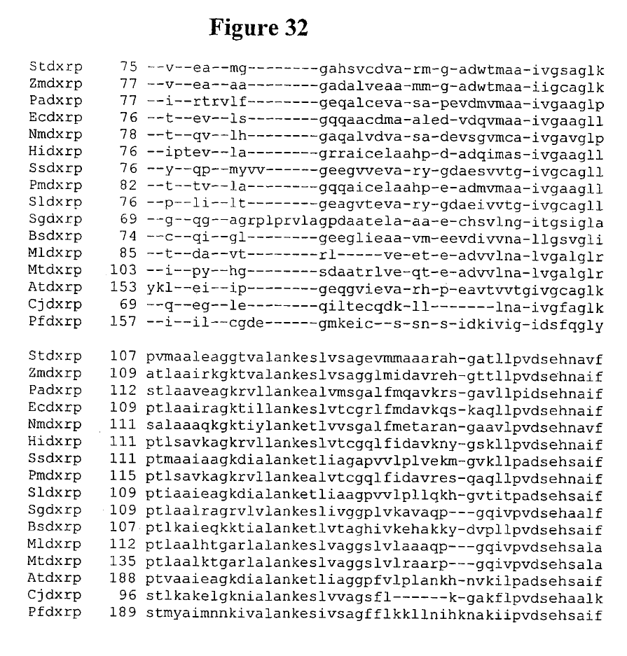

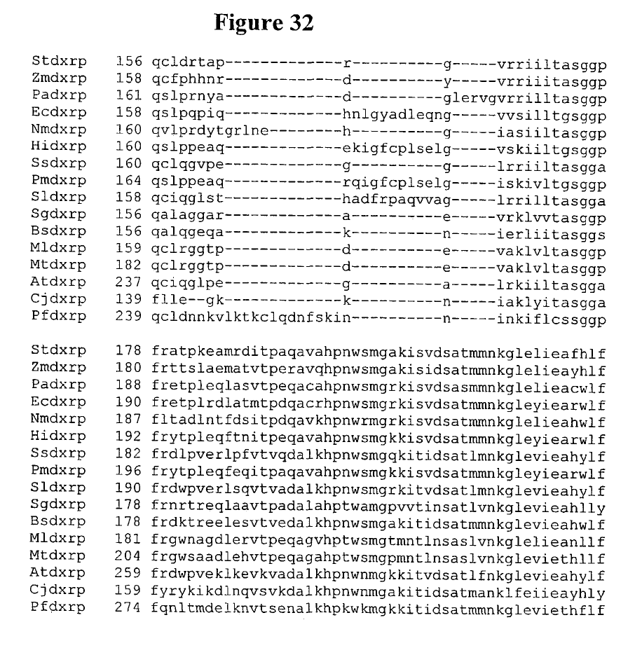

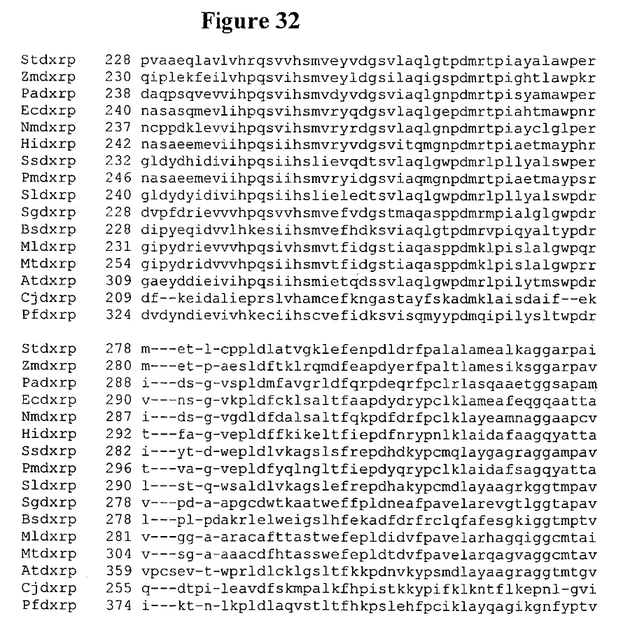

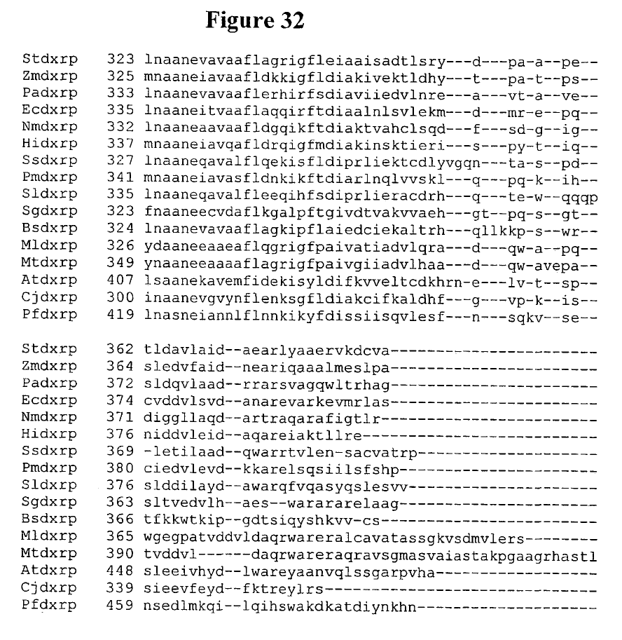

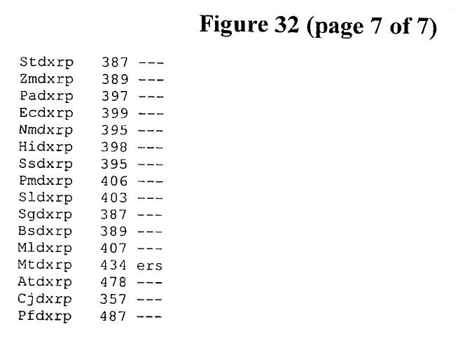

FIG. 32 depicts the amino acid sequence depicted in FIG. 30 (designated Stdxrp) aligned with 15 other amino acid sequences. Examples of variations of the Stdxrp sequence include, without limitation, any variation of the Stdxrp sequence provided in FIG. 32. Such variations are provided in FIG. 32 in that a comparison of the amino acid residue (or lack thereof) at a particular position of the Stdxrp sequence with the amino acid residue (or lack thereof) at the same position of any of the other 15 amino acid sequences depicted in FIG. 32 provides a list of specific changes for the Stdxrp sequence. It will be appreciated that the Stdxrp sequence can contain any number of variations as well as any combination of types of variations. For example, the Stdxrp sequence can contain one variation provided in FIG. 32 or more than one (e.g., 2, 3, 4, 5, 6, 7, 8, 9, 10, 15, 20, 25, 50, 100, or more) of the variations provided in FIG. 32. It is noted that the full-length amino acid sequences depicted in FIG. 32 can be polypeptides having DXR activity.

The invention also provides isolated nucleic acid containing a nucleic acid sequence encoding an amino acid sequence that contains a variant of a portion of the amino acid sequence depicted in FIG. 4, 9, 12, or 30 as described herein.

2. Polypeptides

The invention provides substantially pure polypeptides. The term “substantially pure” as used herein with reference to a polypeptide means the polypeptide is substantially free of other polypeptides, lipids, carbohydrates, and nucleic acid with which it is naturally associated. Thus, a substantially pure polypeptide is any polypeptide that is removed from its natural environment and is at least 60 percent pure. A substantially pure polypeptide can be at least about 65, 70, 75, 80, 85, 90, 95, or 99 percent pure. Typically, a substantially pure polypeptide will yield a single major band on a non-reducing polyacrylamide gel.

Any substantially pure polypeptide having an amino acid sequence encoded by a nucleic acid within the scope of the invention is itself within the scope of the invention. In addition, any substantially pure polypeptide containing an amino acid sequence having a length and a percent identity to the sequence set forth in SEQ ID NO:3 over that length as determined herein is within the scope of the invention provided the point defined by that length and percent identity is within the area defined by points A, B, C, and D of FIG. 26; where point A has an X coordinate less than or equal to 641, and a Y coordinate less than or equal to 100; where point B has an X coordinate less than or equal to 641, and a Y coordinate greater than or equal to 50; where point C has an X coordinate greater than or equal to 25, and a Y coordinate greater than or equal to 50; and where point D has an X coordinate greater than or equal to 5, and a Y coordinate less than or equal to 100. For example, the X coordinate for point A can be 641, 635, 630, 625, 620, or less; and the Y coordinate for point A can be 100, 99, 95, 90, 85, 80, 75, or less. The X coordinate for point B can be 641, 635, 630, 625, 620, or less; and the Y coordinate for point B can be 50, 55, 60, 65, 70, 75, 80, 85, 90, 95, 99 or more. The X coordinate for point C can be 25, 30, 35, 40, 50, 60, 70, 80, 90, 100, 150, 200, or more; and the Y coordinate for point C can be 50, 55, 60, 65, 70, 75, 80, 85, 90, 95, 99 or more. The X coordinate for point D can be 5, 6, 7, 8, 9, 10, 15, 20, 25, 30, 40, 50, 75, 100, or more; and the Y coordinate for point D can be 100, 99, 95, 90, 85, 80, 75, or less. In one embodiment, point A can be (641, 100), point B can be (641, 95), point C can be (400, 95), and point D can be (400, 100).

Any substantially pure polypeptide containing an amino acid sequence having a length and a percent identity to the sequence set forth in SEQ ID NO:39 over that length as determined herein is within the scope of the invention provided the point defined by that length and percent identity is within the area defined by points A, B, C, and D of FIG. 26; where point A has an X coordinate less than or equal to 333, and a Y coordinate less than or equal to 100; where point B has an X coordinate less than or equal to 333, and a Y coordinate greater than or equal to 50; where point C has an X coordinate greater than or equal to 25, and a Y coordinate greater than or equal to 50; and where point D has an X coordinate greater than or equal to 5, and a Y coordinate less than or equal to 100. For example, the X coordinate for point A can be 333, 330, 325, 320, 315, or less; and the Y coordinate for point A can be 100, 99, 95, 90, 85, 80, 75, or less. The X coordinate for point B can be 333, 330, 325, 320, 315, or less; and the Y coordinate for point B can be 50, 55, 60, 65, 70, 75, 80, 85, 90, 95, 99 or more. The X coordinate for point C can be 25, 30, 35, 40, 50, 60, 70, 80, 90, 100, 150, 200, or more; and the Y coordinate for point C can be 50, 55, 60, 65, 70, 75, 80, 85, 90, 95, 99 or more. The X coordinate for point D can be 5, 6, 7, 8, 9, 10, 15, 20, 25, 30, 40, 50, 75, 100, or more; and the Y coordinate for point D can be 100, 99, 95, 90, 85, 80, 75, or less. In one embodiment, point A can be (333, 100), point B can be (333, 95), point C can be (150, 95), and point D can be (150, 100).

Any substantially pure polypeptide containing an amino acid sequence having a length and a percent identity to the sequence set forth in SEQ ID NO:42 over that length as determined herein is within the scope of the invention provided the point defined by that length and percent identity is within the area defined by points A, B, C, and D of FIG. 26; where point A has an X coordinate less than or equal to 337, and a Y coordinate less than or equal to 100; where point B has an X coordinate less than or equal to 337, and a Y coordinate greater than or equal to 50; where point C has an X coordinate greater than or equal to 25, and a Y coordinate greater than or equal to 50; and where point D has an X coordinate greater than or equal to 5, and a Y coordinate less than or equal to 100. For example, the X coordinate for point A can be 337, 335, 330, 325, 320, 315, or less; and the Y coordinate for point A can be 100, 99, 95, 90, 85, 80, 75, or less. The X coordinate for point B can be 337, 335, 330, 325, 320, 315, or less; and the Y coordinate for point B can be 50, 55, 60, 65, 70, 75, 80, 85, 90, 95, 99 or more. The X coordinate for point C can be 25, 30, 35, 40, 50, 60, 70, 80, 90, 100, 150, 200, or more; and the Y coordinate for point C can be 50, 55, 60, 65, 70, 75, 80, 85, 90, 95, 99 or more. The X coordinate for point D can be 5, 6, 7, 8, 9, 10, 15, 20, 25, 30, 40, 50, 75, 100, or more; and the Y coordinate for point D can be 100, 99, 95, 90, 85, 80, 75, or less. In one embodiment, point A can be (337, 100), point B can be (337, 95), point C can be (150, 95), and point D can be (150, 100).

Any substantially pure polypeptide containing an amino acid sequence having a length and a percent identity to the sequence set forth in SEQ ID NO:97 over that length as determined herein is within the scope of the invention provided the point defined by that length and percent identity is within the area defined by points A, B, C, and D of FIG. 26; where point A has an X coordinate less than or equal to 386, and a Y coordinate less than or equal to 100; where point B has an X coordinate less than or equal to 386, and a Y coordinate greater than or equal to 50; where point C has an X coordinate greater than or equal to 25, and a Y coordinate greater than or equal to 50; and where point D has an X coordinate greater than or equal to 5, and a Y coordinate less than or equal to 100. For example, the X coordinate for point A can be 386, 380, 375, 370, 375, 360, 365, 350, 325, 300, or less; and the Y coordinate for point A can be 100, 99, 95, 90, 85, 80, 75, or less. The X coordinate for point B can be 386, 380, 375, 370, 375, 360, 365, 350, 325, 300, or less; and the Y coordinate for point B can be 50, 55, 60, 65, 70, 75, 80, 85, 90, 95, 99 or more. The X coordinate for point C can be 25, 30, 35, 40, 50, 60, 70, 80, 90, 100, 150, 200, 300, 350, or more; and the Y coordinate for point C can be 50, 55, 60, 65, 70, 75, 80, 85, 90, 95, 99 or more. The X coordinate for point D can be 5, 6, 7, 8, 9, 10, 15, 20, 25, 30, 40, 50, 75, 100, 200, 300, 350, or more; and the Y coordinate for point D can be 100, 99, 95, 90, 85, 80, 75, or less. In one embodiment, point A can be (386, 100), point B can be (386, 95), point C can be (350, 95), and point D can be (350, 100).

Any method can be used to obtain a substantially pure polypeptide. For example, common polypeptide purification techniques such as affinity chromotography and HPLC as well as polypeptide synthesis techniques can be used. In addition, any material can be used as a source to obtain a substantially pure polypeptide. For example, tissue from wild-type or transgenic animals can be used as a source material. In addition, tissue culture cells engineered to over-express a particular polypeptide of interest can be used to obtain substantially pure polypeptide. Further, a polypeptide within the scope of the invention can be “engineered” to contain an amino acid sequence that allows the polypeptide to be captured onto an affinity matrix. For example, a tag such as c-myc, hemagglutinin, polyhistidine, or Flag™ tag (Kodak) can be used to aid polypeptide purification. Such tags can be inserted anywhere within the polypeptide including at either the carboxyl or amino termini. Other fusions that could be useful include enzymes that aid in the detection of the polypeptide, such as alkaline phosphatase.

The invention provides polypeptides that contain the entire amino acid sequence depicted in FIG. 4, 9, 12, or 30. In addition, the invention provides polypeptides that contain a portion of the amino acid sequence depicted in FIG. 4, 9, 12, or 30. For example, the invention provides polypeptides that contain a 15 amino acid sequence identical to any 15 amino acid sequence depicted in FIG. 4, 9, 12, or 30 including, without limitation, the sequence starting at amino acid residue number 1 and ending at amino acid residue number 15, the sequence starting at amino acid residue number 2 and ending at amino acid residue number 16, the sequence starting at amino acid residue number 3 and ending at amino acid residue number 17, and so forth. It will be appreciated that the invention also provides polypeptides that contain an amino acid sequence that is greater than 15 amino acid residues (e.g., 16, 17, 18, 19, 20, 21, 22, 23, 24, 25, 26, 27, 28, 29, 30, or more amino acid residues) in length and identical to any portion of the sequence depicted in FIG. 4, 9, 12, or 30. For example, the invention provides polypeptides that contain a 25 amino acid sequence identical to any 25 amino acid sequence depicted in FIG. 4, 9, 12, or 30 including, without limitation, the sequence starting at amino acid residue number 1 and ending at amino acid residue number 25, the sequence starting at amino acid residue number 2 and ending at amino acid residue number 26, the sequence starting at amino acid residue number 3 and ending at amino acid residue number 27, and so forth. Additional examples include, without limitation, polypeptides that contain an amino acid sequence that is 50 or more amino acid residues (e.g., 100, 150, 200, 250, 300, 350, or more amino acid residues) in length and identical to any portion of the sequence depicted in FIG. 4, 9, 12, or 30. Such polypeptides can include, without limitation, those polypeptides containing a amino acid sequence represented in a single line of sequence depicted in FIG. 4, 9, 12, or 30 since each line of sequence depicted in these figures, with the possible exception of the last line, provides a 50 amino acid sequence.

In addition, the invention provides polypeptides that an amino acid sequence having a variation of the amino acid sequence depicted in FIG. 4, 9, 12, or 30. For example, the invention provides polypeptides containing an amino acid sequence depicted in FIG. 4, 9, 12, or 30 that contains a single insertion, a single deletion, a single substitution, multiple insertions, multiple deletions, multiple substitutions, or any combination thereof (e.g., single deletion together with multiple insertions). The invention provides multiple examples of polypeptides containing an amino acid sequence having a variation of an amino acid sequence depicted in FIG. 4, 9, 12, or 30.

FIG. 6 depicts the amino acid sequence depicted in FIG. 4 (designated STdxsp) aligned with 20 other amino acid sequences. Examples of variations of the STdxsp sequence include, without limitation, any variation of the STdxsp sequence provided in FIG. 6. Such variations are provided in FIG. 6 in that a comparison of the amino acid residue (or lack thereof) at a particular position of the STdxsp sequence with the amino acid residue (or lack thereof) at the same position of any of the other 20 amino acid sequences depicted in FIG. 6 provides a list of specific changes for the STdxsp sequence. For example, the “t” at position 1148 of the STdxsp sequence can be substituted with an “s” as indicated in FIG. 6. As also indicated in FIG. 6, the “f” at position 575 of the STdxsp sequence can be substituted with an “m”, “a”, “l”, “i”, “y”, or “v”. For FIG. 6, the nucleic acid numbering of FIG. 2 is used to number the amino acid residue positions of the STdxsp sequence. Thus, the first amino acid residue of the STdxsp sequence starts with number 182 and proceeds in increments of three. It will be appreciated that the STdxsp sequence can contain any number of variations as well as any combination of types of variations. For example, the STdxsp sequence can contain one variation provided in FIG. 6 or more than one (e.g., 2, 3, 4, 5, 6, 7, 8, 9, 10, 15, 20, 25, 50, 100, or more) of the variations provided in FIG. 6. It is noted that the 21 full-length amino acid sequences depicted in FIG. 6 can be polypeptides having DXS activity.

FIG. 14 depicts the amino acid sequence depicted in FIG. 9 (designated RSddsp) and the amino acid sequence depicted in FIG. 12 (designated STddsp) aligned with each other as well as aligned with three other amino acid sequences. For FIG. 14, the nucleic acid numbering of FIG. 7 is used to number the amino acid residue positions of the RSddsp sequence, and the nucleic acid numbering of FIG. 10 is used to number the amino acid residue positions of the STddsp sequence. Thus, the first amino acid residue of the RSddsp and STddsp sequences each start with a number other than 1 and proceed in increments of three. Examples of variations of the RSddsp sequence include, without limitation, any variation of the RSddsp sequence provided in FIG. 14. Examples of variations of the STddsp sequence include, without limitation, any variation of the STddsp sequence provided in FIG. 14. Such variations are provided in FIG. 14 in that a comparison of the amino acid residue (or lack thereof) at a particular position of the RSddsp sequence or the STddsp sequence with the amino acid residue (or lack thereof) at the same position of any of the other amino acid sequences depicted in FIG. 14 provides a list of specific changes for the RSddsp sequence and the STddsp sequence. For example, the “l” at position 762 of the RSddsp sequence or the “l” at position 1007 of the STddsp sequence can be substituted with an “a” as indicated in FIG. 14. Again, it will be appreciated that the RSddsp sequence as well as the STddsp sequence can contain any number of variations as well as any combination of types of variations. For example, the RSddsp sequence can contain one variation provided in FIG. 14 or more than one (e.g., 2, 3, 4, 5, 6, 7, 8, 9, 10, 15, 20, 25, 50, 100, or more) of the variations provided in FIG. 14. Likewise, the STddsp sequence can contain one variation provided in FIG. 14 or more than one (e.g., 2, 3, 4, 5, 6, 7, 8, 9, 10, 15, 20, 25, 50, 100, or more) of the variations provided in FIG. 14. It is noted that the five full-length amino acid sequences depicted in FIG. 14 can be polypeptides having DDS activity.

FIG. 32 depicts the amino acid sequence depicted in FIG. 30 (designated Stdxrp) aligned with 15 other amino acid sequences. Examples of variations of the Stdxrp sequence include, without limitation, any variation of the Stdxrp sequence provided in FIG. 32. Such variations are provided in FIG. 32 in that a comparison of the amino acid residue (or lack thereof) at a particular position of the Stdxrp sequence with the amino acid residue (or lack thereof) at the same position of any of the other 15 amino acid sequences depicted in FIG. 32 provides a list of specific changes for the Stdxrp sequence. It will be appreciated that the Stdxrp sequence can contain any number of variations as well as any combination of types of variations. For example, the Stdxrp sequence can contain one variation provided in FIG. 32 or more than one (e.g., 2, 3, 4, 5, 6, 7, 8, 9, 10, 15, 20, 25, 50, 100, or more) of the variations provided in FIG. 32. It is noted that the full-length amino acid sequences depicted in FIG. 32 can be polypeptides having DXR activity.

The invention also provides polypeptides containing an amino acid sequence that contains a variant of a portion of the amino acid sequence depicted in FIG. 4, 9, 12, or 30 as described herein.

3. Genetically Modified Cells

Any cell containing an isolated nucleic acid within the scope of the invention is itself within the scope of the invention. This includes, without limitation, prokaryotic cells such as cells from the Rhodospirillaceae family (e.g., Rhodobacter cells) and eukaryotic cells such as plant and mammalian cells. It is noted that cells containing an isolated nucleic acid of the invention are not required to express the isolated nucleic acid. In addition, the isolated nucleic acid can be integrated into the genome of the cell or maintained in an episomal state. In other words, cells can be stably or transiently transformed with an isolated nucleic acid of the invention.

Any method can be used to introduce an isolated nucleic acid into a cell. In fact, many methods for introducing nucleic acid into a cell, whether iii vivo or in vitro, are well known to those skilled in the art. For example, calcium phosphate precipitation, electroporation, heat shock, lipofection, microinjection, conjugation, and viral-mediated nucleic acid transfer are common methods that can be used to introduce nucleic acid into a cell. In addition, naked DNA can be delivered directly to cells in vivo as describe elsewhere (U.S. Pat. No. 5,580,859 and U.S. Pat. No. 5,589,466 including continuations thereof). Further, nucleic acid can be introduced into cells by generating transgenic animals.

Any method can be used to identify cells that contain an isolated nucleic acid within the scope of the invention. For example, PCR and nucleic acid hybridization techniques such as Northern and Southern analysis can be used. In some cases, immunohistochemistry and biochemical techniques can be used to determine if a cell contains a particular nucleic acid by detecting the expression of a polypeptide encoded by that particular nucleic acid. For example, detection of polypeptide X-immunoreactivity after introduction of an isolated nucleic acid containing a cDNA that encodes polypeptide X into a cell that does not normally express polypeptide X can indicate that that cell not only contains the introduced nucleic acid but also expresses the encoded polypeptide X from that introduced nucleic acid. In this case, the detection of any enzymatic activities of polypeptide X also can indicate that that cell contains the introduced nucleic acid and expresses the encoded polypeptide X from that introduced nucleic acid.

Any method can be used to direct the expression of an amino acid sequence from a nucleic acid. Such methods are well known to those skilled in the art, and include, without limitation, constructing a nucleic acid such that a regulatory element drives the expression of a nucleic acid sequence that encodes a polypeptide. Typically, regulatory elements are DNA sequences that regulate the expression of other DNA sequences at the level of transcription. Such regulatory elements include, without limitation, promoters, enhancers, and the like. In addition, any method for expressing a polypeptide from an exogenous nucleic acid molecule in microorganisms such as bacteria and yeast can be used. For example, well-known methods for making and using nucleic acid constructs that are capable of expressing exogenous polypeptides within Rhodobacter species (e.g., R. sphaeroides and R. capsulatus) can be used. See, e.g., Dryden and Dowhan, J. Bacteriol., 178(4):1030-1038 (1996); Vasilyeva et al., Applied Biochemistry and Biotechnology, 77-79:337-345 (1999); Graichen et al., J. Bacteriol., 181(14):4216-4222 (1999); Johnson et al., J Bacteriol., 167(2):604-610 (1986); and Duport et al., Gene, 145:103-108 (1994). Further, any methods can be used to identify cells that express an amino acid sequence from a nucleic acid. Such methods are well known to those skilled in the art, and include, without limitation, immunocytochemistry, Western analysis, Northern analysis, and RT-PCR.

The cells described herein can contain a single copy, or multiple copies (e.g., about 5, 10, 20, 35, 50, 75, 100 or 150 copies), of a particular exogenous nucleic acid. For example, a bacterial cell can contain about 50 copies of exogenous nucleic acid X. In addition, the cells described herein can contain more than one particular exogenous nucleic acid. For example, a bacterial cell can contain about 50 copies of exogenous nucleic acid X as well as about 75 copies of exogenous nucleic acid Y. In these cases, each different nucleic acid can encode a different polypeptide having its own unique enzymatic activity. For example, a bacterial cell can contain two different exogenous nucleic acids such that a high level of CoQ(10) is produced. In this example, such a cell can contain a first exogenous nucleic acid that encodes a polypeptide having DXS activity and a second exogenous nucleic acid that encodes a polypeptide having DDS activity. In addition, a single exogenous nucleic acid can encode one or more than one polypeptide. For example, a single nucleic acid can contain sequences that encode three different polypeptides.

In addition to providing cells that contain an isolated nucleic acid of the invention, the invention provides cells (e.g., plant cells, animal cells, and microorganisms) that can be used to produce an isoprenoid compound such as CoQ(10). The term “microorganism” as used herein refers to all microscopic organisms including, without limitation, bacteria, algae, fungi, and protozoa. It is noted that bacteria cells can be membraneous bacteria or non-membraneous bacteria.

The term “non-membraneous bacteria” as used herein refers to any bacteria lacking intracytoplasmic membrane. The term “membraneous bacteria” as used herein refers to any naturally-occurring, genetically modified, or environmentally modified bacteria having an intracytoplasmic membrane. An intracytoplasmic membrane can be organized in a variety of ways including, without limitation, vesicles, tubules, thylalkoid-like membrane sacs, and highly organized membrane stacks. Any method can be used to analyze bacteria for the presence of intracytoplasmic membranes including, without limitation, electron microscopy, light microscopy, and density gradients. See, e.g., Chory et al., J. Bacteriol., 159:540-554 (1984); Niederman and Gibson, Isolation and Physiochemical Properties of Membranes from Purple Photosynthetic Bacteria. In: The Photosynthetic Bacteria, Ed. By Roderick K. Clayton and William R. Sistrom, Plenum Press, pp. 79-118 (1978); and Lueking et al., J. Biol. Chen., 253: 451-457 (1978). Examples of membraneous bacteria that can be used herein include, without limitation, bacteria of the Rhodospirillaceae family such as those in the genus Rhodobacter (e.g., R. sphaeroides, R. capsulatus, R. sulfidophilus, R. adriaticus, and R. veldkampii), the genus Rhodospirillum (e.g., R. rubrum, R. photometricum, R. molischianum, R. fulvum, and R. salinarum), the genus Rhodopseudomonas (e.g., R. palustris, R. viridis, and R. sulfoviridis), the genus Rhodomicrobium, the genus Rhodocyclus, and the genus Rhodopila; bacteria of the Chromatiaceae family such as those in the genus Chromatium, genus Thiocystis, the genus Thiospirillum, the genus Thiocapsa, the genus Lamprobacter, the genus Lalmprocystis, the genus Thiodictyon, the genus Amoebobacter, and the genus Thiopedia; green sulfur bacteria such as those in the genus Chlorobium and the genus Prosthecochloris; bacteria of the Methylococcaceae family such as those in the genus Methylococcus (e.g., M. capsulatus), and the genus Methylomonas (e.g., M. methanica); and particular bacteria of the Nitrobacteraceae family such as those in the genus Nitrobacter (e.g., N. winogradsky and N. hamburgensis), the genus Nitrococcus (e.g., N. miobilis), and the genus Nitrosomonas (e.g., N. europaea).

Membraneous bacteria can be highly membraneous bacteria. The term “highly membraneous bacteria” as used herein refers to any bacterium having more intracytoplasmic membrane than R. sphaeroides (ATCC 17023) cells have after the R. sphaeroides (ATCC 17023) cells have been (1) cultured chemoheterotrophically under aerobic conditions for four days, (2) cultured chemoheterotrophically under oxygen-limited conditions for four hours, and (3) harvested. The aerobic culture conditions involve culturing the cells in the dark at 30° C. in the presence of 25 percent oxygen. The oxygen-limited conditions involve culturing the cells in the light at 30° C. in the presence of 2 percent oxygen. After the four hour culturing step under oxygen-limited conditions, the R. sphaeroides (ATCC 17023) cells are harvested by centrifugation and analyzed.

Typically, any cell (e.g., membraneous bacteria) can be genetically modified such that a particular isoprenoid compound is produced. Such cells can contain exogenous nucleic acid that encodes a polypeptide having enzymatic activity. For example, a microorganism having endogenous DDS activity can be transformed with an exogenous nucleic acid that encodes a polypeptide having DDS activity. In this case, the microorganism can have increased DDS activity which can lead to an increased production of CoQ(10). Thus, a cell can be given an exogenous nucleic acid that encodes a polypeptide having an enzymatic activity that catalyzes the production of a compound normally produced by that cell. In this case, the genetically modified cell can produce more of the compound, or can produce the compound more efficiently, than a similar cell not having the genetic modification. Alternatively, a cell can be given an exogenous nucleic acid that encodes a polypeptide having an enzymatic activity that catalyzes the production of a compound that is not normally produced by that cell.

The invention provides cells containing exogenous nucleic acid that encodes a polypeptide having enzymatic activity that leads to an increased production of CoQ(10). Such cells can contain nucleic acid that encodes a polypeptide having DDS activity. Other examples include, without limitation, cells containing exogenous nucleic acid that encodes polypeptides having DXS, ODS, SDS, DXR, 4-diphosphocytidyl-2C-methyl-D-erythritol synthase (e.g., ispD), 4-diphosphocytidyl-2C-methyl-D-erythritol kinase (e.g., ispE), and/or chorismate lyase (e.g., ubiC) activity. Nucleic acid molecules that encode polypeptides having such enzymatic activities can be obtained as described herein. For example, nucleic acid encoding a polypeptide having chorismate lyase can be cloned using the sequence information provided in GenBank® accession number X66619.

Typically, microorganisms of the invention produce CoQ(10) with the yield (mg of CoQ(10) per g of dry biomass) being at least about 5 (e.g., at least about 6, 7, 8, 9, 10, 11, 12, 13, 14, 15, 20, 25, 30, 35, or more) percent greater than that of a comparable wild-type strain grown under similar conditions. Bacteria can produce more CoQ(10) when grown under anaerobic conditions as compared to aerobic conditions. For example, anaerobically cultured bacteria can produce about 3 to 4 fold more CoQ(10) than aerobically cultured bacteria of the same species. When determining the yield of isoprenoid compound production for a particular cell (e.g., microorganism), any method can be used. See, e.g., Cohen-Bazire et al., J. Cell Comp. Physiol., 49:25-68 (1957); Edlund, J. Chromatogr., 425:87-97 (1988); Rousseau and Varin, J. Chromatogr. Sci., 36:247-52 (1998); and Leray et al., J. Lipid Res., 39:2099-2105 (1998).

The invention provides a cell containing an exogenous nucleic acid that encodes a polypeptide having DXS, DDS, ODS, SDS, DXR, 4-diphosphocytidyl-2C-methyl-D-erythritol synthase (e.g., ispD), 4-diphosphocytidyl-2C-methyl-D-erythritol kinase (e.g., ispE), and/or chorismate lyase (e.g., ubiC) activity. Nucleic acid molecules that encode polypeptides having such enzymatic activities can be obtained as described herein. The invention also provides a cell that contains more than one different exogenous nucleic acid molecule with each different exogenous nucleic acid molecule encoding a polypeptide having a different one of the following enzymatic activities: DXS, DDS, ODS, SDS, DXR, 4-diphosphocytidyl-2C-methyl-D-erythritol synthase (e.g., ispD), 4-diphosphocytidyl-2C-methyl-D-erythritol kinase (e.g., ispE), and/or chorismate lyase (e.g., ubiC) activity. For example, the invention provides a cell containing a first exogenous nucleic acid encoding a polypeptide having DXS activity and a second exogenous nucleic acid encoding a polypeptide having DDS activity.

The invention provides a cell containing an exogenous nucleic acid containing a dxs sequence (e.g., Stdxs sequence), dds sequence (e.g., Stdds or Rsdds sequence), dxr sequence (e.g., Stdxr sequence), ubiC sequence (e.g., EcUbiC sequence), or lytB sequence (e.g., RsLytB sequence). Such nucleic acids can be obtained as described herein. The invention also provides a cell that contains more than one of the following sequences: a dxs sequence (e.g., Stdxs sequence), dds sequence (e.g., Stdds or Rsdds sequence), dxr sequence (e.g., Stdxr sequence), ubiC sequence (e.g., EcUbiC sequence), or lytB sequence (e.g., RsLytB sequence). For example, the invention provides a cell containing a first exogenous nucleic acid containing a dds sequence and a second exogenous nucleic acid containing a dxs sequence. Likewise, the invention provides a cell containing a single exogenous nucleic acid that contains a dds sequence and a dxs sequence.

Typically, a microorganism within the scope of the invention catabolizes a hexose carbon such as glucose. A microorganism, however, can catabolize a pentose carbon (e.g., ribose, arabinose, xylose, and lyxose). In other words, a microorganism within the scope of the invention can either utilize hexose or pentose carbon. In addition, a microorganism within the scope of the invention can use carbon sources such as methanol and/or organic acids (e.g., succinic acid or malic acid).

Any cells described herein can have reduced enzymatic activity such as reduced geranylgeranyl pyrophosphate synthase and/or magnesium protoporphyrin IX chelatase activity. Any cell described herein can have reduced biological activity such as reduced activity of aerobic repressor polypeptides (e.g., PPSR) or oxidation-reduction sensor polypeptides (e.g., CBB3). In the case of multi-subunit molecules such as CBB3, the activity of the oxidation-reduction sensor polypeptide can be reduced by inactivating one or more than one of the subunits. For example, CBB3 activity can be reduced by inactivating a single subunit of CBB3 such as the ccoN subunit.

The term “reduced” as used herein with respect to a cell and a particular activity (e.g., particular enzymatic activity) refers to a lower level of activity than that measured in a comparable cell of the same species. Thus, a R. sphaeroides cell lacking geranylgeranyl pyrophosphate synthase activity is considered to have reduced geranylgeranyl pyrophosphate synthase activity since most, if not all, comparable R. sphaeroides cells have at least some geranylgeranyl pyrophosphate synthase activity. Such reduced enzymatic activities can be the result of lower enzyme concentration, lower specific activity of an enzyme, or combinations thereof.

Many different methods can be used to make a cell having reduced enzymatic and/or biological activity. For example, a R. sphaeroides cell can be engineered to have a disrupted enzyme-encoding locus using common mutagenesis or knock-out technology. Alternatively, antisense technology can be used to reduce enzymatic activity. For example, a R. sphaeroides cell can be engineered to contain a cDNA that encodes an antisense molecule that prevents an enzyme from being made. The term “antisense molecule” as used herein encompasses any nucleic acid that contains sequences that correspond to the coding strand of an endogenous polypeptide. An antisense molecule also can have flanking sequences (e.g., regulatory sequences). Thus, antisense molecules can be ribozymes or antisense oligonucleotides. A ribozyme can have any general structure including, without limitation, hairpin, hammerhead, or axhead structures, provided the molecule cleaves RNA.

Cells having a reduced enzymatic and/or biological activity can be identified using any method. For example, a R. sphaeroides cell having reduced geranylgeranyl pyrophosphate synthase activity can be easily identified using common biochemical methods that measure geranylgeranyl pyrophosphate synthase activity. See, e.g., Math et al., Proc. Natl. Acad. Sci. USA, 89(15):6761-6764 (1992).

The invention provides a cell containing reduced geranylgeranyl diphosphate synthase, aerobic repressor, and/or cbb3-type cytochrome oxidase activity. Such cells can have reduced geranylgeranyl diphosphate synthase, aerobic repressor, and/or cbb3-type cytochrome oxidase activity as a result of disrupting the endogenous sequences that encode polypeptides having these activities. For example, a cell can have reduced geranylgeranyl diphosphate synthase activity as a result of knocking out a portion of the endogenous crtE sequence within a cell's genome; a cell can have reduced aerobic repressor activity as a result of knocking out a portion of the endogenous ppsR sequence within a cell's genome; and a cell can have reduced cbb3-type cytochrome oxidase activity as a result of knocking out a portion of the endogenous ccoN sequence within a cell's genome.

The invention also provides a cell containing non-functional crtE, ppsR, and/or ccoN nucleic acid sequences within its genome such that the encoded polypeptide is either mutated or not expressed. Such cells can be used to produce large amounts of CoQ(10). The sequence of crtE can be as set forth in Genbank® accession number AJ010302. The sequence of ppsR can be as set forth in Genbank® accession number AJ010302 or L19596. The sequence of ccoN can be as set forth in Genbank® accession number U58092. Knockout technology can be used to make cells containing non-functional crtE, ppsR, and/or ccoN nucleic acid sequences.

4. Producing Isoprenoid Compounds

The cells described herein can be used to produce isoprenoid compounds. For example, a microorganism having endogenous DDS activity can be transformed with nucleic acid that encodes a polypeptide having DDS activity such that the microorganism produces more CoQ(10) than had the microorganism not been given that nucleic acid. Once transformed, the microorganism can be used cultured under conditions optimal for CoQ(10) production.

In addition, substantially pure polypeptides having enzymatic activity can be used alone or in combination with cells to produce isoprenoid compounds. For example, a preparation containing a substantially pure polypeptide having DDS activity can be used to catalyze the formation of CoQ(10). Further, cell-free extracts containing a polypeptide having enzymatic activity can be used alone or in combination with substantially pure polypeptides and/or cells to produce isoprenoid compounds. For example, a cell-free extract containing a polypeptide having DXS activity can be used to form 1-deoxyxyulose-5-phosphate, while a microorganism containing polypeptides have the enzymatic activities necessary to catalyze the reactions needed to form CoQ(10) from 1-deoxyxyulose-5-phosphate can be used to produce CoQ(10). Any method can be used to produce a cell-free extract. For example, osmotic shock, sonication, and/or a repeated freeze-thaw cycle followed by filtration and/or centrifugation can be used to produce a cell-free extract from intact cells.

It is noted that a cell, substantially pure polypeptide, and/or cell-free extract can be used to produce a particular isoprenoid compound that is, in turn, treated chemically to produce another compound. For example, a microorganism can be used to produce CoQ(10), while a chemical process is used to modify CoQ(10) into a CoQ(10) derivative such as CoQ10 containing a polar group. Likewise, a chemical process can be used to produce a particular compound that is, in turn, converted into an isoprenoid compound using a cell, substantially pure polypeptide, and/or cell-free extract described herein. For example, a chemical process can be used to produce deoxyxylose-5-phosphate, while a microorganism can be used convert deoxyxylose-5-phosphate into CoQ(10).

Typically, a particular isoprenoid compound is produced by providing a microorganism and culturing the provided microorganism with culture medium such that that isoprenoid compound is produced. In general, the culture media and/or culture conditions can be such that the microorganisms grow to an adequate density and produce the desired compound efficiently. For large-scale production processes, the following methods can be used. First, a large tank (e.g., a 100 gallon, 200 gallon, 500 gallon, or more tank) containing appropriate culture medium with, for example, a glucose carbon source is inoculated with a particular microorganism. After inoculation, the microorganisms are incubated to allow biomass to be produced. Once a desired biomass is reached, the broth containing the microorganisms can be transferred to a second tank. This second tank can be any size. For example, the second tank can be larger, smaller, or the same size as the first tank. Typically, the second tank is larger than the first such that additional culture medium can be added to the broth from the first tank. In addition, the culture medium within this second tank can be the same as, or different from, that used in the first tank. For example, the first tank can contain medium with xylose, while the second tank contains medium with glucose.

Once transferred, the microorganisms can be incubated to allow for the production of the desired isopreniod compound. Once produced, any method can be used to isolate the desired compound. For example, if the microorganism releases the desired isoprenoid compound into the broth, then common separation techniques can be used to remove the biomass from the broth, and common isolation procedures (e.g., extraction, distillation, and ion-exchange procedures) can be used to obtain the isoprenoid compound from the microorganism-free broth. In addition, the desired isoprenoid compound can be isolated while it is being produced, or it can be isolated from the broth after the product production phase has been terminated. If the microorganism retains the desired isoprenoid compound, then the biomass can be collected and treated to release the isoprenoid compound, and the released isoprenoid compound can be isolated.

The invention will be further described in the following examples, which do not limit the scope of the invention described in the claims.

### EXAMPLES

### Example 1

### Cloning Nucleic Acid that Encodes a Sphingomonas trueperi Polypeptide Having Dxs Activity

S. trueperi cells were obtained from the American Type Culture Collection (ATCC Cat. No. 12417). To isolate bacterial genomic DNA, cells were grown in 100-200 mL cultures for 2-3 days at 30° C. on a shaker rotating at 250 rpm. Cultured cells were centrifuged to form a cell pellet, washed by resuspending the pellet in a solution of 10 mM Tris/1 mM EDTA, and centrifuged again as before. The cell pellets were resuspended in 5 mL of GTE buffer per 100 mL of original culture. GTE buffer is 50 mM glucose/25 mM Tris-HCl (pH 8.0)/10 mM EDTA (pH 8.0). The bacterial cell walls were lysed by adding lysozyme (final concentration of 1 mg/mL), Proteinase K (final concentration of 1 mg/mL), and mutanolysin (final concentration of 5.5 μg/mL) to the resuspended cell solution to form a lysing mixture that was incubated for 90 minutes at 37° C. After this incubation, sodium dodecyl sulfate was added to the mixture to a final concentration of 1 percent, and additional Proteinase K was added until the concentration in the solution was 2 mg/mL. After a 1 hour incubation at 50° C., the solution containing the lysed cells was diluted 1:1 with fresh GTE buffer. Once diluted, sodium chloride was added to the solution to a final concentration of 0.15 M. Polypeptides and molecules other than nucleic acids were removed from the lysed bacterial cell solution by adding an equal volume of an organic mixture made up of phenol, chloroform, and isoamyl alcohol at a ratio of 25:24:1 (hereinafter referred to as PCIA). After adding PCIA, the solution was mixed. To separate the organic phase from the DNA-containing aqueous phase, the mixture was centrifuged at 12,000×g for 10 minutes. The aqueous phase was transferred to a clean tube and re-extracted with an equal volume of chloroform alone. The aqueous and organic phases were separated by centrifugation at 3,000×g for 10 minutes. The aqueous phase was again removed to a new tube and treated with 2.5 mg of RNase to degrade any bacterial RNA present. The purified DNA was recovered by adding 2.5 volumes of ethanol to the aqueous phase. After mixing the solution, the precipitated DNA was removed by spooling it on a glass rod. The spooled DNA was rinsed with 70 percent ethanol. Once rinsed, the ethanol was allowed to evaporate by leaving the DNA exposed to the air until dry. The dried DNA was resuspended in a solution of 10 mM Tris (pH 8.5). The resuspended DNA was re-extracted with PCIA followed by chloroform alone as before. The DNA was re-precipitated by adding one-tenth volume of 7.5 M ammonium acetate and 2.5 volumes ethanol, followed by spooling, rinsing, and air drying. The purified DNA was resuspended in 10 mM Tris (pH 8.5).

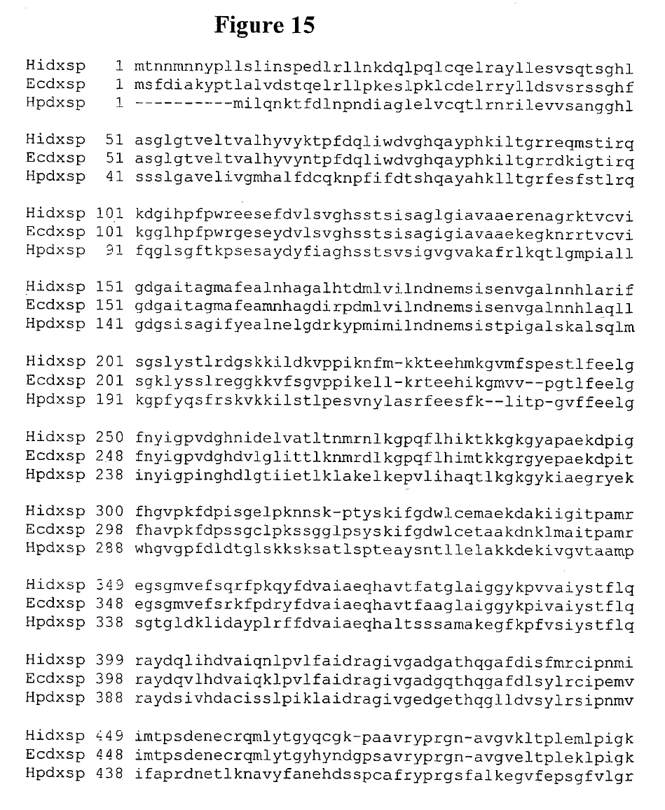

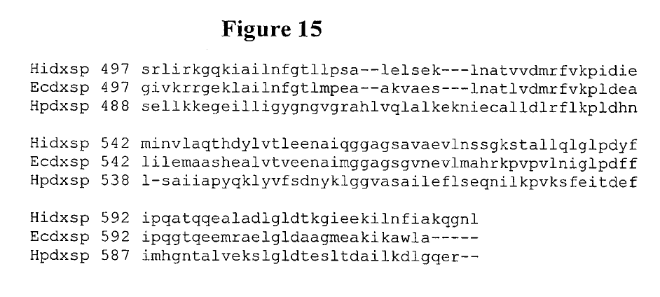

The following polymerase chain reaction (PCR) procedure was used to isolate nucleic acid that encodes a S. trueperi polypeptide having DXS activity. Three degenerate forward PCR primers (F1, F2, and F3) and three degenerate reverse PCR primers (R1, R2, and R3) were designed by comparing sequences of several clones that encode polypeptides have DXS activity (FIG. 15). The sequence of each degenerate primer was as follows:

| | | |
|---|---|---|
| F1: | 5′-RTKATTYTMAAYGAYAAYGAAATG-3′ | (SEQ ID NO:53) |
| F2: | 5′-TTTGAAGARYTVGGYWTTAACTA-3′ | (SEQ ID NO:54) |
| F3: | 5′-RCAYCARGCTTAYSCVCAYAA-3′ | (SEQ ID NO:55) |
| R1: | 5′-CGTGYTGYTCDGCRATHGCBAC-3′ | (SEQ ID NO:56) |
| R2: | 5′-TGYTCDGCRATHGCBACRTCRAA-3′ | (SEQ ID NO:57) |
| R3: | 5′-GGSCCDATRTAGTTAAWRCC-3′ | (SEQ ID NO:58) |

The primers were used in all logical combinations in PCR using Taq polymerase (Roche Molecular Biochemicals, Indianapolis, Ind.) and 1 ng of purified genomic DNA per microliter of reaction nix. Each PCR reaction was conducted using a touchdown PCR program with four cycles at each of the following annealing temperatures: 60° C., 58° C., 56° C., and 54° C., followed by 25 cycles at 52° C. Each cycle had an initial 30 second denaturing step at 94° C. and a 90 second extension step at 72° C. The program had an initial denaturing step of 2 minutes at 94° C. and final extension step of 5 minutes at 72° C.

Between about 2 μM and 12 μM of each PCR primer was used in each reaction, depending on the degree of degeneracy. After each PCR reaction was complete, a portion of each reaction was separated by gel electrophoresis using a 1.5 percent TAE (Tris-acetate-EDTA) agarose gel. The results from the gel electrophoresis indicated that the combination of degenerate primer F3 with degenerate primer R2 produced a nucleic acid molecule of 882 bp (referred to as the F3R2 fragment). The F3R2 fragment was purified away from the agarose gel matrix using the Qiagen Gel Extraction procedure according to the manufacturer's instructions (Qiagen Inc., Valencia, Calif.). A portion of the purified fragment was ligated into the pCRII-TOPO vector. The vector containing the F3R2 fragment was inserted into E. coli TOP10 cells using the TOPO cloning procedure (Invitrogen, Carlsbad, Calif.). The transformed TOP10 cells were plated onto LB agar plates containing 100 μg/mL of ampicillin (Amp) and 50 μg/mL of 5-Bromo-4-Chloro-3-Indolyl-β-D-Galactopyranoside (Xgal). Single white colonies were re-plated onto fresh LB-Amp-Xgal plates and screened by PCR with the F3 and R2 primers to confirm the presence of plasmids with the desired insert. Plasmid DNAs were obtained from bacterial colonies using the QiaPrep Spin Miniprep Kit (Qiagen, Inc). The plasmid DNAs were then quantified and sequenced with the M13 forward and reverse primers. Sequence analysis indicated that the sequence of the F3R2 fragment aligned with sequences from other nucleic acid molecules that encode polypeptides having DXS activity.

To obtain the complete coding sequence for the S. trueperi polypeptide having DXS activity, genome walking was performed as follows. Primers were designed based upon the sequence of the 882 bp F3R2 fragment for walking in both the upstream and downstream directions. These walking primers had the following sequences:

| | |
|---|---|
| GSP1F: | |
| 5′-TCGTGACCAAGAAGGGCAAGGGCTATG-3′ | (SEQ ID NO:59) |
| GSP2F: | |
| 5′-GACAAGTATCACGGCGTCCAGAAGTTC-3′ | (SEQ ID NO:60) |
| GSP1R: | |
| 5′-ATAGCCCTTGCCCTTCTTGGTCACGAC-3′ | (SEQ ID NO:61) |
| GSP2R: | |
| 5′-CGAACGGATCATACTCGCTCTCGCTG-3′ | (SEQ ID NO:62) |

The GSP1F and GSP2F primers are primers that face downstream of the DXS polypeptide start codon, while the GSP1R and GSP2R primers are primers that face in the opposite direction. In addition, GSP2F and GSP2R are nested inside of the GSP1F and GSP1R primers. Genome walking was conducted according to the manual of CLONTECH's Universal Genome Walking kit (CLONTECH Laboratories, Inc., Palo Alto, Calif.) with the exception that Fsp I and Sma I were used instead of Dra I and EcoR V. The genomic DNA used was from S. trueperi. DMSO was added to the PCR mixture until a final concentration of 5 percent was reached. The PCR reactions were performed using a Perkin Elmer 9700 Thermocycler. The first round of PCR consisted of 7 cycles of 2 seconds at 94° C. and 3 minutes at 72° C., followed by 36 cycles of 2 seconds at 94° C. and 3 minutes at 67° C., with a final extension at 67° C. for 4 minutes. The second round of PCR consisted of 5 cycles of 2 seconds at 94° C. and 3 minutes at 72° C., followed by 24 cycles of 2 seconds at 94° C. and 3 minutes at 67° C., with a final extension at 67° C. for 4 minutes. After the PCR was complete, a portion of the reaction mix from each round was separated by gel electrophoresis using a 1.5 percent TAE agarose gel. Good amplification products were obtained with the Pvu II and Stu I libraries using the GSP1F and GSP2F primers and with the Fsp I and Pvu II libraries using the GSP1 R and GSP2R primers. The second round products from each of these libraries were gel purified, cloned using the TOPO cloning procedure (Invitrogen, Carlsbad, Calif.), and sequenced. A 1.7 kilobase (kb) fragment was subcloned from the Pvu IIF library, a 2.8 kb fragment was subcloned from the Stu IF library, a 400 bp fragment was subcloned from the Fsp IR library, and a 330 bp fragment was subcloned from the Pvu IIR library. Each of these subcloned fragments was sequenced. Sequence analysis indicated that each subcloned fragment contained a sequence that overlapped with that of the F3R2 fragment and was similar to other nucleic acid sequences that encode polypeptides having DXS activity.

Because the sequence information obtained by genome walking extended 13 bp upstream of the translational start codon, a second genome walk was conducted to gain additional sequence information. This second walk used GSPB2R, 5′-TGAGGATCTTGTGCGGATAGC-ATTGGTG-3′ (SEQ ID NO:63) as the first round primer and GSPB3R, 5′-AGCGGCGTCTTG-GGTAGGTCAGCCAT-3′ (SEQ ID NO:64) as the second round primer. The second walk was conducted using only the Sma I and Stu I libraries. CLONTECH's Advantage-GC Genomic Polymerase was used for PCR with a 1.0 mM GC Melt concentration according to the manufacturer's specifications. The first round of PCR was conducted using a Perkin Elmer 9700 Thermocycler with an initial denaturing step at 96° C. for 5 seconds followed by 7 cycles consisting of 2 seconds at 94° C. and 3 minutes at 72° C., followed by 36 cycles consisting of 2 seconds at 94° C. and 3 minutes at 66° C., with a final extension at 66° C. for 4 minutes. The second round of PCR had 5 cycles consisting of 2 seconds at 94° C. and 3 minutes at 72° C., followed by 26 cycles consisting of 2 seconds at 94° C. and 3 minutes at 66° C., with a final extension at 66° C. for 4 minutes. Portions of the PCR products from each round were separated by gel electrophoresis using a 1.5 percent TAE agarose gel. The gel electrophoresis revealed the presence of a 250 bp amplification product obtained from the second round of PCR using the Stu I library. This fragment was gel purified, cloned using the TOPO cloning procedure (Invitrogen, Carlsbad, Calif.), and sequenced. An overlap with the previously obtained sequence was found, extending the length of the clone to 181 bp before the start codon. The full-length clone containing coding and non-coding sequence was 3626 bp in length (FIG. 2). The open reading frame was 1926 bp in length (FIG. 3), which encoded a polypeptide with 641 amino acid residues (FIG. 4).

The coding sequence of the DXS polypeptide was amplified by PCR using S. trueperi genomic DNA as template. Primers were designed based on the sequence obtained above. The sequences of the primers were as follows:

| | |
|---|---|
| SHDXF1: | |
| 5′-ATATGGTACCGTGTGACTGACCTGTCCAAC-3′ | (SEQ ID NO:65) |
| SHDXR1: | |
| 5′-AGTCTCTAGAATGTTGGAGATTCAAGGTGG-3′ | (SEQ ID NO:66) |

These primers were designed to introduce a Kpn I restriction site at the beginning of the amplified fragment and an Xba I restriction site at the end of the amplified fragment. The sequence of each restriction site is underlined. The PCR reaction mix contained the following: 100 ng genomic DNA, 2 μL of each primer (SHDXF1 and SHDXR1, each at 50 μM), 10 μL 10×Pfu Plus buffer, 5 μL DMSO, 8 μL dNTPs (10 μM each) and 5 units Pfu polymerase in a final volume of 100 μL. Each PCR reaction was performed in a Perkin Elmer Geneamp PCR system 2400 under the following conditions: an initial denaturation at 94° C. for 5 minutes; 8 cycles of (1) 94° C. for 45 seconds, (2) 55° C. for 45 seconds, and (3) 72° C. for 3 minutes; 21 cycles of (1) 94° C. for 45 seconds, (2) 61° C. for 45 seconds and (3) 72° C. for 3 minutes; and a final extension of 72° C. for 10 minutes. A portion of the PCR reaction was separated by gel electrophoresis using a 0.8 percent TAE gel. The gel electrophoresis revealed a 1.6 kb fragment. This fragment was (1) purified using a Qiagen Gel Extraction kit (Qiagen Inc., Valencia, Calif.), (2) treated with Kpn I and Xba I (New England BioLabs, Inc., Beverly, Mass.), and (3) subcloned into pUC18 that had also been treated with Kpn I and Xba I and gel purified. The resulting construct designated appUC18-SHDXS is depicted in FIG. 18. The ligation was carried out with T4 DNA ligase at 16° C. for 16 hours. Once ligated, 1 μL was used to electroporate E. coli ElectroMAX™ DH10B™ cells (Life Technologies, Inc., Rockville, Md.). The electroporated cells were plated on LB-Amp plates (Amp concentration=100 μg/mL). From these plates, eight individual colonies were chosen at random. The plasmid was isolated from each colony using a QiaPrep Spin Miniprep Kit (Qiagen Inc., Valencia, Calif.). The extracted plasmid DNA was examined for the presence of the 1.6 kb fragment by digesting individual aliquots with one of three different restriction enzymes: EcoR I, BamH I, and Nar I. If the plasmids contained the correct 1.6 kb fragment, the EcoR I digest reaction would result in two fragments (0.77 and 4.13 kb), the BamH I digest reaction would result in one fragment (4.8 kb), and the Nar I digest reaction would result in two fragments (1.9 and 2.9 kb). After treating with the restriction enzymes, the digest reactions were separated by gel electrophoresis using a 0.8 percent TAE agarose gel. All 8 clones yielded digestion fragments consistent with a clone of 1.6 kb.

### Example 2

### Introducing Nucleic Acid that Encodes a Polypeptide Having DXS Activity into Cells

The nucleic acid molecule that encodes a polypeptide having DXS activity and was obtained as described in Example 1 is introduced into cells as follows. First, a construct is made to contain the nucleic acid molecule such that the encoded polypeptide having DXS activity is expressed in a desired host cell. When using prokaryotic cells, a construct functional in prokaryotic cells is used. When using eukaryotic cells, a construct functional in eukaryotic cells is used. Second, the construct is introduced into the desired host cell using appropriate methods. Once introduced, stable transformants are selected.

### Example 3

### Cloning Nucleic Acid that Encodes a Rhodobacter sphaeroides Polypeptide Having DDS Activity

R. sphaeroides ATCC strain 17023 cells were grown in 550 R 8 A H media at 30° C. and 100 rpm. The recipe for 550 R 8 A H media was provided by ATCC. Genomic DNA was isolated from R. sphaeroides cells as described in Example 1.

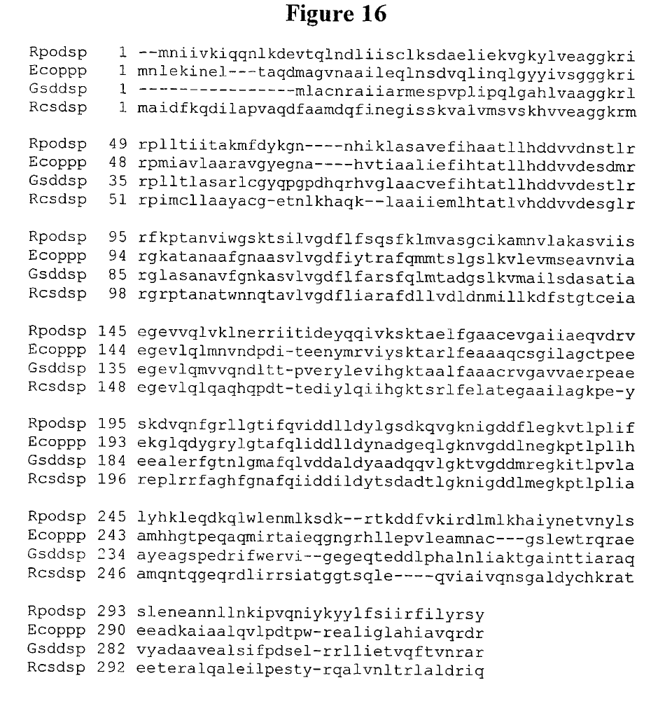

To isolate nucleic acid encoding an R. sphaeroides polypeptide having DDS activity, degenerate primers were designed and used as described in Example 1. Briefly, three degenerate forward primers (F4, F5, and F6) and four degenerate reverse primers (R4, R5, R6, and R7) were designed by comparing sequences of several clones that encode polypeptides have DDS, SDS, or ODS activity (FIG. 16). The sequence of each degenerate primer was as follows:

| | | |
|---|---|---|
| F4: | 5′-GGWGGHAARMGMMTKCGYCC-3′ | (SEQ ID NO:67) |
| F5: | 5′-ACWYTGSTDCATGATGATGT-3′ | (SEQ ID NO:68) |
| F6: | 5′-ACNYTNBTNCAYGAYGAYGT-3′ | (SEQ ID NO:69) |
| R4: | 5′-TYRTCYACSACATCATCATG-3′ | (SEQ ID NO:70) |
| R5: | 5′-TGHAVKACYTCACCYTCRGMAAT-3′ | (SEQ ID NO:71) |
| R6: | 5′-TARTCNARDATRTCRTCDAT-3′ | (SEQ ID NO:72) |
| R7: | 5′-TCRTCNCCNAYNKTYTTNCC-3′ | (SEQ ID NO:73) |

These primers were used in all logical combinations in PCR using Taq polymerase (Roche Molecular Biochemicals, Indianapolis, Ind.) and 1 ng of genomic DNA per microliter of reaction mix. PCR was conducted using the touchdown PCR program as described in Example 1. Between about 4 μM and 8 μM of each PCR primer was used in each reaction, depending on the degree of degeneracy. After each PCR reaction was complete, a portion of each reaction was separated by gel electrophoresis using a 1.5 percent TAE agarose gel. The results from the gel electrophoresis yielded no fragments of the expected size. A second amplification reaction was then performed using each sample from the first round of PCR. Briefly, one μL of reaction mixture from each first round of PCR was used in a 50 μL amplification reaction using the same primer pairs and thermocycling parameters used in the first round of PCR. A portion of each of the second round PCR reactions was separated by gel elecrophoresis using a 1.5 percent TAE agarose gel. The combination of degenerate primers F6 and R5 produced a fragment of 209 bp (referred to as the F6R5 fragment). The F6R5 fragment was isolated from an agarose gel and purified using the Qiagen Gel Extraction procedure (Qiagen Inc., Valencia, Calif.). An aliquot of the purified fragment was ligated to pCRII-TOPO, and the product of the ligation reaction was inserted into TOP10 E. coli cells using a TOPO cloning procedure (Invitrogen, Carlsbad, Calif.). The products of the individual insertion reactions were plated onto LB media containing 100 μg/mL Amp and 50 μg/mL Xgal. Single white colonies that grew on the LB-Amp-Xgal plates were re-plated onto fresh LB-Amp plates and screened in a PCR reaction using the F6 and R5 primers to confirm the presence of the desired insert. Plasmid DNAs were obtained from several colonies using a QiaPrep Spin Miniprep kit (Qiagen, Inc). The obtained plasmid DNAs were quantified and sequenced with the M13 forward and reverse primers. Sequence analysis revealed that the F6R5 fragment contained sequences that aligned with sequences from other nucleic acid molecules that encode polypeptides having polyprenyl diphosphate synthase activity.

Genome walling was performed to obtain a complete coding sequence for the R. sphaeroides DDS polypeptide using procedures similar to those described in Example 1. Briefly, primers were designed based on the sequence of the F6R5 fragment for walking in both the upstream and downstream directions. These primers had the following sequences:

| | |
|---|---|
| GSP3F: | |
| 5′-TGGAAGCTGCGGGCGAAGAGATAGTC-3′ | (SEQ ID NO:74) |
| GSP4F: | |
| 5′-CCCACCAGCACCGAGGATTTGTTGTC-3′ | (SEQ ID NO:75) |
| GSP3R: | |
| 5′-GAACCTGCTGTGGGACAACAAATCCTC-3′ | (SEQ ID NO:76) |
| GSP4R: | |
| 5′-TCGGTGCTGGTGGGCGACTATCTCTTC-3′ | (SEQ ID NO:77) |

The GSP3F and GSP4F primers are primers that face downstream of the DDS polypeptide start codon, while the GSP3R and GSP4R primers are primers that face in the opposite direction. In addition, the GSP4F and GSP4R primers are nested inside the GSP3F and GSP3R primers.

The Pvu II, Fsp I, and Stu I libraries with the GSP3F and GSP4F primers and all four libraries with the GSP3R and GSP4R primers resulted in the production of amplified fragments. A 750 bp fragment from the Pvu I library, a 500 bp fragment from the Fsp I library, a 1.4 kb fragment from the Stu I library, and a 0.9 kb fragment from the Sma I library were all subcloned and sequenced. Sequence analysis indicated that each subcloned fragment contained a sequence that overlapped with the sequence of the F6R5 fragment and was similar to other nucleic acid sequences that encode polypeptides having polyprenyl diphosphate synthase activity. The full-length clone containing coding and non-coding sequence was 1990 bp in length (FIG. 7). The open reading frame was 1002 bp in length (FIG. 8), which encoded a polypeptide with 333 amino acid residues (FIG. 9).

The coding sequence of the DDS polypeptide from R. sphaeroides was amplified by PCR using R. sphaeroides genomic DNA as template. PCR primers were designed based on the sequences obtained as described above. The sequences of the primers were as follows.

| | |
|---|---|
| RDS18F: | |
| 5′-ACTAGAATTCCGCAACAGTTCCTTCATGTC-3′ | (SEQ ID NO:78) |
| RDS18R: | |
| 5′-ATAGAAGCTTACTTGCGGTCGGACTGATAG-3′ | (SEQ ID NO:79) |

These primers were designed to introduce an EcoR I restriction site at the beginning of the amplified fragment and a Hind III restriction site at the end of the amplified fragment. The sequence of each restriction site is underlined. The PCR reaction mix contained the following: 100 ng genomic DNA, 2 μL of each primer (RDS18F and RDS18R, each at 50 μM), 10 μL 10×Pfu Plus buffer, 5 μL DMSO, 8 μL dNTPs (10 mM each) and 5 units Pfu polymerase in a final volume of 100 μL. Each PCR reaction was performed in a Perkin Elmer Geneamp PCR system 2400 under the following conditions: an initial denaturation at 94° C. for 5 minutes; 8 cycles of (1) 94° C. for 45 seconds, (2) 55° C. for 45 seconds, and (3) 72° C. for 3 minutes; 21 Cycles of (1) 94° C. for 45 seconds, (2) 61° C. for 45 seconds, and (3) 72° C. for 3 minutes; and a final extension of 72° C. for 10 minutes. After completing the PCR reactions, each PCR reaction was separated by gel electrophoresis using a 0.8 percent TAE agarose gel. The gel electrophoresis revealed a 1.6 kb fragment. This fragment was (1) purified from the agarose gel using a Qiagen Gel Extraction kit, (2) digested with EcoR I and Hind III (New England BioLabs, Beverly, Mass.), and (3) ligated to pUC18 that had also been digested with EcoR I and Hind III and gel purified. The resulting construct designated appUC18-RSdds is depicted in FIG. 19. The ligation was carried out with T4 DNA ligase at 16° C. for 16 hours. Once ligated, one μL of the ligation reaction was used to electroporate E. coli ElectroMAX™ DH10B™ cells (Life Technologies, Inc., Rockville, Md.). The electroporated cells were plated onto LB-Amp plates (Amp concentration was 100 μg/mL). From these LB-Amp plates, eight individual colonies were selected at random, and the plasmids within these colonies were purified using a Qiaprep Spin Miniprep kit. These purified plasmids were evaluated for the presence of inserts by restriction enzyme analysis. If the plasmids contained the correct 1.6 kb fragment, then an EcoR I and Hind III digest reaction would result in two fragments (2.6 and 1.6 kb), and a BamH I digest reaction would result in one fragment (4.2 kb). After treating with the restriction enzymes, the digest reactions were separated by gel electrophoresis using a 0.8 percent TAE agarose gel. Of the eight clones tested, four contained the desired 1.6 kb fragment.

### Example 4

### Cloning Nucleic Acid that Encodes a Sphingomonas trueperi Polypeptide Having DDS Activity

S. trueperi cells were grown as described in Example 1. In addition, genomic DNA was isolated from S. trueperi cells as described in Example 1.

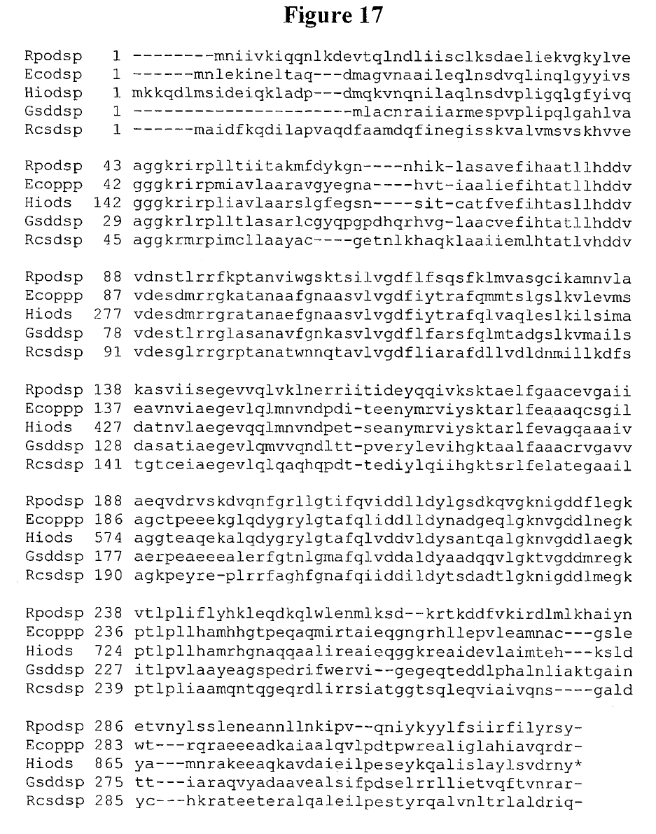

To isolate nucleic acid encoding a polypeptide having DDS activity from S. trueperi, a strategy similar to that described in Example 3 was employed. In this case, four degenerate forward primers (SF1, SF2, SF3, and SF4) and four degenerate reverse primers (SR1, SR2, SR3, and SR4) were designed comparing sequences of several clones that encode polypeptides having polyprenyl diphosphate synthase activity (FIG. 17). Codon usage tables from twelve Sphingomonas species were used to develop an average preferred codon table that was used in primer design. The sequence of each degenerate primer was as follows:

| | | |
|---|---|---|
| SF1: | 5′-CTSSTSCAYGAYGAYGTSGTSGA-3′ | (SEQ ID NO:80) |
| SF2: | 5′-GTSGMVGSSGGSGGSAARC-3′ | (SEQ ID NO:81) |
| SF3: | 5′-CTSMTSCAYGAYGAYGTS-3′ | (SEQ ID NO:82) |
| SF4: | 5′-DSSRTBCTSGTSGGSGAYTT-3′ | (SEQ ID NO:83) |
| SR1: | 5′-VAKRAARTCSCCSACSAGSAC-3′ | (SEQ ID NO:84) |
| SR2: | 5′-SACYTCSCCYTCSGCRAT-3′ | (SEQ ID NO:85) |
| SR3: | 5′-RTCRTCSCCVAYVKTYTTSCC-3′ | (SEQ ID NO:86) |
| SR4: | 5′-SGGSAGSGTVRBYTTSCCYTC-3′ | (SEQ ID NO:87) |

The primers were used in all logical combinations in PCR using Taq polymerase (Roche Molecular Biochemicals, Indianapolis, Ind.) and 1 ng of genomic DNA per microliter of reaction mix. PCR was conducted using the touchdown PCR program as described in Example 1. Between about 4 μM and 20 μM of each PCR primer was used in each reaction depending on the degree of degeneracy. After each PCR reaction was complete, a portion of each reaction was separated by gel electrophoresis using a 1.5 percent TAE agarose gel. Each PCR reaction produced several amplified fragments of the expected sizes based on the coding sequences of other polyprenyl diphosphate synthase polypeptides. These fragments were isolated from TAR agarose gels and purified using the Qiagen Gel Extraction procedure (Qiagen Inc., Valencia, Calif.). An aliquot of each purified fragment was ligated into pCRII-TOPO. The ligated plasmids were then inserted into TOP10 E. coli cells using a TOPO cloning procedure (Invitrogen, Carlsbad, Calif.). The products of each of the individual insertion reactions were plated on LB-Amp-Xgal plates as described in Examples 1 and 3. Single white colonies that grew on the LB-Amp-Xgal plates were re-plated onto fresh LB-Amp-Xgal plates and screened in a PCR reaction using the initial degenerate primers to confirm the presence of the desired insert. Plasmid DNAs having the desired insert were obtained from multiple colonies using a QiaPrep Spin Miniprep Kit (Qiagen, Inc). The obtained plasmid DNAs were then quantified and sequenced using the Ml 3 forward and reverse primers. Sequence analysis revealed that a 201 bp fragment produced using the SF1 and SR2 degenerate primers, a 476 bp fragment produced using the SF1 and SR4 primers, and a 206 bp fragment produced using the SF3 and SR2 primers contained sequences similar to the coding sequences of other polyprenyl diphosphate synthases.

Genome walking was performed to obtain a complete coding sequence for the S. trueperi DDS polypeptide using procedures similar to those described in Example 1. Briefly, primers were designed based on the sequences of the obtained fragments. These primers had the following sequences:

| | |
|---|---|
| GSP5F: | |
| 5′-GTGCTGGTCGGCGACTTCCTGTTCAG-3′ | (SEQ ID NO:88) |
| GSP6F: | |
| 5′-ATCGACCTGTCCGAGGATCGCTATCTC-3′ | (SEQ ID NO:89) |
| GSP5R: | |
| 5′-TCGAACGAGCGGCTGAACAGGAAGTC-3′ | (SEQ ID NO:90) |
| GSP6R: | |
| 5′-TGGCGGGATTGCCCCAGATGATGTTG-3′ | (SEQ ID NO:91) |

The GSP5F and GSP6F primers are primers that face downstream of the DDS start codon, while the GSP5R and GSP6R primers are primers that face in the opposite direction. In addition, the GSP6F and GSP6R primers are nested inside the GSP5F and GSP5R primers.

Genome walking was conducted as described in Example 3 with the exception that the 36 cycles had 3 minute incubations at 66° C. instead of 67° C. and the final extension was performed at 66° C. instead of 67° C. for both the first and second rounds of PCR. Portions of the PCR reactions from each round were separated by gel electrophoresis using a 1.5 percent TAE agarose gel. PCR on the Fsp I and Stu I libraries with the forward primers and of all four libraries with the reverse primers resulted in the production of an amplified fragment. A 1.4 kb fragment from the Fsp I library, a 1.1 kb fragment from the Stu I library (forward primer), a 2.0 kb fragment from the Pvu II library (forward primer), and a 3.0 kb fragment from the Stu I library (reverse primer) were gel purified, cloned using the TOPO cloning procedure, and sequenced as described in Examples 1 and 3. The sequencing analysis revealed that these fragments contained sequences that overlapped with the sequence of the initially obtained fragments and were similar to the coding sequences of other polyprenyl diphosphate synthases. The full-length clone containing coding and non-coding sequence was 1833 bp in length (FIG. 10). The open reading frame was 1014 bp in length (FIG. 11), which encoded a polypeptide with 337 amino acid residues (FIG. 12).

The coding sequence of the DDS polypeptide from S. trueperi was amplified by PCR using S. trueperi genomic DNA as template. PCR primers were designed based on the sequences obtained as described above. The sequences of the primers were as follows.

| | |
|---|---|
| SHDDSF: | |
| 5′-ATTAGGTACCATCAGATAATCGTCGCTCAA-3′ | (SEQ ID NO:92) |
| SHDDSR: | |
| 5′-TATAGGATCCGACATGGACGAGGAAGACGC-3′ | (SEQ ID NO:93) |

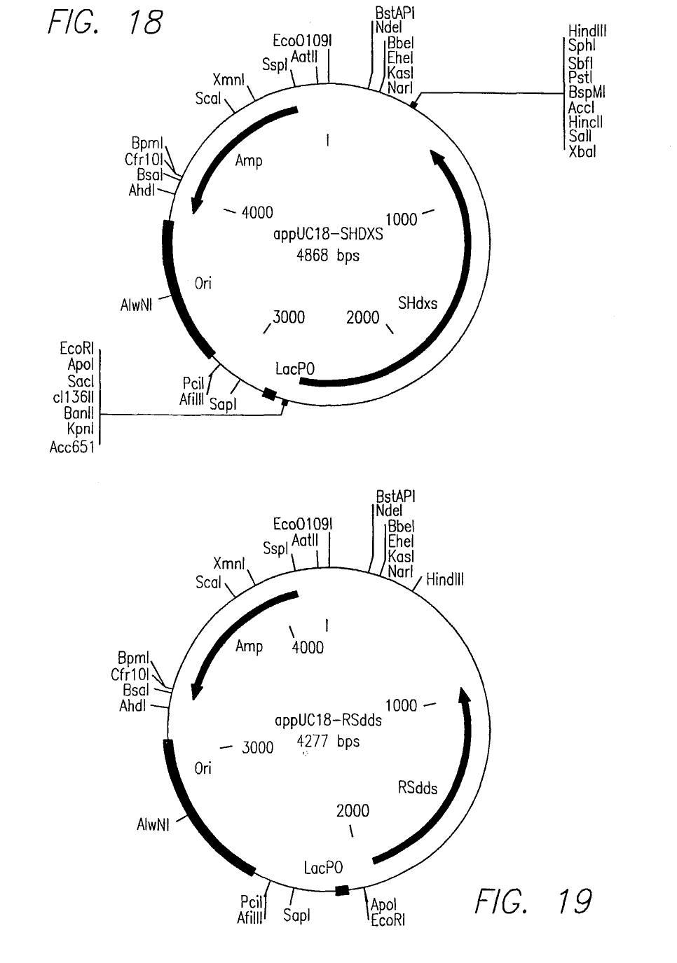

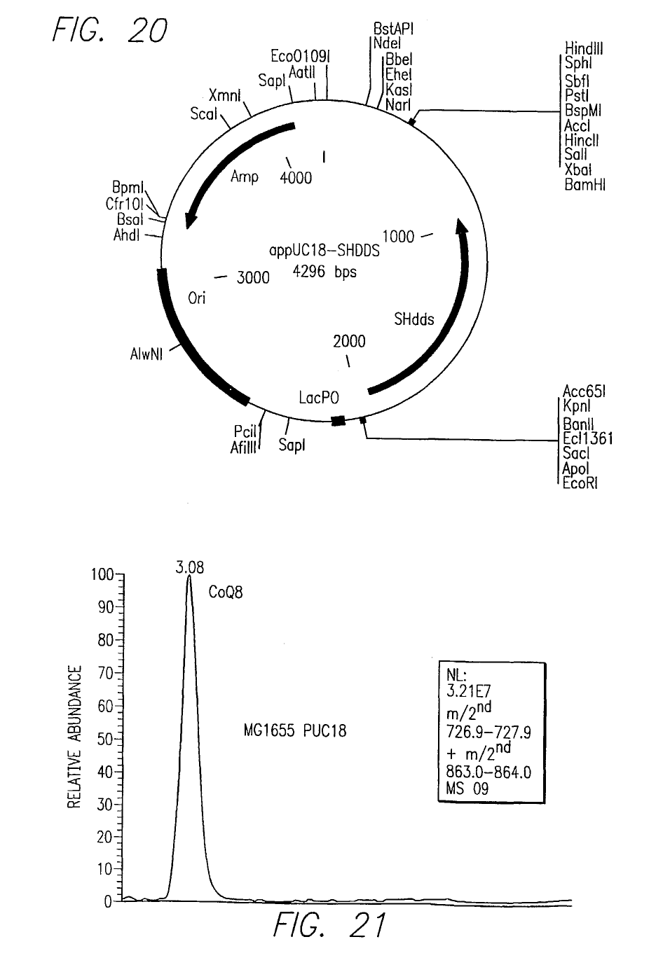

These primers were designed to introduce a Kpn I restriction site at the beginning of the amplified fragment and a BamH I restriction site at the end of amplified fragment. The sequence of each restriction site is underlined. The PCR reactions were performed as described in Example 3 with the exception that primers SHDDSF and SHDDSR were used instead of RDS 18F and RDS 18R. Once the PCR was completed, the PCR reactions were separated by gel electrophoresis using a 0.3 percent TAE agarose gel. The gel electrophoresis revealed a 1.6 kb fragment. This 1.6 kb fragment was (1) purified using a Qiagen Gel Extraction kit, (2) digested with Kpn I and BamH I (New England BioLabs), and (3) ligated into pUC18 that had also been digested with Kpn I and BamH I and gel purified using methods similar to those described in Example 3. The resulting construct designated appUC18-SHDDS is depicted in FIG. 20. This construct was used to transform cells as described in Example 3. The transformed cells were plated onto LB-Amp plates, and eight individual colonies were selected at random. Plasmid DNA was isolated from each colony using a QiaPrep Spin Miniprep kit. The extracted plasmid DNA was tested for the presence of the 1.6 kb fragment using three different restriction digests. If the plasmids contained the 1.6 kb fragment, then a BamH I and Kpn I digest would yield two fragments (2.68 and 1.62 kb), an EcoR I digest would yield two fragments (1.45 and 2.85 kb), and a Ban II digest would yield two fragments (0.48 and 3.8 kb). All eight plasmids tested yielded digestion fragments consistent with a plasmid containing the desired 1.6 kb fragment.

### Example 5

### Measuring CoQ(10)

Harvested cells were suspended in water to have about 0.1 gm dry weight per mL. The suspension was subjected to a French-press, and the resulting in suspension was frozen in 1 mL aliquots until used.

To measure CoQ(10) in a sample, two aliquots were repeatedly thawed and refrozen 4-5 times. Once transferred to a 50 mL centrifuge tube, 1 mL of 5% sodium dodecyl sulfate was added to the thawed material. The material was then flushed with nitrogen. After vortexing for one minute, six ML of ethanol was added to the material, and the resulting mixture was vortexed for one minute. Then, 15 mL of hexane was added to the mixture. After vortexing for five minutes, the mixture was centrifuged at 3000 rpm for ten minutes. Once centrifuged, the hexane layer was removed to a conical flask and flushed with nitrogen. This hexane extraction was repeated two times. The three extracts were pooled into a single tube that was evaporated on a vacuum evaporator until the residue was near dryness. The residue was dissolved in 2 mL of mobile phase by vortexing for 2-3 minutes. Once vortexed, the solution was transferred to a 5 mL volumetric flask. The tube that contained the residue was rinsed two additional times with 1 mL of mobile phase. Each time the rinse solution was transferred to the same 5 mL volumetric flask. After adjusting the total volume to 5 mL, the solution was mixed well and stored at −20° C. until analyzed.

As a control, either water or a culture solution was spiked with standard CoQ(10), extracted as indicated above, and analyzed to determine the recovery of the spiked material. The CoQ(10) standard was a stock solution of CoQ(10), obtained from Sigma. The stock solution was made in HPLC grade ethanol at a concentration of 100 μg/mL, and then diluted to get CoQ(10) solutions ranging from 100 μg/mL to 1 μg/mL.

HPLC analysis was performed with the following parameters. The mobile phase was ethanol:methanol (7:3) or methanol:isopropylether (9:1). The flow rate was 0.75 mL/min. The column was Waters Nova-Pak C18 (3.9×150 mm; 4Um). The detector was a PDA set from 200-300 nm with the resolution at 1.2 m and the maximum absorbance at 275 nm. The run time was 15 minutes, and the injection volume was 50 μL. To calculate the amount of CoQ(10) present, 50 μL of each sample was injected, and the results compared to those obtained using the calibration curve. From these data points, the concentration per gm dry weight was calculated.

### Example 6

### Introducing Nucleic Acid that Encodes a Polypeptide Having DDS Activity into Cells and Measuring Isoprenoid Levels

The following procedures were followed individually for the R. sphaeroides and S. trueperi nucleic acid isolated as described in Examples 3 and 4, respectively.

Plasmid DNA encoding the polypeptide having DDS activity was electroporated into wild type E. coli strain MG1655. The electroporated cells were plated onto LB-Amp plates. A single individual bacterial colony was picked for each DDS coding sequence, and each colony was grown overnight in 2 mL of LB-Amp at 37° C. with 200 rpm shaking. About 0.75 mL of these overnight cultures were used to inoculate flasks containing 75 mL LB-Amp medium (Amp concentration was 100 μg/mL). These second cultures were grown at 37° C. at 200 rpm for 30 hours. Additional Amp (to a final concentration of 50 μg of fresh Amp per mL) was added to each flask after 12 hours of growth. After 30 hours, the bacteria were collected by centrifugation at 8,000 g for 10 minutes. The resulting bacterial cell pellets were washed by adding 20 mL of 10 mM Tris-HCL buffer (pH 8.0), resuspending the cells, and re-centrifuging as before. Each cell pellet was then resuspended in 10 mL of water. About 0.5 mL of each extract was used for dry mass analysis and the remaining cell suspensions (about 9.5 mL) were frozen at −20° C. overnight.

The 9.5 mL cell suspensions were used as follows. First, the cells were thawed on ice and lysed by passing the cell suspensions through a French press three times (14,000 psi pressure). The resulting cell extracts were frozen at −20° C. in 1 mL aliquots and maintained on ice prior to analysis.

High pressure liquid chromatography was performed using Waters' 2690 Alliance integrated system (Waters Corporation, Milford, Mass.). Prior to analysis, all samples and standards were dissolved in HPLC-grade ethanol, loaded into the built-in auto-sampler, and kept at 5°-10° C. in the dark. The separation was carried out using an isocratic elution program of 70:30 ethanol/methanol (v/v) at a flow rate of 1.0 mL/min. The column was a Waters Nova-Pak C18, 3.9-150 mm equipped with a guard column of the same stationary phase. The injection volume was typically 10-25 μL. Total run time was ten minutes.

Under these conditions, retention times were 3.1 and 4.9 minutes for CoQ(8) and CoQ(10), respectively. For quantification purposes, a four-point external calibration curve was calculated using freshly prepared CoQ(10) standards. Calibration levels were 1.0, 4.0, 10.0 and 100.0 μg/mL (ppm). Each standard was injected in triplicate, and the resulting calibration plot was linearly fitted with observed r²'s of >0.999.

For UV and MS detection, a photodiode array (PDA, Model UV6000LP, ThermoQuest Corp., San Jose, Calif.) and an ion trap mass analyzer (LCQ Classic, Finnigan/ThermoQuest Corp., San Jose, Calif.) were connected in series with the chromatograph and without splitting of the effluent. The PDA was operated in scanning mode from 220-300 in. Effluent from the PDA was introduced into the mass analyzer via atmospheric-pressure chemical ionization (APCI) using the following parameters: capillary temperature, 150° C.; capillary voltage, 3 kV; vaporizer temperature, 400° C.; sheath gas (N₂) flow, 80 arbitrary units; auxiliary gas (N₂) flow, 5 arbitrary units; and corona discharge needle, 5 mA/6 kV. Positive-ion detection was performed in full scan (250-1000 m/z), 2 mscans, 500 ms ion injection time.

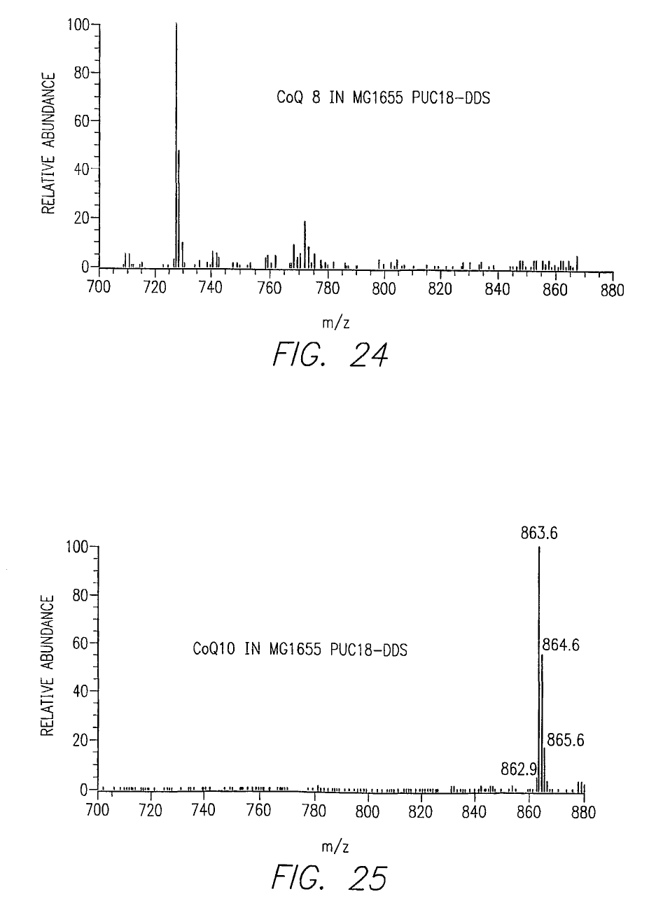

Under these conditions, CoQ(8) yielded a mass spectrum with a base peal(at 727.5 m/z, corresponding to the protonated ‘molecular ion’ as well as several satellite ions from ethanol and/or methanol adducts (FIGS. 23 and 24). Similarly, CoQ(10) yielded a mass spectrum with a base peak at 863.6 m/z corresponding to its protonated ‘molecular ion’ (FIG. 25). Several ethanol and/or methanol satellite adducts were observed as well. Both CoQ(8) and CoQ(10) yielded UV spectra with maxima at 274 nm.

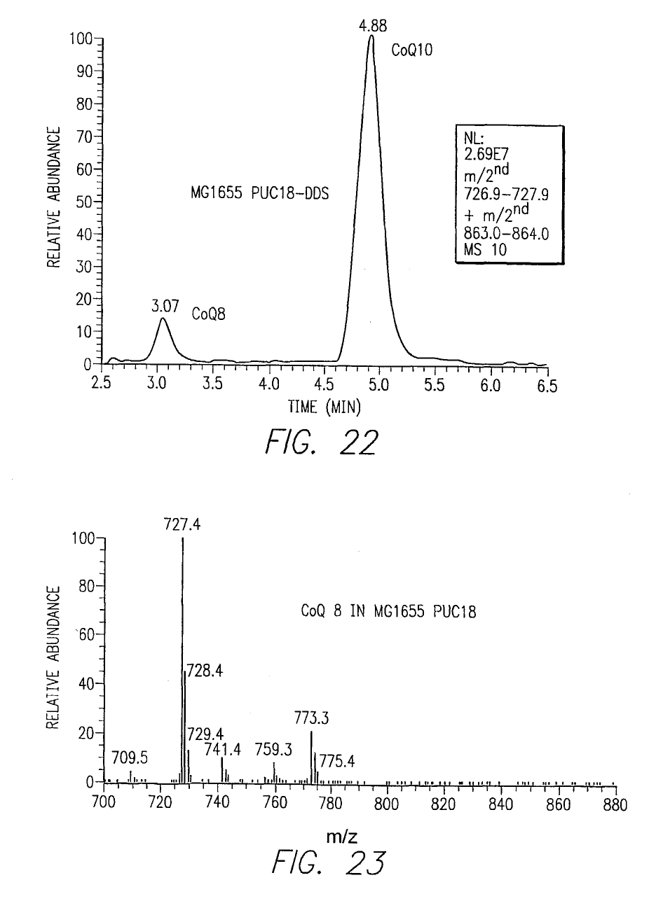

Two samples were analyzed: MG1655 PUC18 and MG1655 PUC18-DDS. MG1655 PUC18 is E. coli strain MG1655 transfected with the PUC18 vector only. MG1655 PUC18-DDS is E. coli strain MG1655 transfected with the PUC18 vector containing nucleic acid that encodes a R. sphaeroides polypeptide having DDS activity. The MG1655 PUC18 specimen contained only CoQ(8) (retention time 3.08 min, FIG. 21) as confirmed by its mass spectrum (FIG. 23), with a base peak at 727.4 ml/z and a UV spectrum with a maximum at 274 nm. The MG1655 PUC18-DDS specimen, however, contained CoQ(8) and CoQ(10) (FIG. 22), both of which were confirmed by matching mass spectra (FIGS. 24 and 25) and UV maxima.

### Example 7

### Cloning Nucleic Acid that Encodes a Sphingomonas trueperi Polypeptide Having DXR Activity

Sphingomonas trueperi ATCC 12417 cultures (100-200 mL) were grown in nutrient broth at 30° C. and 250 rpm for 2-3 days. The cells then were pelleted and washed with a 10 mM Tris: 1.0 mM EDTA solution. The pellets were resuspended in 5 mL of GTE buffer (50 mM glucose, 25 mM Tris HCl (pH 8.0), 10 mM EDTA (pH 8.0)) per 100 mL of culture. Lysozyme and Proteinase K were added to a 1 mg/mL concentration and mutaniolysin was added to 5.5 μg/mL. After a 1.5 hour incubation at 37° C., SDS was added to a final concentration of 1%, and the concentration of Proteinase K was brought to 2 mg/mL. After incubation at 50° C. for one hour, an equal volume of GTE buffer was added, and NaCl was added to a 0.15 M concentration. The mixture was extracted with an equal volume of phenol:chloroform:isoamyl alcohol (25:24:1) and centrifuged at 10,000 rpm for 10 minutes. The supernatant was removed to a clean tube, extracted with an equal volume of chloroform, and centrifuged at 5,000 rpm for 10 minutes. The supernatant was treated with RNAse and precipitated with 2.5 volumes of ethanol. The spooled DNA was washed with 70% ethanol, air dried, and resuspended in 10 MM Tris (pH 8.5). After resuspending, the resuspended DNA was further cleaned by re-extraction with phenol:chloroform:isoamyl alcohol and chloroform, and reprecipation with 1/10 volume 7.4 M NH₄OAc and 2.5 volumes ethanol.

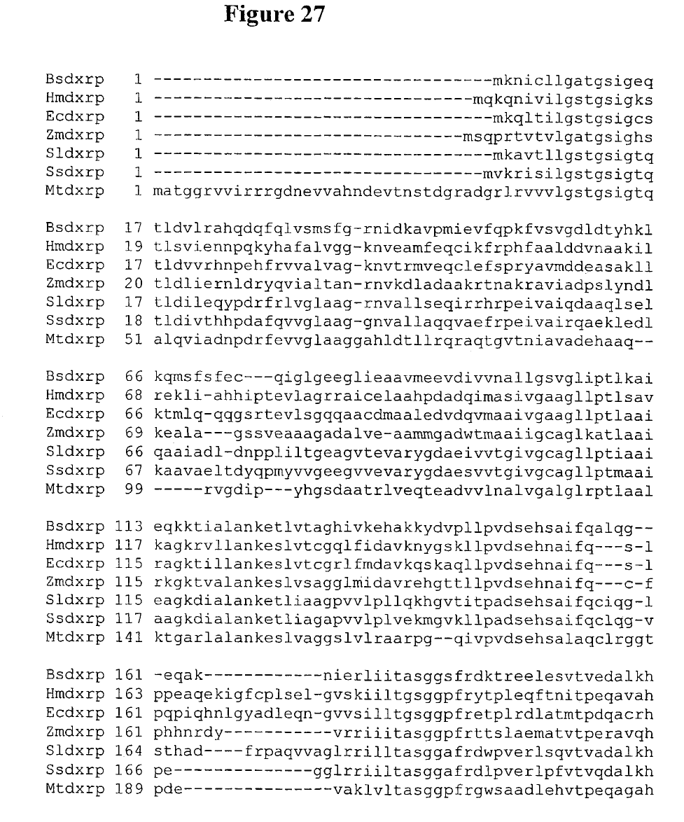

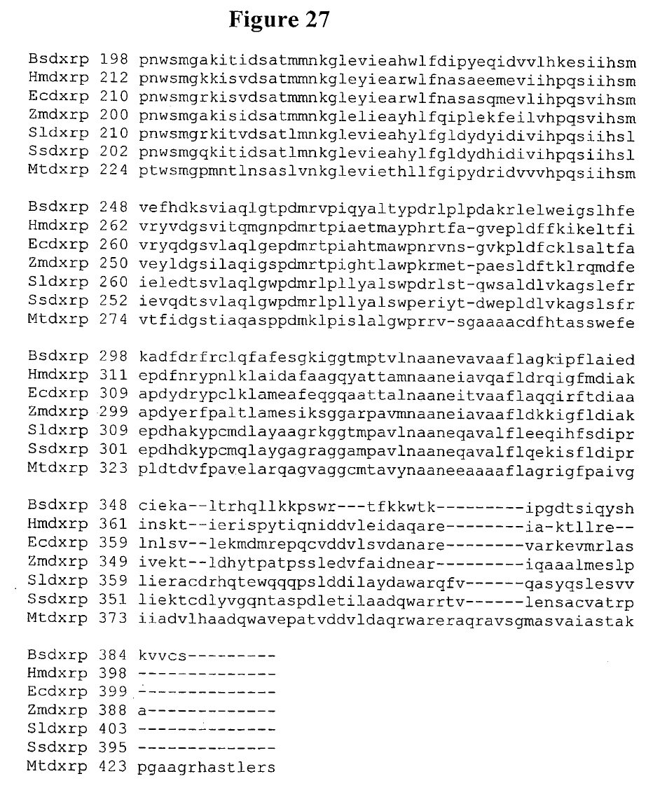

A conserved region of the 1-deoxy-D-xylulose 5-phosphate reductoisomerase (dxr) gene was cloned by PCR. Five degenerate forward and five degenerate reverse PCR primers were designed from conserved protein regions that were revealed by aligning known dxr genes (FIG. 27). The degenerate sequences were designed from the conserved regions using the universal codon table. The primers were used in all logical combinations in PCR using Taq polymerase (Roche Molecular Biochemicals, Indianapolis, Ind.) and 1 ng of genomic DNA/μL reaction mix. PCR was conducted using a touchdown PCR program with 4 cycles at an annealing temperature of 59° C., 4 cycles at 57° C., 4 cycles at 55° C., and 24 cycles at 53° C. Each cycle used an initial 30 second denaturing step at 94° C. and a 1.75 minute extension at 72° C., and the program had an initial denaturing step for 2 minutes at 94° C. and final extension of 5 minutes at 72° C. The amounts of PCR primer used in the reaction were increased 3-12 fold above typical PCR amounts depending on the amount of degeneracy in the 3′ end of the primer. In addition, separate PCR reactions containing each individual primer were made to identify PCR products resulting from single degenerate primers. Fifteen IL of each PCR product was separated on a 1.5% TAE (Tris-acetate-EDTA)-agarose gel. Degenerate primers F2 (5′-CCSGTSGAYWSSGARCAYAACGCS-3′ (SEQ ID NO: 132)) and R7 (5′-ATGATGAACAAGGGSCTSGAR-3′ (SEQ ID NO: 133)) produced a band of about 250 bp, which was the expected size based on dxr genes from other species. This band was not present in the individual F2 and R7 primer control reactions. Degenerate primers F3 (5′-CATCCVAACTGGWMVATGGG-3′ (SEQ ID NO:134)) and R2 (5′-ATYGGYRWWCKCATATCMGG-3′ (SEQ ID NO:135)) produced a band of about 200 bp, which also was the expected size. The F2-R7 and F3-R2 fragments were isolated and purified using a QIAquick Gel Extraction Kit (Qiagen Inc., Valencia, Calif.). Three μL of the purified band was ligated into pCR®II-TOPO vector, which was then transformed by a heat-shock method into TOP 10 E. coli cells using a TOPO cloning procedure (Invitrogen, Carlsbad, Calif.). Transformations were plated on LB media containing 100 μg/mL of ampicillin and 50 μg/mL of 5-Bromo-4-Chloro-3-Indolyl-B-D-Galactopyranoside (X-gal). Individual, white colonies were resuspended in about 20 μL of 10 mM Tris and heated for 10 minutes at 95° C. to break open the bacterial cells. To screen individual colonies, 2 μL of the heated cells was used in a 25 μL PCR reaction as described above using the appropriate degenerate primers. Plasmid DNA was obtained with a QIAprep Spin Miniprep Kit (Qiagen, Inc) from cultures of colonies having the desired insert and used for DNA sequencing with M13R and M13F primers. Sequence analysis revealed that the F2-R7 and F3-R2 fragments overlapped and were homologous to known dxr genes.

Genome walking was performed to obtain the complete coding sequence as follows. The overlapping of the F2-R7 and F3-R2 fragments resulted in a sequence 358 bp in length. The following four primers for conducting genome walking in both upstream and downstream directions were designed using the portion of this sequence that was internal to the degenerate primers:

| | |
|---|---|
| GSP1F | |
| 5′-CGAATGGACGACGGATTGGCGATGGAC-3′ | (SEQ ID NO:136) |
| GSP2F | |
| 5′-TCAGTTCGAGCCCCTTGTTCATCATCGTC- | (SEQ ID NO:137) |

3′

| | |
|---|---|
| GSPIR | |
| 5′-CGAACTGATCGAAGCCTTCCACCTGTTC-3′ | (SEQ ID NO:138) |
| GSP2R | |
| 5′-GGTCCATCGCCAATCCGTCGTCCATTC-3′ | (SEQ ID NO:139) |

The GSP1F and GSP2F primers faced upstream, the GSP1R and GSP2R primers faced downstream, and the GSP2F and GSP2R primers were nested inside the GSP1F and GSP1 R primers. Genome walking was conducted according to the manual for CLONTECH's Universal Genome Walking Kit (CLONTECH Laboratories, Inc., Palo Alto, Calif.) with the exception that the enzymes FspI and SmaI were used in place of DraI and EcoRV. The DraI and EcoRV enzymes were replaced because they cut S. trueperi genomic DNA too infrequently to give fragment lengths amenable to PCR. The PCR mixture contained 5% DMSO. First round PCR was conducted in a Perkin Elmer 9700 Thermocycler with 7 cycles consisting of 2 seconds at 94° C. and 3 minutes at 72° C., and 36 cycles consisting of 2 seconds at 94° C. and 3 minutes at 66° C., with a final extension at 66° C. for 4 minutes. Second round PCR used 5 cycles consisting of 2 seconds at 94° C. and 3 minutes at 72° C., and 26 cycles consisting of 2 seconds at 94° C. and 3 minutes at 66° C., with a final extension at 66° C. for 4 minutes. Nine μL of the first round product and seven μL of the second round product were separated on a 1.5% TAE-agarose gel. A 1.3 Kb band was obtained from the second round product for the SmaI forward reaction, an 800 bp band for the StuI reverse reaction, and a 750 bp band for the PvuII reverse reaction. These fragments were gel purified, cloned, and sequenced. Internal primers were used to amplify and obtain additional sequence of the gene. Sequence analysis revealed that the sequence derived from genome walking overlapped with the original fragments and contained an entire coding sequence homologous to known dxr genes. The full-length clone containing coding and non-coding sequence was 2017 bp in length (FIG. 28). The open reading frame starting with the first GTG site was 1161 bp in length (FIG. 29), which encoded a polypeptide with 386 amino acid residues (FIG. 30).

### Example 8

### Making Recombinant Microorganisms

Rhodobacter sphaeroides (ATCC 35053) was routinely maintained on Luria Bretain (Miller) agar (Fisher scientific) plates. When needed, R. sphaeroides was cultured as follows. A 5 mL culture was grown in a 15 mL culture tube at 30° C. in Innova 4230 Incubator, Shalzer (New Brunswick Scientific, Edison, NJ) with a shaking speed of 250 rpm. Each 5 mL culture was started by inoculating liquid media (Sistrom media supplemented with 20% LB) with a single colony. The liquid media contained the following ingredients per liter: 2.72 g KH₂PO₄, 0.5 g (NH₄)₂SO₄, 0.5 g NaCl, 0.2 g EDTA disodium salt, 0.3 g MgSO_{4·}·7H₂O, 0.033 g CaCl₂·2H₂O, 0.2 mg FeSO₄·7H₂O, 0.02 mL (NH₄)₆Mo₇O₂₄·4H₂O (1% solution), 1 mL Trace element solution, 0.2 mL Vitamin solution, 5 g Luria Bretain Broth Mix, and 8 mL Glucose (50%). The Trace element solution contained the following ingredients per liter: 1.765 g EDTA disodium salt, 10.95 g ZnSO₄·7H₂O, 5 g FeSO_{4·7}H₂O, 1.54 g MnSO₄·H₂O, 0.392 g CuSO₄·5H₂O, 0.284 g Co(NO₃)₂·6H₂O, and 0.114 g H₃BO₃. The Vitamin solution contained the following ingredients per liter: 10 g Nicotinic acid, 5 g Thiamine HCl, and 0.01 g Biotin. The vitamins and glucose were added after the media cooled to room temperature after autoclaving. When necessary, the media was supplemented with one or more of the following antibiotics: Kanamycin (25 μg/mL; final concentration), Spectinomycin (25 μg/mL; final concentration), and/or Streptomycin (25 μg/mL; final concentration).

Electrocompetent R. sphaeroides Cells

Electrocompetent R. sphaeroides cells were made as follows. A 5 mL culture of R. sphaeroides was grown overnight at 30° C. in Sistrom's media supplemented with 20% LB. This culture was diluted 1/100 in 300 mL of the same media and grown to an OD₆₆₀ of 0.5-0.8. The cells were chilled on ice for 10 minutes and then centrifuged for 6 minutes at 7,500 g. The supernatant was discarded, and the cell pellet was resuspended in ice-cold 10% glycerol at half of the original volume. The cells were pelleted by centrifugation for 6 minutes at 7,500 g. The supernatant was again discarded, and cells resuspended in ice-cold 10% glycerol at one quarter of the original volume. The last centrifugation and resuspension steps were repeated, followed by centrifugation for 6 minutes at 7,500 g. The supernatant was decanted, and the cells resuspended in the small volume of glycerol that did not drain out. Additional ice-cold 10% glycerol was added to resuspend the cells, if necessary. Forty μL of the resuspended cells was used in a test electroporation to determine if the cells needed to be concentrated by centrifugation or diluted with 10% ice-cold glycerol. Time constants of 8.5-9.0 milliseconds resulted in good transformation efficiencies. If cells were too dilute, the time constant was greater than 9.0 and transformation efficiencies were low. If cells were too concentrated, the electroporation would spark. Once an acceptable time constant was achieved, cells were aliquoted into cold microfuge tubes and stored at −80° C. All water used for media and glycerol was 18.2 Mohm-cm or higher.

Electrocompetent R. sphaeroides cells were electroporated as follows. One μL of plasmid DNA was gently mixed into 40 μL of R. sphaeroides electrocompetent cells, which were then transferred to an electroporation cuvette with a 0.2 cm electrode gap. Electroporations were conducted using a Biorad Gene Pulser II (Biorad, Hercules, Calif.) with settings at 2.5 kV of energy, 400 ohms of resistance, and 25 μF of capacitance. Cells were recovered in 400 μL SOC media at 30° C. for 6-16 hours. The cells were then plated (200 μL per plate) on the appropriate selective media. Transformation efficiencies averaged about 2,000 transformants/μg of DNA.

Electrocompetent E. coli cells

Electrocompetent E. coli strain S17-1 cells were made as follows. A 5 mL culture of E. coli strain S17-1 was grown overnight at 30° C. in LB media supplemented with 25 μg/mL of streptomycin and 25 μg/mL of spectinomycin. This culture was diluted 1/100 in 300 mL of the same media and grown to an OD₆₆₀ of 0.5-0.8. The cells were chilled on ice for 10 minutes and then centrifuged for 6 minutes at 7,500 g. The supernatant was discarded, and the cell pellet was resuspended in ice-cold 10% glycerol at half of the original volume. The cells were pelleted by centrifugation for 6 minutes at 7,500 g. The supernatant was again discarded, and the cells were resuspended in ice-cold 10% glycerol at one quarter of the original volume. The last centrifugation and resuspension steps were repeated, followed by centrifugation for 6 minutes at 7,500 g. The supernatant was decanted, and the cells resuspended in the small volume of glycerol that did not drain out. Additional ice-cold 10% glycerol was added to resuspend the cells, if necessary. Cells were aliquoted into cold microfuge tubes and stored at −80° C.

Electrocompetent E. coli strain S17-1 cells were electroporated as follows. Forty μL of competent cells was used per electroporation. Electroporation was conducted using a Biorad Gene Pulser II and a standard E. coli protocol: 2.5 kV of energy, 200 ohms of resistance, and 25 μF of capacitance. Electroporated cells were recovered in 250-1000 μL of SOC media for one hour, and 10-200 μL of culture was plated per plate of selective media. Transformation efficiencies averaged about 1.5×10⁴ transformants/μg of DNA.

Constructs

Various clones were overexpressed in R. sphaeroides using the broad-host-range vector pBBR1MCS2 (Kovach et al., Gene, 166:175-176 (1995)) that was engineered to have either an R. sphaeroides rrnb promoter, an R. sphaeroides glnB promoter, or a tet promoter. The pBBR1MCS2 vector is mobilizable and relatively small (5,144 bp), replicates in R. sphaeroides, has a multiple cloning site with lacZα color selection, and carries a kanamycin resistance gene. All restriction enzymes and T4 DNA ligase were obtained from New England Biolabs (Beverly, Mass.) unless otherwise indicated. All plasmid DNA preparations were done using QIAprep Spin Miniprep Kits or Qiagen Maxi Prep Kits, and all gel purifications were done using QIAquick Gel Extraction Kits (Qiagen, Valencia, Calif.).

pMCS2rrnBP

The vector designated pMCS2rrnBP, which contains an R. sphaeroides rrnB promoter, was constructed by inserting a copy of the R. sphaeroides rrnB promoter (rrnBP) into the pBBR1MCS2 vector. The rrnB promoter was isolated from the pTEX124 vector (obtained from S. Kaplan) by digestion with the restriction enzyme BamHI, which releases the promoter as a 363 bp fragment. Alternatively, the rrnB promoter can be obtained by PCR amplifying it from R. sphaeroides genomic DNA using primers based on published rrnB sequence (GenBank® accession number X53854). This fragment was gel purified from a 2% Tris-acetate-EDTA (TAE) agarose gel. The pBBR1MCS2 vector was also digested with BamHI, and the enzyme heat inactivated at 80° C. for 20 minutes. The digested vector was then dephosphorylated with shrimp alkaline phosphatase (Roche Moelcular Biochemicals, Indianapolis, Ind.) and gel purified from a 1% TAE-agarose gel. The prepared vector and the rrnBP fragment were ligated using T4 DNA ligase at 16° C. for 16 hours. One ILL of ligation reaction was used to electroporate 40 μL of E. coli Electromax™ DH10B™ cells (Life Technologies, Inc., Rockville, Md.). Electroporated cells were plated on LB media containing 25 μg/mL of kanamycin (LBK). Plasmid DNA was isolated from cultures of single colonies and was digested with HindIII restriction enzyme to confirm the presence of a single insertion of the rrnb promoter. The sequence of the rrnBP inserts for these colonies was also confirmed by DNA sequencing.

pMCS2glnBP

The vector designated pMCS2glnBP, which contains an R. sphaeroides glnB promoter, was constructed by inserting a copy of the R. sphaeroides glnB promoter (glnBP) into the pBBR1 MCS2 vector. The glnb promoter was PCR amplified from genomic DNA obtained from R. sphaeroides strain 35053. The following primers were designed based on sequence information obtained from GenBank® accession number X71659:

(SEQ ID NO:140)

| | |
|---|---|
| glnBF | 5′-ATTATCTAGAATCCGCCCCGCCTCCACCTC-3′ |

(SEQ ID NO:141)

| | |
|---|---|
| glnBR | 5′-GATGGATCCTGGGTAGGGTCGCTGCTGTCC-3′ |

The primers introduced an XbaI restriction site at the 5′ end and a BamHI restriction site at the 3′ end. The following reaction mix and PCR program was used to amplify the promoter region of the glnB gene.

| Reaction Mix | | PCR program |
|---|---|---|
| Pfu 10X buffer | 10 μL | 94° C. 2 minutes |
| DMSO | 5 μL | 7 cycles of: |
| dNTP mix (10 mM) | 4 μL | 94° C. 30 seconds |
| glnBF (50 μM) | 2 μL | 61° C. 45 seconds |
| glnBP (50 μM) | 2 μL | 72° C. 3 minutes |
| Genomic DNA (50 ng/μL) | 2 μL | 25 cycles of: |
| Pfu enzyme (2.5 U/μL) | 2 μL | 94° C. 30 seconds |
| DI water | 73 μL | 66° C. 45 seconds |
| | | 72° C. 3 minutes |
| Total: | 100 μL | 72° C. 7 minutes |
| | | 4° C. Until used further |

The PCR product was separated on a 1.2% TAE-agarose gel. An about 500 bp fragment was excised and gel purified. The isolated DNA was restricted with XbaI and BamHI, and the resulting digested DNA column purified using a Qiagen gel isolation kit. Three μg of pBBR1MCS2 plasmid DNA was digested with BamHI and XbaI. The digestion was inactivated at 80° C. for 20 minutes. The digested vector was then dephosphorylated with shrimp alkaline phosphatase and gel purified on a 1% TAE-agarose gel. Eighty-six ng of the prepared pBBR1MCS2 vector was ligated with 60 ng of the digested glnBP PCR product using T4 DNA ligase at 14° C. for 14-16 hours. One μL of ligation reaction was used to electroporate 40 μL of E. coli Electromax™ DH10 B™ cells. Electroporated cells were plated on LB media containing 25 μg/mL of kanamycin and 50 μg/mL of Xgal (LBKX). Eight individual, white colonies were selected, and their plasmid DNA isolated using a QIAprep Spin Miniprep Kit. Plasmid DNA isolated from each colony was digested in separation reaction mixtures with PstI and a combination of EcoRI/XbaI. All eight clones had a restriction pattern that indicated the presence of the insert. The sequence of three clones was verified.

pMCS2tetP

The vector designated pMCS2tetP, which contains a tet promoter, was constructed by cloning the promoter for the tetracycline resistance determinants from transposon Tn1721 (Waters et al., Nucleic Acids Research, 11(17):6089-6105 (1983)) into the pBBR1MCS2 vector. The tetA gene promoter (tetP) was amplified using plasmid pRK415 as template. The following primers were designed to introduce an XbaI restriction site at the beginning of the amplified fragment and a BamHI site at the end of the amplified fragment.

| | |
|---|---|
| TETXBAF | |
| 5′-TTATCTAGAACCGTCTACGCCGACCTC- | (SEQ ID NO:142) |
| GTTCAAC-3′ | |
| TETBAMR | |
| 5′-TTAGGATCCCCTCCGCTGGTCCGATTG- | (SEQ ID NO:143) |
| AAC-3′ | |

The PCR mix contained the following: 1×Native Plus Pfu buffer, 20 ng pRK415 plasmid DNA, 0.2 μM of each primer, 0.2 mM of each dNTP, 5% DMSO (v/v), and 10 units of native Pfu DNA polymerase in a final volume of 200 μL. The PCR reaction was performed in a Perkin Elmer Geneamp PCR System 2400 under the following conditions: an initial denaturation at 94° C. for 1 minute; 8 cycles of 94° C. for 30 seconds, 60° C. for 45 seconds, and 72° C. for 45 seconds; 24 cycles of 94° C. for 30 seconds, 66° C. for 45 seconds, and 72° C. for 45 seconds; and a final extension for 7 minutes at 72° C. The amplification product was then separated by gel electrophoresis using a 2%TAE-agarose gel. A 160 bp fragment was excised from the gel and purified. The purified fragment was digested simultaneously with XbaI and BamHI restriction enzymes, and purified with a QIAquick PCR Purification Kit. Three μg of pBBR1MCS2 plasmid DNA was digested with BamHI and XbaI, and the digest was inactivated at 80° C. for 20 minutes. The digested vector was then dephosphorylated with shrimp alkaline phosphatase and gel purified on a 1% TAE-agarose gel.

100 ng of the prepared pBBR1MCS2 vector was ligated with 36 ng of the digested tetP PCR product using T4 DNA ligase at 16° C. for 16 hours. One μL of ligation reaction was used to electroporate 40 μL of E. coli Electromax™ DH5α™ cells. Electroporated cells were plated on LB media containing 25 μg/mL of kanamycin and 50 μg/mL of Xgal (LBKX). Individual, white colonies were resuspended in about 25 μL of 10 mM Tris, and 2 μL of the resuspension was plated on LBKX. The remnant resuspension was heated for 10 minutes at 95° C. to break open the bacterial cells. Two μL of the heated cells was used in a 25 μL PCR reaction using the following primers homologous to the vector and flanking the cloning site:

| | | |
|---|---|---|
| MCS2FS | 5′-AGGCGATTAAGTTGGGTAAC-3′ | (SEQ ID NO:144) |
| MCS2RS | 5′-GACCATGATTACGCCAAG-3′ | (SEQ ID NO:145) |

The PCR mix contained the following: 1×Taq PCR buffer, 0.2 μM each primer, 0.2 mM each dNTP, and 1 unit of Taq DNA polymerase per reaction. The PCR reaction was performed in a MJ Research PTC100 under the following conditions: an initial denaturation at 94° C. for 2 minutes; 32 cycles of 94° C. for 30 seconds, 55° C. for 45 seconds, and 72° C. for 1 minute; and a final extension for 7 minutes at 72° C. All colonies showed a single insertion event. Plasmid DNA was isolated from cultures of two individual colonies and sequenced to confirm the DNA sequence of the tet promoter in the construct.

pMCS2rrnBP/Stdxs

The nucleic acid encoding a S. trueperi polypeptide having DXS activity was cloned in the pMCS2rmBP vector as follows. The S. trueperi dxs gene was amplified by PCR using primers homologous to sequence upstream and downstream of the gene. These primers, STDXSMCSF and STDXSMCSR, were designed to introduce a ClaI restriction site at the beginning of the amplified fragment and a KpnI site at the end of the amplified fragment.

| | |
|---|---|
| STDXSMCSF | |
| 5′-GATAATCGATGTGTGACTGACCTGT- | (SEQ ID NO:146) |
| CCAAC-3′ | |
| STDXSMCSR | |
| 5′-CTTAGGTACCATGTTGGAGATTCAA- | (SEQ ID NO:147) |
| GGTGG-3′ | |

The PCR mix contained the following: 1×Native Plus Pfu buffer, 200 ng S. trueperi genomic DNA, 0.2 μM of each primer, 0.2 mM each dNTP, 5% DMSO (v/v), and 10 units of native Pfu DNA polymerase (Stratagene, La Jolla, Calif.) in a final volume of 200 μ. The PCR reaction was performed in a Perkin Elmer Geneamp PCR System 2400 under the following conditions: an initial denaturation at 94° C. for 1 minute; 8 cycles of 94° C. for 30 seconds, 54° C. for 45 seconds, and 72° C. for 3.5 minutes; 27 cycles of 94° C. for 30 seconds, 60° C. for 45 seconds, and 72° C. for 3.5 minutes; and a final extension for 7 minutes at 72° C. The amplification product was then separated by gel electrophoresis using a 1% TAE-agarose gel. A 2.2 Kb fragment was excised from the gel and purified. The purified fragment was digested with ClaI restriction enzyme, purified with a QIAquick PCR Purification Kit, digested with KpnI restriction enzyme, purified again with a QIAquick PCR Purification Kit, and quantified on a minigel.

Three μg of the pMCS2rrnBP vector was digested with the restriction enzyme ClaI, gel purified on a 1% TAE-agarose gel, digested with KpnI, purified with a QIAquick PCR Purification Kit, dephosphorylated with shrimp alkaline phosphatase, and purified again with a QIAquick PCR Purification Kit. 120 ng of the digested PCR product containing the S. trueperi dxs gene and the 50 ng of the prepared pMCS2rrnBP vector was ligated using T4 DNA ligase at 16° C. for 16 hours. One μL of the ligation reaction was used to electroporate 40 μL of E. coli Electromax™ DH10BTM cells. The electroporated cells were plated onto media. Plasmid DNA was isolated from cultures of individual colonies and evaluated for the presence of the desired insert by restriction enzyme analysis with HindIII and SacI enzymes. The sequence of the Stdxs insert was confirmed by DNA sequencing. The resulting plasmid containing the Stdxs sequence under the control of the rrnB promotor was designated pMCS2rrnBP/Stdxs.

Purified pMCS2rrnBP/Stdxs plasmid DNA derived from a colony having the correct sequence was then electroporated into electrocompetent cells of R. sphaeroides strain 35053. Plasmid DNA was isolated from cultures of individual R. sphaeroides colonies. Restriction patterns of plasmid preparations from R. sphaeroides are difficult to analyze due to the presence of multiple native plasmids in this species. To check the plasmid integrity in R. sphaeroides, one IL of the plasmid preparation from a transformed R. sphaeroides colony was used to re-transform E. coli Electromax™ DH10B™ cells by electroporation. Electroporated cells were plated on LBK media. Plasmid DNA was isolated from cultures of individual colonies and evaluated using SacI and HindIII restriction digests.

pMCS2rrnBP/Stdxs2

A second pMCS2rrnBP plasmid containing the nucleic acid encoding a S. trueperi polypeptide having DXS activity was constructed. This construct was made using the following forward primer designed to introduce the ribosomal binding site (rbs) from the R. sphaeroides dxs I gene along with a ClaI restriction site.

| | | |
|---|---|---|
| SXSCLAF2 | 5′-ACTATCGATGAAGGAAGAGCATGGCTGACCT-ACCCAAGAC-3′ | (SEQ ID NO:146) |

S. trueperi genomic DNA was used as template in a PCR mixture using the primers SXSCLAF2 and STDXSMCSR. The PCR program and reaction mixture used were identical to those described for the pMCS2rrnBP/Stdxs construct. The PCR product was gel purified, digested with ClaI, purified with a QIAquick PCR Purification Kit, digested with restriction enzyme KpnI, and purified again with a QIAquick PCR Purification Kit. 150 ng of digested PCR product was ligated into 50 ng of the prepared pMCS2rrnBP vector using T4 DNA ligase at 16° C. for 16 hours. One μL of the ligation reaction was transformed into E. coli Electromax™ DH10BTM cells, and the electroporated cells were plated onto LBK plates. Plasmid DNA was isolated from cultures of individual colonies and evaluated for the presence of the desired insert by restriction enzyme analysis with HindIII and SacI enzymes. The sequence of the dxs insert was confirmed by DNA sequencing. The resulting plasmid containing the Stdxs sequence under the control of the rrnB promotor and having an R. sphaeroides ribosomal binding site was designated pMCS2rrnBP/Stdxs2.

A confirmed construct was electroporated into R. sphaeroides strain 35053, and the electroporated cells were plated onto LBK media. Individual colonies were resuspended in about 25 μL of 10 mM Tris, and 2 μL of the resuspension was plated on LBK media. The remnant resuspension was heated for 10 minutes at 95° C. to break open the bacterial cells, and two μL of the heated cells used in a 25 μL PCR reaction using the SXSCLAF2 and STDXSMCSR primers. The PCR mix contained the following: 1×Taq PCR buffer, 0.2 μM each primer, 0.2 mM each dNTP, 5% DMSO (v/v), and 1 unit of Taq DNA polymerase (Roche) per reaction. The PCR reaction was performed in a MJ Research PTC100 under the following conditions: an initial denaturation at 94° C. for 2 minutes; 8 cycles of 94° C. for 30 seconds, 54° C. for 1 minute, and 72° C. for 3.5 minutes; 24 cycles of 94° C. for 30 seconds, 60° C. 1 minute, and 72° C. for 3.5 minutes; and a final extension for 7 minutes at 72° C.

pMCS2rrnBP/Rsdds

The nucleic acid encoding a R. sphaeroides polypeptide having DDS activity was cloned in the pMCS2 rrnBP vector as follows. The R. sphaeroides dds gene was PCR amplified using the following primer pair:

(SEQ ID NO:147)

| | |
|---|---|
| RDS18F | 5′-ACTAGAATTCCGCAACAGTTCCTTCATGTC-3′ |

(SEQ ID NO:148)

| | |
|---|---|
| RSDDSMCSR | 5′-CTAGATCGATACTTGCGGTCGGACTGATAG-3′ |

The forward primer was located upstream of the start codon and introduced an EcoRI restriction site, while the reverse primer was located downstream of the stop codon and introduced a ClaI restriction site. Since the forward primer was located upstream, the R. sphaeroides dds maintained its native ribosomal binding site. The following reaction mix and PCR program were used to amplify the R. sphaeroides dds gene.

| Reaction Mix | | Program |
|---|---|---|
| Pfu 10X buffer | 10 μL | 94° C. 2 minutes |
| DMSO | 5 μL | 8 cycles of: |
| dNTP mix (10 mM) | 4 μL | 94° C. 30 seconds |
| RDS18F (50 μM) | 2 μL | 55° C. 45 seconds |
| RSDDSMCSR (50 μM) | 2 μL | 72° C 3 minutes |
| Genomic DNA (50 ng/μL) | 2 μL | 21 cycles of: |
| Pfu enzyme (2.5 U/μL) | 1 μL | 94° C. 30 seconds |
| DI water | 74 μL | 61° C. 45 seconds |
| | | 72° C. 3 minutes |
| Total: | 100 μL | 72° C. 7 minutes |
| | | 4° C. Until used further |

The PCR product was separated on a 1% TAE-agarose gel, and an about 1.8 Kb fragment was excised and gel purified. The isolated DNA was restricted with EcoRI and ClaI, and was column purified using a Qiagen gel isolation kit. Three μg of pMCS2rrnBP vector DNA was digested with EcoRI, and the linear DNA was gel isolated using a Qiagen gel isolation kit. The vector was further digested with ClaI, and the DNA was column purified. The double-digested vector was then dephosphorylated with shrimp alkaline phosphatase and purified using a QIAquick PCR Purification Kit. The EcoRI/ClaI-digested R. sphaeroides dds PCR product was ligated into the prepared vector using T4 DNA ligase for 14-16 hours at 16° C. One μL of the ligation reaction was transformed into E. coli Electromax™ DH10BTM cells, which were then plated on LBK (25 μg/mL) media. Individual colonies were resuspended in about 25 μL of DI water, and 2 μL of the resuspension was plated on LBK. The remnant resuspension was heated for 10 minutes at 95° C. to break open the bacterial cells, and 2 μL of the heated cells was used in a 25 μL PCR reaction using the RDS 18F and RSDDSMCSR primers. The PCR mix contained the following: 1×Taq PCR buffer, 0.2 μM each primer, 0.2 μM each dNTP, 5% DMSO (v/v), and 1 unit of Taq DNA polymerase per reaction. The PCR reaction was performed under the following conditions: an initial denaturation at 94° C. for 2 minutes; 6 cycles of 94° C. for 30 seconds, 55° C. for 45 seconds, and 72° C. for 3 minutes; 25 cycles of 94° C. for 30 seconds, 61° C. 45 seconds, and 72° C. for 3 minutes; and a final extension for 7 minutes at 72° C. The resulting plasmid containing the Rsdds sequence under the control of the rrnB promotor was designated pMCS2rrnBP/Rsdds.

The pMCS2rrnBP/Rsdds plasimd was electroporated into E. coli strain S17-1. This strain contains a chromosomal copy of the transacting elements that mobilize oriT-containing plasmids during conjugation with a second bacterial strain. It also carries a gene conferring resistance to the antibiotics streptomycin and spectinomycin.

Using the S17-1 strain, the pMCS2rrnBP/Rsdds plasmid was transferred to R. sphaeroides 35053 by conjugation. Individual colonies were purified by restreaking on LBK plates. Single colonies were screened by PCR using the RDS 18F and RSDDSMCSR primers to confirm the presence of the insert as described above.

pMCS2rrnBP/Stdds

The nucleic acid encoding a S. trueperi polypeptide having DDS activity was cloned in the pMCS2rrnBP vector as follows. The S. trueperi dds gene was PCR amplified using the following primer pair:

(SEQ ID NO:149)

| | |
|---|---|
| STDDSMCSF | 5′-GTCGCTCGAGATCAGATAATCGTCGCTCAA-3′ |

(SEQ ID NO:150)

| | |
|---|---|
| STDDSMCSR | 5′-ATATGGTACCGACATGGACGAGGAAGACGC-3′ |

The forward primer was located upstream of the start codon and introduced a XhoI restriction site, while the reverse primer was located downstream of the stop codon and introduced a KpnI restriction site. Since the forward primer was located upstream, the S. trueperi dds fragment maintained its native ribosomal binding site. The following reaction mix and PCR program were used to amplify the S. trueperi dds gene.

| Reaction Mix | | Program |
|---|---|---|
| Pfu 10X buffer | 10 μL | 94° C. 2 minutes |
| DMSO | 5 μL | 8 cycles of: |
| dNTP mix (10 mM) | 4 μL | 94° C. 30 seconds |
| SHDDSMCSF (50 μM) | 2 μL | 55° C. 45 seconds |
| SHDDSMCSR (50 μM) | 2 μL | 72° C. 3 minutes |
| Genomic DNA (50 ng/μL) | 2 μL | 21 cycles of: |
| Pfu enzyme (2.5 U/μL) | 1 μL | 94° C. 30 seconds |
| DI water | 74 μL | 61° C. 45 seconds |
| | | 72° C. 3 minutes |
| Total: | 100 μL | 72° C. 7 minutes |
| | | 4° C. Until used further |

The PCR product was separated on a 1% TAE-agarose gel, and an about 1.6 Kb fragment was excised. The DNA was isolated using a Qiagen gel isolation kit. The isolated DNA was restricted with XhoI and KpnI, and was column purified using a Qiagen gel isolation kit. Two μg of pMCS2rrnBP vector DNA was digested with KpnI, and the linear DNA was gel isolated using a Qiagen gel isolation kit. The vector was further digested with XhoI, and the DNA was column purified. The double-digested vector was then dephosphorylated with shrimp alkaline phosphatase and column purified using a Qiagen gel purification kit. The XhoI/KpnI-digested S. trueperi dds PCR product was ligated into the prepared vector using T4 DNA ligase for 14-16 hours at 16° C. One μL of the ligation reaction was transformed into E. coli Electromax™ DH 10BTM cells, which were then plated on LBK (25 μg/mL) media. Individual colonies were resuspended in about 25 μL of DI water, and 2 IL of the resuspension was plated on LBK. The remnant resuspension was heated for 10 minutes at 95° C. to break open the bacterial cells, and 2 μL of the heated cells was used in a 25 μL PCR reaction using the SHDDSMCSF and SHDDSMCSR primers. The PCR mix contained the following: 1×Taq PCR buffer, 0.2 μM each primer, 0.2 mM each dNTP, 5% DMSO (v/v), and 1 unit of Taq DNA polymerase per reaction. The PCR reaction was performed under the following conditions: an initial denaturation at 94° C. for 2 minutes; 6 cycles of 94° C. for 30 seconds, 55° C. for 45 seconds, and 72° C. for 3 minutes; 25 cycles of 94° C. for 30 seconds, 61° C. 45 seconds, and 72° C. for 3 minutes; and a final extension for 7 minutes at 72° C. The resulting plasmid containing the Stdds sequence under the control of the rrnB promotor was designated pMCS2rrnBP/Stdds.

The pMCS2rrnBP/Stdds plasmid was electroporated into E. coli strain S17-1. Using the S17-1 strain, the pMCS2rrnBP/Stdds plasmid was transferred to R. sphaeroides 35053 by conjugation. Individual colonies were purified by restreaking on LBK plates. Single colonies were screened by PCR using the SHDDSMCSF and SHDDSMCSR primers to confirm the presence of the insert as described above.

pMCS2glnBP/Rsdds

The nucleic acid encoding a R. sphaeroides polypeptide having DDS activity was cloned in the pMCS2glnBP vector as follows. The R. sphaeroides dds gene was PCR amplified using the following primer pair.

| | | |
|---|---|---|
| RSDDSF | 5′-TAGAGAATTCGAAGGAAGAGCATGGGATTGGACG-AGGTTTC-3′ | (SEQ ID NO:151) |
| RSDDSR | 5′-TACTACTTGTATGTAGGTACCACTTGCGGTCGGAC-TGATAG-3′ | (SEQ ID NO:152) |

The forward primer introduced an EcoRI restriction site and a ribosomal binding site that was designed based on R. sphaeroides dxs1 gene. The reverse primer introduced a KpnI restriction site. Following reaction mix and PCR program was used to amplify the R. sphaeroides dds gene.

| Reaction Mix | | Program |
|---|---|---|
| Pfu 10X buffer | 10 μL | 94° C. 2 minutes |
| DMSO | 5 μL | 7 cycles of: |
| dNTP mix (10 mM) | 3 μL | 94° C. 30 seconds |
| RSDDSF (100 μM) | 1 μL | 55° C. 45 seconds |
| RSDDSR (100 μM) | 1 μL | 72° C. 3 minutes |
| Genomic DNA (50 ng/μL) | 2 μL | 25 cycles of: |
| Pfu enzyme (2.5 U/μL) | 2 μL | 94° C. 30 seconds |
| DI water | 76 μL | 62° C. 45 seconds |
| | | 72° C. 3 minutes |
| Total: | 100 μL | 72° C. 7 minutes |
| | | 4° C. Until used further |

The PCR product was separated on a 1% TAE-agarose gel, and a fragment about 1.6 Kb in size was excised. The excised DNA was isolated using a Qiagen gel isolation ldt. The isolated DNA was restricted with EcoRI and KpnI and was column purified using a Qiagen gel isolation kit. Three jig of pMCS2glnBP vector DNA was digested with KpnI, and the linear DNA was gel isolated using a Qiagen gel isolation kit. The vector was further digested with EcoRI, and the DNA was column purified. The double-digested vector was then dephosphorylated with shrimp alkaline phosphatase and column purified using a Qiagen gel purification kit. The KpnI/EcoRI-digested R. sphaeroides dds PCR product with the R. sphaeroides dxs I ribosomal binding site described above was ligated into the prepared vector using T4 DNA ligase for 14-16 hours at 16° C. One μL of the ligation reaction was transformed into E. coli Electromax™ DH10B™ cells, which were then plated on LBK (25 μg/mL) media. Individual colonies were resuspended in about 25 μL of DI water, and 2 μL of the resuspension was plated on LBK. The remnant resuspension was heated for 10 minutes at 95° C. to break open the bacterial cells, and 2 μL of the heated cells was used in a 25 μL PCR reaction using the glnBF and RSDDSR primers. The PCR mix contained the following: 1×Taq PCR buffer, 0.2 μM each primer, 0.2 mM each dNTP, 5% DMSO (v/v), and 1 unit of Taq DNA polymerase per reaction. The PCR reaction was performed under the following conditions: an initial denaturation at 94° C. for 2 minutes; 6 cycles of 94° C. for 30 seconds, 55° C. for 45 seconds, and 72° C. for 3 minutes; 25 cycles of 94° C. for 30 seconds, 62° C. 45 seconds, and 72° C. for 3 minutes; and a final extension for 7 minutes at 72° C. A large scale plasmid preparation was done on a culture of a colony containing the Rsdds PCR product, and the glnBP/Rsdds region was sequenced to confirm the lack of nucleotide errors. The resulting plasmid containing the Rsdds sequence under the control of the glnB promotor was designated pMCS2glnBP/Rsdds.

The pMCS2glnBP/Rsdds plasmid DNA was electroporated into electrocompetent R. sphaeroides strain 35053 cells as well as electrocompetent carotenoid-deficient mutant cells of 35053 (ATCC 35053/ΔcrtE). Individual colonies of both strains were screened by PCR using the glnBF and RSDDSR primers to confirm the presence of the insert as described above.

pMCS2glnBP/Stdds

The nucleic acid encoding a S. trueperi polypeptide having DDS activity was cloned in the pMCS2glnBP vector as follows. The S. trueperi dds gene was PCR amplified using the following primer pair.

| | | |
|---|---|---|
| SHDDSECOVF | 5′-GCGTGATATCGAAGGAAGAGCATGAGCGC-AACCGTCCACCG-3′ | (SEQ ID NO:153) |
| SHDDSKPNR | 5′-ACTGCTAGGGTCCGAGGTACCGACATGGACGA-GGAAGACGC-3′ | (SEQ ID NO:154) |

The forward primer introduced an EcoRV restriction site and a ribosomal binding site that was designed based on the R. sphaeroides dxs1 gene. The reverse primer introduced a KpnI restriction site. The following reaction mix and PCR program were used to amplify the S. trueperi dds gene.

| Reaction Mix | | Program |
|---|---|---|
| Pfu 10X buffer | 10 μL | 94° C. 2 minutes |
| DMSO | 5 μL | 7 cycles of: |
| dNTP mix (10 mM) | 3 μL | 94° C. 30 seconds |
| SHDDSECOVF (100 μM) | 1 μL | 58° C. 45 seconds |
| SHDDSKPNR (100 μM) | 1 μL | 72° C. 3 minutes |
| Genomic DNA (50 ng/μL) | 2 μL | 25 cycles of: |
| Pfu enzyme (2.5 U/μL) | 2 μL | 94° C. 30 seconds |
| DI water | 76 μL | 65° C. 45 seconds |
| | | 72° C. 3 minutes |
| Total: | 100 μL | 72° C. 7 minutes |
| | | 4° C. Until used further |

The PCR product was separated on a 1% TAE-agarose gel, and a fragment about 1.2 Kb in size was excised. The excised DNA was isolated using a Qiagen gel isolation kit. The isolated DNA was restricted with EcoRV and KpnI and was column purified using a Qiagen gel isolation lit. Three μg of pMCS2glnBP vector DNA was digested with KpnI, and the linear DNA was gel isolated using a Qiagen gel isolation lit. The vector was further digested with EcoRV, and the DNA was column purified. The double-digested vector was then dephosphorylated with shrimp alkaline phosphatase and column purified using a Qiagen gel purification kit. The KpnI/EcoRV-digested S. trueperi dds PCR product with the R. sphaeroides dxs I ribosomal binding site was ligated into the prepared vector using T4 DNA ligase for 14-16 hours at 16° C. One IL of the ligation reaction was transformed into E. coli Electromax™ DH10BTM cells, which were plated on LBK (25 μg/mL) media. Individual colonies were resuspended in about 25 μL of DI water, and 2 μL of the resuspension was plated on LBK. The remnant resuspension was heated for 10 minutes at 95° C. to break open the bacterial cells, and 2 μL of the heated cells was used in a 25 μL PCR reaction using the glnBF and RSDDSR primers. The PCR mix contained the following: 1×Taq PCR buffer, 0.2 μM each primer, 0.2 mM each dNTP, 5% DMSO (v/v), and 1 unit of Taq DNA polymerase per reaction. The PCR reaction was performed under the following conditions: an initial denaturation at 94° C. for 2 minutes; 6 cycles of 94° C. for 30 seconds, 58° C. for 45 seconds, and 72° C. for 3 minutes; 25 cycles of 94° C. for 30 seconds, 65° C. 45 seconds, and 72° C. for 3 minutes; and a final extension for 7 minutes at 72° C. A large scale plasmid preparation was done on a culture of a colony containing the Stdds PCR product, and the glnBP/Stdds region was sequenced to confirm the lack of nucleotide errors. The resulting plasmid containing the Stdds sequence under the control of the glnB promotor was designated pMCS2glnBP/Stdds.

The pMCS2glnBP/Stdds plasmid DNA was electroporated into electrocompetent cells of R. sphaeroides strain 35053 and a carotenoid-deficient mutant of 35053 (ATCC 35053/ΔcrtE). Individual colonies of both strains were screened by PCR using the glnBF and SHDDSKPNR primers to confirm the presence of the insert as described above.

pMCS2tetP/Stdxs

The nucleic acid encoding a S. trueperi polypeptide having DXS activity was cloned in the pMCS2tetP vector as follows. The pMCS2tetP plasmid DNA was digested with the restriction enzyme KpnI, cleaned with a QIAquick PCR Purification Kit, and digested with the restriction enzyme ClaI. The enzyme reactions were inactivated by heating at 65° C. for 20 minutes. The digested vector DNA was then dephosphorylated with shrimp alkaline phosphatase and gel purified on a 1% TAE-agarose gel. The KpnI/ClaI-digested S. trueperi dxs PCR product described above with the R. sphaeroides dxs1 ribosomal binding site was ligated into the prepared vector using T4 DNA ligase for 16 hours at 16° C. One μL of the ligation reaction was transformed into E. coli Electromax™ DH5α™ cells, which were plated on LBK media. Individual colonies were resuspended in about 25 μL of 10 mM Tris, and 2 μL of the resuspension was plated on LBK. The remnant resuspension was heated for 10 minutes at 95° C. to break open the bacterial cells, and 2 μL of the heated cells was used in a 25 μL PCR reaction using the SXSCLAF2 and SHDXSMCSR primers. The PCR mix contained the following: 1×Taq PCR buffer, 0.2 μM each primer, 0.2 mM each dNTP, 5% DMSO (v/v), and 1 unit of Taq DNA polymerase per reaction. The PCR reaction was performed in a MJ Research PTC100 under the following conditions: an initial denaturation at 94° C. for 2 minutes; 8 cycles of 94° C. for 30 seconds, 54° C. for 1 minute, and 72° C. for 3.5 minutes; 24 cycles of 94° C. for 30 seconds, 60° C. 1 minute, and 72° C. for 3.5 minutes; and a final extension for 7 minutes at 72° C. A large scale plasmid preparation was done on a culture of a colony containing the S. trueperi dxs PCR product, and the tetP/Stdxs region was sequenced to confirm the lack of nucleotide errors. The resulting plasmid containing the Stdxs sequence under the control of the tet promotor was designated pMCS2tetP/Stdxs.

Plasmid DNA (pMCS2tetP/Stdxs) was electroporated into electrocompetent cells of R. sphaeroides strain 35053 and a carotenoid-deficient mutant of 35053 (ATCC 35053/ΔcrtE). Individual colonies of both strains, along with an E. coli control, were screened by PCR using the TETXBAF and STDXSMCSR primers to confirm the presence of the insert as described above.

pMCS2tetP/Rsdds

The nucleic acid encoding a R. sphaeroides polypeptide having DDS activity was cloned in the pMCS2tetP vector as follows. Three μg of plasmid DNA of the pMCS2tetP vector was digested with the restriction enzyme KpnI. The digested DNA was cleaned with a QIAquick PCR Purification Kit and digested with the restriction enzyme EcoRI, after which the enzyme was inactivated by heating at 65° C. for 20 minutes. The digested vector DNA was then dephosphorylated with shrimp alkaline phosphatase and gel purified. Sixty ng of vector DNA was ligated with 120 ng of the KpnI/EcoR I-digested R. sphaeroides dds PCR product described above using T4 DNA ligase at 16° C. for 16 hours. One μL of the ligation reaction was transformed into E. coli Electromax™ DH5α™, which were then plated on LBK media. Individual colonies were resuspended in about 25 μL of 10 mM Tris, and 2 μL of the resuspension was plated on LBK. The remnant resuspension was heated for 10 minutes at 95° C. to break open the bacterial cells, and 2 μL of the heated cells used in a 25 μL PCR reaction using the TETXBAF and RSDDSMCSR primers. The PCR mix contained the following: 1×Taq PCR buffer, 0.2 μM each primer, 0.2 mM each dNTP, 5% DMSO (v/v), and 1 unit of Taq DNA polymerase per reaction. The PCR reaction was performed in a MJ Research PTC100 under the following conditions: an initial denaturation at 94° C. for 2 minutes; 8 cycles of 94° C. for 30 seconds, 55° C. for 1 minute, and 72° C. for 3 minutes; 24 cycles of 94° C. for 30 seconds, 64° C. 1 minute, and 72° C. for 3 minutes; and a final extension for 7 minutes at 72° C. Plasmid DNA was isolated for a colony having the desired insert, and the tetP/Rsdds region was sequenced to confirm the lack of nucleotide errors from PCR. The resulting plasmid containing the Rsdds sequence under the control of the tet promotor was designated pMCS2tetP/Rsdds.

Plasmid DNA (pMCS2tetP/Rsdds) was electroporated into electrocompetent cells of R. sphaeroides strain 35053 and the ATCC 35053/ΔcrtE strain. Individual colonies of both strains, along with an E. coli control, were screened by PCR using the TETXBAF and RSDDSMCSR primers to confirm the presence of the insert as described above.

pMCS2tetP/Stdds

The nucleic acid encoding a S. trueperi polypeptide having DDS activity was cloned in the pMCS2tetP vector as follows. Three μg of pMCS2tetP plasmid DNA was digested with the restriction enzyme KpnI. The digested DNA was gel purified and digested with the restriction enzyme EcoRV. The enzyme was then inactivated by heating at 80° C. for 20 minutes, and the DNA dephosphorylated with shrimp alkaline phosphatase. The dephosphorylated DNA was purified using a QIAquick PCR purification kit. Fifty jig of digested vector DNA was ligated with 150 ng of the KpnI/EcoRV-digested S. trueperi dds PCR product described above using T4 DNA ligase at 16° C. for 16 hours. One μL of the ligation reaction was transformed into E. coli Electromax™ DH10BTM cells, which were then plated on LBK media. Individual colonies were resuspended in about 25 μL of 10 mM Tris, and 2 μL of the resuspension was plated on LBK. The remnant resuspension was heated for 10 minutes at 95° C. to break open the bacterial cells, and 2 μL of the heated cells used in a 25 μL PCR reaction using the TETXBAF and STDDSMCSR primers. The PCR mix contained the following: 1×Taq PCR buffer, 0.2 μM each primer, 0.2 mM each dNTP, 5% DMSO (v/v), and 1 unit of Taq DNA polymerase per reaction. The PCR reaction was performed in a MJ Research PTC100 under the following conditions: an initial denaturation at 94° C. for 2 minutes; 8 cycles of 94° C. for 30 seconds, 55° C. for 1 minute, and 72° C. for 3 minutes; 24 cycles of 94° C. for 30 seconds, 64° C. for 1 minute, and 72° C. for 3 minutes; and a final extension for 7 minutes at 72° C. Plasmid DNA was isolated for a colony having the desired insert and was sequenced in the tetP/Stdds region to confirm the DNA sequence of the insert. The resulting plasmid containing the Stdds sequence under the control of the tet promotor was designated pMCS2tetP/Stdds.

Plasmid DNA (pMCS2tetP/Stdds) was electroporated into electrocompetent cells of R. sphaeroides strain 35053 and the ATCC 35053/ΔcrtE strain. Individual colonies of both strains, along with an E. coli control, were screened by PCR using the TETXBAF and STDDSMCSR primers to confirm the presence of the insert as described above.

pMCS2tetP/Stdxs/Rsdds

Nucleic acid encoding a S. trueperi polypeptide having DXS activity as well as nucleic acid encoding a R. sphaeroides polypeptide having DDS activity was cloned into the pMCS2tetP vector as follows. A vector containing both the S. trueperi dxs gene and the R. sphaeroides dds gene, each behind a tet promoter, was constructed using the pMCS2tetP/Stdxs construct described above as the starting vector. This vector was digested with restriction enzyme XbaI, cleaned with a QIAquick PCR Purification Kit, and digested with the restriction enzyme Bpu10I (Fermentas, Hanover, MD). The enzyme reaction was inactivated by heating for 20 minutes at 80° C. The digested vector DNA was then dephosphorylated using shrimp alkaline phosphatase and gel purified on a 1% TAE-agarose gel.

A PCR product containing a tet promoter region followed by a R. sphaeroides dds gene was amplified using the pMCS2tetP/Rsdds construct described above as template. The PCR mix contained the following: IX Native Plus Pfu buffer, 5 ng plasmid template, 0.2 μM each primer, 0.2 mM each dNTP, 5% DMSO (v/v), and 10 units of native Pfu DNA polymerase in a final volume of 200 μL. The PCR reaction was performed in a MJ Research PTC100 under the following conditions: an initial denaturation at 94° C. for 2 minutes; 8 cycles of 94° C. for 30 seconds, 55° C. for 1 minute, and 72° C. for 3 minutes; 24 cycles of 94° C. for 30 seconds, 64° C. 1 minute, and 72° C. for 3 minutes; and a final extension for 7 minutes at 72° C. The amplification product was then separated by gel electrophoresis using a 1% TAE-agarose gel. A 1.6 Kb fragment was excised from the gel and purified. The purified fragment was digested with Bpu10I, cleaned with a QIAquick PCR Purification Kit, digested with Xba I restriction enzyme, purified again with a QIAquick PCR Purification Kit, and quantified on a minigel.

60 ng of the prepared pMCS2tetP/Stdxs vector was ligated with 70 ng of the digested tetP/Rsdds PCR product using T4 DNA ligase at 16° C. for 16 hours. One μL of ligation reaction was used to electroporate 40 μL of E. coli Electromax™ DH5α™ cells. Electroporated cells were plated on LBK media. Individual colonies were screened by PCR using the RSDDSMCSF and STDXSMCSR primers, which produced a 4.1 Kb band. Individual colonies were resuspended in about 25 μL of 10 mM Tris, and 2 μL of the resuspension was plated on LBK. The remnant resuspension was heated for 10 minutes at 95° C. to break open the bacterial cells, and 2 μL of the heated cells used in a 25 μL PCR reaction. The PCR reaction mix contained 0.2 μM each primer, 1×Genome Advantage (Clontech, Palo Alto, Calif.) reaction buffer, 1 M GCMelt, 1.1 mM Mg(OAc)₂, 0.2 mM each dNTP, and IX Genome Advantage Polymerase. The PCR was conducted in a MJ Research PTC100 and consisted of an initial denaturation at 94° C. for 1.5 minutes; 32 cycles of a 30 second denaturation at 94° C., a 1 minute annealing at 60° C., and a 6.5 minute extension at 72° C.; followed by a final extension at 72° C. for 5 minutes. A large-scale plasmid prep was done for a colony that had the desired insert, and plasmid DNA was sequenced through the tetP/Rsdds region to confirm the lack of nucleotide errors from PCR. The resulting plasmid containing the Stdxs sequence under the control of the tet promotor and the Rsdds sequence under the control of the tet promotor was designated pMCS2tetP/Stdxs/Rsdds.

Plasmid DNA (pMCS2tetP/Stdxs/Rsdds) was electroporated into electrocompetent cells of R. sphaeroides strains 35053 and the ATCC 35053/ΔcrtE. Individual colonies of both strains, along with an E. coli control, were screened by PCR using the RSDDSMCSF and STDDSMCSR primers to confirm the presence of the insert as described above.

pMCS2tetP/Stdxr

Nucleic acid encoding a S. trueperi polypeptide having DXR activity was cloned into the pMCS2tetP vector as follows. The S. trueperi dxr gene was amplified using genomic DNA as template. The following primers were designed to introduce an EcoRV restriction site and a ribosomal binding based on R. sphaeroides dxs1 gene at the beginning of the amplified fragment and a KpnI site at the end of the amplified fragment.

| | | |
|---|---|---|
| SXRRVF | 5′-GATGATATCGAAGGAAGAGCATGGTGAAGCGCGT-CACGGTGT-3′ | (SEQ ID NO:155) |
| SXRKZPNR | 5′-CAAGAGTCAGAAGGTACCCGCCAGAATGGTGAGC-AGGATG-3′ | (SEQ ID NO:156) |

The PCR mix contained the following: 1×Native Plus Pfu buffer, 200 ng genomic DNA, 0.2 μM of each primer, 0.2 μM of each dNTP, 5% DMSO (v/v), and 10 units of native Pfu DNA polymerase in a final volume of 200 μL. The PCR reaction was performed in a MJ Research PTC100 under the following conditions: an initial denaturation at 94° C. for 2 minutes; 8 cycles of 94° C. for 30 seconds, 59° C. for 1 minute, and 72° C. for 3 minutes; 24 cycles of 94° C. for 30 seconds, 64° C. 1 minute, and 72° C. for 3 minutes; and a final extension for 7 minutes at 72° C. The amplification product was then separated by gel electrophoresis using a 1% TAE-agarose gel. A 1.0 Kb fragment was excised from the gel and purified. The purified fragment was digested simultaneously with EcoRV and KpnI restriction enzymes, purified with a QIAquick PCR Purification Kit, and checked on a minigel.

Fifty ng of the EcoRV, KpnI-digested pMCS2tetP vector described above for the pMCS2tetP/Stdds construct was ligated with 75 ng of the digested S. trueperi dxr PCR product using T4 DNA ligase at 20° C. for 4 hours. One μL of ligation reaction was used to electroporate 40 μL of E. coli Electromax™ DH10 B™ cells, which were then plated on LBK media. Individual colonies were selected and screened by PCR using the TETXBAF and SXRKPNR primers. The PCR mix contained the following: 1×Taq PCR buffer, 200 ng genomic DNA, 0.2 μM of each primer, 0.2 mM of each dNTP, 5% DMSO (v/v), and 1 unit of Taq DNA polymerase per 25 μL reaction. The PCR reaction was performed in a MJ Research PTC100 under the following conditions: an initial denaturation at 94° C. for 2 minutes; 32 cycles of 94° C. for 30 seconds, 64° C. 1 minute, and 72° C. for 3 minutes; and a final extension for 7 minutes at 72° C. A large-scale plasmid preparation was done for a colony that had the desired insert, and the tetP/Stdxr region was sequenced to confirm the DNA sequence of the insert. The resulting plasmid containing the Stdxr sequence under the control of the tet promotor was designated pMCS2tetP/Stdxr.

Plasmid DNA (pMCS2tetP/Stdxr) was electroporated into electrocompetent cells of R. sphaeroides strains 35053 and ATCC 35053/ΔcrtE. Individual colonies of both strains, along with an E. coli control, were screened by PCR using the TETXBAF and SXRKPNR primers to confirm the presence of the insert as described above.

pMCS2tetP/Stdxr/Stdds

Nucleic acid encoding a S. trueperi polypeptide having DXR activity as well as nucleic acid encoding a S. trueperi polypeptide having DDS activity was cloned into the pMCS2tetP vector as follows. A vector containing both the S. trueperi dxr and dds genes, each behind a tet promoter, was constructed using the pMCS2tetP/Stdds construct described above as the starting vector. This vector was digested with restriction enzyme XbaI, cleaned with a QIAquick PCR Purification Kit, and digested with the restriction enzyme Bpu10I (Fermentas). The enzyme reaction was inactivated by heating for 20 minutes at 80° C. The digested vector DNA was then dephosphorylated with shrimp alkaline phosphatase and gel purified.

A PCR product containing a tet promoter region followed by a S. trueperi dxr gene was amplified using the pMCS2tetP/Stdxr construct described above as template and primers TETBPUF and SXRXBAR. The SXRXBAR primer, having the following sequence, was designed to introduce an XbaI restriction site on the end of the PCR product.

| | | |
|---|---|---|
| SXRXBAR | 5′-CAAGAGTCAGAATCTAGACGCCAGAATGGTGA-GCAGGATG-3′ | (SEQ ID NO:157) |

The PCR mix contained the following: 1×Native Plus Pfu buffer, 5 ng plasmid template, 0.2 μM each primer, 0.2 nm each dNTP, 5% DMSO (v/v), and 10 units of native Pfu DNA polymerase in a final volume of 200 μL. The PCR reaction was performed in a MJ Research PTC 100 under the following conditions: an initial denaturation at 94° C. for 2 minutes; 8 cycles of 94° C. for 30 seconds, 59° C. for 1 minute, and 72° C. for 3.5 minutes; 24 cycles of 94° C. for 30 seconds, 64° C. 1 minute, and 72° C. for 3.5 minutes; and a final extension for 7 minutes at 72° C. The amplification product was then separated by gel electrophoresis using a 1% TAE-agarose gel. A 1.4 Kb fragment was excised from the gel and purified. The purified fragment was digested with Bpu10I, cleaned with a QIAquick PCR Purification Kit, digested with XbaI restriction enzyme, purified again with a QIAquick PCR Purification Kit, and quantified on a minigel.

Sixty ng of the prepared pMCS2tetP/Stdds vector was ligated with 80 ng of the digested tetP/Stdxr PCR product using T4 DNA ligase at 16° C. for 16 hours. One FL of ligation reaction was used to electroporate 40 μL of E. coli Electromax™ DH10 B™ cells, which were then plated oil LBK media. Individual colonies were screened by PCR using the SXREVF and SDSKPNR primers. Colonies were resuspended in about 25 μL of 10 mM Tris, and 2 μL of the resuspension was plated on LBK media. The remnant resuspension was heated for 10 minutes at 95° C. to break open the bacterial cells, and 2 μL of the heated cells used in a 25 μL PCR reaction. The PCR mix contained the following: 1×Taq PCR buffer, 0.2 μM each primer, 0.2 mM each dNTP, 5% DMSO (v/v), and 1 unit of Taq DNA polymerase per reaction. The PCR reaction was performed in a MJ Research PTC100 under the following conditions: an initial denaturation at 94° C. for 2 minutes; 8 cycles of 94° C. for 30 seconds, 58° C. for 1 minute, and 72° C. for 4.5 minutes; 24 cycles of 94° C. for 30 seconds, 64° C. 1 minute, and 72° C. for 4.5 minutes; and a final extension for 7 minutes at 72° C. A large-scale plasmid preparation was done for a colony that had the desired insert, and the tetP/Stdxr region was sequenced to confirm the lack of nucleotide errors from PCR. The resulting plasmid containing the Stdxr sequence under the control of the tet promotor and the Stdds sequence under the control of the tet promotor was designated pMCS2tetP/Stdxr/Stdds.

Plasmid DNA (pMCS2tetP/Stdxr/Stdds) was electroporated into electrocompetent cells of R. sphaeroides strains 35053 and ATCC 35053/ΔcrtE. Individual colonies of both strains, along with an E. coli control, were screened by PCR using the SXREVF and SDSKPNR primers to confirm the presence of the insert as described above.

pMCS2tetP/EcUbiC

Nucleic acid encoding a E. coli polypeptide having chorismate lyase activity was cloned into the pMCS2tetP vector as follows. The E. coli ubiC gene was amplified using genomic DNA from E. coli strain DH10 B as template. The following primers were designed to introduce an EcoRV restriction site and a ribosomal binding site based on R. sphaeroides dxs1 gene at the beginning of the amplified fragment, and a KpnI site at the end of the amplified fragment.

| | | |
|---|---|---|
| UBICRVF | 5′-CTAGATATCGGAAGGAAGAGCATGTCACAC-CCCGCGTTA-3′ | (SEQ ID NO:158) |
| UBLCKPNR | 5′-TCAGGTACCGTGTCGCCACCCACAACGCC-CATAATG-3′ | (SEQ ID NO:159) |

The PCR mix contained the following: 1×Native Plus Pfu buffer, 200 ng genomic DNA, 0.2 μM each primer, 0.2 mM each dNTP, and 10 units of native Pfu DNA polymerase in a final volume of 200 μL. The PCR reaction was performed in a MJ Research PTC100 under the following conditions: an initial denaturation at 94° C. for 2 minutes; 8 cycles of 94° C. for 30 seconds, 57° C. for 1 minute, and 72° C. for 2.5 minutes; 24 cycles of 94° C. for 30 seconds, 64° C. 1 minute, and 72° C. for 2.5 minutes; and a final extension for 7 minutes at 72° C. The amplification product was then separated by gel electrophoresis using a 1.5% TAE-agarose gel. A 650 bp fragment was excised from the gel and purified. The purified fragment was digested with EcoRV, cleaned with a QIAquick PCR Purification Kit, digested with KpnI restriction enzyme, purified again with a QIAquick PCR Purification Kit, and quantified on a minigel.

Seventy-five ng of the EcoRV, KpnI-digested pMCS2tetP vector described above for the pMCS2tetP/Stdds construct was ligated with 70 ng of the digested ubiC PCR product using T4 DNA ligase at 16° C. for 16 hours. One μL of ligation reaction was used to electroporate 40 μL of E. coli Electromax™ DH5α™ cells, which were then plated on LBK media. Individual colonies were resuspended in about 25 μL of 10 mM Tris, and 2 μL of the resuspension was plated on LBK. The remnant resuspension was heated for 10 minutes at 95° C. to break open the bacterial cells, and 2 μL of the heated cells used in a 25 μL PCR reaction using the TETXBAF and UBICKPNR primers. The PCR mix contained the following: 1×Taq PCR buffer, 0.2 μM each primer, 0.2 mM each dNTP, and 1 unit of Taq DNA polymerase per reaction. The PCR reaction was performed in a MJ Research PTC 100 under the following conditions: an initial denaturation at 94° C. for 2 minutes; 32 cycles of 94° C. for 30 seconds, 62° C. for 1 minute, and 72° C. for 2 minutes; and a final extension for 7 minutes at 72° C. A large-scale plasmid preparation was done for a colony that had the desired insert and the tetP/ubiC region was sequenced to confirm the DNA sequence of the insert. The resulting plasmid containing the UbiC sequence under the control of the tet promotor was designated pMCS2tetP/EcUbiC.

Plasmid DNA (pMCS2tetP/EcUbiC) was electroporated into electrocompetent cells of R. sphaeroides strain 35053 and the ATCC 35053/ΔcrtE strain. Individual colonies of both strains, along with an E. coli control, were screened by PCR using the TETXBAF and UBICKPNR primers to confirm the presence of the insert as described above with the addition of 5% DMSO (v/v) to the PCR reaction.

pMCS2tetP/Stdxs/Rsdds/EcUbiC

Nucleic acid encoding an S. trueperi polypeptide having DXS activity, nucleic acid encoding an R. sphaeroides polypeptide having DDS activity, and nucleic acid encoding an E. coli polypeptide having chorismate lyase activity was cloned into the pMCS2tetP vector as follows. A vector containing the S. trueperi dxs gene, the R. sphaeroides dds gene, and the E. coli ubiC gene, each behind a tet promoter, was constructed using the pMCS2tetP/Stdxs/Rsdds construct described above as the starting vector. This vector was digested with restriction enzyme KpnI, cleaned with a QIAquick PCR Purification Kit, and digested with the restriction enzyme NsiI. The enzyme reaction was inactivated by heating for 20 minutes at 65° C. The digested vector DNA was then dephosphorylated with shrimp alkaline phosphatase and gel purified.

A PCR product containing a tet promoter region followed by an E. coli ubiC gene was amplified using the pMCS2tetP/EcUbiC construct described above as template. The following primers were designed to introduce an KpnI restriction site at the beginning of the amplified fragment and an NsiI site at the end of the amplified fragment.

| | | |
|---|---|---|
| TETKPNF | 5′-TAGGGTACCACCGTCTACGCCGACCT-CGTTCAAC-3′ | (SEQ ID NO:160) |
| UBICNSIR | 5′-TGTATGCATGTCGCCACCCACAACGC-CCATAATG-3′ | (SEQ ID NO:161) |

The PCR mix contained the following: IX Native Plus Pfu buffer, 5 ng plasmid template, 0.2 μM each primer, 0.2 mM each dNTP, 5% DMSO (v/v), and 10 units of native Pfu DNA polymerase in a final volume of 200 μL. The PCR reaction was performed in a MJ Research PTC100 under the following conditions: an initial denaturation at 94° C. for 2 minutes; 8 cycles of 94° C. for 30 seconds, 62° C. for 1 minute, and 72° C. for 2.5 minutes; 24 cycles of 94° C. for 30 seconds, 66° C. 1 minute, and 72° C. for 2.5 minutes; and a final extension for 7 minutes at 72° C. The amplification product was then separated by gel electrophoresis using a 1% TAE-agarose gel. An 850 bp fragment was excised from the gel and purified. The purified fragment was digested with the restriction enzyme NsiI, cleaned with a QIAquick PCR Purification Kit, digested with the restriction enzyme KpnI, purified again with a QIAquick PCR Purification Kit, and quantified on a minigel.

Fifty ng of the prepared pMCS2tetP/Stdxs/Rsdds vector was ligated with 35 ng of the digested tetP/ubiC PCR product using T4 DNA ligase at 16° C. for 16 hours. One μL of ligation reaction was used to electroporate 40 μL of E. Coli Electromax™ DH10 B™ cells, which were then plated on LBK media. Individual colonies were resuspended in about 25 μL of 10 mM Tris, and 2 μL of the resuspension was plated on LBK. The remnant resuspension was heated for 10 minutes at 95° C. to break open the bacterial cells, and 2 μL of the heated cells used in a 25 μL PCR reaction using the SXSCLAF2 and UBICNSIR primers. The PCR reaction mix contained 1×GC-RICH PCR reaction buffer, 1.0 M GC-RICH resolution solution, 0.2 μM each primer, 0.2 mM each dNTP, and 1 unit of GC-RICH enzyme mix per reaction (Roche). The PCR reaction was performed in a MJ Research PTC100 under the following conditions: an initial denaturation at 94° C. for 2 minutes; 8 cycles of 94° C. for 30 seconds, 60° C. for 1 minute, and 72° C. for 5 minutes; 24 cycles of 94° C. for 30 seconds, 64° C. 1 minute, and 72° C. for 5 minutes; and a final extension for 7 minutes at 72° C. A large-scale plasmid preparation was done for a colony that had the desired insert, and plasmid DNA was sequenced through the tetP/ubiC region to confirm the lack of nucleotide errors from PCR. The resulting plasmid containing Stdxs sequence under the control of the tet promotor, the Rsdds sequence under the control of the tet promotor, and the UbiC sequence under the control of the tet promotor was designated pMCS2tetP/Stdxs/Rsdds/EcUbiC.

Plasmid DNA (pMCS2tetP/Stdxs/Rsdds/EcUbiC) was electroporated into electrocompetent cells of R. sphaeroides strains 35053 and ATCC 35053/ΔcrtE. Individual colonies of both strains, along with an E. coli control, were screened by PCR using the SXSCLAF2 and UBICNSIR primers to confirm the presence of the insert as described above.

pMCS2tetP/RsLytB

Nucleic acid encoding a LytB R. sphaeroides polypeptide was cloned into the pMCS2tetP vector as follows. The R. sphaeroides lytB was identified by TBLASTN analysis of its genome using an E. coli lytB sequence as a query. Based on the identified sequence the following primers were designed to PCR amplify the gene:

| | | |
|---|---|---|
| LYTBHINDF | 5′-GACGAAGCTTGAAGGAAGAGCATGCCTCCCCTCA-CCCTCTATC-3′ | (SEQ ID NO:162) |
| LYTBKPNR | 5′-GTCACTGAATGAATGGTACCGCAGCCGAGAACCG-CCAGAAGCC-3′ | (SEQ ID NO:163) |

The primers introduced a HindIII restriction site and ribosomal binding site at the 5′ end, and a KpnI restriction site at the 3′ end. The following reaction mix and PCR program were used to amplify the lytB gene.

| Reaction Mix | | Program |
|---|---|---|
| Pfu 10X buffer | 10 μL | 94° C. 2 minutes |
| DMSO | 5 μL | 7 cycles of: |
| dNTP mix (10 mM) | 3 μL | 94° C. 30 seconds |
| LYTBHINDF (100 μM) | 1 μL | 59° C. 45 seconds |
| LYTBKPNR (100 μM) | 1 μL | 72° C. 3 minutes |
| Genomic DNA (50 ng/μL) | 2 μL | 25 cycles of: |
| Pfu enzyme (2.5 U/μL) | 2 μL | 94° C. 30 seconds |
| DI water | 76 μL | 66° C. 45 seconds |
| | | 72° C. 3 minutes |
| Total: | 100 μL | 72° C. 7 minutes |
| | | 4° C. Until used further |

The PCR product was run on a 1% TAE-agarose gel, and a fragment about 1.1 Kb in size was excised. The excised DNA was isolated using a Qiagen gel isolation kit. The isolated DNA was restricted with HindIII and KpnI, and was column purified using a Qiagen gel isolation kit. Two μg of pMCS2tetP vector DNA was digested with HindIII, and the linear DNA was gel isolated using a Qiagen gel isolation kit. The vector was further digested with KpnI, and the DNA was column purified. The double-digested vector was then dephosphorylated with shrimp alkaline phosphatase and column purified using a Qiagen gel purification kit. The KpnI/HindIII-digested R. sphaeroides lytB PCR product with the R. sphaeroides dxs I ribosomal binding site described above was ligated into the prepared vector using T4 DNA ligase for 14-16 hours at 16° C. One μL of the ligation reaction was transformed into E. coli Electromax™ DH10 B™ cells, which were then plated on LBK (25 μg/mL) media. Individual colonies were resuspended in about 25 μL of DI water, and 2 μL of the resuspension was plated on LBK. The remnant resuspension was heated for 10 minutes at 95° C. to break open the bacterial cells, and 2 μL of the heated cells was used in a 25 μL PCR reaction using the LYTBHINDF and LYTBKPNR primers. The PCR mix contained the following: 1×Taq PCR buffer, 0.2 μM each primer, 0.2 mM each dNTP, 5% DMSO (v/v), and 1 unit of Taq DNA polymerase per reaction. The PCR reaction was performed under the following conditions: an initial denaturation at 94° C. for 2 minutes; 8 cycles of 94° C. for 30 seconds, 59° C. for 1 minute, and 72° C. for 3 minutes; 24 cycles of 94° C. for 30 seconds, 66° C. for 1 minute, and 72° C. for 3 minutes; and a final extension for 7 minutes at 72° C. A large scale plasmid preparation was done on a culture of a colony containing the lytB PCR product, and the tetP/lytB region was sequenced to confirm the lack of nucleotide errors. The resulting plasmid containing the RsLytB sequence under the control of the tet promotor was designated pMCS2tetP/RsLytB.

Plasmid DNA (pMCS2tetP/RsLytB) was electroporated into electrocompetent cells of R. sphaeroides strain 35053 and a carotenoid-deficient mutant of 35053 (ATCC 35053/ΔcrtE). Individual colonies of both strains, along with an E. coli control, were screened by PCR using the TETXBAF and LYTBKPNR primers to confirm the presence of the insert as described above.

pMCS2tetP/Stdxs/Rsdds/RsLytB

Nucleic acid encoding an S. trueperi polypeptide having DXS activity, nucleic acid encoding an R. sphaeroides polypeptide having DDS activity, and nucleic acid encoding LytB from R. sphaeroides were cloned into the pMCS2tetP vector as follows. The R. sphaeroides lytB gene was cloned and expressed along with the R. sphaeroides dds and S. trueperi dxs genes. In this triple expression system, each gene was expressed through its own tetP. The R. sphaeroides lytB gene was PCR amplified along with the tetP using the following primers.

| | | |
|---|---|---|
| TETKPNF | 5′-TAGGGTACCACCGTCTACGCCGACCTC-GTTGAAC-3′ | (SEQ ID NO:164) |
| LYTBNSIR | 5′-AGGCAATGCATGCAGCCGAGAACCGCC-AGAAGCC-3′ | (SEQ ID NO:165) |

The following PCR mix and program were used to PCR amplify the lytB gene along with the tetP.

| Reaction Mix | | Program |
|---|---|---|
| Pfu 10X buffer | 10 μL | 94° C. 2 minutes |
| DMSO | 5 μL | 7 cycles of: |
| dNTP mix (10 mM) | 3 μL | 94° C. 30 seconds |
| TETKPNF (100 μM) | 1 μL | 63° C. 45 seconds |
| LYTBNSIR (100 μM) | 1 μL | 72° C. 3 minutes |
| pMCS2tetP/lytB (10 ng/μL) | 1 μL | 25 cycles of: |
| Pfu enzyme (2.5 U/μL) | 2 μL | 94° C. 30 seconds |
| DI water | 77 μL | 69° C. 45 seconds |
| | | 72° C. 3 minutes |
| Total: | 100 μL | 72° C. 7 minutes |
| | | 4° C. Until used further |

In this PCR reaction, pMCS2tetP/RsLytB plasmid DNA was used as a template. The PCR product was separated on a 1% TAE-agarose gel, and a fragment about 1.4 Kb in size was excised. The excised DNA was isolated using a Qiagen gel isolation kit. The isolated DNA was restricted with NsiI and KpnI, and was column purified using a Qiagen gel isolation kit. Two μg of pMCS2tetP/Stdxs/Rsdds plasmid DNA was digested with NsiI, and the linear DNA was gel isolated using a Qiagen gel isolation kit. The vector was further digested with KpnI, and the DNA was column purified. The double-digested vector was then dephosphorylated with shrimp alkaline phosphatase and column purified using a Qiagen gel purification kit. The KpnI/NsiI-digested PCR product was ligated into the prepared plasmid using T4 DNA ligase for 14-16 hours at 16° C. One μL of the ligation reaction was transformed into E. coli Electromax™ DHI10B™ cells, which were then plated on LBK (25 μg/mL) media. Individual colonies were resuspended in about 25 μL of DI water, and 2 μL of the resuspension was plated on LBK. The remnant resuspension was heated for 10 minutes at 95° C. to break open the bacterial cells, and 2 μL of the heated cells was used in a 25 μL PCR reaction using the SXSCLAF2 and LYTBNSIR primers. The PCR mix contained the following: 1×Taq PCR buffer, 0.2 μM each primer, 0.2 mM each dNTP, 5% DMSO (v/v), and 1 unit of Taq DNA polymerase per reaction. The PCR reaction was performed under the following conditions: an initial denaturation at 94° C. for 2 minutes; 6 cycles of 94° C. for 30 seconds, 59° C. for 45 sec, and 72° C. for 4 minutes; 25 cycles of 94° C. for 30 seconds, 65° C. for 45 seconds, and 72° C. for 4 minutes; and a final extension for 7 minutes at 72° C. A large scale plasmid preparation was done on a culture of a colony containing the correct insert, and the tetP/lytB region was sequenced to confirm the lack of nucleotide errors. The resulting plasmid containing Stdxs sequence under the control of the tet promotor, the Rsdds sequence under the control of the tet promotor, and the LytB sequence under the control of the tet promotor was designated pMCS2tetP/Stdxs/Rsdds/RsLytB.

Plasmid DNA (pMCS2tetP/Stdxs/Rsdds/RsLytB) was electroporated into electrocompetent cells of R. sphaeroides strain 35053 and a carotenoid-deficient mutant of 35053 (ATCC 35053/ΔcrtE). Individual colonies of both strains were screened by PCR using the SXSCLAF2 and LYTBNSIR primers to confirm the presence of the insert as described above.

### Example 9

### Making Recombinant Microorganisms Containing Knock-Outs

Various nucleic acid sequences within the R. sphaeroides genome were knocked out. All restriction enzymes and T4 DNA ligase were obtained from New England Biolabs (Beverly, Mass.) unless otherwise indicated. All plasmid DNA preparations were done using QIAprep Spin Miniprep Kits or Qiagen Maxi Prep Kits, and all gel purifications were done using QIAquick Gel Extraction Kits (Qiagen, Valencia, Calif.).

ATCC 35053/ΔcrtE(kan)

R. sphaeroides cells lacking crtE were made by inserting a kanamycin resistance gene into the crtE sequence as follows. In general, the crtE gene from R. sphaeroides was cloned into a pUC19 vector, and a kanamycin gene (lean) was inserted into the gene to inactivate it. The crtE-kan insert was amplified by PCR and cloned into pSUP203, a mobilizable Co1E1-based plasmid that is not maintained in R. sphaeroides unless it is integrated into a R. sphaeroides replicon. This plasmid was transformed into E. coli strain S17-1, a strain that is able to mobilize oriT-containing plasmids in conjugations with a second bacterial strain. The S17-1 strain was conjugated with R. sphaeroides strain 35053, and colonies were identified in which the crtE-kan insert had replaced the native crtE gene.

The crtE gene from R. sphaeroides strain 17023 was amplified by PCR using primers designed to introduce an SphI restriction site at the beginning of the amplified fragment and an XbaI restriction site at the end of the amplified fragment. The sequences of the primers were as follows.

(SEQ ID NO:166)

| | |
|---|---|
| CRTESPHF | 5′-AAGCATGCGAAAAAGTTGACACCTGTGGAGTC-3′ |

(SEQ ID NO:167)

| | |
|---|---|
| CRTEXBAR | 5′-ACTCTAGAAGCACCTGCGAATGGACGAAG-3′ |

The fragment amplified included the crtE gene along with 85 nucleotides upstream of the translational start codon and 228 nucleotides downstream of the translational stop codon. The PCR reaction mix contained 0.2 μM each primer, 1×GC Genomic PCR Buffer (Clontech, Palo Alto, Calif.), 1 M GC-Melt, 1.1 mM Mg(OAc)₂, 0.2 mM each dNTP, 1×Advantage-GC Genomic Polymerase Mix, and 1 ng of genomic DNA per μL of reaction mix. The PCR was conducted in a Perkin Elmer Geneamp 2400 and consisted of an initial denaturation at 94° C. for 30 seconds; 35 cycles of a 15 second denaturation at 94° C., a one minute annealing at 55° C., and a 3 minute extension at 72° C.; followed by a final extension at 72° C. for 5 minutes. Fifty μL of PCR product was separated on a 1% Tris-Acetate-EDTA (TAE)-agarose gel. A 1180 bp fragment was gel purified, and the purified DNA was digested with XbaI and SphI restriction enzymes (Promega, Madison, Wis.).

pUC 19 vector was digested with the restriction enzymes SphI and XbaI, and gel purified on a 1% TAE-agarose gel. Fifty ng of purified vector was ligated with about 150 ng of digested crtE PCR product for 16 hours at 14° C. using T4 DNA ligase (Roche Molecular Biochemicals, Indianapolis, Ind.). One μL of ligation reaction was transformed into ElectroMAX™ DH10B™ cells (Life Technologies, Gaithersburg, Md.), which were then plated on LB media containing 100 μg/mL ampicillin and 50 μg/mL of 5-Bromo-4-Chloro-3-Indolyl-B-D-Galactopyranoside (LBKX). Individual, white colonies were resuspended in about 20 μL of 10 mM Tris, and 2 μL of the resuspension was plated on LBKX media. The remnant resuspension was heated for 10 minutes at 95° C. to break open the bacterial cells, and 2 μL of the heated cells was used in a 25 μL PCR reaction using the CRTESPHF and CRTEXBAR primers. The PCR reaction mix contained 0.2 μM each primer, IX GC Genomic PCR Buffer, 1 M GCMelt, 1.1 mM Mg(OAc)₂, 0.2 mM each dNTP, and 1×Advantage-GC Genomic Polymerase Mix. The PCR was conducted in a Perkin Elmer Geneamp 2400 and consisted of an initial denaturation at 94° C. for 30 seconds; 35 cycles of a 15 second denaturation at 94° C., a one minute annealing at 55° C., and a 3 minute extension at 72° C.; followed by a final extension at 72° C. for 5 minutes. Plasmid DNA was isolated for colonies having a crtE gene insert and was digested with the restriction enzyme HindIII and with a mixture of SphI and XbaI to confirm vector structure.

One μg of the pUC 19crtE construct was digested with XhoI and StuI restriction enzymes. These enzymes cut a 273 bp fragment of DNA from the center of the crtE gene. The digested DNA was separated on a 1% TAE-agarose gel. A 3.6 Kb fragment representing pUC 19 and the remaining ends of the crtE gene was excised and purified.

The kanamycin resistance gene was amplified by PCR from the PCRII vector (Invitrogen, Carlsbad, Calif.) using primers designed to introduce an StuI restriction site at the beginning of the amplified fragment and an XhoI restriction site at the end of the amplified fragment. The sequences of the primers were as follows.

(SEQ ID NO:168)

| | |
|---|---|
| KANSTUF | 5′-ATAAAGGCCTTACATGGCGATAGCTAGACTG-3′ |

(SEQ ID NO:169)

| | |
|---|---|
| KANXHOR | 5′-AAGGCTCGAGAAGGATCTTACCGCTGTTGAG-3′ |

The PCR reaction mix contained 0.2 μM each primer, 1×Pfu reaction buffer (Stratagene, La Jolla, Calif.), 0.2 mM each dNTP, 8 units Pfu, and 5 ng of the PCRII vector in a 200 μL reaction. The PCR was conducted in a Perkin Elmer Geneamp 2400 and consisted of an initial denaturation at 94° C. for 2 minutes; 8 cycles of a 30 second denaturation at 94° C., a 1 minute annealing at 55° C., and a 2.5 minute extension at 72° C.; 24 cycles of a 30 second denaturation at 94° C., a 1 minute annealing at 55° C., and a 2.5 minute extension at 72° C.; followed by a final extension at 72° C. for 5 minutes. The PCR product was separated on a 1% TAE-agarose gel, and a 1.2 Kb fragment was excised and purified. One μg of purified DNA was digested with XhoI and StuI restriction enzymes and cleaned using a QIAquick PCR Purification Kit.

Fifty ng of the digested pUC19crtE vector DNA was ligated with 75 ng of the digested kan PCR product for 16 hours at 14° C. using T4 DNA ligase (Roche). One μL of ligation mix was electroporated into 40 μL of E. coli ElectroMAX™ DH10B™ electrocompetent cells, which were then plated on LB media containing 100)1 g/mL ampicillin and 50 μg/mL kanamycin (LBAK). Plasmid DNA was isolated from cultures of individual colonies and was digested in separate reactions with the restriction enzymes PstI, SphI, and a StuI/XbaI mixture to confirm correct vector structure.

The crtE gene with the inserted kan gene was amplified by PCR using primers designed to have ScaI restriction sites on both ends of the fragment. The sequences of the primers were as follows.

(SEQ ID NO:170)

| | |
|---|---|
| CRTESCAF | 5′-ATAGTACTGAAAAAGTTGACACCTGTGGAGTC-3′ |

(SEQ ID NO:171)

| | |
|---|---|
| CRTESCAR | 5′-ATAGTACTAGCACCTGCGAATGGACGAAG-3′ |

The PCR reaction mix contained 0.2 μM each primer, 1×GC Genomic PCR Buffer, 1 M GCMelt, 1.1 nM Mg(OAc)₂, 0.2 mM each dNTP, 1×Advantage-GC Genomic Polymerase Mix, and 1 ng of plasmid DNA per μL of reaction mix. The PCR was conducted in a Perkin Elmer Geneamp 9600 and consisted of an initial denaturation at 94° C. for 1 minute; 8 cycles of a 30 second denaturation at 94° C., a 1 minute annealing at 55° C., and a 4 minute extension at 72° C.; 25 cycles of a 30 second denaturation at 94° C., a 1 minute annealing at 60° C., and a 4 minute extension at 72° C.; followed by a final extension at 72° C. for 5 minutes. 200 μL of PCR product was separated on a 1% TAE-agarose gel. A 2.0 Kb fragment was excised and purified. One μg of purified DNA was digested with ScaI restriction enzyme, and the digested DNA was purified using a QIAquick PCR Purification Kit.

2.3 μg of pSUP203 plasmid DNA was digested with Seal restriction enzyme. The digested DNA was separated on a 1% TAE-agarose gel, and a 7.6 Kb fragment was excised and purified. The purified plasmid DNA was then dephosphorylated using calf intestinal alkaline phosphatase (Promega). 75 ng of dephosphorylated plasmid DNA was ligated with 60 ng and 120 ng of the ScaI-digested crtE-kan PCR product for 16 hours at 14° C. using T4 DNA ligase (New England BioLabs). One μL of ligation mix was electroporated into 40 μL of E. Coli ElectroMAX™ DH 10B™ electrocompetent cells, which were then plated on LB media containing 10 μg/mL tetracycline, to which pSUP203 carries a resistance gene, and 25 μg/mL kanamycin. Plasmid DNA was isolated from cultures of individual colonies and digested with ScaI restriction enzyme to check insert size. 100 ng of plasmid DNA derived from a confirmed colony was electroporated into electrocompetent cells of the E. coli strain S17-1. This strain contains a chromosomal copy of the trans-acting elements that mobilize oriT-containing plasmids during conjugation with a second bacterial strain. It also carries a gene conferring resistance to the antibiotics streptomycin and spectinomycin. The transformation reaction was plated on LB media with 10 μg/nL tetracycline, 25 μg/mL kanamycin, and 25 μg/mL streptomycin. Individual colonies were resuspended in about 20 μL of 10 mM Tris and heated for 10 minutes at 95° C. to break open the bacterial cells. Two μL of the heated cells was used in a 25 mL PCR reaction using the CRTESCAF and CRTESCAR primers to confirm the presence of the crtE-kan insert. The PCR reaction mix contained 0.2 μM each primer, 1×GC Genomic PCR Buffer, 1.0 M GCMelt, 1.1 mM Mg(OAc)₂, 0.2 mM each dNTP, and 1×Advantage-GC Genomic Polymerase Mix. The PCR was conducted in a Perkin Elmer Geneamp 9600 and consisted of an initial denaturation at 94° C. for 1 minute; 30 cycles of a 30 second denaturation at 94° C., a 1 minute annealing at 56° C., and a 4 minute extension at 72° C.; followed by a final extension at 72° C. for 5 minutes.

The pSUP203crtE-kan construct was introduced into R. sphaeroides strain 35053 through conjugation with the E. coli S17-1 strain carrying this vector. The S17-1 donor was grown in LB media with 25 μg/mL kanamycin and 25 μg/mL streptomycin at 37° C. for 16 hours. A growing culture of R. sphaeroides strain 35053 was used to inoculate Sistrom's media using ⅕ to 1/10 dilutions, and the subcultures were grown at 30° C. for about 20 hours. For both the S17-1crtE-kan and 35053 genotypes, cells were pelleted from 1.5 mL of culture. Pellets were resuspended and pelleted four times in either 1×Sistrom's salts for the 35053 cells or LB media for the S17-1 cells. The pellets were each resuspended in 1.5 mL of LB, and 200 μL of the S17-1 cells was combined with 1.3 mL of the 35053 cells. This mixture was pelleted, the supernatant removed, and the pellet resuspended in 20 μL of LB media. The resuspended cells were spotted onto an LB plate and incubated at 30° C. for 7.5 hours. The cells were then scraped off the plate, resuspended in 1.5 mL of 1×Sistrom's salts, and plated (200 μL/plate) on Sistrom's media supplemented with 25 μg/mL kanamycin and 10 μg/mL of telluride (SisKTell). The telluride retards the growth of E. coli cells but is detoxified by R. sphaeroides. After 7 days, small black colonies were picked off the plates and streaked to fresh plates of the same media. After 6 days of growth, grayish colonies were patched to LB plates containing 25 μg/mL kanamycin (LBK25) and also to LB plates containing 0.75 μg/mL tetracycline. Desirable double-crossover events, in which the crtE-kan gene was integrated and retained in the genome while the vector DNA was lost, exhibited kanamycin resistance but lacked tetracycline resistance. Colonies resulting from undesirable single-crossover events demonstrated both kanamycin and tetracycline resistance.

The mutants were confirmed using PCR and Southern hybridization as follows. Colonies that exhibited kanamycin resistance, lacked tetracycline resistance, and had a gray phenotype were screened by PCR for the crtE locus using the CRTESCAF and CRTESCAR primers as described above. To confirm that they were R. sphaeroides colonies with a truncated crtE gene rather than E. coli colonies carrying the vector, colonies were also screened using primers specific to the R. sphaeroides ppsR gene and the E. coli dxs gene. Individual colonies were resuspended in about 20 μL of 10 mM Tris, and heated for 10 minutes at 95° C. to break open the bacterial cells. Two μL of the heated cells were used per 25 μL PCR reaction. The PCR reaction mix contained 0.2 μM each primer, 1×GC Genomic PCR Buffer, 1.0 M GCMelt, 1.1 mM Mg(OAc)₂, 0.2 mM each dNTP, and 1×Advantage-GC Genomic Polymerase Mix. The PCR was conducted in a Perkin Elmer Geneamp 9600 and consisted of an initial denaturation at 94° C. for 1 minute; 8 cycles of a 30 second denaturation at 94° C., a 1 minute annealing at 55° C., and a 3.5 minute extension at 72° C.; 22 cycles of a 30 second denaturation at 94° C., a 1 minute annealing at 61° C., and a 3.5 minute extension at 72° C.; followed by a final extension at 72° C. for 5 minutes. All suspected 35053crtE-kan colonies produced a crtE band the same size as the S17-1 crtE-kan control. They all also produced a band of the expected size for the ppsR gene and did not produce a band for the E. coli dxs gene.

To further confirm the presence of double-crossover events, Southern hybridization was conducted on eight 35053crtE-kan colonies as well as R. sphaeroides strains 35053 and 17023. Sequence data for the photosynthetic operon of strain 17023 is available in Genbank and was used to determine restriction enzymes likely to have hybridization patterns that would distinguish mutants from non-mutants. Genomic DNA was isolated from each line using a Gentra Puregene DNA Isolation Kit (Gentra, Minneapolis, Minn.). Two μg of genomic DNA was used in digests with the restriction enzymes ApaI and XhoI. The digests were separated on a 0.8% TAE agarose gel, and the DNA transferred to a nylon membrane. DIG-labeled molecular weight markers II and III (Roche) were also included on the gel/membrane. DIG-labeled probes of the crtE locus were synthesized using a PCR DIG Probe Synthesis Kit (Roche). After baling, membranes were prehybridized in EasyHyb Buffer (Roche) for at least 2 hours and hybridized overnight using 400 nL of a 0.5 DIG labeling reaction per mL of hybridization solution. Detection was conducted using a Wash and Block Buffer Set (Roche). Membranes were washed two times for 5-10 minutes each at room temperature in 2×SSC/0.1% SDS and two times for 15-20 minutes each at 68° C. in 0.1×SSC/0.1% SDS. They were then covered with blocking buffer and placed on a shaker for an hour at room temperature. The blocking buffer was replaced with fresh blocking buffer containing 150 mU of AP conjugate per mL of buffer, and the membranes shaken at room temperature for an additional 30 minutes. Membranes were then washed twice for 15 minutes each at room temperature with washing buffer, followed by a five minute wash with detection buffer. The detection buffer was replaced with fresh detection buffer containing 20 μL of NBT/BCIP solution per mL of buffer. This was placed in the dark at room temperature with no shaking until color developed, after which the buffer was replaced with 10 mM Tris-1 mM EDTA solution.

In the ApaI digest, the mutant lines exhibited a band of about 850 bp larger than the strain 35053 control, which is the size difference expected from the insertion of the kanamycin gene product in the StuI/XhoI sites. For the XhoI digest, strain 35053 exhibited a band of about 700 bp, strain 17023 had a band of about 1100 bp, mutant 7C had a band of 1550 bp, and the remaining mutants had a band of 2050 bp. The reason for the size difference in the XhoI bands for the mutants was unclear, but mutant 7C was used in further studies due to its possession of the expected band size relative to strain 35053. The resulting R. sphaeroides mutant containing a crtE knockout was designated ATCC 35053/ΔcrtE(kan).

ATCC 35053/ΔcrtE

R. sphaeroides cells lacking crtE were made using sacB selection as follows. A truncated crtE gene was cloned into the vector pL01, which is a suicide vector in R. sphaeroides. The pL01 vector carries a kanamycin resistance gene, a B. subtilis sacB gene, an oriT sequence, a CoIEI replicon, and a multiple cloning site (Lenz et al., J. Bacteriol., 176(14):4385-93 (1994)). The pL01crtE plasmid was introduced into R. sphaeroides strain 35053 through conjugation with an E. coli donor. The kanamycin resistance gene was used to select for single-crossover events between the truncated crtE gene and the genomic crtE gene that resulted in incorporation of the pL01crtE DNA into the genome. The presence of the sacB gene on the vector allowed for subsequent selection for the loss of the vector DNA from the genome, as expression of this gene in the presence of sucrose is lethal to E. coli and to R. sphaeroides under certain growth conditions. A portion of the double-crossover events that led to loss of the sacB gene contained the truncated crtE allele. This method of gene knockout is useful because no residual antibiotic resistance gene is left in the genome.

A three-step PCR process was used to create a 249 bp in-frame deletion in the crtE gene. The crtE gene from R. sphaeroides strain 35053 was amplified by PCR using primers designed to introduce an SphI restriction site at the beginning of the amplified fragment and a SacI restriction site at the end of the amplified fragment. The sequences of the primers were as follows.

| | | |
|---|---|---|
| CRTESPHF | 5′-CGTGGCATGCGTGTAAGAAAAAGTTGACA-CCTGTGGAGTC-3′ | (SEQ ID NO:172) |
| CRTESACR | 5′-CTAAGAGCTCAGTTCGGGCTCGGTCTCGC-CTTTCAGGAAG-3′ | (SEQ ID NO:173) |

The PCR reaction mix contained 0.2 μM each primer, 1×Genome Advantage reaction buffer, 1 M GCMelt, 1.1 mM Mg(OAc)₂, 0.2 mM each dNTP, 1×Genome Advantage Polymerase, and 1 ng of genomic DNA per μL of reaction mix. The PCR was conducted in a Perkin Elmer Geneamp 2400 and consisted of an initial denaturation at 94° C. for 2 minutes; 32 cycles of a 30 second denaturation at 94° C., a 45 second annealing at 64° C., and a 3 minute extension at 72° C., followed by a final extension at 72° C. for 7 minutes. 200 μL of PCR product was separated on a 1% TAE-agarose gel, and a 1.5 Kb fragment was excised and purified.

The second round of PCR consisted of two separate reactions: reaction A, which used primers CRTESPHF and CRTERI, and reaction B, which used primers CRTESACR and CRTEFI. The sequences of primers CRTEFI and CRTERI were as follows.

| | | |
|---|---|---|
| CRTEFI | 5′-GAGAGCGAGAGCCAGATCAAGAAGSGGCTGAAGGACATCC-3′ | (SEQ ID NO:174) |
| CRTERI | 5′-GGATGTCCTTCAGCCSCTTCTTGATCTGGCTCTCGCTCTC-3′ | (SEQ ID NO:175) |

The 20 nucleotides on the 3′ ends of this pair of primers are located near the center of the crtE gene, 249 bases apart from each other and facing towards the start (CRTERI) and end (CRTEFI) of the gene. The 20 bp on the 5′ ends of these primers are the reverse complement of the 3′ end of the other primer in the pair. PCR of the two separate reactions was conducted as in the first round, with the exception that 0.05 ng of first round product per μL of reaction mix was used as template. Also, the thermocycler program used a 2 minute initial denaturation at 94° C.; eight cycles of a 30 second denaturation at 94° C., a 45 second annealing at 56° C., and a 3 minute extension at 72° C., followed by eight cycles of a 30 second denaturation at 94° C., a 45 second annealing at 60° C., and a 3 minute extension at 72° C.; followed by 16 cycles of a 30 second denaturation at 94° C., a 45 second annealing at 64° C., and a 3 minute extension at 72° C.; followed by a final extension at 72° C. for 7 minutes. Both PCR products, about 590 and 650 bp in length, were separated on a 1% TAE-agarose gel, excised, and gel purified.

The third round of PCR used the same primers and reaction mixture as the first round of PCR with the exception that a mixture of 10 ng of each second round fragment was used as template rather than genomic DNA (200 μL reaction). The PCR program used was also the same as that used in the first round of PCR with the annealing time lengthened to 1.5 minutes. The 1.2 Kb third-round product was separated on a 1% TAE-agarose gel and purified. Three μg of purified DNA was digested with the restriction enzymes SacI and SphI. The digested DNA was cleaned using a QIAquick PCR Purification Kit and digested with the restriction enzyme StuI. StuI cut within the deleted region and ensured that there was little or no remaining full-length product. The digestion mixture was again cleaned using a QIAquick PCR Purification Kit.

Three μg of the vector pL01 was digested with the restriction enzymes SphI and SacI. The enzymes were inactivated by heating to 65° C. for 20 minutes, and the vector was dephosphorylated using shrimp alkaline phosphatase (Roche). The dephosphorylated vector DNA was gel purified on a 1% TAE-agarose gel.

Sixty-six ng of digested vector DNA was ligated with 80 ng of the digested third-round PCR product at 16° C. for 16 hours using T4 DNA ligase (Roche). One μL of ligation mix was electroporated into 40 μL of E. coli ElectroMAX™ DH5α™ electrocompetent cells (Life Technologies), which were then plated on LB media containing 50 μg/mL kanamycin (LBK50). Plasmid DNA was isolated from cultures of individual colonies and digested with the restriction enzyme SacI and with a mixture of SphI and SadI to confirm correct vector structure.

One μL of plasmid DNA was used to transform electrocompetent cells of the previously described E. coli strain S17-1. The electroporated cells were plated on LB media containing 25 μg/mL of kanamycin, 25 μg/mL of streptomycin, and 25 μg/mL of spectinomycin (LBKSMST). Single colonies were used to start cultures for plasmid DNA isolation and used in conjugation. These colonies were also plated on LB media containing 5% sucrose and 25 μg/mL of kanamycin to ensure that the sacB gene was still functional. Only colonies which exhibited lethality on the sucrose media were used in conjugation. The presence of the correct insert size was confirmed by digestion of plasmid DNA with the restriction enzymes SacI and SphI.

Growing cultures of R. sphaeroides strain 35053 were sub-cultured, using ⅕ and 1/10 volumes of inoculum, in 5 mL Sistrom's media supplemented with 20% LB and grown at 30° C. for 12 hours. The S17-1 donor colonies were grown in LBKSMST media at 37° C. for 12 hours. 1.5-3.0 mL of each culture was pelleted, and the pellets were washed four times with LB media. Relative pellet size was estimated and about 2 volumes of 35053 cells were used to 1 volume of S17-1 cells. The cell mixture was pelleted, resuspended in 20 μL of LB media, spotted on an LB plate, and incubated at 30° C. for 7-15 hours. The cells were then scraped off the surface of the plate and resuspended in 1.5 mL of Sistrom's salts. 200 μL of resuspended cells were plated on each of seven plates of SisKTell media.

Colonies that grew on the plates after about 10 days, representing proposed single-crossover events, were streaked to new plates of the same media. Upon growth, single colonies were streaked out on LBK25 media. Purified colonies were patched to Sistrom's media supplemented with 1×LB, 15% sucrose, 0.5% DMSO (v/v), and 25 μg/mL kanamycin (SisLBK15%SucDMSO). These were grown in an anaerobic chamber (Becton Dickinson, Sparks, Md.) at 30° C. for 5 days to check for lethality of the sacB gene in the proposed single-crossover events. Concurrently, the cultures were patched to SisLB media containing 15% sucrose and 0.5% DMSO (v/v) without kanamycin (SisLB15%SucDMSO). Several of the cultures exhibited both white and red colonies upon growth on this media. Whitish-gray colonies were purified from these cultures and tested by PCR to show that they contained the truncated crtE allele. These colonies were also screened using primers specific to the R. sphaeroides ppsR gene and the E. coli dxs gene as described above. Potential double crossovers were also streaked on LBK25 plates to confirm that they were now sensitive to kanamycin. The resulting R. sphaeroides mutant containing a crtE knockout was designated ATCC 35053/ΔcrtE.

Several discoveries were made using the sacB method to knockout nucleic acid sequenced within the R. sphaeroides genome. First, it was discovered that the cultures used in conjugations, particularly those of the recipient R. sphaeroides strain, should be in exponential growth. Second, it was discovered that when using the S17-1 strain as a vector donor, the use of telluride in the plating medium is unnecessary as this strain is a proline auxotroph and will not grow on Sistrom's media without LB supplementation. Third, it was discovered that potential single crossovers should be screened using two separate PCR reactions. The first reaction should use a primer within the gene of interest together with a primer homologous to upstream sequence. The second reaction should use a primer within the gene of interest together with a primer homologous to downstream sequence. One of these two reactions should produce a truncated fragment. Fourth, it was discovered that single crossovers that have been confirmed to have sacB lethality can be grown aerobically in Sistrom's media for 2 days and then plated on SisLB15%SucDMSO media. The volume plated varies depending on the rate of growth of the strain, but is about one μL or less for strain 35053. This is then grown anaerobically for about 5 days. Fifth, it was discovered that the sacB gene may not completely kill cells with the gene, so there may be a background level of very small colonies. The desired double-crossover colonies, however, are typically larger. These colonies should be purified and screened by PCR to identify whether they contain the truncated or full-length allele. Sixth, it was discovered that using one primer homologous to sequence upstream of the knockout gene and one primer homologous to sequence downstream of the gene is useful in confirming the correct location of the insertion event in addition to determining the allele that is present.

ATCC 35053/ΔppsR(strep)

R. sphaeroides cells lacking PPSR were made by inserting a spectinomycin/streptomycin resistance gene into the ppsR sequence as follows. To PCR amplify the ppsR gene from R. sphaeroides strain 17023, the following primers were designed based on published sequence (GenBank Accession Number L19596).

(SEQ ID NO:176)

| | |
|---|---|
| PPSRF2 | 5′-AGTCAGTACTAACTGGTGAAGACGCTGAAG-3′ |

(SEQ ID NO:177)

| | |
|---|---|
| PPSRR2 | 5′-GATCAGTACTGTGAACGAATACGATACGCA-3′ |

Each primer contained a ScaI restriction site. The ppsR gene was amplified using following reaction mix and PCR amplification program.

| Reaction Mix | | Program |
|---|---|---|
| pfu 10X buffer | 10 μL | 94° C. 5 minutes |
| DMSO | 5 μL | 8 cycles of: |
| dNTP mix (10 mM) | 8 μL | 94° C. 45 seconds |
| PPSRF2 (50 μM) | 2 μL | 54° C. 45 seconds |
| PPSRR2 (50 μM) | 2 μL | 72° C. 3 minutes |
| Genomic DNA (50 ng/μL) | 2 μL | 25 cycles of: |
| pfu enzyme (2.5 U/μL) | 2 μL | 94° C. 45 seconds |
| DI water | 69 μL | 61° C. 45 seconds |
| | | 72° C. 3 minutes |
| Total: | 100 μL | 72° C. 10 minutes |
| | | 4° C. Until used further |

The PCR product was separated on a 0.8% TAE agarose gel, and a band of about 1.8 Kb was cut and gel isolated using Qiagen Gel Isolation kit (Qiagen, Valencia, Calif.). The gel isolated DNA was digested with Seal (New England BioLabs, Beverly, Mass.) for 5 hours. The digested DNA was column purified using Qiagen Gel Isolation kit. The cut DNA was ligated into vector pSUP203 that was also digested with ScaI enzyme.

2.3 μg of pSUP203 plasmid DNA was digested for 4 hours at 37° C. with ScaI restriction enzyme. The digested DNA was separated on a 1% TAE agarose gel. A 7.6 Kb fragment was excised and purified. The purified plasmid DNA was then dephosphorylated using calf intestinal phosphatase (New England Biolabs). 100 ng of dephosphorylated plasmid DNA was ligated with 200 ng of the Seal-digested PpsR DNA for 16 hours at 14° C. using T4 DNA ligase (New England BioLabs). One μL of ligation mix was electroporated into 40 μL of E. coli ElectroMAX™ DH5α™ (Life Technologies, Gaithersburg, Md.) electrocompetent cells, which were then recovered in 1 mL of SOC media for one hour at 37° C. and plated on LB media containing 15 μg/mL tetracycline. Plasmid DNA was isolated from 8 individual colonies using Qiagen spin Mini prep kit and digested with Seal restriction enzyme to check insert size. Four of the colonies had a correct insert. 1.5 μg of the plasmid DNA obtained from confirmed colony was digested with XhoI restriction enzyme (New England BioLabs, Beverly, Mass.). This enzyme has a single restriction site in the open reading frame of ppsR gene. A linear DNA band of about 8.4 Kb was gel isolated using a Qiagen Gel isolation kit. A spectinomycin/streptomycin resistance omega cassette was obtained by digesting plasmid pUI1638 (Obtained from Dr. Samuel Kaplan's laboratory) with XhoI enzyme. The digest was separated on a 0.8% TAE agarose gel, and a DNA band of about 2.1 Kb was gel isolated. This DNA which encoded for spectinomycin/streptomycin resistance gene was ligated to pSUP203/PpsR, which was also restricted with XhoI enzyme. One μL of ligation mix was electroporated into 40 μL of E. coli ElectroMAX™ DH5α™ (Life Technologies, Gaithersburg, Md.) electrocompetent cells, which were then recovered in 1 nL of SOC media for one hour at 37° C. and plated on LB media with 15 μg/mL tetracycline, 25 μg/mL spectionomycin, and 25 μg/mL streptomycin. Plasmid DNA was isolated from 10 individual colonies using Qiagen spin Mini prep kit and digested separately with Seal and XhoI restriction enzyme to check insert size. Five of the colonies had a correct insert. 100 ng of plasmid DNA from a confirmed colony was electroporated into electrocompetent cells of the E. coli strain SM10. This strain contains a chromosomal copy of the trans-acting elements that mobilize oriT-containing plasmids during conjugation with a second bacterial strain. It also carries a gene conferring resistance to the antibiotic kanamycin. The transformation reaction was recovered in 1 mL of SOC media for one hour and plated on LB media with 10 μg/mL tetracycline, 25 μg/mL kanamycin, 25 μg/mL of streptomycin, and 25 μg/mL spectinomycin.

The pSUP203/ppsR-SM-ST construct was conjugated from the E. coli SM10 host into R. sphaeroides strain 35053. The SM10 donor was grown in LB media with 25 μg/mL kanamycin, 25 μg/mL streptomycin, and 25 μg/mL spectinomycin at 37° C. for 16 hours. A growing culture of R. sphaeroides strain 35053 was used to inoculate Sistrom's media in ⅕ to 1/10 dilutions. These cultures were grown for about 20 hours. Cells were pelleted for 1.5 mL of culture of both the SM10pSUP203/PpsR-SM-ST and 35053 genotypes. Pellets were washed four times in Sistrom's media without vitamins and glucose. The pellets were each resuspended in 1.5 mL of Sistrom's media without vitamins and glucose. 200 μL of the SM10pSUP203/PpsR-SM-ST cells were combined with 1.3 mL of the 35053 cells. This mixture was pelleted, the supernatant was removed, and the pellet was resuspended in 20 μL of LB media. The resuspended cells were spotted onto a LB plate that was then incubated at 30° C. for 7 hours. The cells were then scrapped off the LB plate, resuspended in 1.5 mL of 1×Sistrom's media without vitamins and glucose, and plated (200 μL/plate) on Sistrom's media supplemented with 25 μg/mL spectinomycin, 25 μg/mL streptomycin, and 10 μg/mL of telluride. The telluride retards the growth of E. coli cells but is detoxified by R. sphaeroides. After 7-10 days, small black colonies were picked off the plates and streaked to fresh plates of the same media. After 6 days of growth, colonies were patched to LB plates containing 25 μg/mL spectinomycin and 25 μg/mL streptomycin (LBSMST25), and also to LB plates containing 0.75 μg/mL tetracycline. Desirable double-crossover events, in which the PpsR-SM-ST gene is retained in the genome and the vector DNA is lost, would have spectinomycin/streptomycin resistance but lack tetracycline resistance. Colonies resulting from undesirable single-crossover events would demonstrate resistance to all of these antibiotic markers.

Colonies that exhibited only spectinomycin/streptomycin resistance and displayed deep red color were confirmed for double-crossover by Southern hybridization. Southern hybridization was conducted on nineteen potential 35053/PpsR-SM-ST colonies in addition to 35053 and R. sphaeroides strain 17023. Sequence data for the photosynthetic operon of 17023 is available in GenBank and was used to determine restriction enzymes likely to have hybridization patterns that would distinguish mutants from non-mutants. Genomic DNA was isolated from each line using a Gentra Puregene DNA Isolation Kit (Gentra, Minneapolis, Minn.). 2 μg of genomic DNA was used in digests using the restriction enzymes NcoI, ApaI, and XmaI in separate reactions. The digests were separated on a 1% TAE agarose gel, and the DNA was transferred to nylon membrane (Roche Molecular Biochemicals, Indianapolis, Ind.). DIG-labeled molecular weight markers II and III (Roche) were also included on the gel/membrane. DIG-labeled probes of the PpsR locus were made using a PCR DIG Probe Synthesis Kit (Roche). After baking, membranes were prehybridized in EasyHyb Buffer (Roche) for at least 2 hours and hybridized overnight using 400 nL of a 0.5 DIG labeling per mL of hybridization solution. Detection was done using a Roche Wash and Block Buffer Set (Roche). Membranes were washed two times for 5-10 minutes at room temperature in 2×SSC/0.1% SDS and two times for 15-20 minutes at 68° C. in 0.1×SSC/0.1% SDS. They were then covered with blocking buffer and placed on a shaker for an hour at room temperature. The blocking buffer was replaced with fresh blocking buffer containing 150 mU of AP conjugate per mL of buffer, and the membranes shaken at room temperature for an additional 30 minutes. Membranes were then washed twice for 15 minutes at room temperature with washing buffer, followed by a five minutes wash with detection buffer. The detection buffer was replaced with fresh detection buffer containing 20 μL of NBT/BCIP solution per mL of buffer. This was placed in the dark at room temperature with no shaking until sufficient color was developed.

In the NcoI digest, the lanes of colony 9 and 10 exhibited a band about 2 Kb larger than the 35053 control, which is the size difference expected from the insertion of the spectinomycin/streptomycin resistance cassette into the XhoI site For the XmaI digest, 35053 exhibited a single band about 5.5 Kb, while colonies 9, 10, and 5 exhibited two bands whose summed size was about 2 Kb higher than that of 35053. Two bands were observed in colony 9, 10, and 5 because a XmaI was introduced along with the spectinomycin/streptomycin resistance cassette. For ApaI digest, the control 35053 sample exhibited two bands since ppsR gene harbors an ApaI site. Each of these bands was about 2.3 Kb in size. Colony 9, 10, and 5 exhibited three bands, whose summed size was about 2 Kb higher band that of 35053. An extra band was observed in colonies 9, 10, and 5 because an ApaI site was introduced along with the spectinomycin/streptomycin resistance cassette.

The resulting R. sphaeroides mutant containing the ppsR knockout was designated ATCC 35053/AppsR(strep).

ATCC 35053/AppsR

R. sphaeroides cells lacking ppsR were made using sacB selection as follows. A three-step PCR process was used to create a 255 bp in-frame deletion in the PpsR gene, so that there would be no residual antibiotic resistance gene in the genome. The PpsR gene from R. sphaeroides strain 35053 was amplified by PCR using primers designed to introduce an SacI restriction site at the beginning of the amplified fragment and a SphI restriction site at the end of the amplified fragment. The sequences of the primers were as follows.

| | | |
|---|---|---|
| PPSRSACF2 | 5′-GTCAAATGAGCTCCAAACTGGTGAAGACGCTGAAGGACAT-3′ | (SEQ ID NO:178) |
| PPSRSPHR | 5′-CAGTCGGGCATGCGTCCATTTCAGTTGACATACTTCTGTG-3′ | (SEQ ID NO:179) |

The following PCR mix program was used to amplify the PpsR gene.

| Reaction Mix | | Program |
|---|---|---|
| pfu 10X buffer | 10 μL | 94° C. 2 minutes |
| DMSO | 5 μL | 8 cycles of: |
| dNTP mix (10 mM) | 3 μL | 94° C. 30 seconds |
| PPSRSACF2 (100 μM) | 1 μL | 58° C. 45 seconds |
| PPSRSPHR (100 μM) | 1 μL | 72° C. 3 minutes |
| Genomic DNA (50 ng/μL) | 2 μL | 25 cycles of: |
| pfu enzyme (2.5 U/μL) | 2 μL | 94° C. 30 seconds |
| DI water | 76 μL | 64° C. 45 seconds |
| | | 72° C. 3 minutes |
| Total: | 100 μL | 72° C. 7 minutes |
| | | 4° C. Until used further |

100 μL of PCR product was separated on a 1% TAE agarose gel, and a fragment about 1.8 Kb was excised and purified using Qiagen Gel isolation kit.

The second round of PCR consisted of two separate reactions: reaction A, which used primers PPSRSACF2 and PPSRMIDR, and reaction B, which used primers PPSRSPHR and PPSRMIDF. The sequences of primers PPSRMIDF and PPSRMIDR were as follows.

| | | |
|---|---|---|
| PPSRMIDF | 5′-CTCTTGCTCGGCGGCGTGCGGCTCTATCACGAGGGGGTGGA-3′ | (SEQ ID NO:180) |
| PPSRMIDR | 5′-TCCACCCCCTCGTGATAGAGCCGCACGCCGCCGAGCAAGAG-3′ | (SEQ ID NO:181) |

The 20 nucleotides on the 3′ ends of this pair of primers are located near the center of the ppsR gene, 255 bases apart from each other, and facing towards the start (PPSRMIDR) and end (PPSRMIDF) of the gene. The 20 bp on the 5′ ends of these primers are the reverse complement of the 3′ end of the other primer in the pair. The following reaction mix and program were used to conduct these PCR.

| | | AProgram |
|---|---|---|
| Reaction Mix A | | |
| pfu 10X buffer | 10 μL | 94° C. 2 minutes |
| DMSO | 5 μL | 8 cycles of: |
| dNTP mix (10 mM) | 3 μL | 94° C. 30 seconds |
| PPSRSACF2 (100 μM) | 1 μL | 58° C. 45 seconds |
| PPSRMIDR (100 μM) | 1 μL | 72° C. 3 minutes |
| DNA from first round | 1 μL | 25 cycles of: |
| (10 ng/μL) | | 94° C. 30 seconds |
| pfu enzyme (2.5 U/μL) | 2 μL | 64° C. 45 seconds |
| | | 72° C. 3 minutes |
| DI water | 77 μL | 72° C. 7 minutes |
| Total | 100 μL | 4° C. Until further use |
| Reaction Mix B | | |
| pfu 10X buffer | 10 μL | 94° C. 2 minutes |
| DMSO | 5 μL | 8 cycles of: |
| dNTP mix (10 mM) | 2 μL | 94° C. 30 seconds |
| PPSRSPHR (100 μM) | 1 μL | 58° C. 45 seconds |
| PPSRMIDF (100 μM) | 1 μL | 72° C. 3 minutes |
| DNA from first round | 1 μL | 25 cycles of: |
| (5 ng/μL) | | 94° C. 30 seconds |
| pfu enzyme (2.5 U/μL) | 2 μL | 64° C. 45 seconds |
| DI water | 78 μL | 72° C. 3 minutes |
| | | 72° C. 7 minutes |
| Total | 100 μL | 4° C. Until further use |

Both PCR products, about 800-700 bp in length, were separated on a 1% TAE agarose gel, excised, and gel purified using a Qiagen gel isolation kit.

The third round of PCR used primers PPSRSACF2 and PPSRSPHR but used both fragments derived in the second round of PCR as template. The PCR mixture used was the same as in the first round of PCR except that equal molar amounts of the round 2 fragments were used as template. The PCR program used was also the same as that used in the first round of PCR, with the annealing time lengthened to 1.5 minutes. The 1.5 Kb third-round product was separated on a 1% TAE agarose gel and purified using Qiagen gel isolation kit. The purified DNA was digested overnight at 37° C. with the restriction enzymes SacI and SphI.

Three μg of the vector pL01 was digested with the restriction enzymes SphI and SacI at 37° C. for 16 hours. The enzymes were inactivated by heating to 65° C. for 20 minutes. Dephosphorylation of the vector was achieved by adding 4.7 μL of shrimp alkaline phosphatase 10×buffer (Roche) and 2 μL of shrimp alkaline phosphatase to the inactivated digest. This mixture was heated at 37° C. for 10 minutes and then 65° C. for 15 minutes. The dephosphorylated vector DNA was then gel purified on a 1.0% TAE agarose gel.

98 ng of vector DNA was ligated with 210 ng of the digested third round PCR at 14° C. for 14 hours using T4 DNA ligase (Roche). One μL of ligation mix was electroporated into 40 μL of E coli ElectroMAX™ DH5α™ electrocompetent cells (Life Technologies), which were then recovered in 1 mL of SOC media for one hour and plated on LB media with 25 μg/mL kanamycin (LBK25). Plasmid DNA was isolated from eight individual colonies. Plasmid DNA was checked for correct insert with a PCR screen using the PCR protocol from first round.

One μL of plasmid DNA was used to transform electrocompetent cells of E. coli strain S17-1. The electroporated cells were recovered in 1 mL of SOC media for one hour and plated on LB media with 25 μg/mL of kanamycin, 25 μg/mL of streptomycin, and 25 μg/mL of spectinomycin (LBKSMST). Single colonies were used to start cultures for plasmid DNA isolation and used in conjugation. These colonies were also plated on LB media containing 5% or 15% sucrose, and 25 μg/mL of kanamycin to ensure that the sacB gene was still functional. Only colonies that showed lethality on the sucrose media were used in conjugation. The presence of the correct insert size was confirmed by colony PCR.

Growing cultures of R. sphaeroides strain 35053 were subcultured, using ¼ and ⅛ volumes of inoculum, in 5 mL Sistrom's media supplemented with 20% LB and grown at 30° C. for 9 hours. The S17-1 donor colonies were grown in LBKSMST media at 37° C. for 16 hours. 3.0 nL of 35053 and 0.5 mL of S17-1 donor cells were centrifuged and washed four times in Sistrom's media without glucose. Each cell pellet was resuspended into 20 μL LB, and the S17-1 donor suspension was mixed with 35053. The mixture was then spotted on LB, which was incubated at 30° C. for 14-16 hours. The cells were then scraped off the surface of the plate and resuspended in 1.5 mL of Sistrom's salts. 200 μL of resuspended cells were plated on each of the seven Sistrom's media plates that were supplemented with 25 μg/mL of kanamycin.

Colonies that grew on the plates after about 10-14 days, representing proposed single crossover events, were streaked to new plates of the same media. Upon growth, single colonies were transferred to LBK25 media. These cultures were grown for 36 to 48 hours in Sistrom's media supplemented with 20% LB and no kanamycin at 30° C. 0.1 ∥L and 5 μL of this culture was plated on LB media that was supplemented with Sistrom's salts and 15% sucrose. The plates were placed in an anaerobic chamber (Becton Dickinson, Sparks, MD), and the chamber was placed in a 30° C. incubator. After 4-5 days, several colonies showed up on the plates, indicating the occurrence of double-crossover events. Four colonies from each single-crossover strain were purified by streaking on LB agar plates. Single colonies of double-crossover strains were screen by PCR for integration of truncated version of the ppsR gene into the chromosome. For screening, the following primers were used, which were located upstream and downstream of the PpsR gene. The use of upstream and downstream primer confirms both the locus of integration as well as truncation of PpsR gene.

| | |
|---|---|
| PPSRUPF | |
| 5′-GAGCAGCACACTCTGGGAGC-3′ | (SEQ ID NO:182) |
| PPSRDNR | |
| 5′-CCACACAGGTAGGACACCCAC-3′ | (SEQ ID NO:183) |

The following reaction mix and PCR program was used.

| Reaction Mix | | Program |
|---|---|---|
| Taq Mg + 10X buffer | 2.5 μL | 94° C. 2 minutes |
| DMSO | 1.25 μL | 29 cycles of: |
| dNTP mix (10 mM) | 0.5 μL | 94° C. 30 seconds |
| PPSRUPF (100 μM) | 0.125 μL | 61° C. 45 seconds |
| PPSRDNR (100 μM) | 0.125 μL | 72° C. 3 minutes |
| Cell boil mix | 2 μL | 72° C. 7 minutes |
| Taq enzyme (5 U/μL) | 0.2 μL | 4° C. Until further use |
| DI water | 18.3 μL | |
| Total | 25 μL | |

The cell boil mix was prepared by resuspending a single colony in 20-25 μL of water. The suspension was heated at 95° C. for 10 minutes in a PCR machine. The tube was given a quick spin to pellet the solids.

The colonies that exhibited the truncated version of the PpsR gene were further tested for kanamycin sensitivity by streaking them on LB plates that were supplemented with 25 μg/mL of kanamycin. Also, these colonies were PCR screened for the kanamycin resistance gene.

The resulting R. sphaeroides mutant containing the ppsR knockout was designated ATCC 35053/ΔppsR.

ATCC 35053/ΔccoN

R. sphaeroides cells lacking ccoN were made using sacB selection as follows. A mutant of R. sphaeroides strain 2.4.1 having a 546 bp deletion in the ccoN gene (R. sphaeroides 2.4.1/ΔccoN) was obtained from the laboratory of Samuel Kaplan at the University of Texas (Oh and Kaplan, Biochemistry, 38:2688-2696 (1999)). The mutated ccoN locus of this strain was amplified by PCR and cloned into pL01. This plasmid was transformed into E. coli strain S 17-1. The S 17-1 strain was conjugated with R. sphaeroides strain 35053, and colonies were identified in which the truncated locus had replaced the native ccoN gene.

The truncated ccoN gene from R. sphaeroides 2.4.1/ΔccoN was amplified by PCR using primers designed to introduce a SacI restriction site at the beginning of the amplified fragment and a SphI restriction site at the end of the amplified fragment. The sequences of the primers were as follows.

| | | |
|---|---|---|
| CCONSACF | 5′-TCAGAGCTCGTGTGATCGAATGGGGCTTTGTTCCTTGATG-3′ | (SEQ ID NO:184) |
| CCONSPHR | 5′-GAAGCATGCAGGTGATCGACGTGCCACTCGTCCGAATAG-3′ | (SEQ ID NO:185) |

The PCR reaction mix contained 0.2 μM each primer, 1×Native Pfu reaction buffer, 0.2 nM each dNTP, 5% DMSO, and 10 units of Pfu DNA polymerase in a 200 μL reaction. Three μL of the glycerol stock was diluted in 20 μL of 10 mM Tris and heated at 94° C. for 10 minutes, after which 4 μL was added to the PCR reaction. The PCR was conducted in a MJ Research PT100 and consisted of an initial denaturation at 94° C. for 2 minutes; 32 cycles of a 30 second denaturation at 94° C., a 1 minute annealing at 66° C., and a 4 minute extension at 72° C., followed by a final extension at 72° C. for 7 minutes. The PCR product was separated on a 1% TAE-agarose gel, and a 1.6 Kb fragment was excised and purified. Three fig of purified PCR product was digested with SacI restriction enzyme and separated on a 1% TAE gel. A 1.4 Kb band was excised and purified. A SacI restriction site exists about 200 bp from the CCONSPHR end of the original PCR product.

Three μg of the vector pL01 was digested with the restriction enzyme SacI. The enzyme was inactivated by heating to 65° C. for 20 minutes, and the digested vector was dephosphorylated using shrimp alkaline phosphatase. The dephosphorylated vector DNA was gel purified on a 1% TAE-agarose gel.

50 ng of digested vector DNA was ligated with 65 ng of the digested ccoN PCR product at 16° C. for 16 hours using T4 DNA ligase (Roche). One μL of ligation mix was electroporated into 40 μL of E. coli Electromax™ DH5α™ electrocompetent cells, which were then plated on LBK media. Plasmid DNA was isolated from cultures of individual colonies and digested with the restriction enzyme SacI to confirm correct insert size.

The E. coli strain S17-1 contains a chromosomal copy of the trans-acting elements that mobilize oriT-containing plasmids during conjugation with a second bacterial strain. It also carries genes conferring resistance to the antibiotics streptomycin and spectinomycin. In addition, S17-1 is a proline auxotroph and will not grow on unsupplemented Sistrom's media. One μL of DNA of the truncated ccoN construct was used to transform electrocompetent cells of E. coli strain S17-1. The electroporation was plated on LBKSMST. Single colonies were used to start cultures for plasmid DNA isolation and used in conjugation. These colonies were also plated on LB media containing 5% sucrose and 25 μg/mL of kanamycin to ensure that the sacB gene was still functional. Only colonies that exhibited lethality on the sucrose media were used in conjugation. The presence of the correct insert size was confirmed by digestion of plasmid DNA with the restriction enzyme Sacd.

Growing cultures of R. sphaeroides strain 35053 were subcultured in Sistrom's media supplemented with 20% LB to ensure that they were in exponential growth. The S17-1 donor colonies were grown in LBKSMST media at 37° C. overnight or subcultured from growing colonies. 2-4 mL of each culture was centrifuged, and the pellets were washed four times in LB media. Relative pellet size was estimated, and about 2 volumes of 35053 cells were used to 1 volume of S17-1 cells. The cell mixture was then pelleted, resuspended in 20 μL of LB media, and spotted on an LB plate. This plate was incubated at 30° C. for 7-15 hours. The cells were then scraped off the surface of the plate and resuspended in 1.2 mL of Sistrom's salts. 200 μL of resuspended cells were plated on each of six plates of Sistrom's media containing 25 μg/mL of kanamycin (SisK).

Colonies that grew on the plates after about 10 days, representing potential single-crossover events, were streaked to new plates of SisK media. Upon growth, single colonies were transferred to LBK media. Purified colonies were streaked to Sistrom's media supplemented with IX LB, 15% sucrose, 0.5% DMSO (v/v), and 25 μg/mL kanamycin (SisLBK15%SucDMSO). These were grown in an anaerobic chamber (Becton Dickinson, Sparks, Md.) at 30° C. for 5 days to check for lethality of the sacB gene in the single-crossover events. The purified colonies were also screened in two separate PCR reactions. The first reaction used a primer within the gene of interest (CCONR) together with a primer homologous to upstream sequence (CCONUPF2), and the second reaction used a primer within the gene of interest (CCONSACF) together with a primer homologous to downstream sequence (CCONDNR2). Single-crossover events exhibited a truncated fragment in one of the two reactions, depending on whether the crossover occurred upstream or downstream of the deletion. The primer sequences were as follows.

| | | |
|---|---|---|
| CCONUPF2 | 5′-CTCACAACCTCCAACCGATG-3′ | (SEQ ID NO:186) |
| CCONR | 5′-CGATGGTGACCACGAAGAAG-3′ | (SEQ ID NO:94) |
| CCONDNR2 | 5′-CGTAACGCTCGGTCTCGTC-3′ | (SEQ ID NO:129) |

Single-crossover colonies were grown in Sistrom's media supplemented with 20% LB. After 2 days of growth, 0.1-1 μL of the cultures was plated on Sistrom's media supplemented with 1×LB, 0.5% DMSO (v/v), and 15% sucrose (SisLB15%SucDMSO). These cultures were grown anaerobically for about 5 days. The sacB gene did not always completely kill cells with the gene, so there was often a background level of very small colonies. The larger colonies, which represented double-crossover events, were purified on LB media and screened by PCR to identify whether they contained the truncated or full-length allele. The CCONUPF2 and CCONDNR2 primers were used in this PCR screen to ensure that the truncated gene also was inserted in the correct location in the genome. Potential double-crossovers were also streaked on LBK plates to confirm that they were now sensitive to kanamycin.

The resulting R. sphaeroides mutant containing the ccoN knockout was designated ATCC 35053/ΔccoN.

ATCC 35053/ΔcrtE/ΔccoN

R. sphaeroides cells lacking crtE and ccoN were made as follows. The wildtype ccoN allele of a crtE knockout mutant (ATCC 35053/ΔcrtE) was replaced with a truncated ccoN allele as described above. Double-crossover colonies having the truncated ccoN allele were then re-screened by PCR for the crtE and ccoN loci. These colonies were plated on LBK25 and screened by PCR to confirm the loss of the vector from the genome. The resulting R. sphaeroides mutant containing the crtE knockout and ccoN knockout was designated ATCC 35053/ΔcrtE/ΔccoN.

ATCC 35053/ΔcrtE/ΔppsR/ΔccoN

R. sphaeroides cells lacking crtE, ppsR, and ccoN were made as follows. The wildtype ppsR allele of a crtE/ccoN knockout mutant (ATCC 35053/ΔcrtE/ΔccoN) was replaced with a truncated ppsR allele as described above with the following exceptions. After conjugation on an LB plate, the conjugated cells were plated on Sistrom's media containing 25 μg/mL of kanamycin and 0.5% DMSO (SisKDMSO) rather than on SisK. After purification on SisKDMSO and LBKDMSO, single-crossovers were grown aerobically in Sistrom's media supplemented with 1×LB and 0.5% DMSO. After 2 days of growth, the cultures were plated on Sistrom's media supplemented with 1×LB, 15% sucrose, and 0.5% DMSO, and grown anaerobically for 5 days. Potential double-crossover colonies were purified on LBDMSO and screened by PCR using the PPSRUPF and PPSRDNR primers. Colonies having the truncated ppsR allele were then rescreened by PCR for the crtE, ppsR, and ccoN loci. These colonies were also plated on LBKDMSO and screened by PCR to confirm the loss of the vector from the genome. The resulting R. sphaeroides mutant containing the crtE knockout, ppsR knockout, and ccoN knockout was designated ATCC 35053/ΔcrtE/ΔppsR/ΔccoN.

### Example 10

### Making Recombinant Microorganisms that Overexpress a Particular Sequence While a Containing Knock-Out

Any construct developed for the overexpression of genes are transferred to any of the background genotypes developed by gene knockout techniques. For example, the pMCS2tetP/Stdxs/Rsdds/EcUbiC or the pMCS2tetP/Stdxs/Rsdds/RsLytB construct is transferred into the R. sphaeroides ATCC 35053/ΔcrtE/ΔppsR/ΔccoN mutant cells to combine the productive effects of gene overexpression and engineering of gene regulation or carbon flow. The construct is transferred to the desired genotype by electroporation or conjugation. Conjugation of a plasmid into an R. sphaeroides strain follows the procedure described for the isolation of single-crossover events except that, since the efficiency of plasmid transfer is much higher than that of chromosomal integration, a 0.1-1 μL plating volume from the ˜400 μL conjugation recovery is ample to obtain transformed colonies. Single colony PCR is used to check the integrity of the construct in the new background, and evaluations of the productivity of the new strain are made. Genes that are productive are integrated, in one or more copies, into appropriate regions of the chromosome of a productive strain along with or downstream of a highly-expressing promoter.

### Example 11

### Three Liter Fermentations

Cultures of R. sphaeroides ATCC 35053 with various inserted genes or knockouts were grown in 5 mL culture tubes containing Sistrom's media with 4 g/L glucose. After 48 hours of growth at 30° C. with 250 rpm shaking, the entire contents of the tube were used to inoculate a 300 mL baffled shake flask containing Sistrom's media with 4 g/L glucose. After incubation at 30° C. for 48 hours, the entire contents of the flask were added to 2.7 L of Sistrom's media containing 40 g/L glucose in a B. Braun Biotech International Model Biostat B fermenter.

The fermenter was maintained at 30° C., and the cascade was set to maintain the dissolved oxygen (DO) at 40%. The air inflow was maintained at 1 vvm, and the pH was maintained at 7.3 with an automatic feed of 2N NH₄OH. Foaming was controlled by addition of Sigma Antifoam 289. Kanamycin to a concentration of 50 μg/mL was added to fermentations with strains containing the broad host range vector pBBRIMCS2 either with or without an inserted gene. At 24 to 30 hours, when the agitation increase to maintain a DO of 40% had leveled off, the agitation and DO were decoupled, and the agitation was fixed at 240 rpm. The air inflow was lowered to 0.3 vvm. Kanamycin to 50 μg/mL was again added to fermentations containing the expression vector.

The fermentation samples for coenzyme Q10 and spheroidenone analysis were removed at 69 to 75 hours into the fermentation.

Example 12

### Three-Hundred Milliliter Fermentations

Cultures of R. sphaeroides ATCC 35053 with various overexpressed genes or knockouts were grown in 5 mL culture tubes containing Sistrom's media with 4 g/L glucose. After 48 hours of growth at 30° C. with 250 rpm shaking, the entire contents of the tube were used to inoculate a 300 mL baffled shake flask containing Sistrom's media with 4 g/L glucose. After incubation at 30° C. for 48 hours, 30 mL of the flask were added to 270 mL of Sistrom's media containing 40 g/L glucose in a 500 mL Infors AG-CH-4103 fermenter.

The fermenter was maintained at 30° C., and the cascade was set to maintain the dissolved oxygen (DO) at 40%. The air inflow was maintained at 1 vvm, and the pH was maintained at 7.3 with an automatic feed of 2N NH₄OH. Foaming was controlled by addition of Sigma Antifoam 289. Kanamycin to a concentration of 50 μg/mL was added to fermentations with strains containing the broad host range vector pBBRIMCS2 either with or without an inserted gene. At 24 to 30 hours, when the agitation increase to maintain a DO of 40% had leveled off, the agitation and DO were decoupled, and the agitation was fixed at 400 rpm. The air inflow was lowered to 0.3 vvm. Kanamycin to 50 μg/mL was again added to fermentations containing the expression vector.

The fermentation samples for coenzyme Q10 and spheroidenone analysis were removed at 69 to 75 hours into the fermentation.

### Example 13

### Analysis of Spheroidenone

At various times during the fermentation, 15 mL of fermentation volume was withdrawn. The volume of sample needed to obtain 5 mg of dry cell weight (DCW) was used for spheroidenone analysis. The sample was washed one time in water and resuspended in an equal volume of water. The volume of sample calculated in step 1 was added to a 1.8 mL-microfuge tube and was centrifuged at 10,000 rpm for 3 minutes in an IEC MicroMax microfuge. The supernatant was removed, and the pellet was completely resuspended in 1.0 nL of Acetone:Methanol (7:2) and stored at room temperature away from light for 30 minutes. The sample was mixed once during this incubation. After incubation, the sample was centrifuged at 10,000 rpm for 3 minutes, and the extract (supernatant) collected. Samples were stored −20° C. for analysis at a later time. The carotenoid extract was analyzed on a spectrophotometer scanning in the range of 350 nm to 800 nm, and the OD₄₈₀ was recorded. The amount of carotenoid in mg/100 mL of culture was calculated using the following equation:

Spheroidenone (mg)/100 mL culture=((OD₄₈₀−(0.0816*OD₇₇₀))*0.484)/Vol. of sample from step 1

From mg of Spheroidenone/100 mL of culture, the amount of Spheroidenone/mg of dry cell weight (DCW) was calculated using the DCW number as the conversion factor. Care was taken to correct for any dilution factor required while the sample was scanned on the spectrophotometer.

### Example 14

### Analyzing CoQ(10) Levels Produced via Fermentation

100 mL of fermentation broth was removed once per day and placed in a tared 250 mL centrifuge bottle. The samples were centrifuged at 15,000×g for 5 minutes, the supernatant was poured off, and the samples were resuspended in 50 mL cold water. The samples were centrifuged again at 15,000×g for 5 minutes, and the supernatant was poured off. The wet weight of the biomass was determined, and the biomass was resuspended in 1.5 times its weight in water. The samples were stored covered with foil at −80° C. before analysis.

Before analysis, the samples were warmed at 21° C. for 15 minutes. 1.0 mL was withdrawn. Sodium dodecyl sulfate was added to a final concentration of 1.67%. The samples were extracted with 14 mL of a hexane:ethanol (5:2) mixture. The samples were then evaporated to dryness and dissolved in 2 mL of a methanol:ethanol (9:2) mixture. The samples were then analyzed on a Waters Nova-Pak C18 (3.9×150 mm: 4 Um) column with a PDA detector set from 200-300 um. Resolution was at 1.2 nm with a maximum absorbance at 275 nm. The run time was 15 minutes, and the injection volume was 20 μL.

The dry weight of the samples were determined drying an aliquot at 105° C. in an aluminum weighing pan for at least four hours.

### Example 15

### Production of CoQ(10)

The following seven experiments measured the amount of CoQ(10) produced by the indicated microorganisms in a 3 liter scale fermentation.

In experiment 1, the following data were collected after 96 hours of fermentation:

| Strain | Coenzyme Q10 (ppm) dry weight basis |
|---|---|
| ATCC 35053 | 2950 |
| ATCC 35053/ΔcrtE | 6508 |

These results demonstrated that the inactivation of crtE increased the production of COQ(10).

In experiment 2, the following data were collected after 69 to 75 hours of fermentation:

| Strain | Coenzyme Q10 (ppm) dry weight basis |
|---|---|
| ATCC 35053 | 1655 |
| ATCC 35053/ΔppsR(strep) | 3812 |

These results demonstrated that the inactivation of ppsR increased the production of COQ(10).

In experiment 3, the following data were collected after 69 to 75 hours of fermentation:

| | Coenzyme Q10 (ppm) | Spheroidenone (ppm) |
|---|---|---|
| Strain | dry weight basis | dry weight basis |
| ATCC 35053 | 2951 | 1980 |
| ATCC 35053/ΔccoN | 3527 | 2959 |

These results demonstrated that the inactivation of ccoN increased the production of CoQ(10) and spheroidenone.

In experiment 4, the following data were collected after 69 to 75 hours of fermentation:

| | Coenzyme Q10 (ppm) dry |
|---|---|
| Strain | weight basis |
| ATCC 35053/ΔcrtE | 3255 |
| ATCC 35053/ΔcrtE/ΔccoN isolate 8-7 | 7951 |

These results demonstrated that the inactivation of crtE and ccoN increased the production of CoQ(10) as compared to inactivating crtE only.

In experiment 5, the following data were collected after 69 to 75 hours of fermentation:

| | Coenzyme Q10 (ppm) dry |
|---|---|
| Strain | weight basis |
| ATCC 35053/ΔcrtE | 3545 |
| ATCC 35053/ΔcrtE/ΔccoN isolate 111 | 4984 |
| ATCC 35053/ΔcrtE/ΔppsR/ΔccoN | 11,676 |

These results demonstrated that the inactivation of crtE and ccoN increased the production of CoQ(10) as compared to inactivating crtE only. In addition, these results demonstrated that the inactivation of crtE, ccoN, and ppsR increased the production of CoQ(10) as compared to inactivating only crtE and ccoN.

In experiment 6, the following data were collected after 69 to 75 hours of fermentation:

| | Coenzyme Q10 (ppm) dry |
|---|---|
| Strain | weight basis |
| ATCC 35053/ΔcrtE | 3833 |
| ATCC 35053/ΔcrtE/pMCS2tetP/Stdxs | 4928 |
| ATCC 35053/ΔcrtE/pMCS2glnP/Stdxs | 5508 |
| ATCC 35053/ΔcrtE/pMCS2tetP/Stdds | 4652 |

These results demonstrated that the inactivation of crtE together with the addition of Stdxs increased the production of CoQ(10) as compared to inactivating crtE only. In addition, these results demonstrated that the use of the gin promoter with Stdxs resulted in more production of CoQ(10) when compared to the use of the tet promoter with Stdxs. Further, these results demonstrated that the inactivation of crtE together with the addition of Stdds increased the production of CoQ(10) as compared to inactivating crtE only.

In experiment 7, the following data were collected after 69 to 75 hours of fermentation:

| | CoQ(10) (ppm) dry |
|---|---|
| Strain | weight basis |
| ATCC 35053/pMCS2tetP | 3909 |
| ATCC 35053/pMCS2tetP/Stdxs/Rsdds | 5387 |
| ATCC 35053/pMCS2tetP/Stdxs/Rsdds/RsLytB | 5962 |
| ATCC 35053/pMCS2tetP/Stdxs/Rsdds/EcUbiC | 6439 |

These results demonstrated that the addition of Stdxs and Rsdds increased the production of CoQ(10) as compared to adding vector only. In addition, these results demonstrated that the addition of either RsLytB or EcUbiC together with the addition of Stdxs and Rsdds increased the production of CoQ(10) as compared to adding only Stdxs and Rsdds.

The following four experiments measured the amount of CoQ(10) produced by the indicated microorganisms in a 300 nL scale fermentation.

In experiment 1, the following data were collected after 69 to 75 hours of fermentation:

| | CoQ(10) (ppm) dry |
|---|---|
| Strain | weight basis |
| ATCC 35053/pMCS2tetP | 5250 |
| ATCC 35053/pMCS2tetP/Stdxs | 5758 |
| ATCC 35053/pMCS2tetP/Rsdds | 6944 |
| ATCC 35053/pMCS2tetP/Stdxs/Rsdds | 6875 |
| ATCC 35053/pMCS2tetP/Stdxs/Rsdds/EcUbiC | 7808 |

These results demonstrated that the addition of either Stdxs or Rsdds increased the production of CoQ(10) as compared to adding vector only. In addition, these results demonstrated that the addition of Stdxs, Rsdds, and EcUbiC increased the production of CoQ(10) as compared to adding only Stdxs and Rsdds.

In experiment 2, the following data were collected after 69 to 75 hours of fermentation:

| | CoQ(10) (ppm) dry |
|---|---|
| Strain | weight basis |
| ATCC 35053/pMCS2tetP | 5483 |
| ATCC 35053/pMCS2tetP/EcubiC | 6360 |
| ATCC 35053/pMCS2tetP/RsLytB | 5976 |
| ATCC 35053/pMCS2tetP/Stdxs/Rsdds/RsLytB | 6751 |

These results demonstrated that the addition of either EcUbiC or RsLytB increased the production of CoQ(10) as compared to adding vector only. In addition, these results demonstrated that the addition of Stdxs, Rsdds, and RsLytB increased the production of CoQ(10) as compared to adding only RsLytB.

In experiment 3, the following data were collected after 69 to 75 hours of fermentation:

| | CoQ(10) (ppm) dry |
|---|---|
| Strain | weight basis |
| ATCC 35053/pMCS2tetP | 5072 |
| ATCC 35053/pMCS2tetP/Stdxs/Rsdds/RsLytB | 8050 |

These results demonstrated that the addition of Stdxs, Rsdds, and RsLytB increased the production of CoQ(10) as compared to adding vector only.

In experiment 4, the following data were collected after 69 to 75 hours of fermentation:

| | Coenzyme Q10 (ppm) dry |
|---|---|
| Strain | weight basis |
| ATCC 35053/pMCS2tetP | 4503 |
| ATCC 35053/pMCS2tetP/Stdxs/Rsdds | 8833 |

These results demonstrated that the addition of Stdxs and Rsdds increased the production of CoQ(10) as compared to adding vector only.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An isolated nucleic acid comprising a nucleic acid sequence having a length and a percent identity to the sequence set forth in SEQ ID NO: 1 over said length, wherein the point defined by said length and said percent identity is within the area defined by points A, B, C, and D of FIG. 26, wherein point A has coordinates (3626, 100), point B has coordinates (3626, 65), point C has coordinates (50, 65), and point D has coordinates (12, 100).

2. The isolated nucleic acid of claim 1, wherein said point B has coordinates (3626, 85).

3. The isolated nucleic acid of claim 1, wherein said point C has coordinates (100, 65).

4. The isolated nucleic acid of claim 1, wherein said point C has coordinates (50, 85).

5. The isolated nucleic acid of claim 1, wherein said point D has coordinates (15, 100).

6. The isolated nucleic acid of claim 1, wherein said nucleic acid sequence encodes a polypeptide.

7. The isolated nucleic acid of claim 6, wherein said polypeptide has DXS activity.

8. The isolated nucleic acid of claim 1, wherein said nucleic acid sequence is as set forth in SEQ ID NO: 1.

9. An isolated nucleic acid comprising a nucleic acid sequence having a length and a percent identity to the sequence set forth in SEQ ID NO:2 over said length, wherein the point defined by said length and said percent identity is within the area defined by points A, B, C, and D of FIG. 26, wherein point A has coordinates (1926, 100), point B has coordinates (1926, 65), point C has coordinates (50, 65), and point D has coordinates (12, 100).

10. The isolated nucleic acid of claim 9, wherein said nucleic acid sequence encodes a polypeptide.

11. The isolated nucleic acid of claim 10, wherein said polypeptide has DXS activity.

12. An isolated nucleic acid comprising a nucleic acid sequence, wherein said nucleic acid sequence encodes a polypeptide comprising an amino acid sequence, wherein said amino acid sequence has a length and a percent identity to the sequence set forth in SEQ ID NO:3 over said length, wherein the point defined by said length and said percent identity is within the area defined by points A, B, C, and D of FIG. 26, wherein point A has coordinates (641, 100), point B has coordinates (641, 65), point C has coordinates (25, 65), and point D has coordinates (5, 100).

13. The isolated nucleic acid of claim 12, wherein said polypeptide has DXS activity.

14. An isolated nucleic acid comprising a nucleic acid sequence having a length and a percent identity to the sequence set forth in SEQ ID NO:37 over said length, wherein the point defined by said length and said percent identity is within the area defined by points A, B, C, and D of FIG. 26, wherein point A has coordinates (1990, 100), point B has coordinates (1990, 65), point C has coordinates (50, 65), and point D has coordinates (16, 100).

15. The isolated nucleic acid of claim 14, wherein said point B has coordinates (1990, 85).

16. The isolated nucleic acid of claim 14, wherein said point C has coordinates (100, 55).

17. The isolated nucleic acid of claim 14, wherein said point C has coordinates (50, 85).

18. The isolated nucleic acid of claim 14, wherein said point D has coordinates (20, 100).

19. The isolated nucleic acid of claim 14, wherein said nucleic acid sequence encodes a polypeptide.

20. The isolated nucleic acid of claim 19, wherein said polypeptide has DDS activity.

21. The isolated nucleic acid of claim 14, wherein said nucleic acid sequence is as set forth in SEQ ID NO:37.

22. An isolated nucleic acid comprising a nucleic acid sequence having a length and a percent identity to the sequence set forth in SEQ ID NO:38 over said length, wherein the point defined by said length and said percent identity is within the area defined by points A, B, C, and D of FIG. 26, wherein point A has coordinates (1002, 100), point B has coordinates (1002, 65), point C has coordinates (50, 65), and point D has coordinates (16, 100).

23. The isolated nucleic acid of claim 22, wherein said nucleic acid sequence encodes a polypeptide.

24. The isolated nucleic acid of claim 23, wherein said polypeptide has DDS activity.

25. An isolated nucleic acid comprising a nucleic acid sequence, wherein said nucleic acid sequence encodes a polypeptide comprising an amino acid sequence, wherein said amino acid sequence has a length and a percent identity to the sequence set forth in SEQ ID NO:39 over said length, wherein the point defined by said length and said percent identity is within the area defined by points A, B, C, and D of FIG. 26, wherein point A has coordinates (333, 100), point B has coordinates (333, 65), point C has coordinates (25, 65), and point D has coordinates (5, 100).

26. The isolated nucleic acid of claim 25, wherein said polypeptide has DDS activity.

27. Al isolated nucleic acid comprising a nucleic acid sequence having a length and a percent identity to the sequence set forth in SEQ ID NO:40 over said length, wherein the point defined by said length and said percent identity is within the area defined by points A, B, C, and D of FIG. 26, wherein point A has coordinates (1833, 100), point B has coordinates (1833, 65), point C has coordinates (50, 65), and point D has coordinates (16, 100).

28. The isolated nucleic acid of claim 27, wherein said point B has coordinates (1833, 85).

29. The isolated nucleic acid of claim 27, wherein said point C has coordinates (100, 65).

30. The isolated nucleic acid of claim 27, wherein said point C has coordinates (50, 85).

31. The isolated nucleic acid of claim 27, wherein said point D has coordinates (20, 100).

32. The isolated nucleic acid of claim 27, wherein said nucleic acid sequence encodes a polypeptide.

33. The isolated nucleic acid of claim 32, wherein said polypeptide has DDS activity.

34. The isolated nucleic acid of claim 27, wherein said nucleic acid sequence is as set forth in SEQ ID NO:40.

35. An isolated nucleic acid comprising a nucleic acid sequence having a length and a percent identity to the sequence set forth in SEQ ID NO:41 over said length, wherein the point defined by said length and said percent identity is within the area defined by points A, B, C, and D of FIG. 26, wherein point A has coordinates (1014, 100), point B has coordinates (1014, 65), point C has coordinates (50, 65), and point D has coordinates (16, 100).

36. The isolated nucleic acid of claim 35, wherein said nucleic acid sequence encodes a polypeptide.

37. The isolated nucleic acid of claim 36, wherein said polypeptide has DDS activity.

38. An isolated nucleic acid comprising a nucleic acid sequence, wherein said nucleic acid sequence encodes a polypeptide comprising an amino acid sequence, wherein said amino acid sequence has a length and a percent identity to the sequence set forth in SEQ ID NO:42 over said length, wherein the point defined by said length and said percent identity is within the area defined by points A, B, C, and D of FIG. 26, wherein point A has coordinates (337, 100), point B has coordinates (337, 65), point C has coordinates (25, 65), and point D has coordinates (5, 100).

39. The isolated nucleic acid of claim 38, wherein said polypeptide has DDS activity.

40. An isolated nucleic acid comprising a nucleic acid sequence having a length and a percent identity to the sequence set forth in SEQ ID NO:95 over said length, wherein the point defined by said length and said percent identity is within the area defined by points A, B, C, and D of FIG. 26, wherein point A has coordinates (2017, 100), point B has coordinates (2017, 65), point C has coordinates (50, 65), and point D has coordinates (16, 100).

41. The isolated nucleic acid of claim 40, wherein said point B has coordinates (2017, 85).

42. The isolated nucleic acid of claim 40, wherein said point C has coordinates (100, 65).

43. The isolated nucleic acid of claim 40, wherein said point C has coordinates (50, 85).

44. The isolated nucleic acid of claim 40, wherein said point D has coordinates (20, 100).

45. The isolated nucleic acid of claim 40, wherein said nucleic acid sequence encodes a polypeptide.

46. The isolated nucleic acid of claim 45, wherein said polypeptide has DXR activity.

47. The isolated nucleic acid of claim 40, wherein said nucleic acid sequence is as set forth in SEQ ID NO:95.

48. An isolated nucleic acid comprising a nucleic acid sequence having a length and a percent identity to the sequence set forth in SEQ ID NO:96 over said length, wherein the point defined by said length and said percent identity is within the area defined by points A, B, C, and D of FIG. 26, wherein point A has coordinates (1161, 100), point B has coordinates (1161, 65), point C has coordinates (50, 65), and point D has coordinates (16, 100).

49. The isolated nucleic acid of claim 48, wherein said nucleic acid sequence encodes a polypeptide.

50. The isolated nucleic acid of claim 49, wherein said polypeptide has DXR activity.

51. An isolated nucleic acid comprising a nucleic acid sequence, wherein said nucleic acid sequence encodes a polypeptide comprising an amino acid sequence, wherein said amino acid sequence has a length and a percent identity to the sequence set forth in SEQ ID NO:97 over said length, wherein the point defined by said length and said percent identity is within the area defined by points A, B, C, and D of FIG. 26, wherein point A has coordinates (386, 100), point B has coordinates (386, 65), point C has coordinates (25, 65), and point D has coordinates (5, 100).

52. The isolated nucleic acid of claim 51, wherein said polypeptide has DXR activity.

53. An isolated nucleic acid comprising a nucleic acid sequence of at least 12 nucleotides, wherein said isolated nucleic acid hybridizes under hybridization conditions to the sense or antisense strand of a nucleic acid molecule, the sequence of said nucleic acid molecule being the sequence set forth in SEQ ID NO: 1, 2, 37, 38, 40, 41, 95, or 96.

54. The isolated nucleic acid of claim 53, wherein said nucleic acid sequence is at least 50 nucleotides.

55. The isolated nucleic acid of claim 53, wherein said nucleic acid sequence encodes a polypeptide.

56. The isolated nucleic acid of claim 53, wherein said polypeptide has DXS, DDS, or DXR activity.

57. A substantially pure polypeptide comprising an amino acid sequence, wherein said amino acid sequence has a length and a percent identity to the sequence set forth in SEQ ID NO:3 over said length, wherein the point defined by said length and said percent identity is within the area defined by points A, B, C, and D of FIG. 26, wherein point A has coordinates (641, 100), point B has coordinates (641, 65), point C has coordinates (25, 65), and point D has coordinates (5, 100).

58. The substantially pure polypeptide of claim 57, wherein said polypeptide has DXS activity.

59. A substantially pure polypeptide comprising an amino acid sequence, wherein said amino acid sequence has a length and a percent identity to the sequence set forth in SEQ ID NO:39 over said length, wherein the point defined by said length and said percent identity is within the area defined by points A, B, C, and D of FIG. 26, wherein point A has coordinates (333, 100), point B has coordinates (333, 65), point C has coordinates (25, 65), and point D has coordinates (5, 100).

60. The substantially pure polypeptide of claim 59, wherein said polypeptide has DDS activity.

61. A substantially pure polypeptide comprising an amino acid sequence, wherein said amino acid sequence has a length and a percent identity to the sequence set forth in SEQ ID NO:42 over said length, wherein the point defined by said length and said percent identity is within the area defined by points A, B, C, and D of FIG. 26, wherein point A has coordinates (337, 100), point B has coordinates (337, 65), point C has coordinates (25, 65), and point D has coordinates (5, 100).

62. The substantially pure polypeptide of claim 61, wherein said polypeptide has DDS activity.

63. A substantially pure polypeptide comprising an amino acid sequence, wherein said amino acid sequence has a length and a percent identity to the sequence set forth in SEQ ID NO:97 over said length, wherein the point defined by said length and said percent identity is within the area defined by points A, B, C, and D of FIG. 26, wherein point A has coordinates (386, 100), point B has coordinates (386, 65), point C has coordinates (25, 65), and point D has coordinates (5, 100).

64. The substantially pure polypeptide of claim 63, wherein said polypeptide has DXR activity.

65. A host cell comprising an isolated nucleic acid of claim 1, 9, 12, 14, 22, 25, 27, 35, 38, 40, 48, 51, or 53.

66. The host cell of claim 65, wherein said host cell is prokaryotic.

67. The host cell of claim 65, wherein said host cell is selected from the group consisting of Rhodobacter, Sphingomonas, and Escherichia cells.

68. The host cell of claim 65, wherein said host cell comprises an exogenous nucleic acid that encodes a polypeptide having DDS, DXS, ODS, SDS, DXR, 4-diphosphocytidyl-2C-methyl-D-erythritol synthase, 4-diphosphocytidyl-2C-methyl-D-erythritol kinase, or chorismate lyase activity.

69. The host cell of claim 65, wherein said host cell comprises an exogenous nucleic acid comprising an UbiC sequence or LytB sequence.

70. The host cell of claim 65, wherein said host cell comprises an exogenous nucleic acid comprising an UbiC sequence and LytB sequence.

71. The host cell of claim 65, wherein said host cell comprises non-functional crtE sequence, ppsR sequence, or ccoN sequence.

72. The host cell of claim 65, wherein said host cell comprises non-functional crtE sequence, ppsR sequence, and ccoN sequence.

73. A host cell comprising an exogenous nucleic acid and a non-functional crtE sequence, ppsR sequence, or ccoN sequence, wherein said exogenous nucleic acid is within a crtE, ppsR, or ccoN locus of said host cell.

74. A host cell comprising a genomic deletion, wherein said deletion comprises at least a portion of a crtE sequence, ppsR sequence, or ccoN sequence, and wherein said host cell comprises a non-functional crtE sequence, ppsR sequence, or ccoN sequence.

75. A method for increasing production of CoQ(10) in a cell having endogenous DDS activity, said method comprising inserting a nucleic acid molecule comprising a nucleic acid sequence that encodes a polypeptide having DDS activity into said cell such that production of CoQ(10) is increased.

76. The method of claim 75, wherein said nucleic acid molecule comprises an isolated nucleic acid of claim 14, 22, 25, 27, 35, 38, or 53.

77. The method of claim 75, wherein the production of CoQ(10) is increased at least about 5 percent as compared to a control cell lacking said inserted nucleic acid molecule.

78. The method of claim 75, wherein said cell is selected from the group consisting of Rhodobacter and Sphingomonas cells.

79. The method of claim 75, wherein said cell is a membraneous bacterium.

80. The method of claim 75, wherein said cell is a highly membraneous bacterium.

81. The method of claim 75, wherein said method further comprises inserting a second nucleic acid molecule comprising a nucleotide sequence that encodes a polypeptide having DXS activity into said cell.

82. The method of claim 81, wherein said second nucleic acid molecule comprises an isolated nucleic acid of claim 1, 9, or 12.

83. A method for increasing production of CoQ(10) in a cell having endogenous DDS activity, said method comprising inserting a nucleic acid molecule comprising a nucleic acid sequence that encodes a polypeptide having DXS activity into said cell such that production of CoQ(10) is increased.

84. The method of claim 83, wherein the production of CoQ(10) is increased at least about 5 percent as compared to a control cell lacking said inserted nucleic acid molecule.

85. The method of claim 83, wherein said cell is selected from the group consisting of Rhodobacter and Sphingomonas cells.

86. The method of claim 83, wherein said nucleic acid molecule comprises an isolated nucleic acid of claim 1, 9, or 12.

87. The method of claim 83, wherein said cell is a membraneous bacterium.

88. The method of claim 83, wherein said cell is a highly membraneous bacterium.

89. The method of claim 83, wherein said method further comprises inserting a second nucleic acid molecule comprising a nucleotide sequence that encodes a polypeptide having DDS activity into said cell.

90. The method of claim 89, wherein said second nucleic acid molecule comprises an isolated nucleic acid of claim 14, 22, 25, 27, 35, 38, or 53.

91. A method for increasing production of CoQ(10) in a membraneous bacterium, said method comprising inserting a nucleic acid molecule comprising a nucleic acid sequence that encodes a polypeptide having DDS activity into said bacterium such that production of CoQ(10) is increased.

92. A method for increasing production of CoQ(10) in a highly membraneous bacterium, said method comprising inserting a nucleic acid molecule comprising a nucleic acid sequence that encodes a polypeptide having DDS activity into said highly membraneous bacterium such that production of CoQ(10) is increased.

93. A method for making an isoprenoid, said method comprising culturing a cell under conditions wherein said cell produces said isoprenoid, said cell comprising at least one exogenous nucleic acid that encodes at least one polypeptide, wherein said cell produces more of said isoprenoid than a comparable cell lacking said at least one exogenous nucleic acid.

94. The method of claim 93, wherein said cell is selected from the group consisting of Rhodobacter and Sphingomonas cells.

95. The method of claim 93, wherein said isoprenoid is CoQ(10).

96. The method of claim 93, wherein said at least one polypeptide has DDS, DXS, ODS, SDS, DXR, 4-diphosphocytidyl-2C-methyl-D-erythritol synthase, 4-diphosphocytidyl-2C-methyl-D-erythritol kinase, or chorismate lyase activity.

97. The method of claim 93, wherein said at least one polypeptide is a UbiC polypeptide or a LytB polypeptide.

98. The method of claim 93, wherein said cell comprises a non-functional crtE sequence, ppsR sequence, or ccoN sequence.

99. The method of claim 93, wherein said cell comprises a non-functional crtE sequence, ppsR sequence, and ccoN sequence.

100. The method of claim 93, wherein said cell comprising a genomic deletion, wherein said deletion comprises at least a portion of a crtE sequence, ppsR sequence, or ccoN sequence, and wherein said cell comprises a non-functional crtE sequence, ppsR sequence, or ccoN sequence.

101. A method for making an isoprenoid, said method comprising culturing a genetically modified cell under conditions wherein said cell produces said isoprenoid.

102. The method of claim 101, wherein said isoprenoid is CoQ(10).

103. The method of claim 101, wherein said cell comprises an exogenous nucleic acid.

104. The method of claim 101, wherein said cell comprises a genomic deletion.


SEQUENCE LISTING (190 sequences)

SEQ ID NO: 1 - DNA, 3626 bp, Sphingomonas trueperi
  misc_feature (1)...(3626): n = A,T,C or G
ctgcggccagaccacgcatatcgacgacgattcgatcacgaaaaacgtacggtccgcagcccagcacgccggtttttcgccggtccggccggtgatcgaggtgcgcggcaagtgcggcaagtgtgactgacctgtccaacagaccgttcgacttgagactaacgttgcgctaacaaagcccatggctgacctacccaagacgccgctgctcgacacggtcgacacgccgcaggacctccggaagctcgcccccgcccagctgcgccagctggccgacgagcttcgtgccgaaaccatcagtgcggtgggctccaccggcgggcatctaggctccggcctgggcgtcgtcgaactgacggtggcgatccactatgtattcaacacccccgacgaccggctgatctgggacgtcgggcaccaatgctatccgcacaagatcctcaccggtcggcgcgatcggatccgcacgattcgtcagggtggaggcctctccggcttcaccaagcgcagcgagagcgagtatgatccgttcggtgccgcgcactcgtcgacctcgatctcggccgcactcggctttgcgatcgccaacaagctcaacgaggcgccgggcaaggcgatcgcggtgatcggcgacggcgcgatgagcgcgggcatggcctatgaggcgatgaacaacgccgaggccgccggcaaccggctggtggtgatcctcaacgacaacgacatgtcgatcgccccgccggtgggcgggctttcggcctatcttgcgcgcctcatttcctcgtccgaatatctcggcctgcgcgagctcgccaagcgcttcacccgcaagctttcgcgccgcctcaccgcggcagccggcaaggcggaggaattcgcccgcggcatggcgaccggcggcacgctgttcgaggaacttggcttctattatgtcggcccgatcgacggccacaatctcgagcatctgatcccggtgctggagaatgtccgcgacagcgagcagggcccgatcctgatccatgtcgtgaccaagaagggcaagggctatgccccggccgaagcggcggcggacaagtatcacggcgtccagaagttcgacgtgatcaccggggcacaggccaaggcacccccgggcccgcccgcctataccaaggtgttcgccgatgcgctgctcgccgaagcggagcgtgatgcgtcggtctgcgcgatcaccgcggcgatgccctcgggcaccgggctcgacaagttccaggcgacgttccccgatcgcaccttcgacgtgggcattgccgaacagcacgcggtcaccttcgcagcgggccttgccgcgcaggggatgcggccgttctgcgcgatctactcgaccttcctgcagcgcgcctacgaccaggtcgtccacgacgtcgcgatccagaacctgccggtccgcttcgcgatcgaccgcgcgggcctggtcggtgccgacggcgcgacccatgccggcagcttcgacgtgacctatctcgccagcctgcccaatttcgtggtgatggcggccgcggacgaggtcgagctcgtccacatgacccacacggcggcgatgcacgacagcggcccgatcgcgctgcgctatccacgcggcaacggcgtcggactggcgctgcccaaggttccggagcggctggaaatcggcaagggtcgcgtggtccgagagggcaagaaggtagcgatcctgtcgctcggcacgcgccttgcggaagcactaaaggccgccgacacgctcgaggccaagggcctctcgaccaccgtcgccgacctgcgcttcgccaaaccgctcgacgaggatctgatccgccgcctgctcaccacccacgaagtggcggtgacgatcgaggaaggcgcgatcggcggccccggtgcgcatgtgctgacgctcgccagcgataccggcctgatcgacgccggcctcaagctgcgcaccatgcgcctgccggacatattccaggaccaggacaagcccgagaagcagtatgacgaagcggggctgaacgccgccaacatcgtcgacacggtgctgaaggcgctccgctacaacgaggccgagctggccgacggggtgcgggcgtaaacgacgccagatcctccccggaacggggaggggaaccgccgccgaaggcggtggtggaggggccgctgcggcacgcancggtttcccaggctgagagcgatccgcgccttgcggcgcgccccccccaccattcgctggcgcggatggtccccctccccgttccggggaggatctgggtcctgccccaccttgaatctccaacatgcacatgccatgtacatgcacatggctacgcagcttccccagactcgctccagccgcgttgtcgtgctggtatcgcccgaggaaaaacggcgcatttccgccaatgcggaagcggcggacatgacggtcagcgacttcatgcgcaccgccgccgaacgctataccgagccgaccgacgccgagatggcgctgatgcgcgacctgctcgcccagctcgaacaggccaatgcccgcacggacgcggcctttgcccagctcgaagctgcgcgcgccgccgccaccgcgttcgacgaggaggcgtatcgcgccgaggtccgcgaacagctgctgaccgatacctcaatcgactgggatgcgctgtccactgccctttccggctgggcgcgccagtgagcttctggaccgatgcgctacgcgcgctccagcaggtcgcgctgctccagcacaaggtcgagcaggcgctgaccaccgccgaggaagcccgccgccattcaatcgagacgcgcgagcgggtgatccggcttgagacgctgatcgacatcgcgatgagacgccagcccgcagcaccgcctacgccgcctgcgcttcccgaaagtccacaaaccggcagctagcgcccgcttccccgagcgcgtacatcgcggtacgtgctgaaaatgaccatccttcccctcaccgcccgcccccgcgcgctcgcgcactggctgttcgtcgtcgccgcgatgatcgtcgcgatggtcgtggtcgggggcattacccggctcaccgaatcgggcctgtcgatcaccgaatggaagccaatctccggcatcgtgcccccgctcaacgacgcgcagtggcaggccgagttcgaccactacaagcagatcggccagtatgagcagctcaaccagggcatgacgctcggcgggttcaagagcatcttcttctgggaatatatccaccgcctgctcggccggctgatcggcatggtgttcgcgctgccgctgctgtggttcgccgtccgcaagcagatcccgcagggctatggctggcggctggtcgcgctgctcgcgctaggcgggctgcagggcgcgttcggctggtggatggtgaagtcggggctcaaccacacccgcacctcggttagccatttctggctggcgacccacctgatgaccgcactgttcacgctgggcggcatcgtctggacgatgctcgacctgcgcgcgcttgccgccaaccatgccgagcgccctgcccgactgaccgggctcggcgcgggcgtgctggtactgctggcggtccagctcttctacggggcgctggtagcagg

SEQ ID NO: 2 - DNA, 1926 bp, Sphingomonas trueperi
atggctgacctacccaagacgccgctgctcgacacggtcgacacgccgcaggacctccggaagctcgcccccgcccagctgcgccagctggccgacgagcttcgtgccgaaaccatcagtgcggtgggctccaccggcgggcatctaggctccggcctgggcgtcgtcgaactgacggtggcgatccactatgtattcaacacccccgacgaccggctgatctgggacgtcgggcaccaatgctatccgcacaagatcctcaccggtcggcgcgatcggatccgcacgattcgtcagggtggaggcctctccggcttcaccaagcgcagcgagagcgagtatgatccgttcggtgccgcgcactcgtcgacctcgatctcggccgcactcggctttgcgatcgccaacaagctcaacgaggcgccgggcaaggcgatcgcggtgatcggcgacggcgcgatgagcgcgggcatggcctatgaggcgatgaacaacgccgaggccgccggcaaccggctggtggtgatcctcaacgacaacgacatgtcgatcgccccgccggtgggcgggctttcggcctatcttgcgcgcctcatttcctcgtccgaatatctcggcctgcgcgagctcgccaagcgcttcacccgcaagctttcgcgccgcctcaccgcggcagccggcaaggcggaggaattcgcccgcggcatggcgaccggcggcacgctgttcgaggaacttggcttctattatgtcggcccgatcgacggccacaatctcgagcatctgatcccggtgctggagaatgtccgcgacagcgagcagggcccgatcctgatccatgtcgtgaccaagaagggcaagggctatgccccggccgaagcggcggcggacaagtatcacggcgtccagaagttcgacgtgatcaccggggcacaggccaaggcacccccgggcccgcccgcctataccaaggtgttcgccgatgcgctgctcgccgaagcggagcgtgatgcgtcggtctgcgcgatcaccgcggcgatgccctcgggcaccgggctcgacaagttccaggcgacgttccccgatcgcaccttcgacgtgggcattgccgaacagcacgcggtcaccttcgcagcgggccttgccgcgcaggggatgcggccgttctgcgcgatctactcgaccttcctgcagcgcgcctacgaccaggtcgtccacgacgtcgcgatccagaacctgccggtccgcttcgcgatcgaccgcgcgggcctggtcggtgccgacggcgcgacccatgccggcagcttcgacgtgacctatctcgccagcctgcccaatttcgtggtgatggcggccgcggacgaggtcgagctcgtccacatgacccacacggcggcgatgcacgacagcggcccgatcgcgctgcgctatccacgcggcaacggcgtcggactggcgctgcccaaggttccggagcggctggaaatcggcaagggtcgcgtggtccgagagggcaagaaggtagcgatcctgtcgctcggcacgcgccttgcggaagcactaaaggccgccgacacgctcgaggccaagggcctctcgaccaccgtcgccgacctgcgcttcgccaaaccgctcgacgaggatctgatccgccgcctgctcaccacccacgaagtggcggtgacgatcgaggaaggcgcgatcggcggccccggtgcgcatgtgctgacgctcgccagcgataccggcctgatcgacgccggcctcaagctgcgcaccatgcgcctgccggacatattccaggaccaggacaagcccgagaagcagtatgacgaagcggggctgaacgccgccaacatcgtcgacacggtgctgaaggcgctccgctacaacgaggccgagctggccgacggggtgcgggcgtaa

SEQ ID NO: 3 - protein, 641 aa, Sphingomonas trueperi
MADLPKTPLLDTVDTPQDLRKLAPAQLRQLADELRAETISAVGSTGGHLGSGLGVVELTVAIHYVFNTPDDRLIWDVGHQCYPHKILTGRRDRIRTIRQGGGLSGFTKRSESEYDPFGAAHSSTSISAALGFAIANKLNEAPGKAIAVIGDGAMSAGMAYEAMNNAEAAGNRLVVILNDNDMSIAPPVGGLSAYLARLISSSEYLGLRELAKRFTRKLSRRLTAAAGKAEEFARGMATGGTLFEELGFYYVGPIDGHNLEHLIPVLENVRDSEQGPILIHVVTKKGKGYAPAEAAADKYHGVQKFDVITGAQAKAPPGPPAYTKVFADALLAEAERDASVCAITAAMPSGTGLDKFQATFPDRTFDVGIAEQHAVTFAAGLAAQGMRPFCAIYSTFLQRAYDQVVHDVAIQNLPVRFAIDRAGLVGADGATHAGSFDVTYLASLPNFVVMAAADEVELVHMTHTAAMHDSGPIALRYPRGNGVGLALPKVPERLEIGKGRVVREGKKVAILSLGTRLAEALKAADTLEAKGLSTTVADLRFAKPLDEDLIRRLLTTHEVAVTIEEGAIGGPGAHVLTLASDTGLIDAGLKLRTMRLPDIFQDQDKPEKQYDEAGLNAANIVDTVLKALRYNEAELADGVRA

SEQ ID NO: 4 - DNA, 2208 bp, Chlamydomonas reinhardtii
atgctgcgtggtgctgtttctcacggccctgcggtcgccgaccgggctgccgctggccccgcccgctgcgctgctcccgtcgcccgtggtgtgcgcagcgcagcgcccacgcgtcagcgtcgcgcggaggcttcggtcaatgccccgcgggcgggcccggccggtagctactcgggcgagtgggataagctttcagtggaggagattgatgagtggcgcgatgtgggcccgaagacgcccctgctggacactgtcaattacccggtgcacctgaagaacttcaacaatgagcagctgaagcagctctgcaaggagctgcgcagtgacatcgtgcacaccgtctctcgcaccggtggacaccttagcagcagcctgggcgtggtggagctgacggtggctatgcactatgtattcaacaccccggaggacaagattatttgggacgtgggccaccaggcgtatggccacaagatcctgactggccgtcgcaagggtatggccacgattcgccagaccaacggcctttcgggcttcacgaagcgcgacgagagcgagtacgaccctttcggcgctggccacagctccacctcgatttcggcggctctgggtatggcggtgggccgcgacgttaagggcaagaagaacagtgtgatcgctgtcatcggcgacggcgccatcaccgggggtatggcctatgaggccatgaaccatgcgggcttcctggacaagaacatgattgtgattctgaacgacaaccagcaggtgtcgctgcccacgcagtacaacaacaagaaccaggaccccgtgggcgccctgtccagcgccctggcgcgcctgcaggccaaccggcccctgcgcgagctgcgcgagattgccaagggcgtgaccaagcagctgcctgacgttgtccagaaggcaactgctaagattgacgagtatgctcgcggcatgatcagcggcactggctccacgctgtttgaggagctgggcctgtactacatcggccctgtggacggccacaacctggacgacctcatcgccgtgctcagcgaggtgcgcagcgccgagaccgtgggcccggtgctggtgcacgtggtaacggagaagggccgcggctacctgcccgccgagacggcgcaggacaagatgcacggtgtggtcaagttcgacccccgcaccggcaagcaggtgcaggccaagacgaaggccatgtcgtacacgaactacttcgcggacgcgctgacggcggaggcggagcgcgacagccgcatcgtggcggtgcacgcggccatggcgggcggcaccggcctgtaccggttcgagaagaagttcccggaccgcacctttgacgtgggcattgcggagcagcacgccgtgacctttgctgccggcctggcgtgcgagggcctggtgcccttctgcaccatctacagtaccttcatgcagcgcggttacgaccagatcgtgcacgacgtgtccctgcagaagctgcctgtgcgcttcgctatggaccgcgctggcctggtgggcgctgacggctccacgcactgcggcgccttcgacgtgacgttcatggcgtcgctgccgcacatgatcaccatggctccctcgaacgaggcggagctcatcaacatggtggccacctgcgccgccatcgacgacgcgccctcgtgcttccgcttcccccgcggcaacggcctgggcctggacctggccgcctacggcatcagcaaggacctgaagggtgtgcccctcgaggtgggcaagggtgttgtccgccgccagggcaaggacgtgtgcctggtggcgtacggcagcagtgtgaacgaggcgctggccgcggcggacatgctggagcgcgatggcgtgtccaccaccgtcattgacgcgcgcttctgcaagcctctggacaccaagctgatccgctcggctgccaaggagcaccctgtcatgatcaccatcgaggagggctccgtgggtggcttcgctgcgcacgtgatgcagttcctcgcactggagggcctgctggacggcgggctcaagttccggcccatgacgctgccggaccgctacatcgaccacggcgactaccgcgaccagctggccatggccggcctcaccagccagcacatcgcctccaccgcgctcaccaccctggggcgcgccaaggacgccgccaagttctcactgtcagcgctgcaagcgtaa

SEQ ID NO: 5 - DNA, 1849 bp, Campylobacter jejuni
atgagtaaaaaatttgcccatactcaagaagagttagaaaagctaagtttaaaagaattagaaaatttagcagcatctatgcgtgaaaaaatcatacaagttgtgagtaaaaatggtgggcatttaagttcaaatttgggtgctgtagaacttagtatagcaatgcatttggtttttgatgcaaaaaaagatccttttatttttgatgtgtcgcatcagtcttatacacacaagcttttaagcggaaaagaagaaatatttgatactttaagacaaatcaatggtttaagtggttatacaaaacctagcgagggagattattttgtagcagggcattctagtacctctatttctttggcagtaggtgcttgtaaggctattgctttaaagggtgaaaagcgtattcctgttgctttgattggagatggtgctttaagtgcgggtatggcctatgaggctttaaatgaattgggtgattctaaatttccttgcgtaatacttttaaatgataatgaaatgagtatttcaaaaccaattggagcaatttcaaagtatctttctcaggctatggcaacgcagttttatcaaagttttaaaaagcgtattgctaaaatgttggatatattgcctgatagtgctacttatatggccaagcgttttgaagagagttttaaacttattacccctgggcttttgtttgaagaattagggcttgaatatatagggcctattgatggacataatttaggtgaaattatttctgcattaaaacaagcaaaagctatgcaaaagccttgtgtgatacatgctcaaaccataaagggtaaaggctatgctttagctgaaggaaaacatgctaaatggcacggggtgggagcctttgatatagatagtggagagagtgttaaaaaaagtgatactaaaaaatctgctactgaaattttttctaagaatttgcttgatttagcctcaaaatatgaaaatattgttggggttacggcggctatgccaagtggaacaggtcttgataagcttatagaaaaatatccaaatcgtttttgggatgtggctattgcagaacagcatgcagtaacttctatggccgctatggcaaaagaaggatttaaaccttttattgcaatatatagcacctttttgcagcgtgcttatgatcaagtgatccatgattgtgcgattatgaaatttaaatgtggtttttgctatggatagggcagggatagtaggcgaagatggggagacgcatcaaggtgtttttgatcttagttttttagctcctttgccaaatttcactcttttagccccaagagatgaacaaatgatgcaaaatataatggagtatgcttatttacatcaaggacctattgctttgcgttatcctagagggagttttattttggataaagaatttaatccttgtgagataaaacttggtaaggcacaatggcttgtaaaaaataatagtgaaattgcttttttaggttatggacaaggtgtggcaaaagcgtggcaagtcttaagagccttgcaagaaatgaataataatgctaatttgattgatttaatttttgctaaacctttagatgaagagcttttgtgtgagcttgctaaaaaaagtaaaatttggtttatttttagtgaaaatgttaaaattggcggtatagaaagtttaattaataattttttacaaaaatatgatttgcatgtaaaagttgttagctttgaatatgaagacaaatttattgaacatggaaaaacaagtgaggtggaaaaaaatctagaaaaagatgtcaatagtttgttgacgaaagttttaaaattttatcattaa

SEQ ID NO: 6 - DNA, 1884 bp, Pseudomonas aeruginosa
atgcccaagacgctccatgagattccccgcgagcgccccgccacgcccctgctcgaccgcgcctcttcgccggccgaactgcgccggctgggcgaggcggacctggaaaccctggccgacgagctgcgccagtacctgctgtataccgtcggccagaccggcggtcatttcggcgccggcctcggcgtggtcgagctgaccattgccctgcactacgtcttcgacactccggacgaccgcctggtctgggacgtcggccaccaggcctatccgcacaagatcctcaccgagcgccgcgagctgatgggcaccctgcgccagaagaacggcctggcggccttcccgcgccgcgcagagagcgagtacgacaccttcggcgtcggccactccagcacctccatcagcgccgccctgggcatggccatcgccgcccgcctgcaaggcaaggagcgtaagtcggtggccgtgatcggcgacggtgcgctgaccgccggcatggccttcgaggcactcaaccacgcctcggaagtcgacgccgacatgctggtgatcctcaacgacaacgacatgtcgatctcgcacaacgtcggcgggctctccaactacctggcgaagatcctctccagccgcacctatagcagcatgcgcgagggcagcaagaaggtgctctcgcgcctgcccggggcctgggagatcgcccggcgcaccgaggaatacgccaagggcatgctggtccccggcaccctgttcgaggagctcggctggaattacatcggcccgatcgacggccacgacctgccgaccctggtggctaccctgcgcaacatgcgcgacatgaagggcccgcagttcctccatgtggtgaccaagaaaggcaagggcttcgccccggccgaactggatccgatcggctaccacgcgatcaccaagctggaagctcccggcagtgcgccgaagaagaccggcggacccaagtattccagcgtcttcggccagtggctgtgcgacatggccgcccaggacgcgcgcctgctcggcatcaccccggcgatgaaggaaggttccgacctggtggccttcagcgaacgttatccggaacgctacttcgacgtcgccatcgccgaacagcatgccgtgaccctggccgccggcatggcctgcgagggcatgaagccggtggtagcgatctactcgaccttcctccagcgcgcctacgaccagttgatccatgacgtcgccgtgcagcacctcgacgtgctgttcgccatcgaccgcgccggcctggtcggcgaggacggcccgacccacgccggtagcttcgacatctcctacctgcgctgcatccccggcatgctggtgatgacccccagcgacgaggacgagctgcgcaagctgctcaccaccggctacctgttcgatggcccggccgcggtgcgctatccgcgcggcagcggccccaaccatccgatcgatccggacctgcaaccggtggagatcggcaagggcgtggtccgtcggcgcggcggcagggtcgcactgctggtcttcggcgtgcagttggcggaggcgatgaaggtcgccgaaagcctcgacgccacggtcgtcgacatgcgtttcgtcaaacccctcgacgaagccctggtacgcgaattggcgggcagccacgaactgctggtgaccatcgaggaaaacgccgtgatgggcggcgccggctcggcggtcggcgagttcctcgccagcgagggcctcgaagtcccgctgctgcaactgggcctgcccgactactacgtcgaacacgccaagcccagcgagatgctcgccgaatgcggcctggatgccgcgggcatcgaaaaggcagtacgccagcgtctcgaccggcagtag

SEQ ID NO: 7 - DNA, 2160 bp, Lycopersicon esculentum
atggctttgtgtgcttatgcatttcctgggattttgaacaggactggtgtggtttcagattcttctaaggcaacccctttgttctctggatggattcatggaacagatctgcagtttttgttccaacacaagcttactcatgaggtcaagaaaaggtcacgtgtggttcaggcttccttatcagaatctggagaatactacacacagagaccgccaacgcctattttggacactgtgaactatcccattcatatgaaaaatctgtctctgaaggaacttaaacaactagcagatgaactaaggtcagatacaattttcaatgtatcaaagactgggggtcaccttggctcaagtcttggtgttgttgagctgactgttgctcttcattatgtcttcaatgcaccgcaagataggattctctgggatgttggtcatcagtcttatcctcacaaaatcttgactggtagaagggacaagatgtcgacattaaggcagacagatggtcttgcaggatttactaagcgatcggagagtgaatatgattgctttggcaccggccacagttccaccaccatctcagcaggcctagggatggctgttggtagagatctaaaaggaagaaacaacaatgttattgccgtaataggtgatggtgccatgacagcaggtcaagcttatgaagccatgaataatgctggttacctggactctgacatgattgttatcttaaacgacaatagacaagtttctttacctactgctactctggatgggccagttgctcctgttggagctctaagtagtgctttgagcaggttacagtctaataggcctctcagagaactaagagaagtcgcaaagggagttactaagcagattggtggtcctatgcatgagcttgctgcaaaagttgatgaatatgctcgtggcatgattagtggttctggatcaacattgtttgaagaacttggactttactatattggtcctgtggatggtcacaacattgatgatctaattgcgattctcaaagaggttagaagtactaaaacaacaggtccagtactgatccatgttgtcactgagaaaggcagaggttatccatatgctgagagagctgcagataagtatcatggagttgccaagtttgatccagcaacaggaaagcaattcaaagccagtgccaagacacagtcctatacaacatattttgccgaggctttaattgcagaagcagaagcagataaagacattgttgcaatccatgctgccatggggggtgggaccggaatgaaccttttccatcgtcgcttcccaacaaggtgttttgatgttggaatagcagaacaacatgcagtaacctttgctgctggattggcttgtgaaggcattaaacctttctgtgcaatctattcgtctttcatgcagagggcttatgaccaggtagtgcatgacgttgatttgcaaaagctgcccgtgaggtttgcaatggacagagcaggtcttgttggagcagatggtccaacacattgtggtgcatttgatgttacttacatggcatgtcttcctaacatggttgtaatggctccttctgatgaagcggagctatttcacatggtagcaactgctgccgccattgatgacagaccaagttgttttagatacccaagaggaaatgggatcggtgtagagcttccggctggaaacaaaggaattcctcttgaggttggtaaaggtaggatattgattgagggggagagagtggctctattgggatatggctcagcagtgcagaactgtttggatgctgctattgtgctagaatcccgcggcttacaagtaacagttgcagatgcacgtttctgcaaaccactggaccatgccctcataaggagccttgcaaaatcacatgaagtgctaatcactgtcgaagaaggatcaattggaggttttggatctcatgttgttcagttcatggccttagatgggcttcttgatggcaagttgaagtggagaccaatagttcttcctgatcgatacattgaccatggatctcctgttgatcagttggcggaagctggcctaacaccatctcacattgcagcaacagtatttaacatacttggacaaaccagagaggctctagaggtcatgacataa

SEQ ID NO: 8 - DNA, 1916 bp, Mycobacterium tuberculosis
atgctgcaacagatccgcgggcccgctgatctgcagcacctttcccaggcgcagcttcgggagctggccgccgagatccgtgagttcctgatccacaaggttgccgccacgggggggcatctggggccgaacctgggagtggtggaactcaccttggcgctgcaccgggtattcgactcgccgcacgatccgatcatcttcgacaccggtcaccaggcctacgtccacaagatgttgaccggacgcagccaggacttcgcaaccctgcgtaagaagggcgggttgtcggggtatccgtctcgtgccgagagcgagcacgactgggtggagtcgagccacgccagcgcggcgctgtcgtacgcggacgggttggccaaggcgttcgagttgaccggacaccgcaaccggcatgtggtcgcggtggtcggtgacggtgcgctcaccggcggtatgtgctgggaggcgctgaacaatatcgccgcatcccgccggccggtgattatcgtggtcaacgacaatgggcgcagctacgcgcccacaatcgggggcgtcgccgaccatctggccacgctgcggctgcagccggcctacgagcaggcgctggagacgggccgcgacctggtgcgcgcggtgccgcttgtcggcggtctgtggtttcgatcctgcacagcgtcaaggccggcatcaaggactcgctgtcgccgcagttgctgttcaccgacctcgggttgaagtacgtcggcccggtcgacggccatgacgagcgggcggtggaggtcgcgctgcgcagcgcgcggcgcttcggtgcaccggtgatcgtgcacgtcgtcacccgcaagggcatgggctacccgccggccgaggccgaccaggccgagcagatgcattccacggtcccgatcgatccggccaccggacaagccaccaaggtggccggcccaggctggacggcgaccttctctgatgcacttatcggctacgcccagaaacgccgtgacatcgtggccattaccgcggccatgccgggccccaccgggctgaccgcgttcgggcagcgcttcccggatcgattgttcgacgtcgggatcgccgagcaacacgcgatgacgtcggcggccgggttggcgatgggtgggctgcaccccgtggtggcgatctactcgacgttcctgaaccgggcgttcgaccagatcatgatggatgtggcgctgcacaagctgccggtcaccatggtgctggaccgtgccgggatcaccggtagcgacggcgccagccacaacggaatgtgggacttgtcgatgctgggtatcgtgcccggcatccgggtggcagcgcccagagacgccacccggttgcgtgaagaactcggcgaggcgctcgacgtcgacgacggcccgacggcgttacggttccccaaaggtgatgtgggagaagatatttcggctttggagcggcgtggaggcgtggatgtgctggcggcgcccgccgatggtttgaaccacgacgtcctgttggtggccatcggcgcgttcgcaccgatggcgttggcggtggccaagcggctgcacaaccaggggatcggtgtgacggtgatcgacccgcgctgggtgttgccggtgtctgacggtgtgcgcgaactggcggtgcagcacaagctgctcgtcacgctagaggacaacggggtcaacggtggggcggggtcagcggtgtcggccgcgctgcggcgcgcggagatcgacgtgccctgccgcgatgtcgggttgccgcaggagttctacgagcacgcgtctcgaagcgaggtgctggccgatctggggcttaccgaccaggacgtggcccggcggatcaccggctgggtcgccgcgctgggtaccggggtgtgtgcgtccgacgcgattccagaacatctcgactaa

SEQ ID NO: 9 - DNA, 1914 bp, Rhodobacter sphaeroides
atgaccgacagaccctgcacgccgacgctcgaccgggtgacgctcccggtggacataaagggcctcacggaccgtgagttgcgctcgctggccgacgagctgcgggccgaaacgatctcggccgtgtcggtgacgggcgggcatctgggcgcaggcctcggcgtggtggagttgacggttgcgctgcatgcgatcttcgatgcgccccgcgacaagatcatctgggacgtgggccaccagtgctacccccacaagatcctgaccgggcggcgcgaccgcatccgcaccctgcggcagggcgggggtctctcgggcttcaccaagcgctccgagagcccctatgactgtttcggcgcgggccattcctcgacctcgatctcggccgcggtgggctttgccgcggcacgcgagatgggcggcgacacgggcgacgcggtggcggtgatcggcgacggctcgatgtcggccggcatggccttcgaggcgctgaaccacggcgggcacctgaagaaccgggtgatcgtgatcctgaacgacaacgagatgagcatcgcgccgccggtgggggcgctgtcgtcctatctctcgcggctctatgcgggcgcgccgttccaggacttcaaggcggccgccaagggagcgctcgggcttctgcccgaaccgttccaggagggcgcgcgccgcgccaaggagatgctgaagagcgtcaccgtcggcggcacgctcttcgaggagctgggtttctcctatgtcggcccgatcgacgggcacgatctcgaccagcttctgccggtgctgcggaccgtcaagcagcgggcgcatgcgccggtgctgatccatgtcatcaccaagaagggcaggggctatgctccggccgaggccgcgcgcgaccgtggccatgccacgaacaagttcaacgtcctgaccggcgcgcaggtgaagccggtctcgaacgccccctcctataccaaggtcttcgcccagagcctcatcaaggaggccgaggtcgacgagcggatctgcgcggtgacggccgccatgccggacgggacggggctcaacctcttcggcgagcggtttccgaagcgcaccttcgatgtgggcatcgcggaacagcatgcggtgaccttctcggcggcgcttgcggcaggcggcatgcggcccttctgcgccatctattccaccttcctccagcgcggctacgaccagatcgtgcatgacgtggcaatccagcgcctgccggtgcgctttgccatcgaccgcgccggcctcgtgggggcggacggcgccacccatgcgggctcgttcgatgtggccttcctgtcgaacctgcccggcatcgtggtgatggccgccgccgacgaggccgagctcgtccatatggtagccaccgccgccgcccatgacgaagggcccatcgccttccgctatccgcgcggcgacggcgtgggggtcgaggtgccggtgaagggcgtgccgctccagatcggccgtggccgggtggtgagcgagggcacgcgaatcgcgctcctgtccttcggcacccgtctggccgaggtgcaggtggccgccgaggcgctggctgcgcgcgggatctctcccacggttgcggatgcgcgctttgcaaagccgctcgaccgggatctgatcctgcagctcgcggcccatcacgaggcgctcattaccatcgaggagggcgccatcggcggcttcggcagccatgtggcgcagcttctggccgaggccggggtcttcgaccgcggcttccggtatcgctcgatggtgctgcccgacacgttcatcgaccacaacagcgccgaagtgatgtatgccaccgccgggctgaatgcggccgacatagagcggaaggcgctggagacgctgggggtggaggtcctcgcccgccgcgcctga

SEQ ID NO: 10 - DNA, 1947 bp, Rhodobacter sphaeroides
atgaccaatcccaccccgcgacccgaaaccccgcttttggatcgcgtctgctgcccggccgacatgaaggcgctgagtgacgccgaactggagcggctggccgacgaagtgcgttccgaggtgatttcggtcgttgccgagacgggaggacatctggggtcctcgctgggggtggttgagctgactgtcgcgctgcatgcggtcttcaacacgcccaccgacaagctcgtctgggacgtgggccaccagtgctacccccacaagatcctcaccggccggcgcgagcagatgcgcaccctgcgccagaagggcggcctctcgggcttcaccaagcgctcggaatccgcctacgacccgttcggcgcggctcattcctcgacctcgatctcggccgcgctcggctttgccatgggtcgcgagctgggccagcccgtgggcgacacgatcgccgtgatcggcgacggctccatcaccgcgggcatggcctacgaggcactgaaccacgcgggccatctgaacaagcgcctgttcgtgatcctgaacgacaatgacatgagcatcgcgccgcccgtgggggcgcttgcgcgctatctcgtgaatctctcctcgaaggcgcccttcgccacgctgcgcgcggccgccgacgggctcgaggcctcgctgccggggccgctccgcgacggggcgcgccgggcgcgccagctcgtgaccgggatgccgggcgggggcacgctcttcgaggagctgggcttcacctatgtcggccccatcgacggccacgacatggaggcgctcctccagacgctgcgcgcggcgcgggcccggaccacggggccggtgctcatccatgtggtcacgaagaagggcaagggttacgcccccgccgagaatgcccccgacaagtatcacggggtgaacaagttcgaccccgtcacgggcgagcagaagaagtcggtggccaacgcgccgaactacaccaaggtcttcggctccaccctgaccgaggaggccgcgcgcgatccgcgcatcgtggcgatcaccgccgctatgccctcgggcaccggcgtcgacatcatgcagaagcgtttcccgaaccgcgtcttcgacgtgggcatcgccgagcagcatgccgtgaccttcgcggccggcctcgccggggccgggatgaagcccttctgcgcgatctattcctcgttcctgcaacggggttacgaccagatcgcccatgacgtggcgctgcagaaccttcccgtccgcttcgtgatcgaccgggcggggctcgtgggggccgatggcgcgacccatgcgggggccttcgacgttggcttcatcacttcgctgcccaacatgaccgtgatggccgcggccgacgaggccgagctcatccacatgatcgccaccgccgtggccttcggcgagggccccatcgccttccgcttcccgcggggcgagggggtgggcgtcgagatgcccgagcgcgggacggtgctggagcccggccggggccgcgtggtgcgcgaagggacggatgtcgcgatcctctccttcggcgcgcatctgcacgaggccttgcaggcggcgaaacttctcgaggccgagggggtgagcgtgaccgtggccgacgcccgcttctcgcgcccgctcgacacggggcacatcgaccagctcgtgcgccatcacgcggcgctggtaacggtggagcagggggccatgggcggcttcggcgcctatgtcatgcactgtctcgccaattccggcggcttcgacgggggcctcgcgctccgggtcatgacgctgcccgaccgcttcatcgagcaggcgagccccgaggacatgtatgccgatgcggggctgcgggccgaggatatcgcggccaccgcgcggggcgcgctcgcccgggggcgcgtgatgccgctccggcagacggcaaagccgcgggcggtctga

SEQ ID NO: 11 - DNA, 1911 bp, Synechococcus sp. PC 6301
atgcatctcagcgaaattacccatcccaaccagctccacgggttgtcggttgctcagcttgagcaaattggccaccagattcgtgagaagcacctgcagacggttgcagcgaccggtgggcacctcgggccgggcttgggcgtggtggaattgaccctagcgctttaccaaacgctcgatctcgatcgcgacaaagtggtttgggacgttggccaccaagcctatccccacaagctgctgacagggcgctatcacaacttccataccttgcggcaaaaggatggcattgcgggctacccgaagcgcacggaaaaccgcttcgatcatttcggtgccggtcacgcttccaccagtatttctgctggcctcggtatggctctagcacgggatgcccagggcgaagactaccgatgtgtcgctgtgattggtgatggatcgctcaccggtggcatggccttggaagccatcaaccacgctggtcacttgcccaaaacacggctgttggtcgtgctcaacgacaatgacatgtcgatctcgcccaacgtgggtgcgctctctcgctatctgaataagattcgggttagtgagccgatgcagttgctcaccgatggtttgacccaggggatgcaacaaattcccttcgtcggcggcgccattacccaaggctttgagccggttaaggaaggcatgaagcgcctctcctacagcaagattggggcggtctttgaagagctgggcttcacctacatggggccagtggatggtcacaaccttgaagaactgatcgccaccttccgcgaagcgcacaaacacaccggaccagtactcgtccacgttgccacaaccaagggtaagggctatccctacgctgaagaagatcaggttggctatcatgcccaaaatccctttgatctggcgacagggaaggctaaaccagcttcaaaaccgaagccgcctagctattccaaagtgtttggccaaaccctgacgaccttggccaagagcgatcgccgcattgtcgggattacggctgcgatggcgacaggcaccggcttggacattctccagaaggcgctgccgaagcaatacatcgatgttggcattgccgaacagcacgccgtggtgctagctgccggtatggcctgcgatggcatgcgtccggtggtggcaatctattccaccttcctgcagcgggcctttgatcaagtcatccacgacgtttgtatccaaaagctgcccgtcttcttctgcctcgatcgcgcggggatagttggcgcggatggcccgactcaccaaggcatgtacgacattgcttacctgcggctgattcccaacatggtgctgatggcaccgaaagatgaggccgaactgcagcggatgctagtgacgggtattgaatacgacggcccgatcgccatgcgtttcccgcgcgggaatggtattggcgtacccctgccggaagaaggctgggagtcgctcccgattgggaaagcagagcaactgcgccaaggcgatgatttgctgatgttggcttacggctcgatggtctatccggccctgcagacggcagaactgctgaatgagcacggcatctcagctactgtgatcaatgcccgcttcgccaagcccttagatgaggaactgattgtgccgctggcgcgccagatcggcaaagtcgtcacctttgaggaaggctgcctacccggcggctttggctccgcgattatggagtccttgcaggcccatgatctgcaggttccggtgttgccgatcggtgttcccgatctcttggtggaacatgccagccctgatgaatctaaacaggagttgggcctgacgccgcgtcagatggccgatcgcatcctcgaaaagtttggaagccgtcaacggattggtgctgcttcggcttga

SEQ ID NO: 12 - DNA, 1863 bp, Escherichia coli
atgagttttgatattgccaaatacccgaccctggcactggtcgactccacccaggagttacgactgttgccgaaagagagtttaccgaaactctgcgacgaactgcgccgctatttactcgacagcgtgagccgttccagcgggcacttcgcctccgggctgggcacggtcgaactgaccgtggcgctgcactatgtctacaacaccccgtttgaccaattgatttgggatgtggggcatcaggcttatccgcataaaattttgaccggacgccgcgacaaaatcggcaccatccgtcagaaaggcggtctgcacccgttcccgtggcgcggcgaaagcgaatatgacgtattaagcgtcgggcattcatcaacctccatcagtgccggaattggtattgcggttgctgccgaaaaagaaggcaaaaatcgccgcaccgtctgtgtcattggcgatggcgcgattaccgcaggcatggcgtttgaagcgatgaatcacgcgggcgatatccgtcctgatatgctggtgattctcaacgacaatgaaatgtcgatttccgaaaatgtcggcgcgctcaacaaccatctggcacagctgctttccggtaagctttactcttcactgcgcgaaggcgggaaaaaagttttctctggcgtgccgccaattaaagagctgctcaaacgcaccgaagaacatattaaaggcatggtagtgcctggcacgttgtttgaagagctgggctttaactacatcggcccggtggacggtcacgatgtgctggggcttatcaccacgctaaagaacatgcgcgacctgaaaggcccgcagttcctgcatatcatgaccaaaaaaggtcgtggttatgaaccggcagaaaaagacccgatcactttccacgccgtgcctaaatttgatccctccagcggttgtttgccgaaaagtagcggcggtttgccgagctattcaaaaatctttggcgactggttgtgcgaaacggcagcgaaagacaacaagctgatggcgattactccggcgatgcgtgaaggttccggcatggtcgagttttcacgtaaattcccggatcgctacttcgacgtggcaattgccgagcaacacgcggtgacctttgctgcgggtctggcgattggtgggtacaaacccattgtcgcgatttactccactttcctgcaacgcgcctatgatcaggtgctgcatgacgtggcgattcaaaagcttccggtcctgttcgccatcgaccgcgcgggcattgttggtgctgacggtcaaacccatcagggtgcttttgatctctcttacctgcgctgcataccggaaatggtcattatgaccccgagcgatgaaaacgaatgtcgccagatgctctataccggctatcactataacgatggcccgtcagcggtgcgctacccgcgtggcaacgcggtcggcgtggaactgacgccgctggaaaaactaccaattggcaaaggcattgtgaagcgtcgtggcgagaaactggcgatccttaactttggtacgctgatgccagaagcggcgaaagtcgccgaatcgctgaacgccacgctggtcgatatgcgttttgtgaaaccgcttgatgaagcgttaattctggaaatggccgccagccatgaagcgctggtcaccgtagaagaaaacgccattatgggcggcgcaggcagcggcgtgaacgaagtgctgatggcccatcgtaaaccagtacccgtgctgaacattggcctgccggacttctttattccgcaaggaactcaggaagaaatgcgcgccgaactcggcctcgatgccgctggtatggaagccaaaatcaaggcctggctggcataa

SEQ ID NO: 13 - DNA, 1914 bp, Neisseria meningitidis
atgaacccaagccccctactcgacctgattgacagcccgcaagatttgcgccgtctggacaaaaaacagctgccgcgccttgccggcgagttgcgcacctttctgctggaatctgtcgggcagaccggcgggcatttcgccagcaatttgggcgcggtcgagctgacggttgcgctgcactacgtttacaacacgcccgaagacaagctggtgtgggatgtcggacaccaaagctatccgcacaaaattcttaccggacgtaaaaaccagatgcacaccatgcgccaatatggcggtttggcgggttttccgaaacgttgcgagtccgagtacgacgcgttcggcgtggggcattcctccacctccatcggcgcggcgttgggcatggcggcggcggacaaacagttgggcagcgaccgccgcagcgtcgccatcatcggcgacggcgcgatgacggcgggtcaggcgtttgaagccttgaactgcgcgggcgatatggatgtggatttgctggtcgtcctcaacgacaacgaaatgtcgatttcccccaacgtcggtgcgttgcccaaataccttgccagcaacgtcgtgcgcgatatgcacggactgttgagtaccgtcaaagcgcaaacgggcaaggtattagacaaaatacccggcgcgatggagtttgcccaaaaagtcgaacataaaatcaaaacccttgccgaagaagccgaacacgccaaacagtcactgtctttgtttgaaaacttcggcttccgctataccggccccgtggacggacacaacgtcgaaaatctggtcgatgtattggaagacctgcgcggacgcaaaggcccgcagcttctgcacgtcatcaccaaaaagggcaacggctacaaactcgccgaaaacgatcccgtcaaataccacgccgtcgccaacctgcctaaagaaagcgcggcgcaaatgccgtctgaaaaagaacccaagcccgccgccaaaccgacctatacccaagtgttcggcaaatggctgtgcgaccgggcggcggcagattcccgactggttgcgattacccccgccatgcgcgagggcagcggcttggttgagtttgaacaacgattccccgaccgctatttcgatgtcggcatcgccgagcagcacgccgttacctttgccggcggtttggcttgcgaagggatgaagcccgtcgtggcgatttattccacctttttacaacgcgcctacgaccaactggtgcacgacatcgccctgcaaaacctgcccgttttgtttgccgtcgaccgcgcgggcatcgtcggcgcggacggcccgacccatgccggtttgtacgatttaagctttttgcgctgcattccgaatatgattgtcgccgcgccgagcgatgaaaatgaatgccgcctgctgctttcgacctgctatcaggcagacgcgcccgccgccgtccgctatccgcgcggcacgggtacgggcgtgccggtttcagacggcatggaaaccgtggaaatcggcaagggcattatccgccgcgaaggtgagaaaaccgcattcattgccttcggcagtatggtcgcccctgcattggcggtcgccggaaaactgaacgccaccgtcgccgatatgcgcttcgtcaaaccgatagacgaagagttgattgtccgccttgcccgaagccacgaccgcatcgttacccttgaagaaaacgccgaacagggcggcgcaggcagcgcggtgctggaagtgttggcgaaacacggcatctgcaaacccgtcttgcttttgggcgttgccgataccgtaaccggacacggcgatccgaaaaaacttttagacgatttgggcttgagtgccgaagcggtggaacggcgtgtgcgcgcgtggctgtcggatcgggatgcggcaaattaa

SEQ ID NO: 14 - DNA, 1878 bp, Haemophilus influenzae
atgactaacaatatgaacaattatcctcttttatctttaattaattctccagaagatttgcgtcttttaaataaagatcagctaccacaactctgtcaagaattacgtgcttatcttttagaatctgttagtcaaactagcggacatttagcgtcaggtttaggcactgtagagctaaccgttgcgctgcattatgtatataagacgccatttgatcagttaatttgggatgtgggacatcaagcttatccacataaaatcctaacgggtcgccgagagcaaatgtccacaattcgccaaaaagacggtattcatccttttccttggcgtgaagaaagtgaatttgatgtattaagtgttggtcactcctctacgtctattagtgcgggattaggcattgccgttgccgcagaacgagaaaatgcaggtagaaaaacagtatgcgtaatcggtgatggcgcaattactgcgggaatggcatttgaggcattaaatcacgcgggggcattgcatacagatatgttagttattttaaatgataacgaaatgtctatttcagaaaacgttggtgcattaaataatcatcttgcgcgtattttctctggctctctttactctacgcttcgtgatggcagtaaaaaaatccttgataaagttcctccaatcaaaaattttatgaaaaaaaccgaagaacatatgaaaggtgtaatgttttcgccagaaagtacattatttgaagaactcggttttaactatattggcccagtggatgggcataacattgatgaattagtggctacgcttacgaatatgcgtaatctgaaaggcccacaatttttgcatataaaaacgaaaaaaggtaaaggatacgcacccgcagaaaaagatccgattggtttccacggtgtacctaaatttgatccaatcagtggcgaattgcccaaaaacaatagtaaaccaacttattcgaaaatttttggcgattggctatgtgaaatggcagaaaaagatgccaaaattataggtatcacacctgcaatgcgtgagggttcaggtatggtagaattttcccaacgcttcccaaaacaatattttgacgtagcgattgcagaacagcacgctgtcacgtttgccacaggacttgcaattggcggatataaacctgtcgtcgcaatttactcgacatttttacaacgtgcttacgatcaattaattcacgatgttgccattcaaaatctccctgtgctatttgcaattgatcgagcagggatagttggtgcagatggggctacacatcaaggtgcattcgatattagctttatgcgttgcattccaaatatgatcattatgacgccgagtgatgaaaatgaatgccgtcaaatgctctatacaggttatcaatgtggaaaacctgcggcagtgcgctaccctcgcggaaatgccgttggtgtaaaacttactcctttagaaatgcttcctattggtaaatcacgtttaattcgaaaaggtcaaaaaattgcgattttaaattttggtactctattaccatccgctttagagttatcagaaaaactcaatgcaacggttgtcgatatgcgttttgtgaaaccgattgatattgaaatgattaatgtgcttgcacaaactcacgattatttggtcacattggaagaaaatgcaattcaaggtggagcgggatctgctgttgcggaagtactaaattcatcaggaaaatcaaccgcacttttacaacttggcttgccagattattttattccacaagcgacacagcaagaagcattggcagatttaggattggatacaaaaggcattgaagaaaaaattctcaactttattgcaaaacaaggtaatttataa

SEQ ID NO: 15 - protein, 627 aa, Pseudomonas aeruginosa
MPKTLHEIPRERPATPLLDRASSPAELRRLGEADLETLADELRQYLLYTVGQTGGHFGAGLGVVELTIALHYVFDTPDDRLVWDVGHQAYPHKILTERRELMGTLRQKNGLAAFPRRAESEYDTFGVGHSSTSISAALGMAIAARLQGKERKSVAVIGDGALTAGMAFEALNHASEVDADMLVILNDNDMSISHNVGGLSNYLAKILSSRTYSSMREGSKKVLSRLPGAWEIARRTEEYAKGMLVPGTLFEELGWNYIGPIDGHDLPTLVATLRNMRDMKGPQFLHVVTKKGKGFAPAELDPIGYHAITKLEAPGSAPKKTGGPKYSSVFGQWLCDMAAQDARLLGITPAMKEGSDLVAFSERYPERYFDVAIAEQHAVTLAAGMACEGMKPVVAIYSTFLQRAYDQLIHDVAVQHLDVLFAIDRAGLVGEDGPTHAGSFDISYLRCIPGMLVMTPSDEDELRKLLTTGYLFDGPAAVRYPRGSGPNHPIDPDLQPVEIGKGVVRRRGGRVALLVFGVQLAEAMKVAESLDATVVDMRFVKPLDEALVRELAGSHELLVTIEENAVMGGAGSAVGEFLASEGLEVPLLQLGLPDYYVEHAKPSEMLAECGLDAAGIEKAVRQRLDRQ

SEQ ID NO: 16 - DNA, 1896 bp, Streptomyces sp. CL190
gtgacgattctggagaacatccggcaaccacgcgacctgaaggcgctgcccgaggagcagctgcacgaactgtccgaggagatcaggcagttcctggtgcacgcggtcaccagaaccggcggtcatctgggacccaacctgggggtggtggagctgaccatcgccctgcaccgggtcttcgagtcgcccgtcgaccgcatcctgtgggacaccggccaccagagctacgtacacaagctgctgacgggacgtcaggacttctccaagctgcgcggcaagggcggcctgtccggctacccctcgcgcgaggagtccgagcacgacgtcatcgagaacagccacgcctccaccgccctcggctgggccgacggactcgccaaggcccgccgggtgcagggggagaagggccatgtcgtcgccgtcatcggcggacgggcgctgaccggcggcatggcctgggaggccctgaacaacatcgcggccgccaaggaccagccgctgatcatcgtcgtcaacgacaacgagcgctcctacgcgcccaccatcggcggcctcgccaaccacctggccaccctgcgcaccaccgacggctacgagaaggtcctcgcctggggcaaggacgtcctgctgcgtacccccatcgtcggccaccccctctacgaggccctgcacggcgccaagaagggcttcaaggacgccttcgccccgcagggcatgttcgaggacctgggcctgaagtacgtcggccccatcgacgggcacgacatcggcgcggtcgagtccgcgctgcgccgcgccaagcgcttccacgggccggtgctggtgcactgcctcaccgtcaagggccgcggctacgaacccgccctcgcccacgaggaggaccacttccacaccgtcggcgtgatggacccgctcacctgtgagcccctctcgcccaccgacggcccgtcctggacctcggtgttcggcgacgagatcgtacggatcggcgcggagcgcgaggacatcgtcgcgatcaccgccgcgatgctccacccggtggggctcgccaggttcgccgaccgcttcccggaccgggtctgggacgtcggcatcgccgagcagcacgcggccgtgtccgcggccgggctcgccaccggcggactgcacccggtcgtcgccgtctacgccaccttcctcaaccgcgccttcgaccagctcctgatggacgtcgccctgcaccgctgcggtgtgaccttcgtcctggaccgggccggcgtcacgggcgtcgacggcgcctcgcacaacggcatgtgggacatgtccgtcctccaggtcgtgcccggcctcaggatcgccgccccgcgcgacgccgaccacgtgcgcgcccagctgcgggaggcggtcgccgtggacgacgcgccgacgctgatccgcttcccgaaggagtccgtcggcccgcggatcccggccctcgaccgggtcggcggcctcgatgtgctgcaccgcgacgagcggcccgaggtgctgctggtcgccgtgggcgtcatggcacaggtctgcctccagaccgccgagctgctccgggcccgcggcatcggatgcacggtcgtcgacccgcgctgggtcaagcccgtcgaccccgtgctgcccccactcgccgccgagcaccggctcgtcgccgtcgtggaggacaacagccgggccgccggggtcggttcggcggtcgccctggcgctcggggacgccgatgtcgacgtaccggtgcgccgcttcggcatccccgagcagttcctcgcgcacgccaggcgcggtgaggtgctcgccgacatcgggctgaccccggtggagatcgccgggcggatcggcgcgagcctgcccgtgcgggaggaaccggccgaggagcagcccgcatga

SEQ ID NO: 17 - DNA, 1857 bp, Helicobacter pylori
gtgattttgcaaaataaaacttttgatttaaaccctaacgatattgcaggcttggagttggtgtgtcaaacgctacggaatcgtattttagaagtggtgagcgctaatggggggcatttaagctcttctttaggggctgtggagctgattgtgggcatgcatgccttatttgattgccaaaaaaaccctttcatttttgacacttcgcaccaagcttacgcccacaagcttttaaccgggcgctttgaaagctttagcactttaaggcaattcaagggtttgagcggctttactaaacccagcgagagcgcatacgattatttcatcgccgggcatagttccacttcggtgtctataggcgttggggtggctaaagctttttgtttgaaacaagcgctaggcatgcccatagctttattaggcgatgggagcattagtgcagggattttttatgaagccttaaacgaactgggcgataggaaataccccatgatcatgattttaaacgataatgaaatgagtatcagcacgcctattggagccttatccaaagcccttagccagctgatgaaaggcccgttttaccagtctttccgctctaaagttaaaaaaatcttaagcaccttacctgaaagcgtgaattacttagcgagtcgttttgaagaatctttcaagctcatcaccccgggcgtgttttttgaagaattaggcattaactatatagggcctattaatgggcatgatttgagcgcgattattgaaaccttaaaattagccaaagagcttaaagagccggtgctaatccatgcgcaaaccttaaagggcaaaggctataagatcgctgaagggcgctatgaaaaatggcatggggtggggccttttgatttggataccggcttgtctaaaaaatccaaaagcgcaatcttatcgcccactgaagcgtattctaacacccttttagaattagctaaaaaagatgaaaaaatcgtaggcgtaaccgcggcgatgcctagcggcacaggattagacaaactcattgacgcttaccctttgcgcttttttgatgtcgctatcgctgagcaacacgctttaacttctagcagcgctatggctaaagaggggtttaaaccttttgtgagcatctattctacttttttgcagagggcttatgattctattgtgcatgacgcttgtatttctagcttgccgattaaattagccattgacagggctgggattgtgggcgaagatggcgagacgcaccaagggcttttagacgtgtcgtatttgcgctctatccctaacatggtcatttttgccccacgagacaatgagactttaaaaaacgccgtgcgttttgccaatgaacacgattcaagcccttgcgcgttccgataccctagggggtcgtttgcgttaaaagagggggtttttgagcctagcggttttgttttaggccaaagcgaattgttgaaaaaagagggcgaaattttactcataggctatggtaatggcgtggggcgggcgcatttagtccaactggctttaaaagaaaaaaacatagaatgcgctctcttggatctcaggtttttaaagcctttagatccaaatttaagcgcgatcgttgccccttatcaaaagctctatgtttttagcgataattacaagcttggaggggtggctagcgcgattttagagtttttgagcgaacaaaatattttaaagcctgttaaaagctttgaaatcattgatgaatttatcatgcatgggaacaccgctttagtggaaaaatccttaggattagacacagagagtttgactgacgctattttaaaagatttaggacaagagagatga

SEQ ID NO: 18 - protein, 628 aa, Aquifex aeolicus
MLEKYEILKDYKGPFDIKNYDYETLQKLAQEVRDYIINVTSKNGGHVGPSLGVVELTIALLRVFNPPEDVIVWDIGHQGYPWKILTDRKEQFPTLRQYKGISGFLRREESIYDAFGAGHSSTSISAALGFRIGKDLKGEKEDYVIAVIGDGALTAGMAYEALNNAGHIRPDRFIVILNDNEMSISPNVGAISTYLNRIISGHFVQETRQKIKNFLQHFGETPLRIMKLTEEFLKGLISPGVIFEELGFNYIGPIDGHDIKALEDTLNNVKDIKGPVLLHVYTKKGKGYKPAEENPVKWHGVAPYKVESGEIIKKSSPPTWTSVFGKALVELAERDEKIVAITPAMREGSGLVEFAKRFPDRFFDVGIAEQHACTFAAGLAAEGLRPVAAYYSTFLQRAYDQVIHDVALQNLPVTFAIDRAGLVGDDGPTHHGVFDLSYLRCVPNMVVCAPKDEQELRDLLYTGIYSGKPFALRYPRGAAYGVPTEGFKKIEIGTWEELLEGEDCVILAVGYPVYQALRAAEKLYKEGIRVGVVNARFVKPMDEKMLRDLANRYDTFITVEDNTVVGGFGSGVLEFFAREGIMKRVINLGVPDRFIEHGKQDILRNLVGIDAEGIEKAVRDALKGGRLI

SEQ ID NO: 19 - protein, 633 aa, Bacillus subtilis
MDLLSIQDPSFLKNMSIDELEKLSDEIRQFLITSLSASGGHIGPNLGVVELTVALHKEFNSPKDKFLWDVGHQSYVHKLLTGRGKEFATLRQYKGLCGFPKRSESEHDVWETGHSSTSLSGAMGMAAARDIKGTDEYIIPIIGDGALTGGMALEALNHIGDEKKDMIVILNDNEMSIAPNVGAIHSMLGRLRTAGKYQWVKDELEYLFKKIPAVGGKLAATAERVKDSLKYMLVSGMFFEELGFTYLGPVDGHSYHELIENLQYAKKTKGPVLLHVITKKGKGYKPAETDTIGTWHGTGPYKINTGDFVKPKAAAPSWSGLVSGTVQRMAREDGRIVAITPAMPVGSKLEGFAKEFPDRMFDVGIAEQHAATMAAAMAMQGMKPFLAIYSTFLQRAYDQVVHDICRQNANVFIGIDRAGLVGADGETHQGVFDIAFMRHIPNMVLMMPKDENEGQHMVHTALSYDEGPIAMRFPRGNGLGVKMDEQLKTIPIGTWEVLRPGNDAVILTFGTTIEMAIEAAEELQKEGLSVRVVNARFIKPIDEKMMKSILKEGLPILTIEEAVLEGGFGSSILEFAHDQGEYHTPIDRMGIPDRFIEHGSVTALLEEIGLTKQQVANRIRLLMPPKTHKGIGS

SEQ ID NO: 20 - protein, 735 aa, Chlamydomonas reinhardtii
MLRGAVSHGPAVADRAAAGPARCAAPVARGVRSAAPTRQRRAEASVNAPRAGPAGSYSGEWDKLSVEEIDEWRDVGPKTPLLDTVNYPVHLKNFNNEQLKQLCKELRSDIVHTVSRTGGHLSSSLGVVELTVAMHYVFNTPEDKIIWDVGHQAYGHKILTGRRKGMATIRQTNGLSGFTKRDESEYDPFGAGHSSTSISAALGMAVGRDVKGKKNSVIAVIGDGAITGGMAYEAMNHAGFLDKNMIVILNDNQQVSLPTQYNNKNQDPVGALSSALARLQANRPLRELREIAKGVTKQLPDVVQKATAKIDEYARGMISGTGSTLFEELGLYYIGPVDGHNLDDLIAVLSEVRSAETVGPVLVHVVTEKGRGYLPAETAQDKMHGVVKFDPRTGKQVQAKTKAMSYTNYFADALTAEAERDSRIVAVHAAMAGGTGLYRFEKKFPDRTFDVGIAEQHAVTFAAGLACEGLVPFCTIYSTFMQRGYDQIVHDVSLQKLPVRFAMDRAGLVGADGSTHCGAFDVTFMASLPHMITMAPSNEAELINMVATCAAIDDAPSCFRFPRGNGLGLDLAAYGISKDLKGVPLEVGKGVVRRQGKDVCLVAYGSSVNEALAAADMLERDGVSTTVIDARFCKPLDTKLIRSAAKEHPVMITIEEGSVGGFAAHVMQFLALEGLLDGGLKFRPMTLPDRYIDHGDYRDQLAMAGLTSQHIASTALTTLGRAKDAAKFSLSALQA

SEQ ID NO: 21 - protein, 615 aa, Campylobacter jejuni
MSKKFAHTQEELEKLSLKELENLAASMREKIIQVVSKNGGHLSSNLGAVELSIAMHLVFDAKKDPFIFDVSHQSYTHKLLSGKEEIFDTLRQINGLSGYTKPSEGDYFVAGHSSTSISLAVGACKAIALKGEKRIPVALIGDGALSAGMAYEALNELGDSKFPCVILLNDNEMSISKPIGAISKYLSQAMATQFYQSFKKRIAKMLDILPDSATYMAKRFEESFKLITPGLLFEELGLEYIGPIDGHNLGEIISALKQAKAMQKPCVIHAQTIKGKGYALAEGKHAKWHGVGAFDIDSGESVKKSDTKKSATEIFSKNLLDLASKYENIVGVTAAMPSGTGLDKLIEKYPNRFWDVAIAEQHAVTSMAAMAKEGFKPFIAIYSTFLQRAYDQVIHDCAIMNLNVVFAMDRAGIVGEDGETHQGVFDLSFLAPLPNFTLLAPRDEQMMQNIMEYAYLHQGPIALRYPRGSFILDKEFNPCEIKLGKAQWLVKNNSEIAFLGYGQGVAKAWQVLRALQEMNNNANLIDLIFAKPLDEELLCELAKKSKIWFIFSENVKIGGIESLINNFLQKYDLHVKVVSFEYEDKFIEHGKTSEVEKNLEKDVNSLLTKVLKFYH

SEQ ID NO: 22 - protein, 719 aa, Lycopersicon esculentum
MALCAYAFPGILNRTGVVSDSSKATPLFSGWIHGTDLQFLFQHKLTHEVKKRSRVVQASLSESGEYYTQRPPTPILDTVNYPIHMKNLSLKELKQLADELRSDTIFNVSKTGGHLGSSLGVVELTVALHYVFNAPQDRILWDVGHQSYPHKILTGRRDKMSTLRQTDGLAGFTKRSESEYDCFGTGHSSTTISAGLGMAVGRDLKGRNNNVIAVIGDGAMTAGQAYEAMNNAGYLDSDMIVILNDNRQVSLPTATLDGPVAPVGALSSALSRLQSNRPLRELREVAKGVTKQIGGPMHELAAKVDEYARGMISGSGSTLFEELGLYYIGPVDGHNIDDLIAILKEVRSTKTTGPVLIHVVTEKGRGYPYAERAADKYHGVAKFDPATGKQFKASAKTQSYTTYFAEALIAEAEADKDIVAIHAAMGGGTGMNLFHRRFPTRCFDVGIAEQHAVTFAAGLACEGIKPFCAIYSSFMQRAYDQVVHDVDLQKLPVRFAMDRAGLVGADGPTHCGAFDVTYMACLPNMVVMAPSDEAELFHMVATAAAIDDRPSCFRYPRGNGIGVELPAGNKGIPLEVGKGRILIEGERVALLGYGSAVQNCLDAAIVLESRGLQVTVADARFCKPLDHALIRSLAKSHEVLITVEEGSIGGFGSHVVQFMALDGLLDGKLKWRPIVLPDRYIDHGSPVDQLAEAGLTPSHIAATVFNILGQTREALEVMT

SEQ ID NO: 23 - protein, 643 aa, Mycobacterium leprae
MLEQIRRPADLQHLSQQQLRDLAAEIRELLVHKVAATGGHLGPNLGVVELTLALHRVFDSPHDPIIFDTGHQAYVHKMLTGRCQDFDSLRKKAGLSGYPSRAESEHDWVESSHASTALSYADGLAKAFELAGNRNRHVVAVVGDGALTGGMCWEALNNIAATPRPVVIVVNDNGRSYAPTIGGVADHLATLRLQPAYERLLEKGRDALHSLPLIGQIAYRFMHSVKAGIKDSLSPQLLFTDLGLKYVGPVDGHDEHAVEVALRKARGFGGPVIVHVVTRKGMGYPPAEADQAEQMHTCGVMDPTTGQPTKIAAPDWTAIFSDALIGYAMKRRDIVAITAAMPGPTGLTAFGQCFPDRLFDVGIAEQHAMTSAAGLAMGRMHPVVAIYSTFLNRAFDQIMMDVALHKLPVTMVIDRAGITGSDGPSHNGMWDLSMLGIVPGMRVAAPRDAIRLREELGEALDVDDGPTAIRFPKGDVCEDIPALKRRSGVDVLAVPATGLAQDVLLVGVGVFASMALAVAKRLHNQGIGVTVIDPRWVLPVCDGVLELAHTHKLIVTLEDNGVNGGVGAAVSTALRQVEIDTPCRDVGLPQEFYDHASRSEVLADLGLTDQDVARRITGWVVAFGHCGSGDDAGQYGPRSSQTM

SEQ ID NO: 24 - protein, 638 aa, Mycobacterium tuberculosis
MLQQIRGPADLQHLSQAQLRELAAEIREFLIHKVAATGGHLGPNLGVVELTLALHRVFDSPHDPIIFDTGHQAYVHKMLTGRSQDFATLRKKGGLSGYPSRAESEHDWVESSHASAALSYADGLAKAFELTGHRNRHVVAVVGDGALTGGMCWEALNNIAASRRPVIIVVNDNGRSYAPTIGGVADHLATLRLQPAYEQALETGRDLVRAVPLVGGLWFRFLHSVKAGIKDSLSPQLLFTDLGLKYVGPVDGHDERAVEVALRSARRFGAPVIVHVVTRKGMGYPPAEADQAEQMHSTVPIDPATGQATKVAGPGWTATFSDALIGYAQKRRDIVAITAAMPGPTGLTAFGQRFPDRLFDVGIAEQHAMTSAAGLAMGGLHPVVAIYSTFLNRAFDQIMMDVALHKLPVTMVLDRAGITGSDGASHNGMWDLSMLGIVPGIRVAAPRDATRLREELGEALDVDDGPTALRFPKGDVGEDISALERRGGVDVLAAPADGLNHDVLLVAIGAFAPMALAVAKRLHNQGIGVTVIDPRWVLPVSDGVRELAVQHKLLVTLEDNGVNGGAGSAVSAALRRAEIDVPCRDVGLPQEFYEHASRSEVLADLGLTDQDVARRITGWVAALGTGVCASDAIPEHLD

SEQ ID NO: 25 - protein, 641 aa, Rhodobacter capsulatus
MSATPSRTPHLDRVTGPADLKAMSIADLTALASEVRREIVEVVSQTGGHLGSSLGVVELTVALHAVFNSPGDKLIWDVGHQCYPHKILTGRRSRMLTLRQAGGISGFPKRSESPHDAFGAGHSSTSISAALGFAVGRELGQPVGDTIAIIGDGSITAGMAYEALNHAGHLKSRMFVILNDNDMSIAPPVGALQHYLNTIARQAPFAALKAAAEGIEMHLPGPVRDGARRARQMVTAMPGGATLFEELGFDYIGPVDGHDMAELVETLRVTRARASGPVLIHVCTTKGKGYAPAEGAEDKLHGVSKFDIETGKQKKSIPNAPNYTAVFGERLTEEAARDQAIVAVTAAMPTGTGLDIMQKRFPRRVFDVGIAEQHAVTFAAGMAAAGLKPFLALYSSFVQRGYDQLVHDVALQNLPVRLMIDRAGLVGQDGATHAGAFDVSMLANLPNFTVMAAADEAELCHMVVTAAAHDSGPIALRYPRGEGRGVEMPERGEVLEIGKGRVMTEGTEVAILSFGAHLAQALKAAEMLEAEGVSTTVADARFCRPLDTDLIDRLIEGHAALITLEQGAMGGFGAMVLHYLARTGQLEKGRAIRTMTLPDCYIDHGSPEEMYAWAGLTANDIRDTALAAARPSKSVRIVHSA

SEQ ID NO: 26 - protein, 637 aa, Rhodobacter sphaeroides
MTDRPCTPTLDRVTLPVDIKGLTDRELRSLADELRAETISAVSVTGGHLGAGLGVVELTVALHAIFDAPRDKIIWDVGHQCYPHKILTGRRDRIRTLRQGGGLSGFTKRSESPYDCFGAGHSSTSISAAVGFAAAREMGGDTGDAVAVIGDGSMSAGMAFEALNHGGHLKNRVIVILNDNEMSIAPPVGALSSYLSRLYAGAPFQDFKAAAKGALGLLPEPFQEGARRAKEMLKSVTVGGTLFEELGFSYVGPIDGHDLDQLLPVLRTVKQRAHAPVLIHVITKKGRGYAPAEAARDRGHATNKFNVLTGAQVKPVSNAPSYTKVFAQSLIKEAEVDERICAVTAAMPDGTGLNLFGERFPKRTFDVGIAEQHAVTFSAALAAGGMRPFCAIYSTFLQRGYDQIVHDVAIQRLPVRFAIDRAGLVGADGATHAGSFDVAFLSNLPGIVVMAAADEAELVHMVATAAAHDEGPIAFRYPRGDGVGVEVPVKGVPLQIGRGRVVSEGTRIALLSFGTRLAEVQVAAEALAARGISPTVADARFAKPLDRDLILQLAAHHEALITIEEGAIGGFGSHVAQLLAEAGVFDRGFRYRSMVLPDTFIDHNSAEVMYATAGLNAADIERKALETLGVEVLARRA

SEQ ID NO: 27 - protein, 648 aa, Rhodobacter sphaeroides
MTNPTPRPETPLLDRVCCPADMKALSDAELERLADEVRSEVISVVAETGGHLGSSLGVVELTVALHAVFNTPTDKLVWDVGHQCYPHKILTGRREQMRTLRQKGGLSGFTKRSESAYDPFGAAHSSTSISAALGFAMGRELGQPVGDTIAVIGDGSITAGMAYEALNHAGHLNKRLFVILNDNDMSIAPPVGALARYLVNLSSKAPFATLRAAADGLEASLPGPLRDGARRARQLVTGMPGGGTLFEELGFTYVGPIDGHDMEALLQTLRAARARTTGPVLIHVVTKKGKGYAPAENAPDKYHGVNKFDPVTGEQKKSVANAPNYTKVFGSTLTEEAARDPRIVAITAAMPSGTGVDIMQKRFPNRVFDVGIAEQHAVTFAAGLAGAGMKPFCAIYSSFLQRGYDQIAHDVALQNLPVRFVIDRAGLVGADGATHAGAFDVGFITSLPNMTVMAAADEAELIHMIATAVAFGEGPIAFRFPRGEGVGVEMPERGTVLEPGRGRVVREGTDVAILSFGAHLHEALQAAKLLEAEGVSVTVADARFSRPLDTGHIDQLVRHHAALVTVEQGAMGGFGAYVMHCLANSGGFDGGLALRVMTLPDRFIEQASPEDMYADAGLRAEDIAATARGALARGRVMPLRQTAKPRAV

SEQ ID NO: 28 - protein, 636 aa, Synechococcus sp. PCC 6301
MHLSEITHPNQLHGLSVAQLEQIGHQIREKHLQTVAATGGHLGPGLGVVELTLALYQTLDLDRDKVVWDVGHQAYPHKLLTGRYHNFHTLRQKDGIAGYPKRTENRFDHFGAGHASTSISAGLGMALARDAQGEDYRCVAVIGDGSLTGGMALEAINHAGHLPKTRLLVVLNDNDMSISPNVGALSRYLNKIRVSEPMQLLTDGLTQGMQQIPFVGGAITQGFEPVKEGMKRLSYSKIGAVFEELGFTYMGPVDGHNLEELIATFREAHKHTGPVLVHVATTKGKGYPYAEEDQVGYHAQNPFDLATGKAKPASKPKPPSYSKVFGQTLTTLAKSDRRIVGITAAMATGTGLDILQKALPKQYIDVGIAEQHAVVLAAGMACDGMRPVVAIYSTFLQRAFDQVIHDVCIQKLPVFFCLDRAGIVGADGPTHQGMYDIAYLRLIPNMVLMAPKDEAELQRMLVTGIEYDGPIAMRFPRGNGIGVPLPEEGWESLPIGKAEQLRQGDDLLMLAYGSMVYPALQTAELLNEHGISATVINARFAKPLDEELIVPLARQIGKVVTFEEGCLPGGFGSAIMESLQAHDLQVPVLPIGVPDLLVEHASPDESKQELGLTPRQMADRILEKFGSRQRIGAASA

SEQ ID NO: 29 - protein, 640 aa, Synechocystis sp. PCC 6803
MHISELTHPNELKGLSIRELEEVSRQIREKHLQTVATSGGHLGPGLGVVELTVALYSTLDLDKDRVIWDVGHQAYPHKMLTGRYHDFHTLRQKDGVAGYLKRSESRFDHFGAGHASTSISAGLGMALARDAKGEDFKVVSIIGDGALTGGMALEAINHAGHLPHTRLMVILNDNEMSISPNVGAISRYLNKVRLSSPMQFLTDNLEEQIKHLPFVGDSLTPEMERVKEGMKRLVVPKVGAVIEELGFKYFGPIDGHSLQELIDTFKQAEKVPGPVFVHVSTTKGKGYDLAEKDQVGYHAQSPFNLSTGKAYPSSKPKPPSYSKVFAHTLTTLAKENPNIVGITAAMATGTGLDKLQAKLPKQYVDVGIAEQHAVTLAAGMACEGIRPVVAIYSTFLQRGYDQIIHDVCIQKLPVFFCLDRAGIVGADGPTHQGMYDIAYLRCIPNLVLMAPKDEAELQQMLVTGVNYTGGAIAMRYPRGNGIGVPLMEEGWEPLEIGKAEILRSGDDVLLLGYGSMVYPALQTAELLHEHGIEATVVNARFVKPLDTELILPLAERIGKVVTMEEGCLMGGFGSAVAEALMDNNVLVPLKRLGVPDILVDHATPEQSTVDLGLTPAQMAQNIMASLFKTETESVVAPGVS

SEQ ID NO: 30 - protein, 608 aa, Thermotoga maritima
MLLDEIKRMSYDELKRLAEDIRKRITEVVLKNGGHLASNLGTIELTLALYRVFDPREDAIIWDTGHQAYTHKILTGRDDLFHTIRTFGGLSGFVTRRESPLDWFGTGHAGTSIAAGLGFEKAFELLGEKRHVVVVIGDGALTSGMALEALNQLKNLNSKMKIILNDNGMSISPNVGGLAYHLSKLRTSPIYLKGKKVLKKVLEKTEIGFEVEEEMKYLRDSLKGMIQGTNFFESLGLKYFGPFDGHNIELLEKVFKRIRDYDYSSVVHVVTKKGKGFTAAEENPTKYHSASPSGKPKMLSYSELLGHTLSRVAREDKKIVAITAAMADGTGLSIFQKEHPDRFFDLGITEQTCVTFGAALGLHGMKPVVAIYSTFLQRAYDQIIHDVALQNAPVLFAIDRSGVVGEDGPTHHGLFDINYLLPVPNMKIISPSSPEEFVNSLYTVLKHLDGPVAIRYPKESFYGEVESLLENMKEIDLGWKILKRGREAAIIATGTILNEVLKIPLDVTVVNALTVKPLDTAVLKEIARDHDLIITVEEAMKIGGFGSFVAQRLQEMGWQGKIVNLGVEDLFVPHGGRKELLSMLGLDSEGLTKTVLTYIKARSREGKV

SEQ ID NO: 31 - protein, 620 aa, Escherichia coli
MSFDIAKYPTLALVDSTQELRLLPKESLPKLCDELRRYLLDSVSRSSGHFASGLGTVELTVALHYVYNTPFDQLIWDVGHQAYPHKILTGRRDKIGTIRQKGGLHPFPWRGESEYDVLSVGHSSTSISAGIGIAVAAEKEGKNRRTVCVIGDGAITAGMAFEAMNHAGDIRPDMLVILNDNEMSISENVGALNNHLAQLLSGKLYSSLREGGKKVFSGVPPIKELLKRTEEHIKGMVVPGTLFEELGFNYIGPVDGHDVLGLITTLKNMRDLKGPQFLHIMTKKGRGYEPAEKDPITFHAVPKFDPSSGCLPKSSGGLPSYSKIFGDWLCETAAKDNKLMAITPAMREGSGMVEFSRKFPDRYFDVAIAEQHAVTFAAGLAIGGYKPIVAIYSTFLQRAYDQVLHDVAIQKLPVLFAIDRAGIVGADGQTHQGAFDLSYLRCIPEMVIMTPSDENECRQMLYTGYHYNDGPSAVRYPRGNAVGVELTPLEKLPIGKGIVKRRGEKLAILNFGTLMPEAAKVAESLNATLVDMRFVKPLDEALILEMAASHEALVTVEENAIMGGAGSGVNEVLMAHRKPVPVLNIGLPDFFIPQGTQEEMRAELGLDAAGMEAKIKAWLA

SEQ ID NO: 32 - protein, 637 aa, Neisseria meningitidis
MNPSPLLDLIDSPQDLRRLDKKQLPRLAGELRTFLLESVGQTGGHFASNLGAVELTVALHYVYNTPEDKLVWDVGHQSYPHKILTGRKNQMHTMRQYGGLAGFPKRCESEYDAFGVGHSSTSIGAALGMAAADKQLGSDRRSVAIIGDGAMTAGQAFEALNCAGDMDVDLLVVLNDNEMSISPNVGALPKYLASNVVRDMHGLLSTVKAQTGKVLDKIPGAMEFAQKVEHKIKTLAEEAEHAKQSLSLFENFGFRYTGPVDGHNVENLVDVLEDLRGRKGPQLLHVITKKGNGYKLAENDPVKYHAVANLPKESAAQMPSEKEPKPAAKPTYTQVFGKWLCDRAAADSRLVAITPAMREGSGLVEFEQRFPDRYFDVGIAEQHAVTFAGGLACEGMKPVVAIYSTFLQRAYDQLVHDIALQNLPVLFAVDRAGIVGADGPTHAGLYDLSFLRCIPNMIVAAPSDENECRLLLSTCYQADAPAAVRYPRGTGTGVPVSDGMETVEIGKGIIRREGEKTAFIAFGSMVAPALAVAGKLNATVADMRFVKPIDEELIVRLARSHDRIVTLEENAEQGGAGSAVLEVLAKHGICKPVLLLGVADTVTGHGDPKKLLDDLGLSAEAVERRVRAWLSDRDAAN

SEQ ID NO: 33 - protein, 625 aa, Haemophilus influenzae
MTNNMNNYPLLSLINSPEDLRLLNKDQLPQLCQELRAYLLESVSQTSGHLASGLGTVELTVALHYVYKTPFDQLIWDVGHQAYPHKILTGRREQMSTIRQKDGIHPFPWREESEFDVLSVGHSSTSISAGLGIAVAAERENAGRKTVCVIGDGAITAGMAFEALNHAGALHTDMLVILNDNEMSISENVGALNNHLARIFSGSLYSTLRDGSKKILDKVPPIKNFMKKTEEHMKGVMFSPESTLFEELGFNYIGPVDGHNIDELVATLTNMRNLKGPQFLHIKTKKGKGYAPAEKDPIGFHGVPKFDPISGELPKNNSKPTYSKIFGDWLCEMAEKDAKIIGITPAMREGSGMVEFSQRFPKQYFDVAIAEQHAVTFATGLAIGGYKPVVAIYSTFLQRAYDQLIHDVAIQNLPVLFAIDRAGIVGADGATHQGAFDISFMRCIPNMIIMTPSDENECRQMLYTGYQCGKPAAVRYPRGNAVGVKLTPLEMLPIGKSRLIRKGQKIAILNFGTLLPSALELSEKLNATVVDMRFVKPIDIEMINVLAQTHDYLVTLEENAIQGGAGSAVAEVLNSSGKSTALLQLGLPDYFIPQATQQEALADLGLDTKGIEEKILNFIAKQGNL

SEQ ID NO: 34 - protein, 1205 aa, Plasmodium falciparum
MIFNYVFFKNFVPVVLYILLIIYINLNGMNNKNQIKTEKIYIKKLNRLSRKNSLCSSKNKIACLFDIGNDDNRNTTYGYNVNVKNDDINSLLKNNYSNKLYMDKRKNINNVISTNKISGSISNICSRNQKENEQKRNKQRCLTQCHTYNMSHEQDKLANDNNRNNKKNFNLLFINYFNLKRMKNSLLNKDNFFYCKEKKLSFLHKAYKKKNCTFQNYSLKRKSNRDSHKLFSGEFDDYTNNNALYESEKKEYITLNNNNKNNNNKNNDNKNNDNNDYNNNNSCNNLGERSNHYDNYGGDNNNPCNNNNDKYDIGKYFKQINTFINIDEYKTIYGDEIYKEIYELYVERNIPEYYERKYFSEDIKKSVLFDIDKYNDVEFEKAIKEEFINNGVYINNIDNTYYKKENILIMKKILHYFPLLKLINNPSDLKKLKKQYLPLLAHELKIFLFFIVNITGGHFSSVLSSLEIQLLLLYIFNQPYDNVIYDIGHQAYVHKILTGRKLLFLSLRNKKGISGFLNIFESIYDKFGAGHSSTSLSAIQGYYEAEWQVKNKEKYGNGDIEISDNANVTNNERIFQKGIHNDNNINNNINNNNYINPSDVVGRENTNVPNVRNDNHNVDKVHIAIIGDGGLTGGMALEALNYISFLNSKILIIYNDNGQVSLPTNAVSISGNRPIGSISDHLHYFVSNIEANAGDNKLSKNAKENNIFENLNYDYIGVVNGNNTEELFKVLNNIKENKLKRATVLHVRTKKSNDFINSKSPISILHSIKKNEIFPFDTTILNGNIHKENKIEEEKNVSSSTKYDVNNKNNKNNDNSEIIKYEDMFSKETFTDIYTNEMLKYLKKDRNIIFLSPAMLGGSGLVKISERYPNNVYDVGIAEQHSVTFAAAMAMNKKLKIQLCIYSTFLQRAYDQIIHDLNLQNIPLKVIIGRSGLVGEDGATHQGIYDLSYLGTLNNAYIISPSNQVDLKRALRFAYLDKDHSVYIRIPRMNILSDKYMKGYLNIHMKNESKNIDVNVDINDDVDKYSEEYMDDDNFIKSFIGKSRIIKMDNENNNTNEHYSSRGDTQTKKKKVCIFNMGSMLFNVINAIKEIEKEQYISHNYSFSIVDMIFLNPLDKNMIDHVIKQNKHQYLITYEDNTIGGFSTHFNNYLIENNYITKHNLYVHNIYLSNEPIEHASFKDQQEVVKMDKCSLVNRIKNYLKNNPT

SEQ ID NO: 35 - protein, 631 aa, Streptomyces sp. CL 190
MTILENIRQPRDLKALPEEQLHELSEEIRQFLVHAVTRTGGHLGPNLGVVELTIALHRVFESPVDRILWDTGHQSYVHKLLTGRQDFSKLRGKGGLSGYPSREESEHDVIENSHASTALGWADGLAKARRVQGEKGHVVAVIGGRALTGGMAWEALNNIAAAKDQPLIIVVNDNERSYAPTIGGLANHLATLRTTDGYEKVLAWGKDVLLRTPIVGHPLYEALHGAKKGFKDAFAPQGMFEDLGLKYVGPIDGHDIGAVESALRRAKRFHGPVLVHCLTVKGRGYEPALAHEEDHFHTVGVMDPLTCEPLSPTDGPSWTSVFGDEIVRIGAEREDIVAITAAMLHPVGLARFADRFPDRVWDVGIAEQHAAVSAAGLATGGLHPVVAVYATFLNRAFDQLLMDVALHRCGVTFVLDRAGVTGVDGASHNGMWDMSVLQVVPGLRIAAPRDADHVRAQLREAVAVDDAPTLIRFPKESVGPRIPALDRVGGLDVLHRDERPEVLLVAVGVMAQVCLQTAELLRARGIGCTVVDPRWVKPVDPVLPPLAAEHRLVAVVEDNSRAAGVGSAVALALGDADVDVPVRRFGIPEQFLAHARRGEVLADIGLTPVEIAGRIGASLPVREEPAEEQPA

SEQ ID NO: 36 - protein, 618 aa, Helicobacter pylori
MILQNKTFDLNPNDIAGLELVCQTLRNRILEVVSANGGHLSSSLGAVELIVGMHALFDCQKNPFIFDTSHQAYAHKLLTGRFESFSTLRQFKGLSGFTKPSESAYDYFIAGHSSTSVSIGVGVAKAFCLKQALGMPIALLGDGSISAGIFYEALNELGDRKYPMIMILNDNEMSISTPIGALSKALSQLMKGPFYQSFRSKVKKILSTLPESVNYLASRFEESFKLITPGVFFEELGINYIGPINGHDLSAIIETLKLAKELKEPVLIHAQTLKGKGYKIAEGRYEKWHGVGPFDLDTGLSKKSKSAILSPTEAYSNTLLELAKKDEKIVGVTAAMPSGTGLDKLIDAYPLRFFDVAIAEQHALTSSSAMAKEGFKPFVSIYSTFLQRAYDSIVHDACISSLPIKLAIDRAGIVGEDGETHQGLLDVSYLRSIPNMVIFAPRDNETLKNAVRFANEHDSSPCAFRYPRGSFALKEGVFEPSGFVLGQSELLKKEGEILLIGYGNGVGRAHLVQLALKEKNIECALLDLRFLKPLDPNLSAIVAPYQKLYVFSDNYKLGGVASAILEFLSEQNILKPVKSFEIIDEFIMHGNTALVEKSLGLDTESLTDAILKDLGQER

SEQ ID NO: 37 - DNA, 1990 bp, Rhodobacter sphaeroides
cgacggcccggtagccccggcgcggctgcagcaccgtcagacgtccgccgagaaagccgtcggaagtcaattcgtccggggcgaacatcagggggtcgtcgggatgccgttgtcggacatcacccggcaggcgcgatcccagtcttcttccgggacaaacagacgccgcggcaatatgccgatggagccttcgaggacgctcatgtggacgtccaccggaaaggcgtctatatcctcgccctgaaggagcgcggtggcgaaggcgatgatcgtcgggtcggtcgtgcgcaacagttccttcatgtcggggacattgtcggcaacgcctcggtttgtcgaggccggttcgtcgaccgggtggcaggatcgggatgggattggacgaggtttcgcaaaagccgcatgaacggctcgccgcgtggctggccgaggacatggccgccgtcaacgggctgatccgcgagcggatggcctcgaaacacgcgccccgcattcccgaggtcacggcgcatctggtcgaggccggcggcaagcggctgcggccgctcctgacgctcgccgcggcgcggctgtgcggctacgaggggccctatcacatccatctggccgcgacggtggagttcatccacacggcgacgctgcttcacgacgatgtggtggacgaaagccaccgccgccgcggcaaacccacggcgaacctgctgtgggacaacaaatcctcggtgctggtgggcgactatctcttcgcccgcagcttccagctgatggtcgagaccggctcgcttcgcgtgatggacatcctcgccaatgcctcggccaccatctccgagggcgaggtgctgcagctgaccgcggcccaggatctgcgcacgaccgaggacatccacctgcaggtggtgcgcggcaagacggccgcgctctttgccgcggcaaccgaggtgggcggcgtggtcgcgggcgtgcccgaggcgcaggtcgaggcgctccacgcctacggggacgcgctggggatcgccttccagatcgtcgacgacctcctcgattatggcggcgtggatgcccagatcggcaagaacaccggcgacgacttccgcgaacgcaagctgacgctgccggtcatcaaggcggtggcccaggccgatgccgaggagcgcgccttctggcagcgggtgatcgagaagggcgaccagcgcgagggtgacctcgagcaagcccatgcgatcatgtcccgccacggcgccatggaggccgcccggcaggatgcgctccgctgggtcacggtggcgcgcgaggcactcggccagctgccggagcacccgctgcgcgagatgctgcacgatctggccgatttcgtggtcgaacgcatcgcctgatcccttccgggcgctctgccccggcgcagcgcaggatcccgcgctgcgcccctttcggccttccgacagtccctctgccgcgggaggccggcctcgcctgagaagccgcactggccgccggtcttcccccgaaccgctcccgggcctgctcggaaggcgtccgccgcaaaagcccccgcgggggggccccaccggcggccatcaggaagagaccgttgaagcggcccgctcgaatcctgtcgcgcccccccccgaccgggcggctctccgatccgtgttcgctcggcgatggacagccgttccctgtccgttcatgatggcgccatgcagacccttaccgttcccgattccggcctcgccccctcctgcccggccaaaggctcgcccgcggcgtctgccgccatctgcgcagccatgatttcgtctcggtggtcgaactcgtgcccgcgcccggcctcagggtcgacgtgatggcgctggggcccaagggcgagatctgggtggtggaatgcaaatcctcgcgcgcggactatcagtccgaccgcaagtggcagggctatctcgactggtgcgaccgcttcttcttcgcggtggacgaggaccagcccgggccgtcg

SEQ ID NO: 38 - DNA, 1002 bp, Rhodobacter sphaeroides
atgggattggacgaggtttcgcaaaagccgcatgaacggctcgccgcgtggctggccgaggacatggccgccgtcaacgggctgatccgcgagcggatggcctcgaaacacgcgccccgcattcccgaggtcacggcgcatctggtcgaggccggcggcaagcggctgcggccgctcctgacgctcgccgcggcgcggctgtgcggctacgaggggccctatcacatccatctggccgcgacggtggagttcatccacacggcgacgctgcttcacgacgatgtggtggacgaaagccaccgccgccgcggcaaacccacggcgaacctgctgtgggacaacaaatcctcggtgctggtgggcgactatctcttcgcccgcagcttccagctgatggtcgagaccggctcgcttcgcgtgatggacatcctcgccaatgcctcggccaccatctccgagggcgaggtgctgcagctgaccgcggcccaggatctgcgcacgaccgaggacatccacctgcaggtggtgcgcggcaagacggccgcgctctttgccgcggcaaccgaggtgggcggcgtggtcgcgggcgtgcccgaggcgcaggtcgaggcgctccacgcctacggggacgcgctggggatcgccttccagatcgtcgacgacctcctcgattatggcggcgtggatgcccagatcggcaagaacaccggcgacgacttccgcgaacgcaagctgacgctgccggtcatcaaggcggtggcccaggccgatgccgaggagcgcgccttctggcagcgggtgatcgagaagggcgaccagcgcgagggtgacctcgagcaagcccatgcgatcatgtcccgccacggcgccatggaggccgcccggcaggatgcgctccgctgggtcacggtggcgcgcgaggcactcggccagctgccggagcacccgctgcgcgagatgctgcacgatctggccgatttcgtggtcgaacgcatcgcctga

SEQ ID NO: 39 - protein, 333 aa, Rhodobacter sphaeroides
MGLDEVSQKPHERLAAWLAEDMAAVNGLIRERMASKHAPRIPEVTAHLVEAGGKRLRPLLTLAAARLCGYEGPYHIHLAATVEFIHTATLLHDDVVDESHRRRGKPTANLLWDNKSSVLVGDYLFARSFQLMVETGSLRVMDILANASATISEGEVLQLTAAQDLRTTEDIHLQVVRGKTAALFAAATEVGGVVAGVPEAQVEALHAYGDALGIAFQIVDDLLDYGGVDAQIGKNTGDDFRERKLTLPVIKAVAQADAEERAFWQRVIEKGDQREGDLEQAHAIMSRHGAMEAARQDALRWVTVAREALGQLPEHPLREMLHDLADFVVERIA

SEQ ID NO: 40 - DNA, 1833 bp, Sphingomonas trueperi
ggatcgcgcagcgcctcggccacgcgcaccatcagcagcagattgccgttcggcagccgcgcgaagccggggttgaaggcgccaaggacataggtcgcgtcgtccaccccctcgcgcagcggtgagcgggtcaggtcgacattgtcgggccggaagatcagataatcgtcgctcaagcgcttgccccctcgggtttcacgcccagcaacggggtcaggccccgggggttccggcttcagcgccggcttcctgggcctggcggtggtgccggatcacctcgtcgatgatgaagcgcaggaatttctcggaaaattcggggtcgagatcggcatcctgcgccagcgcgcgcagccgggcgatctgcgcctcctcgcggccgggatcggcgggcggcagcccggattcggccttgtagcgccccaccgcctgggtcaccttgaaccgctcggcgagcatgaagacgagcgccgcatcgatattgtcgatgctctggcgatagcgggtcagcgtcgcgtcggtcatgcgaatctcctttgccgctgcggcacggccatgcaagcacctcttgcctttgcaatgcacaaaggccagaggctcgttgcatatgagcgcaaccgtccaccgcctgggctcgcgaacccagccttcgctcgatccgatcatggcgctggtcgcccaggacatgaacctggtgaacgcggtgatcctcgatcgcatgcagtccgagatcccgctgatccccgaactcgccggccatctgatcgctggcggcggcaagcggatgcggccgatgctgacgctcgccagcgcccggctgctcggctattcgggcacgcgccaccacaagctggcggcggcagtggagttcatccacaccgcgacgctgctgcatgacgacgtggtcgacagctcggacctgcgccgcggccgccgcaccgccaacatcatctggggcaatcccgccagcgtgctggtcggcgacttcctgttcagccgctcgttcgagctgatggtcgaggccgaaagcctcaaggcgctgcacatcctgtcgaacgccagcgcggtgatcgccgagggcgaagtcaaccagctgaccgcggtgcgccggatcgacctgtccgaggatcgctatctcgacatcatcggcgccaagactgcggcgctgttcgccgccgcctgccgggtggcgggcgtggtcgccgagcgtcccgaggcggaggaactcgcgctcgacgcctatggccgcaacctcggcatcgctttccagctggtcgacgacgcgatcgactatgtctcggacgcgtcgacgatgggcaaggatgccggcgacgatttccgcgaaggcaagatgacgctgccggtggtcctggcgtacgcgcgcggcgacgaggcggaacgcggcttctggaaggaagcgatttcgggccgccgcatctcggacgaggatttcgccgaggcgatccggctggtgcagagctgccgcgcggtggacgacacgctcgcccgtgcccgccattacggccagctcgcgatcgatgcgctgggcggcttccgcgcctgcgaggcgaaggacgcgatggtcgaggcggtcgaattcgcggtggcgcgcgcctactgacgcgcgccgaccggagcatttccgggtggatcgcttgcgatccaaggctcgggaaatgcgaccatcaaaaagcttccggggattacgcctcggtcgacttttcttcgccctcgtcctcgtcgacttcgagcgcgtcttcctcgtccatgtcgagcactacctcgatgccctcgacgatcaggtcgagctgctcgtagctcgccgtcatctcgatc

SEQ ID NO: 41 - DNA, 1014 bp, Sphingomonas trueperi
atgagcgcaaccgtccaccgcctgggctcgcgaacccagccttcgctcgatccgatcatggcgctggtcgcccaggacatgaacctggtgaacgcggtgatcctcgatcgcatgcagtccgagatcccgctgatccccgaactcgccggccatctgatcgctggcggcggcaagcggatgcggccgatgctgacgctcgccagcgcccggctgctcggctattcgggcacgcgccaccacaagctggcggcggcagtggagttcatccacaccgcgacgctgctgcatgacgacgtggtcgacagctcggacctgcgccgcggccgccgcaccgccaacatcatctggggcaatcccgccagcgtgctggtcggcgacttcctgttcagccgctcgttcgagctgatggtcgaggccgaaagcctcaaggcgctgcacatcctgtcgaacgccagcgcggtgatcgccgagggcgaagtcaaccagctgaccgcggtgcgccggatcgacctgtccgaggatcgctatctcgacatcatcggcgccaagactgcggcgctgttcgccgccgcctgccgggtggcgggcgtggtcgccgagcgtcccgaggcggaggaactcgcgctcgacgcctatggccgcaacctcggcatcgctttccagctggtcgacgacgcgatcgactatgtctcggacgcgtcgacgatgggcaaggatgccggcgacgatttccgcgaaggcaagatgacgctgccggtggtcctggcgtacgcgcgcggcgacgaggcggaacgcggcttctggaaggaagcgatttcgggccgccgcatctcggacgaggatttcgccgaggcgatccggctggtgcagagctgccgcgcggtggacgacacgctcgcccgtgcccgccattacggccagctcgcgatcgatgcgctgggcggcttccgcgcctgcgaggcgaaggacgcgatggtcgaggcggtcgaattcgcggtggcgcgcgcctactga

SEQ ID NO: 42 - protein, 337 aa, Sphingomonas trueperi
MSATVHRLGSRTQPSLDPIMALVAQDMNLVNAVILDRMQSEIPLIPELAGHLIAGGGKRMRPMLTLASARLLGYSGTRHHKLAAAVEFIHTATLLHDDVVDSSDLRRGRRTANIIWGNPASVLVGDFLFSRSFELMVEAESLKALHILSNASAVIAEGEVNQLTAVRRIDLSEDRYLDIIGAKTAALFAAACRVAGVVAERPEAEELALDAYGRNLGIAFQLVDDAIDYVSDASTMGKDAGDDFREGKMTLPVVLAYARGDEAERGFWKEAISGRRISDEDFAEAIRLVQSCRAVDDTLARARHYGQLAIDALGGFRACEAKDAMVEAVEFAVARAY

SEQ ID NO: 43 - DNA, 1137 bp, Schizosaccharomyces pombe
atgattcagtatgtatatttaaaacatatgaggaaattatggagtcttggaaaagtccgttcgactgttcttcggttttctactacgaaccgcaatgcttcacatttaattaaaaacgagttggaacaaatctcaccagggattcgtcaaatgctgaattcaaattcagaatttcttgaagagtgttctaaatattataccattgctcaaggaaaacaaatgcgtccttctcttgttttgctgatgtccaaagctacaagcttgtgccatggtattgatcggtccgtagtgggcgacaaatatattgatgatgatgatttaagatcattttcgacgggtcaaattcttccttctcaattgagattagcacaaataaccgagatgatccatatagcaagtttgctgcatgacgatgtgattgatcacgctaatgtccgtagaggctcaccttcaagcaatgttgctttcggtaatcgacggtcaatccttgcgggtaatttcatccttgcacgggcttcgactgctatggcccgccttcgaaatccccaagttacggagttgttagctacagtgatagcagacttggttcgaggtgagtttttgcagctaaaaaatactatggatccttcatctttggaaataaaacaatcaaattttgactattatattgaaaaaagttttttgaaaacagccagtttaatttccaaaagctgcaaggcttctacaatcctcggacaatgttctcctactgtagcaacagctgctggagaatacggtcgatgcattggtactgcttttcaactaatggatgacgtgttggactatacgtcgaaagatgatactttaggaaaggcggctggtgcagatttgaagctagggttggctacagctcccgtcctctttgcatggaaaaagtatccagaacttggtgcaatgattgtgaatagattcaatcatccttctgatatccaacgggctcgttctttggttgagtgcactgatgctatcgagcaaaccatcacttgggcaaaagaatatatcaaaaaagccaaagattcccttctgtgtctccctgattcacctgcaaggaaggcactttttgcgttggctgataaagtaataacgagaaagaagtga

SEQ ID NO: 44 - DNA, 948 bp, Gluconobacter suboxydans
atgctggcctgcaaccgggcgatcatcgcccggatggaaagtccggttcccctgatcccgcagcttggcgcccatcttgtcgcggcgggaggcaagcgccttcgcccgctgctgacgctggcctccgcacgtctgtgcggttatcagccgggtccggaccatcagcgtcatgtcgggctcgccgcctgcgttgagttcattcataccgccacactgctgcatgatgatgtcgtggatgagagcacgttgcgtcgggggctggcttcggccaatgccgtgttcggcaacaaggcgtccgtgctggtaggtgacttcctgttcgcccgctcgttccagcttatgacagcagacggctccctgaaggtcatggcgatcctgtcggatgcatcggcgacaattgctgaaggtgaagtccttcagatggtcgtgcagaacgaccttacgacgcctgtagaacgctatcttgaagtcattcacggcaagacggctgcgctgtttgcggctgcctgccgtgtcggcgctgtcgtggccgagcgtccggaagcagaagaggaagctctggagcggtttggcaccaatctgggtatggcgttccagcttgttgatgatgccctggattatgccgcagaccagcaggttttgggcaagaccgttggtgatgacatgcgtgaaggcaagatcaccctgccggtcctggccgcctatgaggctggctcgccggaagatcgtattttctgggagcgcgtcattggagaaggggagcagactgaggacgatctgcctcatgctctgaacctgattgcaaagacgggtgcgatcaatacgacgatcgcccgcgcgcaggtctatgccgacgcagctgttgaagccctgtccattttcccggatagcgaactgcgccgccttctgatcgaaacggttcagttcacggtgaatcgggcccgctaa

SEQ ID NO: 45 - DNA, 978 bp, Rhodobacter capsulatus
atggccatcgatttcaagcaagatattctcgctcctgttgctcaagattttgcagcgatggaccagtttattaatgaaggaatcagctccaaggtcgcactggtcatgtcagtcagcaagcatgtcgttgaagcaggtggaaagcgcatgcgtccgattatgtgcttgctggccgcttatgcctgtggtgaaaccaatttaaagcatgcacagaagctggcggccattattgaaatgctgcatacggcgactctggtacatgatgatgatgtagatgagtctggcttacgccgtggcagaccaacagcaaatgcgacatggaataaccagactgcggtactggtgggggattttctgattgcccgggcatttgatctgctggttgatctggacaatatgatcctgttaaaggacttctctacaggaacctgtgagattgctgagggtgaagtattgcagttgcaggcacagcatcagccagatacaacagaagatatttatttacagattattcacggtaaaacctcacggttgttcgaactggcgaccgaaggcgctgcaatactggcaggcaaacctgaataccgtgaacctttacgtcgttttgccggacactttggcaatgcttttcagattattgatgatattctggattacacttcagatgctgatacgctcggcaaaaatattggcgatgacttgatggaaggcaaacccaccctgccgctgattgcagcaatgcaaaatactcaaggtgaacagcgcgacctgatccgtcgcagcattgccactggcggtacttcacagcttgaacaagttattgcgattgtacaaaattcgggagcgctggattattgccataagcgtgctactgaagaaaccgagcgagcattacaggcactagaaatattacctgagagtacttaccggcaggcgctggttaacttgacccgcttagctttagaccgaatccaataa

SEQ ID NO: 46 - protein, 315 aa, Gluconobacter suboxydans
MLACNRAIIARMESPVPLIPQLGAHLVAAGGKRLRPLLTLASARLCGYQPGPDHQRHVGLAACVEFIHTATLLHDDVVDESTLRRGLASANAVFGNKASVLVGDFLFARSFQLMTADGSLKVMAILSDASATIAEGEVLQMVVQNDLTTPVERYLEVIHGKTAALFAAACRVGAVVAERPEAEEEALERFGTNLGMAFQLVDDALDYAADQQVLGKTVGDDMREGKITLPVLAAYEAGSPEDRIFWERVIGEGEQTEDDLPHALNLIAKTGAINTTIARAQVYADAAVEALSIFPDSELRRLLIETVQFTVNRAR

SEQ ID NO: 47 - protein, 378 aa, Schizosaccharomyces pombe
MIQYVYLKHMRKLWSLGKVRSTVLRFSTTNRNASHLIKNELEQISPGIRQMLNSNSEFLEECSKYYTIAQGKQMRPSLVLLMSKATSLCHGIDRSVVGDKYIDDDDLRSFSTGQILPSQLRLAQITEMIHIASLLHDDVIDHANVRRGSPSSNVAFGNRRSILAGNFILARASTAMARLRNPQVTELLATVIADLVRGEFLQLKNTMDPSSLEIKQSNFDYYIEKSFLKTASLISKSCKASTILGQCSPTVATAAGEYGRCIGTAFQLMDDVLDYTSKDDTLGKAAGADLKLGLATAPVLFAWKKYPELGAMIVNRFNHPSDIQRARSLVECTDAIEQTITWAKEYIKKAKDSLLCLPDSPARKALFALADKVITRKK

SEQ ID NO: 48 - protein, 325 aa, Rhodobacter capsulatus
MAIDFKQDILAPVAQDFAAMDQFINEGISSKVALVMSVSKHVVEAGGKRMRPIMCLLAAYACGETNLKHAQKLAAIIEMLHTATLVHDDDVDESGLRRGRPTANATWNNQTAVLVGDFLIARAFDLLVDLDNMILLKDFSTGTCEIAEGEVLQLQAQHQPDTTEDIYLQIIHGKTSRLFELATEGAAILAGKPEYREPLRRFAGHFGNAFQIIDDILDYTSDADTLGKNIGDDLMEGKPTLPLIAAMQNTQGEQRDLIRRSIATGGTSQLEQVIAIVQNSGALDYCHKRATEETERALQALEILPESTYRQALVNLTRLALDRIQ

SEQ ID NO: 49 - protein, 325 aa, Rhodobacter capsulatus
MAIDFKQDILAPVAQDFAAMDQFINEGISSKVALVMSVSKHVVEAGGKRMRPIMCLLAAYACGETNLKHAQKLAAIIEMLHTATLVHDDVVDESGLRRGRPTANATWNNQTAVLVGDFLIARAFDLLVDLDNMILLKDFSTGTCEIAEGEVLQLQAQHQPDTTEDIYLQIIHGKTSRLFELATEGAAILAGKPEYREPLRRFAGHFGNAFQIIDDILDYTSDADTLGKNIGDDLMEGKPTLPLIAAMQNTQGEQRDLIRRSIATGGTSQLEQVIAIVQNSGALDYCHKRATEETERALQALEILPESTYRQALVNLTRLALDRIQ

SEQ ID NO: 50 - protein, 327 aa, Rickettsia prowazeki
MNIIVKIQQNLKDEVTQLNDLIISCLKSDAELIEKVGKYLVEAGGKRIRPLLTIITAKMFDYKGNNHIKLASAVEFIHAATLLHDDVVDNSTLRRFKPTANVIWGSKTSILVGDFLFSQSFKLMVASGCIKAMNVLAKASVIISEGEVVQLVKLNERRIITIDEYQQIVKSKTAELFGAACEVGAIIAEQVDRVSKDVQNFGRLLGTIFQVIDDLLDYLGSDKQVGKNIGDDFLEGKVTLPLIFLYHKLEQDKQLWLENMLKSDKRTKDDFVKIRDLMLKHAIYNETVNYLSSLENEANNLLNKIPVQNIYKYYLFSIIRFILYRSY

SEQ ID NO: 51 - protein, 323 aa, Escherichia coli
MNLEKINELTAQDMAGVNAAILEQLNSDVQLINQLGYYIVSGGGKRIRPMIAVLAARAVGYEGNAHVTIAALIEFIHTATLLHDDVVDESDMRRGKATANAAFGNAASVLVGDFIYTRAFQMMTSLGSLKVLEVMSEAVNVIAEGEVLQLMNVNDPDITEENYMRVIYSKTARLFEAAAQCSGILAGCTPEEEKGLQDYGRYLGTAFQLIDDLLDYNADGEQLGKNVGDDLNEGKPTLPLLHAMHHGTPEQAQMIRTAIEQGNGRHLLEPVLEAMNACGSLEWTRQRAEEEADKAIAALQVLPDTPWREALIGLAHIAVQRDR

SEQ ID NO: 52 - protein, 329 aa, Haemophilus influenzae
MKKQDLMSIDEIQKLADPDMQKVNQNILAQLNSDVPLIGQLGFYIVQGGGKRIRPLIAVLAARSLGFEGSNSITCATFVEFIHTASLLHDDVVDESDMRRGRATANAEFGNAASVLVGDFIYTRAFQLVAQLESLKILSIMADATNVLAEGEVQQLMNVNDPETSEANYMRVIYSKTARLFEVAGQAAAIVAGGTEAQEKALQDYGRYLGTAFQLVDDVLDYSANTQALGKNVGDDLAEGKPTLPLLHAMRHGNAQQAALIREAIEQGGKREAIDEVLAIMTEHKSLDYAMNRAKEEAQKAVDAIEILPESEYKQALISLAYLSVDRNY

SEQ ID NO: 53 - DNA, 24 bp, Artificial Sequence
  Primer
rtkattytmaaygayaaygaaatg

SEQ ID NO: 54 - DNA, 23 bp, Artificial Sequence
  Primer
tttgaagarytvggywttaacta

SEQ ID NO: 55 - DNA, 21 bp, Artificial Sequence
  Primer
rcaycargcttayscvcayaa

SEQ ID NO: 56 - DNA, 22 bp, Artificial Sequence
  Primer
cgtgytgytcdgcrathgcbac

SEQ ID NO: 57 - DNA, 23 bp, Artificial Sequence
  Primer
tgytcdgcrathgcbacrtcraa

SEQ ID NO: 58 - DNA, 20 bp, Artificial Sequence
  Primer
ggsccdatrtagttaawrcc

SEQ ID NO: 59 - DNA, 27 bp, Artificial Sequence
  Primer
tcgtgaccaagaagggcaagggctatg

SEQ ID NO: 60 - DNA, 27 bp, Artificial Sequence
  Primer
gacaagtatcacggcgtccagaagttc

SEQ ID NO: 61 - DNA, 27 bp, Artificial Sequence
  Primer
atagcccttgcccttcttggtcacgac

SEQ ID NO: 62 - DNA, 26 bp, Artificial Sequence
  Primer
cgaacggatcatactcgctctcgctg

SEQ ID NO: 63 - DNA, 28 bp, Artificial Sequence
  Primer
tgaggatcttgtgcggatagcattggtg

SEQ ID NO: 64 - DNA, 26 bp, Artificial Sequence
  Primer
agcggcgtcttgggtaggtcagccat

SEQ ID NO: 65 - DNA, 30 bp, Artificial Sequence
  Primer
atatggtaccgtgtgactgacctgtccaac

SEQ ID NO: 66 - DNA, 30 bp, Artificial Sequence
  Primer
agtctctagaatgttggagattcaaggtgg

SEQ ID NO: 67 - DNA, 20 bp, Artificial Sequence
  Primer
ggwgghaarmgmmtkcgycc

SEQ ID NO: 68 - DNA, 20 bp, Artificial Sequence
  Primer
acwytgstdcatgatgatgt

SEQ ID NO: 69 - DNA, 20 bp, Artificial Sequence
  Primer
acnytnbtncaygaygaygt

SEQ ID NO: 70 - DNA, 20 bp, Artificial Sequence
  Primer
tyrtcyacsacatcatcatg

SEQ ID NO: 71 - DNA, 23 bp, Artificial Sequence
  Primer
tghavkacytcaccytcrgmaat

SEQ ID NO: 72 - DNA, 20 bp, Artificial Sequence
  Primer
tartcnardatrtcrtcdat

SEQ ID NO: 73 - DNA, 20 bp, Artificial Sequence
  Primer
tcrtcnccnaynktyttncc

SEQ ID NO: 74 - DNA, 26 bp, Artificial Sequence
  Primer
tggaagctgcgggcgaagagatagtc

SEQ ID NO: 75 - DNA, 26 bp, Artificial Sequence
  Primer
cccaccagcaccgaggatttgttgtc

SEQ ID NO: 76 - DNA, 27 bp, Artificial Sequence
  Primer
gaacctgctgtgggacaacaaatcctc

SEQ ID NO: 77 - DNA, 27 bp, Artificial Sequence
  Primer
tcggtgctggtgggcgactatctcttc

SEQ ID NO: 78 - DNA, 30 bp, Artificial Sequence
  Primer
actagaattccgcaacagttccttcatgtc

SEQ ID NO: 79 - DNA, 30 bp, Artificial Sequence
  Primer
atagaagcttacttgcggtcggactgatag

SEQ ID NO: 80 - DNA, 23 bp, Artificial Sequence
  Primer
ctsstscaygaygaygtsgtsga

SEQ ID NO: 81 - DNA, 19 bp, Artificial Sequence
  Primer
gtsgmvgssggsggsaarc

SEQ ID NO: 82 - DNA, 18 bp, Artificial Sequence
  Primer
ctsmtscaygaygaygts

SEQ ID NO: 83 - DNA, 20 bp, Artificial Sequence
  Primer
dssrtbctsgtsggsgaytt

SEQ ID NO: 84 - DNA, 21 bp, Artificial Sequence
  Primer
vakraartcsccsacsagsac

SEQ ID NO: 85 - DNA, 18 bp, Artificial Sequence
  Primer
sacytcsccytcsgcrat

SEQ ID NO: 86 - DNA, 21 bp, Artificial Sequence
  Primer
rtcrtcsccvayvktyttscc

SEQ ID NO: 87 - DNA, 21 bp, Artificial Sequence
  Primer
sggsagsgtvrbyttsccytc

SEQ ID NO: 88 - DNA, 26 bp, Artificial Sequence
  Primer
gtgctggtcggcgacttcctgttcag

SEQ ID NO: 89 - DNA, 27 bp, Artificial Sequence
  Primer
atcgacctgtccgaggatcgctatctc

SEQ ID NO: 90 - DNA, 26 bp, Artificial Sequence
  Primer
tcgaacgagcggctgaacaggaagtc

SEQ ID NO: 91 - DNA, 26 bp, Artificial Sequence
  Primer
tggcgggattgccccagatgatgttg

SEQ ID NO: 92 - DNA, 30 bp, Artificial Sequence
  Primer
attaggtaccatcagataatcgtcgctcaa

SEQ ID NO: 93 - DNA, 30 bp, Artificial Sequence
  Primer
tataggatccgacatggacgaggaagacgc

SEQ ID NO: 94 - DNA, 20 bp, Artificial Sequence
  Primer
cgatggtgaccacgaagaag

SEQ ID NO: 95 - DNA, 2017 bp, Sphingomonas trueperi
ggcccgggctggtggggtttctggcgctggggctggtgttcggcgcgttcttcttcgtcgcgatcgtgacgcggaacgccaagctggcggcggggcaggtctatgtcgggctgccggtgctcgcgctgctgctgctccgcgaccatccgcagggctttgccgcgacgctgtggacgatggcgatcgtctgggtgtgcgacagcggcgcctattttgccggtcgcgcgatcggtgggcccaagctcgcgccctcgatcagcccgaacaagacctgggcggggctgatcggcgggttggttgccgcgatcctgttctccgccggctatgtcgcgctggcgccggggagcgcgatcggctggtggctggtcgcggtgtcgccgctggtagccttcgcctcgcagatcggcgacctgtacgagagccatctcaagcgggtcgcgggcgtgaaggattcgagcaacctgctgcccggccatggcggcattctcgaccggctcgacggccttgtcttcgcagccccggttgcagctttgttttttgcgatccatcatcaggtggtcgtgggaggatactggtggtgaagcgcgtcacggtgttgggggcgaccggctcggtcggcacctcgacgctggatctgatcgaacgaaatccgcacgccttcgaagtcgtggcgctgaccgcaaattgcgatgtcgagaagctggctgccgcggcgatccgcacgcgcgcgcgctgcgccgtggtcgccgacgagaaatgcctgccggcgctacaggagcggctggccggcagcggtgtcgaggcgatgggcggggcgcattcggtgtgcgacgtggcgcggatgggtgctgactggacgatggctgcgatcgtcggcagcgcagggctcaagccggtgatggccgcgctggaggccggtggcaccgtcgcgctcgcgaacaaggagtcgctcgtctcggcgggtgaggtgatgatggcggcggcccgcgcgcatggcgcgacgctgctgccggtcgattcggagcacaatgcggtgttccagtgcctcgatcgcaccgcgcccaggggcgtccgccggatcatccttaccgccagcggtggtccgttccgcgcgacgccgaaggaagcgatgcgcgacatcacccccgcacaggcggtggcgcatcccaactggtcgatgggcgccaagatctcggtcgactccgcgacgatgatgaacaaggggctcgaactgatcgaagccttccacctgttcccggtcgccgccgagcaactggccgtgctggtccatcgccaatccgtcgtccattcgatggtggaatatgtcgacggatcggtgctggcccagctcggcacgcccgacatgcgcacgccgatcgcctatgcgctggcttggcccgagcggatggagacgctgtgcccgccgctcgaccttgccacggtgggtaagctcgagttcgaaaatcccgatctcgatcgcttcccggcgctcgcgctggcgatggaggcattgaaggcgggcggggcgcgtccggccattctcaatgccgccaacgaagtcgccgtcgcggcctttctcgccgggcggatcggattccttgaaattgccgcaatctctgccgatacgctgtctcgctatgacccggccgcgccggaaacgctcgatgccgtgctggcgatcgacgcggaggcgcggctttacgcggctgagcgagtgaaggactgcgtcgcttgatccaatcccccggcatcctgctcaccattctggcgttcgcgctggtgatcgggccgctcgtgttcctgcacgagctgggacattatctggcgggccgcctcttcggggtgaaggccgaggaattctcgatcggcttcggccgcgagatcgccggcaccaccgatcgccgcggcacgcgctggaagttcagcctgttgccgctgggcggctatgtccgcttcgccggcgacatgaacccggcgagccagccttcgcccgaatggctgcagaccagcccgggcc

SEQ ID NO: 96 - DNA, 1161 bp, Sphingomonas trueperi
gtggtgaagcgcgtcacggtgttgggggcgaccggctcggtcggcacctcgacgctggatctgatcgaacgaaatccgcacgccttcgaagtcgtggcgctgaccgcaaattgcgatgtcgagaagctggctgccgcggcgatccgcacgcgcgcgcgctgcgccgtggtcgccgacgagaaatgcctgccggcgctacaggagcggctggccggcagcggtgtcgaggcgatgggcggggcgcattcggtgtgcgacgtggcgcggatgggtgctgactggacgatggctgcgatcgtcggcagcgcagggctcaagccggtgatggccgcgctggaggccggtggcaccgtcgcgctcgcgaacaaggagtcgctcgtctcggcgggtgaggtgatgatggcggcggcccgcgcgcatggcgcgacgctgctgccggtcgattcggagcacaatgcggtgttccagtgcctcgatcgcaccgcgcccaggggcgtccgccggatcatccttaccgccagcggtggtccgttccgcgcgacgccgaaggaagcgatgcgcgacatcacccccgcacaggcggtggcgcatcccaactggtcgatgggcgccaagatctcggtcgactccgcgacgatgatgaacaaggggctcgaactgatcgaagccttccacctgttcccggtcgccgccgagcaactggccgtgctggtccatcgccaatccgtcgtccattcgatggtggaatatgtcgacggatcggtgctggcccagctcggcacgcccgacatgcgcacgccgatcgcctatgcgctggcttggcccgagcggatggagacgctgtgcccgccgctcgaccttgccacggtgggtaagctcgagttcgaaaatcccgatctcgatcgcttcccggcgctcgcgctggcgatggaggcattgaaggcgggcggggcgcgtccggccattctcaatgccgccaacgaagtcgccgtcgcggcctttctcgccgggcggatcggattccttgaaattgccgcaatctctgccgatacgctgtctcgctatgacccggccgcgccggaaacgctcgatgccgtgctggcgatcgacgcggaggcgcggctttacgcggctgagcgagtgaaggactgcgtcgcttga

SEQ ID NO: 97 - protein, 386 aa, Sphingomonas trueperi
VVKRVTVLGATGSVGTSTLDLIERNPHAFEVVALTANCDVEKLAAAAIRTRARCAVVADEKCLPALQERLAGSGVEAMGGAHSVCDVARMGADWTMAAIVGSAGLKPVMAALEAGGTVALANKESLVSAGEVMMAAARAHGATLLPVDSEHNAVFQCLDRTAPRGVRRIILTASGGPFRATPKEAMRDITPAQAVAHPNWSMGAKISVDSATMMNKGLELIEAFHLFPVAAEQLAVLVHRQSVVHSMVEYVDGSVLAQLGTPDMRTPIAYALAWPERMETLCPPLDLATVGKLEFENPDLDRFPALALAMEALKAGGARPAILNAANEVAVAAFLAGRIGFLEIAAISADTLSRYDPAAPETLDAVLAIDAEARLYAAERVKDCVA

SEQ ID NO: 98 - protein, 388 aa, Bacillus subtilis
MKNICLLGATGSIGEQTLDVLRAHQDQFQLVSMSFGRNIDKAVPMIEVFQPKFVSVGDLDTYHKLKQMSFSFECQIGLGEEGLIEAAVMEEVDIVVNALLGSVGLIPTLKAIEQKKTIALANKETLVTAGHIVKEHAKKYDVPLLPVDSEHSAIFQALQGEQAKNIERLIITASGGSFRDKTREELESVTVEDALKHPNWSMGAKITIDSATMMNKGLEVIEAHWLFDIPYEQIDVVLHKESIIHSMVEFHDKSVIAQLGTPDMRVPIQYALTYPDRLPLPDAKRLELWEIGSLHFEKADFDRFRCLQFAFESGKIGGTMPTVLNAANEVAVAAFLAGKIPFLAIEDCIEKALTRHQLLKKPSWRTFKKWTKIPGDTSIQYSHKVVCS

SEQ ID NO: 99 - protein, 397 aa, Haemophilus influenzae
MQKQNIVILGSTGSIGKSTLSVIENNPQKYHAFALVGGKNVEAMFEQCIKFRPHFAALDDVNAAKILREKLIAHHIPTEVLAGRRAICELAAHPDADQIMASIVGAAGLLPTLSAVKAGKRVLLANKESLVTCGQLFIDAVKNYGSKLLPVDSEHNAIFQSLPPEAQEKIGFCPLSELGVSKIILTGSGGPFRYTPLEQFTNITPEQAVAHPNWSMGKKISVDSATMMNKGLEYIEARWLFNASAEEMEVIIHPQSIIHSMVRYVDGSVITQMGNPDMRTPIAETMAYPHRTFAGVEPLDFFKIKELTFIEPDFNRYPNLKLAIDAFAAGQYATTAMNAANEIAVQAFLDRQIGFMDIAKINSKTIERISPYTIQNIDDVLEIDAQAREIAKTLLRE

SEQ ID NO: 100 - protein, 398 aa, Escherichia coli
MKQLTILGSTGSIGCSTLDVVRHNPEHFRVVALVAGKNVTRMVEQCLEFSPRYAVMDDEASAKLLKTMLQQQGSRTEVLSGQQAACDMAALEDVDQVMAAIVGAAGLLPTLAAIRAGKTILLANKESLVTCGRLFMDAVKQSKAQLLPVDSEHNAIFQSLPQPIQHNLGYADLEQNGVVSILLTGSGGPFRETPLRDLATMTPDQACRHPNWSMGRKISVDSATMMNKGLEYIEARWLFNASASQMEVLIHPQSVIHSMVRYQDGSVLAQLGEPDMRTPIAHTMAWPNRVNSGVKPLDFCKLSALTFAAPDYDRYPCLKLAMEAFEQGQAATTALNAANEITVAAFLAQQIRFTDIAALNLSVLEKMDMREPQCVDDVLSVDANAREVARKEVMRLAS

SEQ ID NO: 101 - protein, 388 aa, Zymonas mobilis
MSQPRTVTVLGATGSIGHSTLDLIERNLDRYQVIALTANRNVKDLADAAKRTNAKRAVIADPSLYNDLKEALAGSSVEAAAGADALVEAAMMGADWTMAAIIGCAGLKATLAAIRKGKTVALANKESLVSAGGLMIDAVREHGTTLLPVDSEHNAIFQCFPHHNRDYVRRIIITASGGPFRTTSLAEMATVTPERAVQHPNWSMGAKISIDSATMMNKGLELIEAYHLFQIPLEKFEILVHPQSVIHSMVEYLDGSILAQIGSPDMRTPIGHTLAWPKRMETPAESLDFTKLRQMDFEAPDYERFPALTLAMESIKSGGARPAVMNAANEIAVAAFLDKKIGFLDIAKIVEKTLDHYTPATPSSLEDVFAIDNEARIQAAALMESLPA

SEQ ID NO: 102 - protein, 402 aa, Synechococcus leopoliensis
MKAVTLLGSTGSIGTQTLDILEQYPDRFRLVGLAAGRNVALLSEQIRRHRPEIVAIQDAAQLSELQAAIADLDNPPLILTGEAGVTEVARYGDAEIVVTGIVGCAGLLPTIAAIEAGKDIALANKETLIAAGPVVLPLLQKHGVTITPADSEHSAIFQCIQGLSTHADFRPAQVVAGLRRILLTASGGAFRDWPVERLSQVTVADALKHPNWSMGRKITVDSATLMNKGLEVIEAHYLFGLDYDYIDIVIHPQSIIHSLIELEDTSVLAQLGWPDMRLPLLYALSWPDRLSTQWSALDLVKAGSLEFREPDHAKYPCMDLAYAAGRKGGTMPAVLNAANEQAVALFLEEQIHFSDIPRLIERACDRHQTEWQQQPSLDDILAYDAWARQFVQASYQSLESVV

SEQ ID NO: 103 - protein, 394 aa, Synechocystis sp. PCC 6803
MVKRISILGSTGSIGTQTLDIVTHHPDAFQVVGLAAGGNVALLAQQVAEFRPEIVAIRQAEKLEDLKAAVAELTDYQPMYVVGEEGVVEVARYGDAESVVTGIVGCAGLLPTMAAIAAGKDIALANKETLIAGAPVVLPLVEKMGVKLLPADSEHSAIFQCLQGVPEGGLRRIILTASGGAFRDLPVERLPFVTVQDALKHPNWSMGQKITIDSATLMNKGLEVIEAHYLFGLDYDHIDIVIHPQSIIHSLIEVQDTSVLAQLGWPDMRLPLLYALSWPERIYTDWEPLDLVKAGSLSFREPDHDKYPCMQLAYGAGRAGGAMPAVLNAANEQAVALFLQEKISFLDIPRLIEKTCDLYVGQNTASPDLETILAADQWARRTVLENSACVATRP

SEQ ID NO: 104 - protein, 436 aa, Mycobacterium tuberculosis
MATGGRVVIRRRGDNEVVAHNDEVTNSTDGRADGRLRVVVLGSTGSIGTQALQVIADNPDRFEVVGLAAGGAHLDTLLRQRAQTGVTNIAVADEHAAQRVGDIPYHGSDAATRLVEQTEADVVLNALVGALGLRPTLAALKTGARLALANKESLVAGGSLVLRAARPGQIVPVDSEHSALAQCLRGGTPDEVAKLVLTASGGPFRGWSAADLEHVTPEQAGAHPTWSMGPMNTLNSASLVNKGLEVIETHLLFGIPYDRIDVVVHPQSIIHSMVTFIDGSTIAQASPPDMKLPISLALGWPRRVSGAAAACDFHTASSWEFEPLDTDVFPAVELARQAGVAGGCMTAVYNAANEEAAAAFLAGRIGFPAIVGIIADVLHAADQWAVEPATVDDVLDAQRWARERAQRAVSGMASVAIASTAKPGAAGRHASTLERS

SEQ ID NO: 105 - DNA, 1191 bp, Pseudomonas aeruginosa
atgagtcgaccgcagcggatcagcgtgctcggcgcgaccggctcgatcggcctgagcaccctggacgtcgtccagcgtcatcccgatcgttacgaagccttcgccctgactggcttcagccgcctggccgaactcgaggcgctgtgcctcaggcaccgccccgtctatgcggtggtgccggagcaggccgcggcgattgccttgcagggctcgctcgccgcggcgggtatccgcacccgggtgctgttcggcgagcaggcgttgtgcgaagtggccagcgcgcccgaagtggacatggtaatggcggccatcgtcggcgccgccgggctgccgtcgaccctggcggccgtcgaggccggcaagcgcgtactgctggccaacaaggaggcgctggtgatgtccggcgcgctgttcatgcaggcggtcaagcgcagcggcgcggtgctcctgccgatcgacagcgagcacaacgcgatcttccagtcgctgccgcgcaattatgccgatggcctggagcgggtcggcgtgcgccggatcctcttgaccgcctccggcggcccgttccgcgagacgccgctggagcaactcgcttcggtgacgccggagcaggcttgtgcgcacccgaactggtcgatggggcgtaagatttccgtcgactccgccagcatgatgaacaaggggctcgaactgatcgaggcgtgctggctgttcgacgcccagccgagccaggtcgaggtggtgatccacccgcagagcgtgatccactcgatggtggactacgtcgacggttcggtgatcgcccagctcggcaatccggacatgcgcacgccgatttcctatgccatggcctggccggagcgaatcgattccggcgtttcgccgctggatatgttcgccgtcggtcgcctggatttccagcgccccgacgagcagcgcttcccctgcctgcgcctggcgagccaggccgcggaaaccggcggcagcgccccggccatgctgaatgccgcgaacgaggtggccgtggccgcatttctcgagcggcacatccgcttcagcgacatcgcggttatcatcgaggacgtgctgaaccgcgaggcggtgaccgcagtcgaatcgctcgatcaggtcctggctgccgatcgccgcgcgcgttcggtcgccgggcaatggttgacccggcacgccggctag

SEQ ID NO: 106 - DNA, 1167 bp, Zygomonas mobilis
atgagtcagccaagaacagtcactgttttaggggcgaccggatccattggtcattcaacactggatttaatcgaacggaatttagatcggtatcaggtcatcgctttgaccgccaaccgcaatgtcaaagatctggccgatgcggcgaaaagaacgaatgccaagcgggcggttatcgctgacccgtcgctttataatgatctgaaagaggctttggccggaagctctgttgaggcagccgcgggtgctgatgccttggtcgaagccgccatgatgggtgccgattggacaatggcagccattatcggttgcgccggtctaaaagcgacgcttgcagctattcgcaagggcaaaacggtcgctttagcgaataaggaatccttagtttcagctggcggattgatgatcgatgccgtgcgggaacatggcacgacgcttctccccgtcgattccgagcataacgctattttccaatgcttcccgcatcataaccgcgactatgttcgccggattattattacggccagcggaggtcccttcagaacaacgtctcttgccgaaatggcaacggtcacgccagaacgcgcggttcagcatcccaactggtcaatgggtgccaagatttctatcgattctgctacaatgatgaataaggggcttgaattgatagaagcctatcatctcttccagattccattagaaaaatttgaaattttggttcatcctcagtcagttattcactccatggtggaatatttggatggttctatccttgcccagatcggtagtcctgatatgagaacaccgatcggtcatactttggcttggccaaagcggatggaaacaccagccgaatcgttggattttaccaaattgcgccagatggattttgaagcaccagattatgaacgttttccggcattaactttggcaatggaatccatcaaatcaggtggggctcgtcctgctgtaatgaatgccgctaatgaaatagctgtggcggccttccttgataagaaaatcggttttcttgatatcgctaaaattgtcgagaaaacattagatcattatacacccgcaaccccgtcttctttggaagatgtctttgcgatcgacaatgaagcgcggatacaagccgctgctttaatggagagtttgcccgcgtga

SEQ ID NO: 107 - DNA, 1161 bp, Streptomyces griseolosporeus
ttggtcattctcggctcgaccggctcgatcggcacccaggccatcgacgtggtgctccgcaaccccggccggttcaaggtggtcgcgctgtccgcggccggcggcgcggtggagctgctcgccgagcaggccgtcgcactgggcgtgcacaccgtcgcggtggccgacccggccgccgaggaagccgctgcgcgaggccctggcggccaaggcgcagggcgcccgctgccgcgggtgctggcgggcccggacgcggcgaccgagctggccgcggcggagtgccactcggtgctgaacggcatcaccggttcgatcggcctggccccgacgctggccgcgctgcgggccggccgggtgctggtgctggcgaacaaggagtcgctgatcgtcggcggtccgctggtgaaggcggtggcgcagcccggccagatcgtgccggtggactccgagcacgccgcgctgttccaggcgctggccggcggcgcccgcgcggaggtccgcaagctggtggtgaccgccagcggcggcccgttccgcaaccgcacccgtgagcagctggcggccgtcacgccggccgacgcgctggcgcacccgacctgggcgatgggcccggtggtgacgatcaactcggcgaccctggtgaacaagggcctggaggtgatcgaggcgcacctgctgtacgacgtgccgttcgaccggatcgaggtggtggtccatccgcagtcggtcgttcattcgatggtggaattcgtggacggttcgacgatggcccaggccagcccgccggacatgcgcatgccgatcgcgctgggcctcggctggccggaccgggtgccggacgccgcccccggctgcgactggaccaaggccgcgacctgggagttcttcccgctggacaacgaggcgttcccggcggtcgagctggcccgcgaggtgggtacgctcggcgggaccgccccggcggtcttcaatgccgccaacgaggaatgtgtggacgctttcctgaagggcgcactgcccttcaccggaatcgtggacactgtggcgaaggtggtcgccgaacacggcacaccgcaatcgggaacttcgctcacggtggaggacgtactccacgcggagagctgggcccgggcccgggcccgcgagctggcggccggctga

SEQ ID NO: 108 - DNA, 1185 bp, Neisseria meningitidis
atgacaccacaagtcctgaccatattaggcagtaccggcagcataggcgaaagcacgctggacgttgtctcccgccaccccgaaaaattccgcgtattcgcgctggcagggcataagcaggtcgagaaattggcggctcaatgtcaaacgttccaccccgaatatgccgtcgttgccgatgccgaacacgccgcccggcttgaagccctgttgaaacgcgacggcacggcgactcaggttttacacggcgcgcaggcattggttgacgttgcctctgccgacgaagtcagcggtgtcatgtgcgccatcgtcggggcggtggggctgccttccgcgctcgcagcggcgcaaaaaggcaaaaccatttatctggcgaacaaagagacgctggtggtttccggcgcgttgtttatggaaaccgcccgtgcaaacggcgcggcagtgctgcccgtcgacagcgaacacaacgccgttttccaagttttgccgcgcgattacacaggtcgcctgaacgaacacggcatcgcttcgattatcctgaccgcttccggcggcccgtttctgaccgccgatttaaacacgttcgacagcattacgcccgaccaagcggtcaaacaccccaattggcgtatgggacgcaaaatctccgtcgattccgccaccatgatgaacaaaggtttggagctgattgaagcgcattggctgttcaactgtccgcccgacaaactcgaagtcgtcatccatccgcaatctgtgatacacagcatggtgcgctaccgcgacggctccgtgttggcgcaactgggcaatcccgatatgcgaacgcctatcgcttattgtttgggtttgcccgagcgcatcgattcgggtgtcggcgacctggatttcgacgcattgtccgcgctgaccttccaaaagcccgactttgaccgcttcccctgcctgaagctcgcctatgaagccatgaacgcaggcggagccgcgccctgcgtattgaacgccgccaacgaagccgccgtcgccgcctttttggacggacagattaagtttaccgacattgccaaaaccgtcgcccattgtctttcacaagacttttcagacggcataggcgacataggggggctcttggcgcaagatgcccggacacgcgcacaagcgcgggcatttatcggcacactgcgctga

SEQ ID NO: 109 - DNA, 1197 bp, Escherichia coli
atgaagcaactcaccattctgggctcgaccggctcgattggttgcagcacgctggacgtggtgcgccataatcccgaacacttccgcgtagttgcgctggtggcaggcaaaaatgtcactcgcatggtagaacagtgcctggaattctctccccgctatgccgtaatggacgatgaagcgagtgcgaaacttcttaaaacgatgctacagcaacagggtagccgcaccgaagtcttaagtgggcaacaagccgcttgcgatatggcagcgcttgaggatgttgatcaggtgatggcagccattgttggcgctgctgggctgttacctacgcttgctgcgatccgcgcgggtaaaaccattttgctggccaataaagaatcactggttacctgcggacgtctgtttatggacgccgtaaagcagagcaaagcgcaattgttaccggtcgatagcgaacataacgccatttttcagagtttaccgcaacctatccagcataatctgggatacgctgaccttgagcaaaatggcgtggtgtccattttacttaccgggtctggtggccctttccgtgagacgccattgcgcgatttggcaacaatgacgccggatcaagcctgccgtcatccgaactggtcgatggggcgtaaaatttctgtcgattcggctaccatgatgaacaaaggtctggaatacattgaagcgcgttggctgtttaacgccagcgccagccagatggaagtgctgattcacccgcagtcagtgattcactcaatggtgcgctatcaggacggcagtgttctggcgcagctgggggaaccggatatgcgtacgccaattgcccacaccatggcatggccgaatcgcgtgaactctggcgtgaagccgctcgatttttgcaaactaagtgcgttgacatttgccgcaccggattatgatcgttatccatgcctgaaactggcgatggaggcgttcgaacaaggccaggcagcgacgacagcattgaatgccgcaaacgaaatcaccgttgctgcttttcttgcgcaacaaatccgctttacggatatcgctgcgttgaatttatccgtactggaaaaaatggatatgcgcgaaccacaatgtgtggacgatgtgttatctgttgatgcgaacgcgcgtgaagtcgccagaaaagaggtgatgcgtctcgcaagctga

SEQ ID NO: 110 - DNA, 1209 bp, Synechococcus leopoliensis
gtgaaagcagtgacactgctcggttcaaccggctcgatcgggacacaaaccctagacattcttgagcagtatcccgatcgctttcgcctcgtagggctggcggctggtcgtaatgtggcgctgttgtcggagcaaattcggcggcaccgaccagagattgtggcgattcaagatgcagctcagctgtcggaactgcaagcggcgatcgcagaccttgataatccgccgctcatcctgaccggtgaggcaggtgtcacggaagtggctcgctacggtgatgccgagattgtggtcactggcattgtcggttgcgctggtctgctacccacgatcgccgcgatcgaagccggcaaggatatcgcccttgccaacaaagaaaccctgattgcagcaggcccagtggtcctgccactcctgcaaaagcacggtgtcaccattacgcctgccgactccgagcactccgcgatctttcagtgcatccaagggctttcaacccatgctgattttcggcctgctcaagtcgtggcagggctgcgacggattctcctgactgccagtggcggcgcttttcgggactggccggtcgaacggctgtcgcaagtaactgtcgcagatgcgctcaagcatcccaactggtcgatggggcgcaagattaccgtcgactccgccaccttgatgaataaaggcctcgaggtgatcgaagcccactatctcttcggcttggattacgactacatcgacatcgtcatccatccccagagcatcatccactcgctgattgagctagaagatacctccgtcttggcgcaattgggctggccggatatgcgactgcccttgctctacgccctctcctggcccgatcgcctctctactcaatggtcggcgctcgatctggtcaaagcgggcagcttggagttccgggaaccggatcacgccaaatacccctgcatggacttggcctacgccgccggtcgcaaaggcggcacaatgccagccgtcttgaatgcggcgaatgagcaagccgtcgccctcttcctagaggagcaaattcacttctcggatattccgcgcctgattgaacgtgcctgcgatcgccaccaaacggagtggcaacagcaaccgagcttggatgacattttggcctacgacgcttgggcacggcagtttgtgcaagctagctatcaaagtctggaatccgtcgtttag

SEQ ID NO: 111 - DNA, 1221 bp, Mycobacterium leprae
gtgaacaatccgatcgaggggcacgctggcggccgcctccgcgtgctggtgttgggaagtactggctcaattggcacccaggcgctggaagttatcgccgccaatccggaccgtttcgaggtagtcgggctggccgccgggggcgcgcagctggacacgctgctgaggcagcgcgccgcgaccggcgtcaccaatatcgccatcgctgacgatcgcgcggctcagctggccggcgacatcccttaccacgggaccgatgcggtcacccggctggttgaggagactgaggctgacgttgtcctcaatgcgctggtcggggcattgggtctgcgacccacactggctgcactgcacacgggcgcgcgattggcgttggccaacaaggaatcgctggtagctggcggttcgctggtgttggccgcggcgcagccaggccagatcgtgcccgtagactcggaacactccgcgctggcgcaatgcctgcgcggtggtacccccgacgaagttgctaagttagtgctaaccgcctccggcgggccgtttcgtggctggaacgccggcgacttggagcgcgttacacccgagcaggcgggcgtccatccgacttggtcaatggggacgatgaacacgctgaactcagcgtctctggttaacaaggggctcgagctcatcgaagccaacctgttgttcggcattccctacgaccgcattgaggtggttgtgcaccctcagtcaattgttcattcgatggtgacattcatcgacggctcgacgatcgcccaagccagccctccggacatgaagctacctatttctttggcgttgggctggccacagcgggtgggtggcgctgctcgagcctgtgctttcactaccgcatctacctgggaattcgagccgctggacatcgatgtttttcccgcagtcgagctggcccggcacgctggacagatcggcggctgtatgaccgccatttacgatgctgctaatgaggaggctgcagaggccttcctccaaggtcggatcggcttccccgccatcgtcgcaacaatcgcggatgtgttgcagcgtgccgaccaatgggctccccaatggggtgagggacccgctactgtggatgatgtactcgacgcgcagcgctgggcccgtgagcgagcgttgtgtgcggtagcaacagcgagttctggaaaggtctctgacatggtcttagaaaggtcctaa

SEQ ID NO: 112 - DNA, 1218 bp, Pasteurella multocida
atgagtattagttattttatgaaaaagatcgttattttaggttcaactggatcgattggtaccagtactttatccgtgattacacataatcctgataagtaccaagtgtttgcgttagttggtggacgtaatgtagagctaatgtttcaacaatgtttgacattccaaccgtcgtttgctgcgttagatgacgatgtcgcagccaaaatgttggcagagaaactgaaagcccaccaaagccaaacaacagtcttagcaggacagcaagccatttgtgagttagcggcacatcctgaagcagatatggtaatggctgcgattgtgggggcggcgggattattgcctactttgtctgcggtgaaagctggaaaacgtgtactattagcaaataaagaagccttggtaacttgcgggcaattatttattgatgcagtgcgtgaatctcaagcacaattgttaccagtagatagtgaacataatgcgattttccaatcccttccgcctgaagcgcaaagacaaattgggttttgcccgctttctgaattagggatcagtaagattgtgttaacgggatccggtggtccattccgttatacccctctggagcaatttgaacagatcaccccagcacaagcagttgcgcatcctaattggtcaatggggaaaaagatctctgtcgattccgctaccatgatgaataaagggttggaatatattgaagcacgctggttatttaatgcctcggcagaagaaatggaagttattattcatcctcaatccattattcattctatggtacgttacatcgatgggtccgtgattgctcaaatggggaatcctgatatgcgtacaccgattgcggaaaccatggcatatccaagtcggaccgttgctggcgttgagcccttggatttttaccaactgaatggattaacctttattgagccagactatcaacgttatccatccatggatcttgcttatgctgctggacgagctggaggcacaaccacgacagcaatgaatgcagcgaatgaaatcgcggtagcgtctttcttagacaataagattaaattcacagatattgcgcgactaaatcagttagtcgtgagcaaattgcaaccacaaaaaattcattgcatagaagatgtacttgaggtagataaaaaggcaagggaattatctcagtcaatcattttaagtttttcacatccgtaa

SEQ ID NO: 113 - DNA, 1434 bp, Arabidopsis thaliana
atgatgacattaaactcactatctccagctgaatccaaagctatttctttcttggatacctccaggttcaatccaatccctaaactctcaggtgggtttagtttgaggaggaggaatcaagggagaggttttggaaaaggtgttaagtgttcagtgaaagtgcagcagcaacaacaacctcctccagcatggcctgggagagctgtccctgaggcgcctcgtcaatcttgggatggaccaaaacccatctctatcgttggatctactggttctattggcactcagacattggatattgtggctgagaatcctgacaaattcagagttgtggctctagctgctggttcgaatgttactctacttgctgatcaggtaaggagatttaagcctgcattggttgctgttagaaacgagtcactgattaatgagcttaaagaggctttagctgatttggactataaactcgagattattccaggagagcaaggagtgattgaggttgcccgacatcctgaagctgtaaccgttgttaccggaatagtaggttgtgcgggactaaagcctacggttgctgcaattgaagcaggaaaggacattgctcttgcaaacaaagagacattaatcgcaggtggtcctttcgtgcttccgcttgccaacaaacataatgtaaagattcttccggcagattcagaacattctgccatatttcagtgtattcaaggtttgcctgaaggcgctctgcgcaagataatcttgactgcatctggtggagcttttagggattggcctgtcgaaaagctaaaggaagttaaagtagcggatgcgttgaagcatccaaactggaacatgggaaagaaaatcactgtggactctgctacgcttttcaacaagggtcttgaggtcattgaagcgcattatttgtttggagctgagtatgacgatatagagattgtcattcatccgcaaagtatcatacattccatgattgaaacacaggattcatctgtgcttgctcaattgggttggcctgatatgcgtttaccgattctctacaccatgtcatggcccgatagagttccttgttctgaagtaacttggccaagacttgacctttgcaaactcggttcattgactttcaagaaaccagacaatgtgaaatacccatccatggatcttgcttatgctgctggacgagctggaggcacaatgactggagttctcagcgccgccaatgagaaagctgttgaaatgttcattgatgaaaagataagctatttggatatcttcaaggttgtggaattaacatgcgataaacatcgaaacgagttggtaacatcaccgtctcttgaagagattgttcactatgacttgtgggcacgtgaatatgccgcgaatgtgcagctttcttctggtgctaggccagttcatgcatga

SEQ ID NO: 114 - DNA, 1071 bp, Campylobacter jejuni
atgatactttttggaagtacgggcagtataggagtaaatgctcttaaacttgctgctttaaaaaacattcccatttctgctttagcttgtggggataacatcgctcttttaaatgagcaaatcgcaaggtttaaacccaaatttgtttccataaaagattcaaaaaataagcatttagttaaacacgatagagtttttatagggcaagaaggtttagagcaaattttaacagaatgtcaagataagcttttactcaatgccattgtaggttttgcaggacttaaaagcactttaaaggctaaagagcttggcaaaaacatagctttagctaacaaagaaagtcttgtagtagctgggagttttttgaaaggggctaaatttttacccgttgatagtgagcatgcagctttaaaatttttactcgaaggtaaaaaaaatatagcaaaactttatatcacagcaagtggtggagctttttataggtataaaatcaaagatttaaatcaagtcagtgtcaaagatgctttaaaacatcctaattggaacatgggagcaaagatcactatagatagtgcgactatggcaaataagctttttgagattatagaggcttatcatttatatgattttaaagaaattgatgctttaatagaaccaagatctttagtgcatgcaatgtgtgaatttaaaaatggagctagcacggcgtatttttcaaaagcagatatgaaactagctatttcagatgctatatttgaaaaacaagatacgcctattttagaggctgttgattttagcaaaatgcctgctttaaaatttcatccaatcagcacaaaaaaatatcctatttttaagcttaaaaatacatttttaaaagagccaaatttaggtgttatcatcaatgctgctaatgaagttggtgtttataattttttagaaaataaaagtggatttttagacattgctaaatgcatttttaaagcccttgatcattttggagtacctaaaatttcaagcatagaagaagtttttgagtatgattttaaaacaagagagtatttaaggagttaa

SEQ ID NO: 115 - DNA, 1467 bp, Plasmodium falciparum
atgaagaaatatatttatatatattttttcttcatcacaataactattaatgatttagtaataaataatacatcaaaatgtgtttccattgaaagaagaaaaaataacgcatatataaattatggtataggatataatggaccagataataaaataacaaagagtagaagatgtaaaagaataaagttatgcaaaaaggatttaatagatattggtgcaataaagaaaccaattaatgtagcaatttttggaagtactggtagtataggtacgaatgctttaaatataataagggagtgtaataaaattgaaaatgtttttaatgttaaagcattgtatgtgaataagagtgtgaatgaattatatgaacaagctagagaatttttaccagaatatttgtgtatacatgataaaagtgtatatgaagaattaaaagaactggtaaaaaatataaaagattataaacctataatattgtgtggtgatgaagggatgaaagaaatatgtagtagtaatagtatagataaaatagttattggtattgattcttttcaaggattatattctactatgtatgcaattatgaataataaaatagttgcgttagctaataaagaatccattgtctctgctggtttctttttaaagaaattattaaatattcataaaaatgcaaagataatacctgttgattcagaacatagtgctatatttcaatgtttagataataataaggtattaaaaacaaaatgtttacaagacaatttttctaaaattaacaatataaataaaatatttttatgttcatctggaggtccatttcaaaatttaactatggacgaattaaaaaatgtaacatcagaaaatgctttaaagcatcctaaatggaaaatgggtaagaaaataactatagattctgcaactatgatgaataaaggtttagaggttatagaaacccattttttatttgatgtagattataatgatatagaagttatagtacataaagaatgcattatacattcttgtgttgaatttatagacaaatcagtaataagtcaaatgtattatccagatatgcaaatacccatattatattctttaacatggcctgatagaataaaaacaaatttaaaacctttagatttggctcaggtttcaactcttacatttcataaaccttctttagaacatttcccgtgtattaaattagcttatcaagcaggtataaaaggaaacttttatccaactgtactaaatgcgtcaaatgaaatagctaacaacttatttttgaataataaaattaaatattttgatatttcctctataatatcgcaagttcttgaatctttcaattctcaaaaggtttcggaaaatagtgaagatttaatgaagcaaattctacaaatacattcttgggccaaagataaagctaccgatatatacaacaaacataattcttcatag

SEQ ID NO: 116 - protein, 388 aa, Zymononas mobilis; identical to SEQ ID NO: 101

SEQ ID NO: 117 - protein, 396 aa, Pseudomonas aeruginosa
MSRPQRISVLGATGSIGLSTLDVVQRHPDRYEAFALTGFSRLAELEALCLRHRPVYAVVPEQAAAIALQGSLAAAGIRTRVLFGEQALCEVASAPEVDMVMAAIVGAAGLPSTLAAVEAGKRVLLANKEALVMSGALFMQAVKRSGAVLLPIDSEHNAIFQSLPRNYADGLERVGVRRILLTASGGPFRETPLEQLASVTPEQACAHPNWSMGRKISVDSASMMNKGLELIEACWLFDAQPSQVEVVIHPQSVIHSMVDYVDGSVIAQLGNPDMRTPISYAMAWPERIDSGVSPLDMFAVGRLDFQRPDEQRFPCLRLASQAAETGGSAPAMLNAANEVAVAAFLERHIRFSDIAVIIEDVLNREAVTAVESLDQVLAADRRARSVAGQWLTRHAG

SEQ ID NO: 118 - protein, 398 aa, Escherichia coli; identical to SEQ ID NO: 100

SEQ ID NO: 119 - protein, 394 aa, Neisseria meningitidis
MTPQVLTILGSTGSIGESTLDVVSRHPEKFRVFALAGHKQVEKLAAQCQTFHPEYAVVADAEHAARLEALLKRDGTATQVLHGAQALVDVASADEVSGVMCAIVGAVGLPSALAAAQKGKTIYLANKETLVVSGALFMETARANGAAVLPVDSEHNAVFQVLPRDYTGRLNEHGIASIILTASGGPFLTADLNTFDSITPDQAVKHPNWRMGRKISVDSATMMNKGLELIEAHWLFNCPPDKLEVVIHPQSVIHSMVRYRDGSVLAQLGNPDMRTPIAYCLGLPERIDSGVGDLDFDALSALTFQKPDFDRFPCLKLAYEAMNAGGAAPCVLNAANEAAVAAFLDGQIKFTDIAKTVAHCLSQDFSDGIGDIGGLLAQDARTRAQARAFIGTLR

SEQ ID NO: 120 - protein, 397 aa, Haemophilus influenzae; identical to SEQ ID NO: 99

SEQ ID NO: 121 - protein, 394 aa, Synechocystis sp. PCC 6803; identical to SEQ ID NO: 103

SEQ ID NO: 122 - protein, 405 aa, Pasteurella multocida
MSISYFMKKIVILGSTGSIGTSTLSVITHNPDKYQVFALVGGRNVELMFQQCLTFQPSFAALDDDVAAKMLAEKLKAHQSQTTVLAGQQAICELAAHPEADMVMAAIVGAAGLLPTLSAVKAGKRVLLANKEALVTCGQLFIDAVRESQAQLLPVDSEHNAIFQSLPPEAQRQIGFCPLSELGISKIVLTGSGGPFRYTPLEQFEQITPAQAVAHPNWSMGKKISVDSATMMNKGLEYIEARWLFNASAEEMEVIIHPQSIIHSMVRYIDGSVIAQMGNPDMRTPIAETMAYPSRTVAGVEPLDFYQLNGLTFIEPDYQRYPCLKLAIDAFSAGQYATTAMNAANEIAVASFLDNKIKFTDIARLNQLVVSKLQPQKIHCIEDVLEVDKKARELSQSIILSFSHP

SEQ ID NO: 123 - protein, 402 aa, Synechococcus leopoliensis; identical to SEQ ID NO: 102

SEQ ID NO: 124 - protein, 386 aa, Streptomyces griseolosporeus
MVILGSTGSIGTQAIDVVLRNPGRFKVVALSAAGGAVELLAEQAVALGVHTVAVADPAAEEAAARGPGGQGAGRPLPRVLAGPDAATELAAAECHSVLNGITGSIGLAPTLAALRAGRVLVLANKESLIVGGPLVKAVAQPGQIVPVDSEHAALFQALAGGARAEVRKLVVTASGGPFRNRTREQLAAVTPADALAHPTWAMGPVVTINSATLVNKGLEVIEAHLLYDVPFDRIEVVVHPQSVVHSMVEFVDGSTMAQASPPDMRMPIALGLGWPDRVPDAAPGCDWTKAATWEFFPLDNEAFPAVELAREVGTLGGTAPAVFNAANEECVDAFLKGALPFTGIVDTVAKVVAEHGTPQSGTSLTVEDVLHAESWARARARELAAG

SEQ ID NO: 125 - protein, 388 aa, Bacillus subtilis; identical to SEQ ID NO: 98

SEQ ID NO: 126 - protein, 406 aa, Mycobacterium leprae
MNNPIEGHAGGRLRVLVLGSTGSIGTQALEVIAANPDRFEVVGLAAGGAQLDTLLRQRAATGVTNIAIADDRAAQLAGDIPYHGTDAVTRLVEETEADVVLNALVGALGLRPTLAALHTGARLALANKESLVAGGSLVLAAAQPGQIVPVDSEHSALAQCLRGGTPDEVAKLVLTASGGPFRGWNAGDLERVTPEQAGVHPTWSMGTMNTLNSASLVNKGLELIEANLLFGIPYDRIEVVVHPQSIVHSMVTFIDGSTIAQASPPDMKLPISLALGWPQRVGGAARACAFTTASTWEFEPLDIDVFPAVELARHAGQIGGCMTAIYDAANEEAAEAFLQGRIGFPAIVATIADVLQRADQWAPQWGEGPATVDDVLDAQRWARERALCAVATASSGKVSDMVLERS

SEQ ID NO: 127 - protein, 436 aa, Mycobacterium tuberculosis; identical to SEQ ID NO: 104

SEQ ID NO: 128 - protein, 477 aa, Arabidopsis thaliana
MMTLNSLSPAESKAISFLDTSRFNPIPKLSGGFSLRRRNQGRGFGKGVKCSVKVQQQQQPPPAWPGRAVPEAPRQSWDGPKPISIVGSTGSIGTQTLDIVAENPDKFRVVALAAGSNVTLLADQVRRFKPALVAVRNESLINELKEALADLDYKLEIIPGEQGVIEVARHPEAVTVVTGIVGCAGLKPTVAAIEAGKDIALANKETLIAGGPFVLPLANKHNVKILPADSEHSAIFQCIQGLPEGALRKIILTASGGAFRDWPVEKLKEVKVADALKHPNWNMGKKITVDSATLFNKGLEVIEAHYLFGAEYDDIEIVIHPQSIIHSMIETQDSSVLAQLGWPDMRLPILYTMSWPDRVPCSEVTWPRLDLCKLGSLTFKKPDNVKYPSMDLAYAAGRAGGTMTGVLSAANEKAVEMFIDEKISYLDIFKVVELTCDKHRNELVTSPSLEEIVHYDLWAREYAANVQLSSGARPVHA

SEQ ID NO: 129 - DNA, 19 bp, Artificial Sequence
  Primer
cgtaacgctcggtctcgtc

SEQ ID NO: 130 - protein, 356 aa, Campylobacter jejuni
MILFGSTGSIGVNALKLAALKNIPISALACGDNIALLNEQIARFKPKFVSIKDSKNKHLVKHDRVFIGQEGLEQILTECQDKLLLNAIVGFAGLKSTLKAKELGKNIALANKESLVVAGSFLKGAKFLPVDSEHAALKFLLEGKKNIAKLYITASGGAFYRYKIKDLNQVSVKDALKHPNWNMGAKITIDSATMANKLFEIIEAYHLYDFKEIDALIEPRSLVHAMCEFKNGASTAYFSKADMKLAISDAIFEKQDTPILEAVDFSKMPALKFHPISTKKYPIFKLKNTFLKEPNLGVIINAANEVGVYNFLENKSGFLDIAKCIFKALDHFGVPKISSIEEVFEYDFKTREYLRS

SEQ ID NO: 131 - protein, 486 aa, Plasmodium falciparum
MKKYIYIYFFFITITINDLVINNTSKCVSIERRKNNAYINYGIGYNGPDNKITKSRRCKRIKLCKKDLIDIGAIKKPINVAIFGSTGSIGTNALNIIRECNKIENVFNVKALYVNKSVNELYEQAREFLPEYLCIHDKSVYEELKELVKNIKDYKPIILCGDEGMKEICSSNSIDKIVIGIDSFQGLYSTMYAIMNNKIVALANKESIVSAGFFLKKLLNIHKNAKIIPVDSEHSAIFQCLDNNKVLKTKCLQDNFSKINNINKIFLCSSGGPFQNLTMDELKNVTSENALKHPKWKMGKKITIDSATMMNKGLEVIETHFLFDVDYNDIEVIVHKECIIHSCVEFIDKSVISQMYYPDMQIPILYSLTWPDRIKTNLKPLDLAQVSTLTFHKPSLEHFPCIKLAYQAGIKGNFYPTVLNASNEIANNLFLNNKIKYFDISSIISQVLESFNSQKVSENSEDLMKQILQIHSWAKDKATDIYNKHN

SEQ ID NO: 132 - DNA, 24 bp, Artificial Sequence
  Primer
ccsgtsgaywssgarcayaacgcs

SEQ ID NO: 133 - DNA, 21 bp, Artificial Sequence
  Primer
atgatgaacaagggsctsgar

SEQ ID NO: 134 - DNA, 20 bp, Artificial Sequence
  Primer
catccvaactggwmvatggg

SEQ ID NO: 135 - DNA, 20 bp, Artificial Sequence
  Primer
atyggyrwwckcatatcmgg

SEQ ID NO: 136 - DNA, 27 bp, Artificial Sequence
  Primer
cgaatggacgacggattggcgatggac

SEQ ID NO: 137 - DNA, 29 bp, Artificial Sequence
  Primer
tcagttcgagccccttgttcatcatcgtc

SEQ ID NO: 138 - DNA, 28 bp, Artificial Sequence
  Primer
cgaactgatcgaagccttccacctgttc

SEQ ID NO: 139 - DNA, 27 bp, Artificial Sequence
  Primer
ggtccatcgccaatccgtcgtccattc

SEQ ID NO: 140 - DNA, 30 bp, Artificial Sequence
  Primer
attatctagaatccgccccgcctccacctc

SEQ ID NO: 141 - DNA, 30 bp, Artificial Sequence
  Primer
gatggatcctgggtagggtcgctgctgtcc

SEQ ID NO: 142 - DNA, 34 bp, Artificial Sequence
  Primer
ttatctagaaccgtctacgccgacctcgttcaac

SEQ ID NO: 143 - DNA, 30 bp, Artificial Sequence
  Primer
ttaggatcccctccgctggtccgattgaac

SEQ ID NO: 144 - DNA, 20 bp, Artificial Sequence
  Primer
aggcgattaagttgggtaac

SEQ ID NO: 145 - DNA, 18 bp, Artificial Sequence
  Primer
gaccatgattacgccaag

SEQ ID NO: 146 - DNA, 30 bp, Artificial Sequence
  Primer
gataatcgatgtgtgactgacctgtccaac

SEQ ID NO: 147 - DNA, 30 bp, Artificial Sequence
  Primer
cttaggtaccatgttggagattcaaggtgg

SEQ ID NO: 148 - DNA, 30 bp, Artificial Sequence
  Primer
ctagatcgatacttgcggtcggactgatag

SEQ ID NO: 149 - DNA, 30 bp, Artificial Sequence
  Primer
gtcgctcgagatcagataatcgtcgctcaa

SEQ ID NO: 150 - DNA, 30 bp, Artificial Sequence
  Primer
atatggtaccgacatggacgaggaagacgc

SEQ ID NO: 151 - DNA, 41 bp, Artificial Sequence
  Primer
tagagaattcgaaggaagagcatgggattggacgaggtttc

SEQ ID NO: 152 - DNA, 41 bp, Artificial Sequence
  Primer
tactacttgtatgtaggtaccacttgcggtcggactgatag

SEQ ID NO: 153 - DNA, 41 bp, Artificial Sequence
  Primer
gcgtgatatcgaaggaagagcatgagcgcaaccgtccaccg

SEQ ID NO: 154 - DNA, 41 bp, Artificial Sequence
  Primer
actgctagggtccgaggtaccgacatggacgaggaagacgc

SEQ ID NO: 155 - DNA, 42 bp, Artificial Sequence
  Primer
gatgatatcgaaggaagagcatggtgaagcgcgtcacggtgt

SEQ ID NO: 156 - DNA, 40 bp, Artificial Sequence
  Primer
caagagtcagaaggtacccgccagaatggtgagcaggatg

SEQ ID NO: 157 - DNA, 40 bp, Artificial Sequence
  Primer
caagagtcagaatctagacgccagaatggtgagcaggatg

SEQ ID NO: 158 - DNA, 39 bp, Artificial Sequence
  Primer
ctagatatcggaaggaagagcatgtcacaccccgcgtta

SEQ ID NO: 159 - DNA, 36 bp, Artificial Sequence
  Primer
tcaggtaccgtgtcgccacccacaacgcccataatg

SEQ ID NO: 160 - DNA, 34 bp, Artificial Sequence
  Primer
tagggtaccaccgtctacgccgacctcgttcaac

SEQ ID NO: 161 - DNA, 34 bp, Artificial Sequence
  Primer
tgtatgcatgtcgccacccacaacgcccataatg

SEQ ID NO: 162 - DNA, 43 bp, Artificial Sequence
  Primer
gacgaagcttgaaggaagagcatgcctcccctcaccctctatc

SEQ ID NO: 163 - DNA, 43 bp, Artificial Sequence
  Primer
gtcactgaatgaatggtaccgcagccgagaaccgccagaagcc

SEQ ID NO: 164 - DNA, 34 bp, Artificial Sequence
  Primer
tagggtaccaccgtctacgccgacctcgttgaac

SEQ ID NO: 165 - DNA, 34 bp, Artificial Sequence
  Primer
aggcaatgcatgcagccgagaaccgccagaagcc

SEQ ID NO: 166 - DNA, 32 bp, Artificial Sequence
  Primer
aagcatgcgaaaaagttgacacctgtggagtc

SEQ ID NO: 167 - DNA, 29 bp, Artificial Sequence
  Primer
actctagaagcacctgcgaatggacgaag

SEQ ID NO: 168 - DNA, 31 bp, Artificial Sequence
  Primer
ataaaggccttacatggcgatagctagactg

SEQ ID NO: 169 - DNA, 31 bp, Artificial Sequence
  Primer
aaggctcgagaaggatcttaccgctgttgag

SEQ ID NO: 170 - DNA, 32 bp, Artificial Sequence
  Primer
atagtactgaaaaagttgacacctgtggagtc

SEQ ID NO: 171 - DNA, 29 bp, Artificial Sequence
  Primer
atagtactagcacctgcgaatggacgaag

SEQ ID NO: 172 - DNA, 40 bp, Artificial Sequence
  Primer
cgtggcatgcgtgtaagaaaaagttgacacctgtggagtc

SEQ ID NO: 173 - DNA, 40 bp, Artificial Sequence
  Primer
ctaagagctcagttcgggctcggtctcgcctttcaggaag

SEQ ID NO: 174 - DNA, 40 bp, Artificial Sequence
  Primer
gagagcgagagccagatcaagaagsggctgaaggacatcc

SEQ ID NO: 175 - DNA, 40 bp, Artificial Sequence
  Primer
ggatgtccttcagccscttcttgatctggctctcgctctc

SEQ ID NO: 176 - DNA, 30 bp, Artificial Sequence
  Primer
agtcagtactaactggtgaagacgctgaag

SEQ ID NO: 177 - DNA, 30 bp, Artificial Sequence
  Primer
gatcagtactgtgaacgaatacgatacgca

SEQ ID NO: 178 - DNA, 40 bp, Artificial Sequence
  Primer
gtcaaatgagctccaaactggtgaagacgctgaaggacat

SEQ ID NO: 179 - DNA, 40 bp, Artificial Sequence
  Primer
cagtcgggcatgcgtccatttcagttgacatacttctgtg

SEQ ID NO: 180 - DNA, 41 bp, Artificial Sequence
  Primer
ctcttgctcggcggcgtgcggctctatcacgagggggtgga

SEQ ID NO: 181 - DNA, 41 bp, Artificial Sequence
  Primer
tccaccccctcgtgatagagccgcacgccgccgagcaagag

SEQ ID NO: 182 - DNA, 20 bp, Artificial Sequence
  Primer
gagcagcacactctgggagc

SEQ ID NO: 183 - DNA, 21 bp, Artificial Sequence
  Primer
ccacacaggtaggacacccac

SEQ ID NO: 184 - DNA, 40 bp, Artificial Sequence
  Primer
tcagagctcgtgtgatcgaatggggctttgttccttgatg

SEQ ID NO: 185 - DNA, 39 bp, Artificial Sequence
  Primer
gaagcatgcaggtgatcgacgtgccactcgtccgaatag

SEQ ID NO: 186 - DNA, 20 bp, Artificial Sequence
  Primer
ctcacaacctccaaccgatg

SEQ ID NO: 187 - DNA, 20 bp, Artificial Sequence
  Exemplary motif
aggtcgtgtactgtcagtca

SEQ ID NO: 188 - DNA, 20 bp, Artificial Sequence
  Exemplary motif
acgtggtgaactgccagtga

SEQ ID NO: 189 - DNA, 40 bp, Artificial Sequence
  Primer
actatcgatgaaggaagagcatggctgacctacccaagac

SEQ ID NO: 190 - DNA, 30 bp, Artificial Sequence; identical to SEQ ID NO: 78
  Primer